(19) United States
(12) Patent Application Publication
(10) Pub. No.: US 2008/0004253 A1
(43) Pub. Date: Jan. 3, 2008

(54) Thiazolopyrimidine modulators of TRPV1

(76) Inventors: Bryan James Branstetter, Carlsbad, CA (US); James Guy Breitenbucher, Escondido, CA (US); Alec D. Lebsack, San Diego, CA (US); Wei Xiao, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/824,202

(22) Filed: Jun. 28, 2007

Related U.S. Application Data
(60) Provisional application No. 60/818,153, filed on Jun. 30, 2006.

(51) Int. Cl. A61K 31/55; A61K 31/541; A61K 31/5377; A61K 31/519; C07D 498/02

(52) U.S. Cl. 514/210.21; 514/260.1; 514/227.8; 514/234.5; 514/252.16; 514/217.06; 544/60; 544/120; 544/255; 540/600

(57) ABSTRACT

Certain TRPV1-modulating thiazolopyrimidine compounds are described. The compounds may be used in pharmaceutical compositions and methods for treating disease states, disorders, and conditions mediated by TRPV1 activity, such as pain, arthritis, itch, cough, asthma, or inflammatory bowel disease.

### CROSS-REFERENCE TO RELATED APPLICATION

This application claims priority to U.S. Provisional Application No. 60/818,153, filed Jun. 30, 2006, the disclosure of which is incorporated by reference.

### FIELD OF THE INVENTION

The present invention relates to certain thiazolopyrimidine compounds, pharmaceutical compositions containing them, and methods of using them for the treatment of disease states, disorders, and conditions mediated by TRPV1 activity.

### BACKGROUND OF THE INVENTION

Transient receptor potential (TRP) channel proteins constitute a large and diverse family of proteins that are expressed in many tissues and cell types. One TRP channel protein of particular interest is the vanilloid receptor 1 (TRPV1 or VR1), a non-selective Ca⁺² channel that is the molecular target of vanilloid compounds (e.g., capsaicin and resiniferatoxin). Such vanilloid compounds are known to selectively depolarize nociceptors, specialized primary afferent neurons involved in the signaling pathway that leads to the sensation of pain. TRPV1 is activated by a diverse range of stimuli, including vanilloids, membrane depolarization, heat, stretch, low pH, inflammatory mediators (e.g., lipoxygenase metabolites), and endocannabinoid compounds. Because heightened activity of nociceptors contributes to unwanted pain, inflammatory conditions, thermoregulation, and control of smooth muscle tone and reflexes in mammals, modulation of signaling in this pathway is important in treatment and prophylaxis of various clinical syndromes (Caterina, M. J., Pain 2003, 105(1-2), 5-9; Caterina, M. J. et. al., Annu. Rev. Neurosci. 2001, 24, 487-517; Tominaga, M. et. al., J. Neurobiol. 2004, 61, 3-12; Voets, T. et. al., Nature 2004, 430, 748-754).

Because of TRPV1's connection with the sensory nervous system, TRPV1 agonists and antagonists may be therapeutically useful in the treatment or prophylaxis of disease states, disorders, and conditions mediated by TRPV1 activity, such as: i) pain (e.g., acute, chronic, inflammatory, or neuropathic pain); ii) itch (Kim et al., Neurosci. Lett. 2004, 361, 159) and various inflammatory disorders (Stucky, C. L. et. al., Neuroscience 1998, 84, 1257; Moore, B. A. et. al., Am. J. Physiol. Gastrointest. Liver Physiol. 2002, 282, G1045; Kwak, J. Y. et. al., Neuroscience 1998, 86, 619; Morris, V. H. et. al., Pain 1997, 71, 179; Greiff, L. et. al., Thorax 1995, 50, 225); iii) inner ear disorders (Balaban, C. D. et al., Hear. Res. 2003, 175, 165-70; Zheng, J. et al., J. Neurophys. 2003, 90, 444-55); iv) fever and other disorders or symptoms affected by thermoregulation (Jancso-Gabor et al., J. Physiol. 1970, 206, 495; Swanson et al., J. Med. Chem. 48, 1857; Iida et al., Neurosci. Lett. 2005, 378, 28); v) tracheobronchial and diaphragmatic dysfunction; and vi) gastrointestinal and urinary tract disorders (Lazzeri, M. et al., Eur. Urology 200, 792-798; Apostolidis, A. et. al., Urology 2005, 65, 400-405). Additionally, TRPV1 modulators may be therapeutically useful in the treatment or prophylaxis of anxiety (Marsch, R. et al., J. Neurosci. 2007, 27(4), 832-839); eye-related disorders (such as glaucoma, vision loss, and increased intraocular pressure) (Calkins, D. J. et al., Abstract from ARVO 2006 Annual Meeting, Program #1557, Poster #B93); baldness (e.g., by stimulating hair growth) (Bodo, E. et al., Am. J. Pathol. 2005, 166(4), 985-998); diabetes (including insulin-resistant diabetes or diabetic conditions mediated by insulin sensitivity or secretion) (Razavi, R. et al., Cell 2006, 127(6), 1097-1099; Akiba, Y. et al., Biochem. Biophy. Res. Commun. 2004, 321(1), 219-225).

Acidosis is a well-established feature of cerebral ischaemia. Tissue pH may fall to 6 or lower, sufficient to activate TRPV1 channels expressed in the CNS. TRPV1 antagonists therefore may be useful in the treatment of disorders associated with reduced blood flow to the CNS or CNS hypoxia, such as head trauma, spinal injury, thromboembolic or hemorrhagic stroke, transient ischaemic attacks, cerebral vasospasm, hypoglycaemia, cardiac arrest, status epilepticus, perinatal asphyxia, Alzheimer's disease, and Huntington's Disease.

Certain thiazole carboxamides have been described as vanilloid receptor modulators (Xi et al., Bioorg. Med. Chem. Lett. 2005, 15, 5211-5217; U.S. Pat. Appl. Publ. 2004/157845). Certain thiazolopyrimidines have been described as CCR2b receptor antagonists (U.S. Pat. Appl. Publ. 2005/117890). Synthetic methods for the preparation of various thiazolopyrimidines have been described by Freeman et al. (J. Org. Chem. 1991, 56(15), 4645-4648) and by Liu et al. (J. Org. Chem. 2005, 70, 10194-10197 and references cited therein).

There remains a desire for potent TRPV1 modulators with suitable pharmaceutical properties.

### SUMMARY OF THE INVENTION

Certain thiazolopyrimidine derivatives have now been found to have TRPV1-modulating activity. In particular, the invention is directed to the general and preferred embodiments defined, respectively, by the independent and dependent claims appended hereto, which are incorporated by reference herein.

Thus, in one general aspect, the invention relates to compounds of Formula (I):

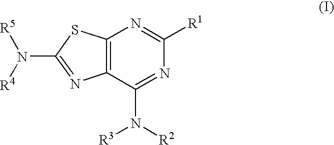

wherein:
R¹ is —H; —NR^{a}R^{b}; a —C₁₋₆alkyl, —OC₁₋₆alkyl, —S—C₁₋₆alkyl, or —SO₂—C₁₋₆alkyl group unsubstituted or substituted with an —OH, —OC₁₋₄alkyl, —NR^{e}R^{f}, or halo substituent; or a monocyclic cycloalkyl or phenyl group unsubstituted or substituted with a —C₁₋₆alkyl, —OH, —OC₁₋₄alkyl, —NR^{e}R^{f}, or halo substituent;
   where R^{a} and R^{b} are each independently —H; —C₁₋₆alkyl; a —C₂₋₄alkyl group substituted with one or two —OH, —OC₁₋₄alkyl, —NR^{c}R^{d}, or halo substituents; or a saturated monocyclic cycloalkyl, —C₁alkyl-(saturated monocyclic cycloalkyl), saturated monocyclic heterocycloalkyl, —C₁alkyl-(saturated monocyclic heterocycloalkyl), phenyl, or benzyl group unsubstituted or substituted with one, two, or three moieties independently selected from the group consisting of —C₁₋₆alkyl, —OH, —OC₁₋₄alkyl, —NR^{p}R^{q}, and halo substituents; or
      R^{a} and R^{b} taken together with the nitrogen of attachment in —NR^{a}R^{b} form a saturated monocyclic heterocycloalkyl group unsubstituted or substituted with one, two, or three moieties independently selected from the group consisting of —C₁₋₆alkyl, —C₁₋₂alkyl-OH, —C₁₋₂alkyl-OC₁₋₂alkyl, —OH, —OC₁₋₄alkyl, —NR^{p}R^{q}, halo, —CO₂H, and benzyl substituents;
      where R^{c} and R^{d} are each independently —H or —C₁₋₆alkyl; or R^{c} and R^{d} taken together with the nitrogen of attachment in —NR^{c}R^{d} form a saturated monocyclic heterocycloalkyl unsubstituted or substituted with methyl;
      where R^{p} and R^{q} are each independently —H or —C₁₋₆alkyl; or R^{p} and R^{q} taken together with the nitrogen of attachment in —NR^{p}R^{q} form a saturated monocyclic heterocycloalkyl unsubstituted or substituted with methyl;
      where R^{e} and R^{f} are each independently —H or —C₁₋₆alkyl; or R^{e} and R^{f} taken together with their nitrogen of attachment in —NR^{e}R^{f} form a saturated monocyclic heterocycloalkyl unsubstituted or substituted with methyl;
R² is —H or —C₁₋₆alkyl;
R³ is a monocyclic cycloalkyl, phenyl, benzyl, phenethyl, indanyl, quinolinyl, monocyclic five-membered heteroaryl, monocyclic six-membered heteroaryl, or —C₁alkyl-(monocyclic heteroaryl) group unsubstituted or substituted with one, two, or three R^{g} substituents;
   where each R^{g} substituent is —C₁₋₆alkyl, —OH, —OC₁₋₆alkyl, —O-(saturated monocyclic heterocycloalkyl), phenoxy, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH; or
      two adjacent R^{g} substituents taken together form —OC₁₋₂alkylO—, —C₂₋₆alkylO—, or —C₂₋₆alkylN(R^{h})—;
      where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl unsubstituted or substituted with methyl;
      where each R^{j} is independently —H, —C₁₋₆alkyl, or —CF₃; or both R^{j} substituents taken together with the carbon to which they are attached form a monocyclic cycloalkyl ring;
R⁴ is —H or —C₁₋₆alkyl; and
R⁵ is a phenyl, monocyclic five-membered heteroaryl, or monocyclic six-membered heteroaryl group unsubstituted or substituted with one, two, or three R^{k} substituents;
   where each R^{k} substituent is independently —C₁₋₆alkyl unsubstituted or substituted with one or two —OH groups, —C₁₋₂alkyl-N(R^{l})R^{m}, —OH, —OC₁₋₆alkyl, phenyl, phenoxy, —CN, —NO₂, —N(R^{l})R^{m}, —C(O)N(R^{l})R^{m}, —N(R^{l})C(O)R^{m}, —N(R^{l})SO₂C₁₋₆alkyl, —N(R^{l})SO₂CF₃, —C(O)C₁₋₆alkyl, —S(O)₀₋₂—C₁₋₆alkyl, —SO₂CF₃, —SO₂N(R^{l})R^{m}, —SCF₃, halo, —CF₃, —OCF₃, —CO₂H, or —CO₂C₁₋₆alkyl; or
      two adjacent R^{k} substituents taken together form —OC₁₋₂alkylO— or ═N—S—N═;
      where R^{l} and R^{m} are each independently —H or —C₁₋₆alkyl; or R^{l} and R^{m} taken together with their nitrogen of attachment in —NR^{l}R^{m} form a saturated monocyclic heterocycloalkyl unsubstituted or substituted with methyl;
         and pharmaceutically acceptable salts, pharmaceutically acceptable prodrugs, and pharmaceutically active metabolites of the compounds of Formula (I) (collectively, “active agents”).

In a further general aspect, the invention relates to pharmaceutical compositions each comprising: (a) an effective amount of at least one active agent as defined above; and (b) a pharmaceutically acceptable excipient.

In another general aspect, the invention is directed to a method of treating a subject suffering from or diagnosed with a disease, disorder, or medical condition (collectively, “indications”) mediated by TRPV1 activity (e.g., pain (acute, chronic, inflammatory, or neuropathic pain); itch or various inflammatory disorders; inner ear disorders; fever or other conditions or disorders of thermoregulation; tracheobronchial or diaphragmatic dysfunction; gastrointestinal or urinary tract disorders; or disorders associated with reduced blood flow to the CNS or CNS hypoxia), comprising administering to the subject in need of such treatment an effective amount of at least one active agent as defined above.

Preferred embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following detailed description, including the following glossary of terms and the concluding examples. For the sake of brevity, the disclosures of the publications, including patents, cited in this specification are herein incorporated by reference.

The terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a/symbol), ethyl (Et), n-propyl, isopropyl, butyl (nBu), isobutyl, sec-butyl, tert-butyl (tBu), pentyl, isopentyl, tert-pentyl, hexyl, isohexyl, and so on.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-enyl, but-2-enyl, but-3-enyl, 2-methylprop-2-enyl, hex-2-enyl, and so on.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities (depicted without their bonds of attachment):

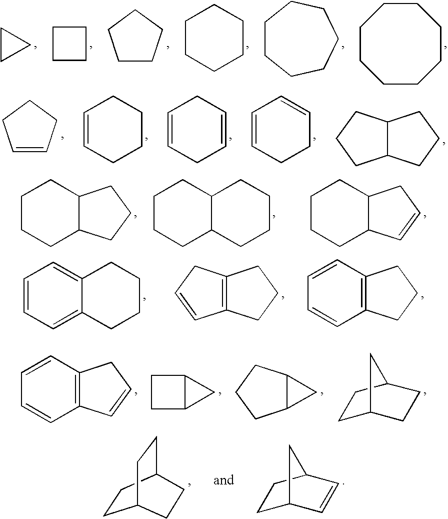

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples (depicted without their bonds of attachment) include:

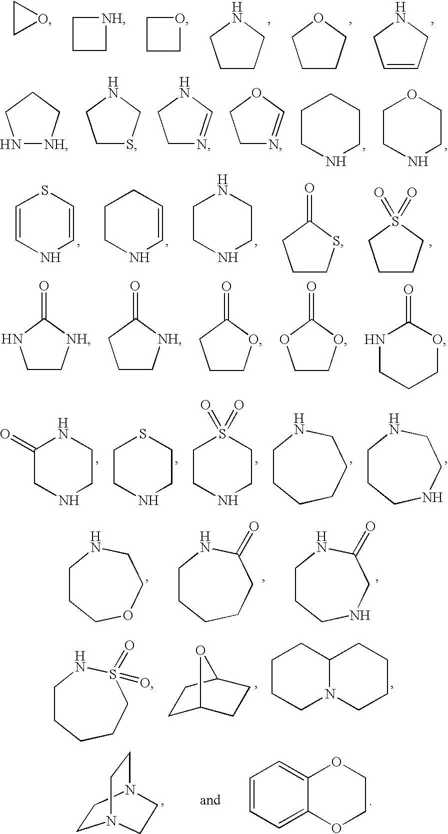

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities (depicted without their bonds of attachment):

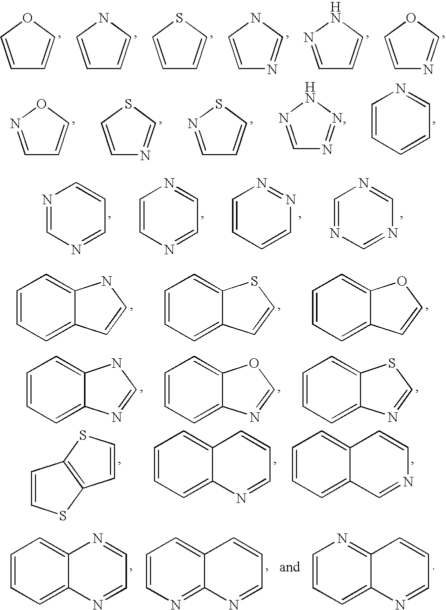

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or form's. In particular, compounds of any formula given herein may have asymmetric centers and therefore exist in different enantiomeric forms. All optical isomers and stereoisomers of the compounds of any general structural formula, and mixtures thereof, are considered within the scope of the formula. Thus, any general formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any general formula given herein is intended to embrace hydrates, solvates, and polymorphs of such compounds, and mixtures thereof.

Any general formula given herein is also intended to represent unlabeled forms as well as isotopically labeled forms of the compounds. Isotopically labeled compounds have structures of the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example ²H or ³H), detection or imaging techniques (such as positron emission tomography (PET) or single-photon emission computed tomography (SPECT)) including drug or substrate tissue distribution assays, or in radioactive treatment of patients. For example, an ¹⁸F or ¹¹C labeled compound may be preferred for PET or SPECT studies. Further, substitution with heavier isotopes such as deuterium (i.e., ²H) may afford certain therapeutic advantages resulting from greater metabolic stability, for example, increased in vivo half-life or reduced dosage requirements. Isotopically labeled compounds can generally be prepared by carrying out the procedures disclosed in the schemes or in the examples and preparations described below by substituting a readily available isotopically labeled reagent for a non-isotopically labeled reagent.

When referring to a formula given herein, the selection of a particular moiety from a list of possible species for a specified variable is not intended to define the moiety for the variable appearing elsewhere. In other words, where a variable appears more than once in a formula, the choice of the species from a specified list is independent of the choice of the species for the same variable elsewhere in the formula unless otherwise indicated.

In certain preferred embodiments of compounds of Formula (I), R¹ is —H, methyl, —CH₂— (monocyclic cycloalkyl), or —NR^{a}R^{b}; where R^{a} and R^{b} are each independently —H; —C₁₋₆alkyl; a —C₂₋₃alkyl group substituted with an —OH, —OC₁₋₄alkyl, or —NR^{c}R^{d} substituent (where R^{c} and R^{d} are each independently —H or —C₁₋₆alkyl); or a saturated monocyclic cycloalkyl or —C₁alkyl-(saturated monocyclic cycloalkyl) group unsubstituted or substituted with a methyl, —OH, or —OC₁₋₄alkyl substituent; or R^{a} and R^{b} taken together with the nitrogen of attachment in —NR^{a}R^{b} form a saturated monocyclic heterocycloalkyl group unsubstituted or substituted with a methyl, —OH, or —OC₁₋₄alkyl substituent.

In certain preferred embodiments, R^{g} is —C₁₋₄alkyl, methoxy, —CF₃, halo, —C(CH₃)₂CONH₂, 1-hydroxy-cyclopropyl, —SO₂CH₃, —SO₂CF₃, or —SO₂N(R^{h})Rⁱ; where R^{h} and Rⁱ are each independently —H or —C₁₋₆alkyl.

In various preferred embodiments, each R^{k} substituent is independently —H, chloro, methyl, —CH₂OH, or —CH₂N(R^{l})R^{m}, where R^{l} and R^{m} are each independently —H or —C₁₋₆alkyl.

In other preferred embodiments of compounds of Formula (I), R¹ is —H or a methyl, ethyl, propyl, or isopropyl group unsubstituted or substituted with a —OH, —OC₁₋₄alkyl, —NR^{e}R^{f}, or halo substituent; or a cyclopropyl, cyclobutyl, cyclopentyl, or cyclohexyl group unsubstituted or substituted with a —C₁₋₆alkyl, —OH, —OC₁₋₄alkyl, —NR^{e}R^{f}, or halo substituent. In further preferred embodiments, R¹ is —NR^{a}R^{b} or a methoxy, ethoxy, propyloxy, isopropyloxy, methanesulfanyl, ethanesulfanyl, propylsulfanyl, isopropylsulfanyl, methanesulfonyl, ethanesulfonyl, propylsulfonyl, or isopropylsulfonyl group unsubstituted or substituted with a —OH, —OC₁₋₄alkyl, —NR^{e}R^{f}, or halo substituent.

Preferably, R^{a} and R^{b} are each independently —H; methyl, ethyl, propyl, isopropyl, butyl, isobutyl, sec-butyl, tert-butyl, pentyl, isopentyl, or hexyl; an ethyl or propyl group substituted with an —OC₁₋₄alkyl or —NR^{c}R^{d} substituent; or a cyclopropyl, cyclobutyl, cyclopentyl, cyclohexyl, cycloheptyl, cyclopropylmethyl, cyclopentylmethyl, aziridinyl, pyrrolidinyl, tetrahydrofuranyl, piperidinyl, tetrahydropyranyl, piperazinyl, morpholinyl, thiomorpholinyl, 1,1-dioxo-1λ⁶-thiomorpholin-4-yl, or phenyl group unsubstituted or substituted with a —C₁₋₆alkyl, —OC₁₋₄alkyl, or halo substituent. In further preferred embodiments, R^{a} and R^{b} are each independently —H, methyl, methoxyethyl, cyclopropylmethyl, or phenyl. In alternative embodiments, R^{a} and R^{b} taken together with the nitrogen of attachment form an azetidinyl, pyrrolidinyl, piperidinyl, 2-oxo-piperidin-1-yl, piperazinyl, oxo-piperazinyl, morpholinyl, thiomorpholinyl, 1,1-dioxo-1λ⁶-thiomorpholin-4-yl, 1,1-dioxo-1λ⁶-[1,2]thiazinan-2-yl, or azepanyl group unsubstituted or substituted with a —C₁₋₆alkyl, —OH, or —CO₂H substituent.

In preferred embodiments, R^{c} and R^{d} are each independently —H, methyl, or ethyl.

Preferably, R^{p} and R^{q} are each independently —H, methyl, or ethyl.

In preferred embodiments, R^{e} and R^{f} are each independently —H, methyl, or ethyl.

Preferably, R¹ is —H, methyl, isopropyl, methanesulfanyl, methanesulfonyl, methoxy, phenyl, phenoxy, dimethylamino, azetidinyl, pyrrolidinyl, piperidinyl, azepanyl, morpholinyl, 4-isopropyl-piperazin-1-yl, 2-methoxyethylamino, (2-methoxyethylamino)methylamino, cyclopropylmethylamino, or phenylamino. In further preferred embodiments, R¹ is —H or methyl.

In preferred embodiments, R² is —H or methyl.

Preferably, R³ is a cyclopentyl, cyclohexyl, phenyl, indanyl, furanyl, thiophenyl, pyrrolyl, oxazolyl, thiazolyl, pyridyl, pyrimidinyl, or pyrazinyl group unsubstituted or substituted with one or two R^{g} substituents. In further preferred embodiments, R³ is a phenyl or pyridyl group substituted with one or two R^{g} substituents.

In certain preferred embodiments, each R^{g} substituent is independently methyl, isopropyl, tert-butyl, —OH, —OCH₃, phenoxy, —CN, —NO₂, —NH₂, —C(O)CH₃, —SO₂CF₃, —SO₂NH₂, —SCF₃, chloro, bromo, —CF₃, —OCF₃, —CO₂CH₃, —C(CH₃)₂—CN, or —C(CH₃)₂—OH; or two adjacent R^{g} substituents taken together form —OC₁₋₂alkylO—. In further preferred embodiments, each R^{g} substituent is independently methyl, tert-butyl, —OH, —OCH₃, —CN, —SCF₃, chloro, —CF₃, —OCF₃, —CO₂CH₃, or —C(CH₃)₂—CN.

Preferably, R^{h} and Rⁱ are each independently —H, methyl, or ethyl.

In some preferred embodiments, R^{j} is —H, methyl, or ethyl.

Preferably, R⁴ is —H, methyl, or ethyl.

In various preferred embodiments, R⁵ is a phenyl, furanyl, thiophenyl, isoxazolyl, or pyridyl group substituted with one or two R^{k} substituents. In further preferred embodiments, R⁵ is a phenyl or pyridyl group ortho-substituted with one or two R^{k} substituents. For example, R⁵ is preferably a phenyl or pyridyl group substituted as depicted below:

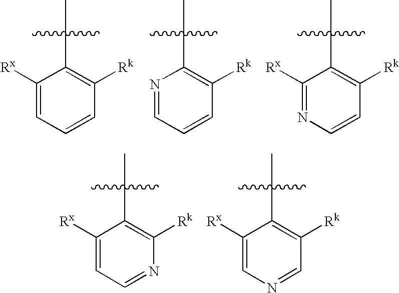

where R^{x} is H or an R^{k} substituent.

Preferably, each R^{k} substituent is independently methyl, ethyl, propyl, isopropyl, —OH, —OCH₃, phenyl, phenoxy, —CN, —NO₂, —NH₂, methylamino, dimethylamino, —NHSO₂CH₃, —C(O)CH₃, —SO₂NH₂, —SO₂CF₃, —SCF₃, chloro, bromo, —CF₃, —OCF₃, —CO₂H, or —CO₂CH₃. In further preferred embodiments, each R^{k} substituent is independently methyl, —CF₃, chloro, phenyl, —SO₂CH₃, or —CO₂CH₃.

In preferred embodiments, R^{l} and R^{m} are each independently —H, methyl, or ethyl.

In a preferred subgeneric embodiment, the compounds are of the following Formula (I′):

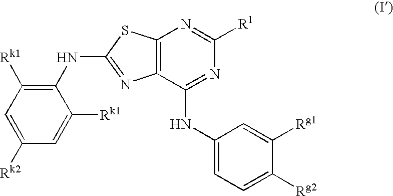

wherein:
R¹ is —H, methyl, —CH₂— (monocyclic cycloalkyl), or —NR^{a}R^{b};
   where R^{a} and R^{b} are each independently —H; —C₁₋₆alkyl; a —C₂₋₃alkyl group substituted with an —OH, —OC₁₋₄alkyl, or —NR^{c}R^{d} substituent; or a saturated monocyclic cycloalkyl or —C₁alkyl-(saturated monocyclic cycloalkyl) group unsubstituted or substituted with a methyl, —OH, or —OC₁₋₄alkyl substituent; or
      R^{a} and R^{b} taken together with the nitrogen of attachment in —NR^{a}R^{b} form a saturated monocyclic heterocycloalkyl group unsubstituted or substituted with a methyl, —OH, or —OC₁₋₄alkyl substituent;
      where R^{c} and R^{d} are each independently —H or —C₁₋₆alkyl;
R^{g1} is —H or halo;
R^{g2} is —C₁₋₄alkyl, methoxy, —CF₃, —SO₂CH₃, —SO₂CF₃, or —SO₂N(R^{h})Rⁱ;
   where R^{h} and Rⁱ are each independently —H or —C₁₋₆alkyl;
both R^{k1} are chloro or methyl; and
R^{g2} is —H, —CH₂OH, or —CH₂N(R^{h})Rⁱ;
   where R^{h} and Rⁱ are each independently —H or —C₁₋₆alkyl. Preferably, each R^{k1} is chloro and R^{g2} is —CF₃.

The compositions of matter or active agents of the invention include also pharmaceutically acceptable salts of the compounds represented by Formula (I) and methods of treatment using such salts. Pharmaceutically acceptable salts of the compounds described above are preferred, and those of the specific compounds exemplified herein are further preferred.

A “pharmaceutically acceptable salt” is intended to mean a salt of a free acid or base of a compound represented by Formula (I) that is non-toxic, biologically tolerable, or otherwise biologically suitable for administration to the subject. See generally, Berge et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Useful pharmaceutically acceptable salts are those that are pharmacologically effective and suitable for contact with the tissues of patients without undue toxicity, irritation, or allergic response.

A compound may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, and any compatible mixture of acids such as those given as examples herein.

If the compound is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of the invention. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of the compound. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of the compounds as amides or alkyl esters. Examples of amides include those derived from ammonia, primary C₁₋₆alkyl amines and secondary di(C₁₋₆alkyl)amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl)esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I) or (II). A “pharmaceutically active metabolite” means a pharmacologically active product of metabolism in the body of the compound or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) or (II) and their pharmaceutically acceptable salts, pharmaceutically acceptable prodrugs, and pharmaceutically active metabolites (collectively, “active agents”) of the present invention are useful as TRPV1 modulators in the methods of the invention. The active agents may be used in the inventive methods for the treatment of medical conditions, diseases, or disorders, including symptoms or disease states, mediated through modulation of TRPV1, such as those described herein.

Accordingly, the invention relates to methods of using the active agents to treat subjects diagnosed with or suffering from a disease, disorder, or condition mediated through TRPV1 activity, such as: i) pain (acute, chronic, inflammatory, or neuropathic pain); ii) itch or various inflammatory disorders; iii) inner ear disorders; iv) fever or other disorders of thermoregulation; v) tracheobronchial or diaphragmatic dysfunction; vi) gastrointestinal or urinary tract disorders; or vii) disorders associated with reduced blood flow to the CNS or CNS hypoxia. Diseases, disorders, and conditions are intended to include symptoms and indications.

In a preferred embodiment, an active agent of the present invention is administered to treat pain. Certain types of pain may be considered a disease or disorder, while other types may be considered symptoms of various diseases or disorders, and pain may include various etiologies. Exemplary types of pain treatable with a TRPV1-modulating agent according to the invention include pain arising from or caused by: osteoarthritis, rotator cuff disorders, arthritis (e.g., rheumatoid arthritis or inflammatory arthritis), fibromyalgia, migraine and headache (e.g. cluster headache, sinus headache, or tension headache; see, Goadsby Curr. Pain Headache Reports 2004, 8, 393), sinusitis, oral mucositis, toothache, dental trauma, dental extractions, dental infections, burn, sunburn, dermatitis, psoriasis, eczema, insect sting or bite, burn pain (Bolkskei et al., Pain 2005, in press), musculoskeletal disorders, bony fractures, ligamentous sprains, plantar fasciitis, costochondritis, tendonitis, bursitis, tennis elbow, pitcher's elbow, patellar tendonitis, repetitive strain injury, myofascial syndrome, muscle strain, myositis, temporomandibular joint disorder, amputation, low back pain, spinal cord injury, neck pain, whiplash, bladder spasms, GI tract disorders, interstitial cystitis, urinary tract infection, urethral colic, renal colic, pharyngitis, cold sores, stomatitis, external otitis, otitis media (Chan et al., Lancet 2003, 361, 385), burning mouth syndrome, mucositis, esophageal pain, esophageal spasms, abdominal disorders, gastroesophageal reflux disease, pancreatitis, enteritis, irritable bowel disorder, inflammatory bowel disease, Crohn's disease, ulcerative colitis, colon distension, abdominal constriction, diverticulosis, diverticulitis, intestinal gas, hemorrhoids, anal fissures, anorectal disorders, prostatitis, epididymitis, testicular pain, proctitis, rectal pain, cholecystitis, labor, childbirth, endometriosis, menstrual cramps, pelvic pain, vulvodynia, vaginitis, orolabial and genital infections (e.g. herpes simplex), pleurisy, pericarditis, non-cardiac chest pain, contusions, abrasions, skin incision (Honore, P. et al., J. Pharmacol. Exp. Ther. 2005, 314, 410-21), postoperative pain, peripheral neuropathy, central neuropathy, diabetic neuropathy, acute herpetic neuralgia, post-herpetic neuralgia, trigeminal neuralgia, glossopharyngeal neuralgia, atypical facial pain, gradiculopathy, HIV associated neuropathy, physical nerve damage, causalgia, reflex sympathetic dystrophy, sciatica, cervical, thoracic or lumbar radiculopathy, brachial plexopathy, lumbar plexopathy, neurodegenerative disorders, occipital neuralgia, intercostal neuralgia, supraorbital neuralgia, inguinal neuralgia, meralgia paresthetica, genitofemoral neuralgia, carpal tunnel syndrome, Morton's neuroma, post-mastectomy syndrome, post-thoracotomy syndrome, post-polio syndrome, Guillain-Barré syndrome, Raynaud's syndrome, coronary artery spasm (Printzmetal's or variant angina), visceral hyperalgesia (Pomonis, J. D. et al. J. Pharmacol. Exp. Ther. 2003, 306, 387; Walker, K. M. et al., J. Pharmacol. Exp. Ther. 2003, 304(1), 56-62), thalamic pain, cancer (e.g. pain caused by cancer, by treatment of cancer by radiation or chemotherapy, or by nerve or bone lesions associated with cancer (see, Menendez, L. et al., Neurosci. Lett. 2005, 393 (1), 70-73; Asai, H. et al., Pain 2005, 117, 19-29), or bone destruction pain (see, Ghilardi, J. R. et al., J. Neurosci. 2005, 25, 3126-31)), infection, or metabolic disease. Additionally, the compounds may be used to treat pain indications such as visceral pain, ocular pain, thermal pain, dental pain, capsaicin-induced pain (as well as other symptomatic conditions induced by capsaicin such as cough, lachrymation, and bronchospasm).

In another preferred embodiment, active agents are administered to treat: itch, which may arise from various sources, such as dermatological or inflammatory disorders; or inflammatory disorders selected from the group consisting of: renal or hepatobiliary disorders, immunological disorders, medication reactions and unknown/idiopathic conditions. Inflammatory disorders treatable with an inventive agent include, for example, inflammatory bowel disease (IBD), Crohn's disease, and ulcerative colitis (Geppetti, P. et al., Br. J. Pharmacol. 2004, 141, 1313-20; Yiangou, Y. et al., Lancet 2001, 357, 1338-39; Kimball, E. S. et al., Neurogastroenterol. Motil., 2004, 16, 811), osteoarthritis (Szabo, A. et al., J. Pharmacol. Exp. Ther. 2005, 314, 111-119), psoriasis, psoriatic arthritis, rheumatoid arthritis, myasthenia gravis, multiple sclerosis, scleroderma, glomerulonephritis, pancreatitis, inflammatory hepatitis, asthma, chronic obstructive pulmonary disease, allergic rhinitis, uveitis, and cardiovascular manifestations of inflammation including atherosclerosis, myocarditis, pericarditis, and vasculitis.

In another preferred embodiment, inner ear disorders are treated with an inventive active agent. Such disorders include, for example, hyperacusis, tinnitus, vestibular hypersensitivity, and episodic vertigo.

In another preferred embodiment, tracheobronchial and diaphragmatic dysfunctions are treated with an inventive active agent, including, for example, asthma and allergy-related immune responses (Agopyan, N. et al., Am. J. Physiol. Lung Cell Mol. Physiol. 2004, 286, L563-72; Agopyan, N. et al., Toxicol. Appl. Pharmacol. 2003, 192, 21-35), cough (e.g., acute or chronic cough, or cough caused by irritation from gastroesophageal reflux disease; see, Lalloo, U. G. et al., J. Appl. Physiol. 1995, 79(4), 1082-7), bronchospasm, chronic obstructive pulmonary disease, chronic bronchitis, emphysema, and hiccups (hiccoughs, singultus).

In yet another preferred embodiment, gastrointestinal and urinary tract disorders are treated with an inventive active agent, such as, bladder overactivity, inflammatory hyperalgesia, visceral hyperreflexia of the urinary bladder, hemorrhagic cystitis (Dinis, P. et al., J. Neurosci. 2004, 24, 11253-11263), interstitial cystitis (Sculptoreanu, A. et al., Neurosci. Lett. 2005, 381, 42-46), inflammatory prostate disease, prostatitis (Sanchez, M. et al., Eur. J. Pharmacol. 2005, 515, 20-27), nausea, vomiting, intestinal cramping, intestinal bloating, bladder spasms, urinary urgency, defecation urgency and urge incontinence.

In another preferred embodiment, disorders associated with reduced blood flow to the CNS or CNS hypoxia are treated with an inventive agent. Such disorders include, for example, head trauma, spinal injury, thromboembolic or hemorrhagic stroke, transient ischaemic attacks, cerebral vasospasm, hypoglycaemia, cardiac arrest, status epilepticus, perinatal asphyxia, Alzheimer's disease, and Huntington's Disease.

In other embodiments, active agents are administered to treat other diseases, disorders, or conditions mediated through TRPV1 activity, such as: anxiety; learning or memory disorders; eye-related disorders (such as glaucoma, vision loss, increased intraocular pressure, and conjunctivitis); baldness (e.g., by stimulating hair growth); diabetes (including insulin-resistant diabetes or diabetic conditions mediated by insulin sensitivity or secretion); obesity (e.g., through appetite suppression); dyspepsia; biliary colic; renal colic; painful bladder syndrome; inflamed esophagus; upper airway disease; urinary incontinence; acute cystitis; and envenomations (such as marine, snake, or insect stings or bites, including jellyfish, spider, or stingray envenomations).

In especially preferred embodiments of the therapeutic methods of the invention, effective amounts of the TRPV1 modulators of the present invention are administered to treat pain, itch, cough, asthma, or inflammatory bowel disease.

The term “treat” or “treating” as used herein is intended to refer to administration of an active agent or composition of matter of the invention to a subject to effect a therapeutic or prophylactic benefit through modulation of TRPV1 activity. Treating includes reversing, ameliorating, alleviating, inhibiting the progress of, lessening the severity of, or preventing a disease, disorder, or condition (or one or more symptoms of such disease, disorder or condition) mediated through modulation of TRPV1 activity. The term “subject” refers to a mammalian patient in need of such treatment, such as a human. “Modulators” include both inhibitors and activators, where “inhibitors” refer to compounds that decrease, prevent, inactivate, desensitize or down-regulate TRPV1 expression or activity, and “activators” are compounds that increase, activate, facilitate, sensitize, or up-regulate TRPV1 expression or activity.

In treatment methods according to the invention, an effective amount of at least one active agent according to the invention is administered to a subject suffering from or diagnosed as having such a disease, disorder, or condition. An “effective amount” means an amount or dose generally sufficient to bring about the desired therapeutic or prophylactic benefit in patients in need of such treatment for the designated disease, disorder, or condition. Effective amounts or doses of the active agents of the present invention may be ascertained by routine methods such as modeling, dose escalation studies, or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status, and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, or QID). For a 70-kg human, an illustrative range for a suitable dosage amount is from about 0.05 to about 7 g/day, or about 0.2 to about 2.5 g/day. Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment methods described above. The additional active ingredients may be coadministered separately with an active agent or included with such an agent in a pharmaceutical composition according to the invention. In an exemplary embodiment, additional active ingredients are those that are known or discovered to be effective in the treatment of conditions, disorders, or diseases mediated by TRPV1 activity, such as another TRPV1 modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention. In one illustrative embodiment, a composition for treating pain according to the invention may contain one or more additional active ingredients selected from opioids, NSAIDs (e.g., ibuprofen, cyclooxygenase-2 (COX-2) inhibitors, and naproxen), gabapentin, pregabalin, tramadol, acetaminophen, and aspirin. In another illustrative embodiment, a composition for treating pain according to the invention may contain one or more additional active ingredients selected from alpha-2 adrenergic agonists (e.g., brimonidine, clonidine, dexmedetomidine, mivazerol, guanabenz, guanfacine, or methyldopa).

The active agents of the invention are used, alone or in combination with one or more additional active ingredients, to formulate pharmaceutical compositions of the invention. A pharmaceutical composition of the invention also comprises a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an active agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques now known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil, sesame oil, or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to effect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables in the formulas depicted in the schemes below are as defined above in reference to Formula (I).

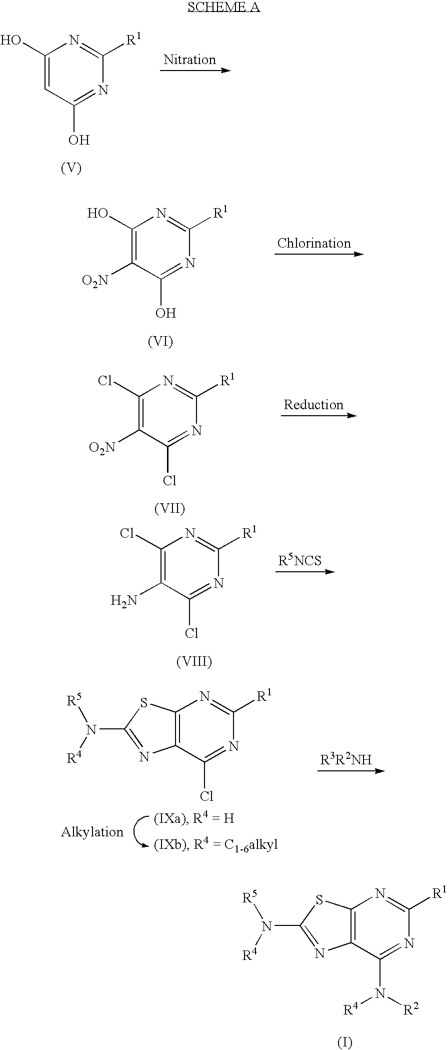

Referring to general Scheme A, compounds of Formula (I) may be prepared from pyrimidine-diols (V), which are commercially available or may be prepared according to known general processes. Nitration to form nitropyrimidines (VI) may be accomplished according to general techniques known in the art. Suitable conditions include treatment with glacial acetic acid and nitric acid at a temperature between about 0° C. and about 60° C. Conversion to dichloropyrimidines (VII) may also be performed according to general techniques known in the art. Preferred conditions involve reaction of nitropyrimdines (VI) with POCl₃ or PCl₃, in a solvent such as acetonitrile, N,N-dimethylaniline, or N,N-diethylaniline, with heating to a temperature between about 50° C. and about 120° C. Reduction of the nitro group to provide an amine (VIII) may be performed using a suitable reducing agent, such as SnCl₂, hydrazine, or Zn/NH₄Cl, in a solvent such as acetone, ethanol (EtOH), water, or a mixture thereof. Exemplary conditions include treatment with Zn (about 5-7 equivalents) and aqueous NH₄Cl (about 15 equivalents) in acetone/water. For some embodiments, amines of formula (VIII) are commercially available. The thiazolopyrimidine core may be formed by condensation with isothiocyanates R⁵NCS, in the presence of a suitable base, such as 1,8-diazabicyclo[5.4.0]undec-7-ene (DBU) or Cs₂CO₃, in a solvent such as acetonitrile, at a temperature from about room temperature (rt) and about 70° C., to form compounds of formula (IXa) (See: Player, M. et al. J. Org. Chem. 2005, 70, 10194). Exemplary conditions include treatment with Cs₂CO₃ (about 2 equivalents) in acetonitrile at about 50° C. Optional alkylation of amines (IXa) with C₁₋₆alkyl chlorides, bromides, iodides, or the like, the presence of a suitable base such as NaH, in a solvent such as N,N-dimethylformamide (DMF) or ethylene glycol dimethyl ether (DME), provides amines (IXb). Chloro-pyrimidines (IX) may then be reacted with aromatic amines R³R²NH (where R³ is phenyl, monocyclic five-membered heteroaryl, or monocyclic six-membered heteroaryl), in the presence of an acid catalyst, preferably p-toluenesulfonic acid, methanesulfonic acid, HCl, or trifluoroacetic acid (TFA), in a solvent such as toluene, dioxane, acetonitrile, isopropanol, water, or a mixture thereof, at a temperature from about 70 to about 150° C., optionally using microwave irradiation or a sealed tube, to provide compounds of Formula (I). Alternatively, reaction with aromatic amines R³R²NH is accomplished under palladium coupling conditions. Preferred conditions involve treatment of chloro-pyrimidines (IX) with aromatic amines R³R²NH and HCl in isopropanol at reflux temperature. Chloro-pyrimidines (IX) may be reacted with non-aromatic amines R³R²NH in solvents such as toluene, dioxane, or t-amyl-OH, at temperatures from about rt to about 150° C., to provide compounds of Formula (I).

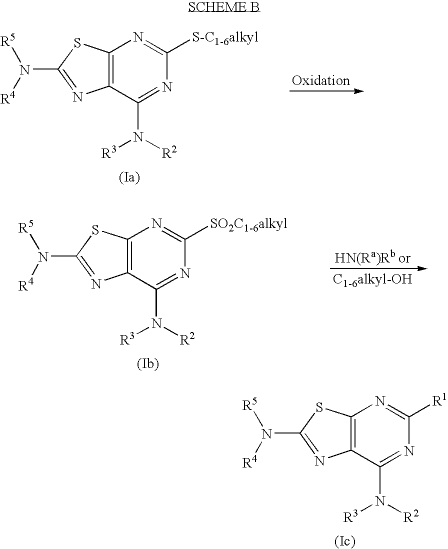

As depicted in general Scheme B, compounds of Formula (I) where R¹ is —S—C₁₋₆alkyl (Ia) may be converted into other compounds of Formula (I), such as (Ib) and (Ic). Oxidation of thioethers (Ia) yields sulfones (Ib), and may be accomplished by reaction with a suitable oxidizing agent such as OXONE™, meta-chloroperbenzoic acid (mCPBA), or dimethyldioxirane, in a solvent such as CH₂Cl₂, methanol (MeOH), tetrahydrofuran (THF), water, or a mixture thereof. Exemplary conditions include treatment with oxone (about 3 equivalents) in MeOH/THF/water at about 40° C. Displacement of the sulfone substituent to obtain a compound of formula (Ic) where R¹ is —O—C₁₋₆alkyl is attained by reaction with an alcohol HO—C₁₋₆alkyl, preferably used as the solvent, in the presence of a suitable base, such as NaH, KOtBu, NaO—C₁₋₆alkyl, or NH₃, at a temperature between rt and the reflux temperature of the solvent, and optionally using a sealed tube. Exemplary conditions include heating with NaOMe in MeOH at 80° C. in a sealed tube. Displacement of the sulfone substituent with amines HN(R^{a})R^{b} yields compounds of formula (Ic) where R¹ is —NR^{a}R^{b}, and may be performed neat or in alcoholic solvents such as MeOH, EtOH, tBuOH, n-BuOH, or t-amyl-OH, or a mixture thereof, or in a solvent such as toluene or benzene, at temperatures from about rt to about 150° C., and optionally using a sealed tube. Preferably, the reaction is in n-BuOH and t-amyl-OH as the solvent, and at a temperature of about 130° C. in a sealed tube.

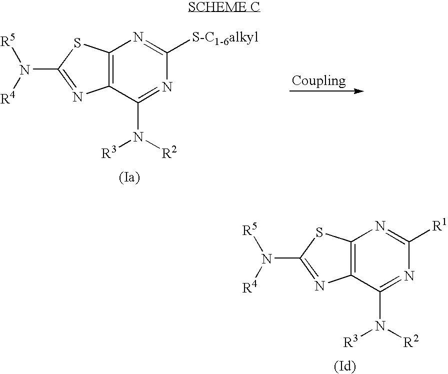

Referring to general Scheme C, compounds of Formula (I) where R¹ is phenyl, C₁₋₆alkyl, or monocyclic cycloalkyl (Id), may be prepared by coupling of thioethers (Ia) with boronic acids R¹—B(OH)₂, in the presence of a suitable catalyst such as a nickel (II) (e.g., NiCl₂) or palladium catalyst (e.g., Pd₂(dba)₃), with or without copper salt additives.

Compounds of Formula (I) may be converted to their corresponding salts using general methods described in the art. For example, amines of Formula (I) may be treated with trifluoroacetic acid, HCl, sulfuric acid, phosphoric acid, or citric acid in a solvent such as Et₂O, CH₂Cl₂, THF, MeOH, or isopropanol to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation techniques, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using known techniques such as chromatography or crystallization.

The following specific examples are provided to illustrate various preferred embodiments of active agents according to the invention.

### EXAMPLES

### Chemistry:

In the examples below, the following experimental and analytical protocols were followed unless otherwise indicated.

Where solutions were “concentrated”, they were concentrated using a rotary evaporator under reduced pressure. Unless otherwise specified, reaction solutions were stirred at room temperature (rt) under a N_{2(g)} atmosphere.

Microwave reactions were carried out in either a CEM Discover® or a Biotage Initiator™ Microwave at specified temperatures.

Where solutions were dried, they were dried over MgSO₄ or Na₂SO₄.

Normal phase purification was typically done by normal phase flash column chromatography (FCC) with RediSep® silica gel columns using ethyl acetate (EtOAc)/hexanes as eluent unless otherwise specified.

Preparative Reversed-Phase high performance liquid chromatography (HPLC) was performed on a Shimadzu® instrument with a Phenomenex Gemini column (C18; 5 μm, 150×21.2 mm) or Waters Xterra RP18 OBD column (5 μm, 100×30 mm), a flow rate of 30 mL/min (Gemini) or 80 mL/min (Waters), detection at λ=254 nm. The eluent was 0.05% TFA in an acetonitrile/H₂O gradient, ramped over 20 min.

Unless otherwise indicated, Example compounds were obtained as free bases following FCC or as trifluoroacetic acid salts following reverse phase HPLC purification.

NMR spectra were obtained on Bruker model DRX spectrometers. The format of ¹H NMR data below is: chemical shift in ppm downfield of the tetramethylsilane reference (multiplicity, coupling constant J in Hz, integration).

Mass spectra were obtained on an Agilent series 1100 MSD using electrospray ionization (ESI) in either positive or negative modes as indicated. Calculated mass corresponds to the exact mass.

Chemical names were generated using ChemDraw Ultra 6.0.2 (CambridgeSoft Corp., Cambridge, Mass.) or ACD/Name Version 9 (Advanced Chemistry Development, Toronto, Ontario, Canada).

### Intermediate 1: (7-Chloro-thiazolo[5,4-d]pyrimidin-2-yl)-(2,6-dichloro-phenyl)-amine

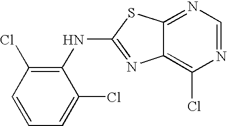

To a solution of 4,6-dichloro-5-aminopyrimidine (4.0 g, 24 mmol) in MeCN (100 mL) was added Cs₂CO₃ (16 g, 49 mmol) followed by 2,6-dichlorophenyl thioisocyanate (5 g, 24 mmol). The resulting mixture was stirred at 50° C. in a sealed tube. After 12 hours (h), the mixture was cooled to rt and concentrated. The residue purified directly by FCC to afford a colorless solid (5.5 g, 69%). MS (ESI): mass calcd. for C₁₁H₅Cl₃N₄S, 329.9; m/z found, 330.9 [M+H]⁺. ¹H NMR (CDCl₃): δ 9.48 (br s, 1H), 8.59 (s, 1H), 7.53 (d, J=8.2 Hz, 2H), 7.38 (app dd, J=8.5, 7.7 Hz, 1H).

The following Intermediates 2 through 11 were prepared using methods similar to that described for Intermediate 1, with the appropriate substituent changes in the reactant materials.

### Intermediate 2: (7-Chloro-5-methyl-thiazolo[5,4-d]pyrimidin-2-yl)-(2,6-dichloro-phenyl)-amine

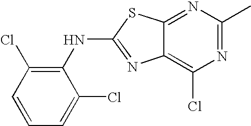

MS (ESI): mass calcd. for C₁₂H₇Cl₃N₄S, 343.9; m/z found, 345.3 [M+H]⁺. ¹H NMR (CDCl₃): δ 9.09 (br s, 1H), 7.51 (d, J=7.6 Hz, 2H), 7.35 (app dd, J=8.8, 7.6 Hz, 1H), 2.70 (s, 3H).

### Intermediate 3: (7-Chloro-thiazolo[5,4-d]pyrimidin-2-yl)-(2,6-dimethyl-phenyl)-amine

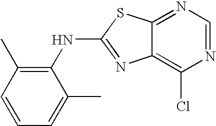

MS (ESI): mass calcd. for C₁₃H₁₁ClN₄S, 290.0; m/z found, 291.4[M+H]⁺. ¹H NMR (CDCl₃): δ 8.75 (br s, 1H), 8.54 (s, 1H), 7.29 (app dd, J=8.2, 6.8 Hz, 1H), 7.23 (d, J=6.8 Hz, 2H), 2.35 (s, 6H).

### Intermediate 4: (2-Chloro-6-methyl-phenyl)-(7-chloro-thiazolo[5,4-d]pyrimidin-2-yl)-amine

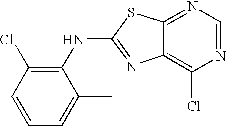

MS (ESI): mass calcd. for C₁₂H₈Cl₂N₄S, 309.9; m/z found, 310.8 [M+H]⁺. ¹H NMR (CDCl₃): δ 9.44 (br s, 1H), 8.56 (s, 1H), 7.44-7.42 (m, 1H), 7.36-7.29 (m, 2H), 2.43 (s, 3H).

### Intermediate 5: (7-Chloro-thiazolo[5,4-d]pyrimidin-2-yl)-(2-chloro-6-trifluoromethyl-phenyl)-amine

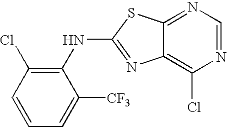

MS (ESI): mass calcd. for C₁₂H₅Cl₂F₃N₄S, 363.9; m/z found, 365.2 [M+H]⁺. ¹H NMR (CDCl₃): δ 10.32 (br s, 1H), 8.56 (s, 1H), 7.85 (d, J=8.0 Hz, 1H), 7.77 (d, J=8.0 Hz, 1H), 7.58 (t, J=8.0 Hz, 1H).

### Intermediate 6: (2-Chloro-phenyl)-(7-chloro-thiazolo[5,4-d]pyrimidin-2-yl)-amine

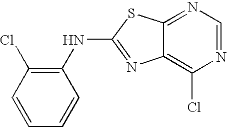

MS (ESI): mass calcd. for C₁₁H₆Cl₂N₄S, 295.9; m/z found, 297.3 [M+H]⁺. ¹H NMR (CDCl₃): δ 8.66 (s, 1H), 8.22 (dd, J=8.1, 1.5 Hz, 1H), 7.99 (br s, 1H), 7.50 (dd, J=8.1, 1.5 Hz, 1H), 7.44-7.39 (m, 1H), 7.21-7.18 (m, 1H).

### Intermediate 7: (7-Chloro-thiazolo[5,4-d]pyrimidin-2-yl)-o-tolyl-amine

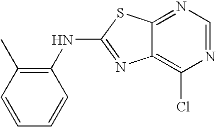

MS (ESI): mass calcd. for C₁₂H₉ClN₄S, 276.0; m/z found, 277.4 [M+H]⁺. ¹H NMR (CDCl₃): δ 8.58 (s, 1H), 8.29 (br s, 1H), 7.52-7.49 (m, 1H), 7.37-7.31 (m, 3H), 2.36 (s, 3H).

### Intermediate 8: (7-Chloro-thiazolo[5,4-d]pyrimidin-2-yl)-(4-trifluoromethyl-phenyl)-amine

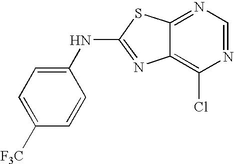

MS (ESI): mass calcd. for C₁₂H₆ClF₃N₄S, 330.0; m/z found, 331.2 [M+H]⁺.

### Intermediate 9: (7-Chloro-thiazolo[5,4-d]pyrimidin-2-yl)-phenyl-amine

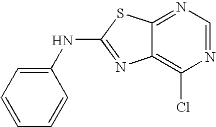

MS (ESI): mass calcd. for C₁₁H₇ClN₄S, 262.0; m/z found, 263.3 [M+H]⁺.

### Intermediate 10: (7-Chloro-thiazolo[5,4-d]pyrimidin-2-yl)-(3,5-dimethyl-isoxazol-4-yl)-amine

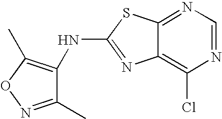

MS (ESI): mass calcd. for C₁₀H₈ClN₅OS, 281.0; m/z found, 282.3 [M+H]⁺.

### Intermediate 11: (7-Chloro-5-methyl-thiazolo[5,4-d]pyrimidin-2-yl)-(5-methyl-3-phenyl-isoxazol-4-yl)-amine

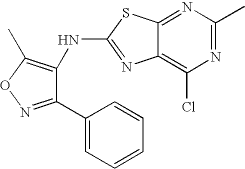

MS (ESI): mass calcd. for C₁₆H₁₂ClN₅OS, 357.0; m/z found, 358.3 [M+H]⁺.

### Intermediate 12: (7-Chloro-5-methylsulfanyl-thiazolo[5,4-d]pyrimidin-2-yl)-(2,6-dichloro-phenyl)-amine

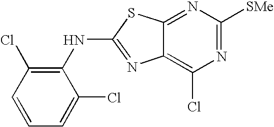

Step A: 2-Methylsulfanyl-5-nitro-pyrimidine-4,6-diol. 2-Methylsulfanyl-pyrimidine-4,6-diol (10 g, 63 mmol) was added portion-wise to a stirring solution of glacial acetic acid (25 mL) and concentrated nitric acid (10 mL) at 50° C. After 3 h, the reaction mixture was poured onto crushed ice and the product was isolated by filtration as a yellow solid (6 g, 49%). MS (ESI): mass calcd. for C₅H₅N₃O₄S, 203.0; m/z found, 202.4 [M−H].

Step B: 4,6-Dichloro-2-methylsulfanyl-pyrimidin-5-ylamine. N,N-Diethylaniline (3.3 mL) was added dropwise to a stirred mixture of 2-methylsulfanyl-5-nitro-pyrimidine-4,6-diol (3.4 g, 17 mmol) and POCl₃ (15 mL) at rt. After 15 minutes (min), the reaction mixture was heated to 105° C. and stirred for 1 h. The cooled reaction mixture was poured onto ice (100 g) and then extracted with Et₂O (3×100 mL). The combined extracts were dried and concentrated, and the residue was purified directly by FCC to afford 4,6-dichloro-2-methylsulfanyl-5-nitro-pyrimidine as a colorless solid (3.5 g, 87%).

To a solution of the 4,6-dichloro-2-methylsulfanyl-5-nitro-pyrimidine (1 g, 4.2 mmol) in EtOH (20 mL) was added SnCl₂.2H₂O (3.8 g, 17 mmol). The mixture was heated to 90° C. After 2 h, the reaction mixture was cooled and the solution was concentrated. The residue was treated with satd. aq. NaHCO₃ until a pH of 8 resulted. The resulting mixture was extracted with EtOAc (3×100 mL). The combined organic extracts were dried and concentrated. The residue was purified directly by FCC to afford a colorless solid (723 mg, 87%). MS (ESI): mass calcd. for C₅H₅Cl₂N₃S, 208.9; m/z found, 210.3 [M+H]⁺. ¹H NMR ((CD₃)₂SO): δ 5.89 (s, 2H), 2.45 (s, 3H).

Step C. The title compound was prepared from 4,6-dichloro-2-methylsulfanyl-pyrimidin-5-ylamine using a method analogous to that described for Intermediate 1. MS (ESI): mass calcd. for C₁₂H₇Cl₃N₄S₂, 375.9; m/z found, 377.2 [M+H]⁺.

### Intermediate 13: 3,5-Dichloro-4-isothiocyanato-benzonitrile

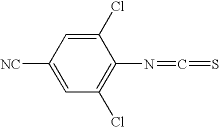

To a solution of 4-amino-3,5-dichloro-benzonitrile (2.1 g, 11 mmol) and DMF (0.3 mL, 3.2 mmol) in 1,2-dichlorobenzene (15 mL) was added thiophosgene (2.6 g, 23 mmol). The resulting solution was heated to 160° C. and held at that temperature for 10 min then cooled to rt. The room temperature solution was purified by a plug of silica (220 g) using 10% EtOAc-hexanes (750 mL) to afford the title compound as a colorless solid (2.4 g, 95%). MS (ESI): mass calcd. for C₈H₂Cl₂N₂S, 227.9; m/z found, 229.0 [M+H]⁺. ¹H NMR (CDCl₃): δ 7.63 (s, 2H). ¹³C NMR (CDCl₃): 6143.7, 133.5, 133.6, 131.5, 115.8, 111.0.

### Intermediate 14: 3,5-Dichloro-4-isothiocyanato-pyridine

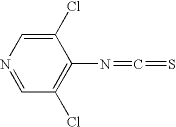

To a solution of 3,5-dichloro-pyridin-4-ylamine (1.7 g, 10 mmol), iPr₂NEt (2.7 g, 20 mmol) in CH₂Cl₂ was added thiophosgene (1.2 g, 10 mmol) at 0° C. After 1 h, the solution was allowed to warm to rt and stirred for 72 h. The resulting solution was concentrated and the crude residue was purified by FCC to afford a yellow solid (640 mg, 31%). MS (ESI): mass calcd. for C₆H₂Cl₂N₂S, 203.9; m/z found, 204.0 [M+H]⁺.

### Intermediate 15: 1-Methyl-1,2,3,4-tetrahydro-quinolin-7-ylamine

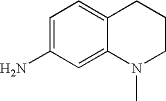

The title compound was prepared analogously to methods reported in Hamann, L. G., et. al., J. Med. Chem., 1998, 41, 623, and Higuchi, R. I., et. al., Bioorg. Med. Chem. Lett. 1999, 9, 1335.

### Intermediate 16: 1,4,4-Trimethyl-1,2,3,4-tetrahydro-quinolin-7-ylamine

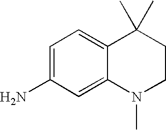

The title compound was prepared analogously to methods reported in Hamann, L. G., et. al., J. Med. Chem., 1998, 41, 623, and Higuchi, R. I., et. al., Bioorg. Med. Chem. Lett. 1999, 9, 1335.

### Intermediate 17: N-(4-Amino-phenyl)-N-methyl-methanesulfonamide

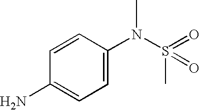

To a solution of methyl-(4-nitro-phenyl)-amine (1.0 g, 6.8 mmol) and DMF (30 mL) was added NaH (60% in mineral oil; 788 mg, 19.7 mmol) portionwise. After 10 min, methanesulfonyl chloride (0.76 mL, 9.85 mmol) was added dropwise to the solution. After 1 h, the resulting solution was partitioned between H₂O (50 mL) and EtOAc (50 mL). The aqueous layer was extracted with EtOAc (3×75 mL). The combined organic layers were dried, filtered, and concentrated. The crude residue was used immediately in the next step. To a mixture of N-methyl-N-(4-nitro-phenyl)-methanesulfonamide (1.8 g, 7.8 mmol), ammonium formate (2.9 g, 47 mmol), and MeOH (30 mL) was added Pd/C (10% by weight, 832 mg, 0.78 mmol). The resulting mixture was heated to 60° C. After 12 h, the reaction was cooled, filtered through a pad of diatomaceous earth, eluting with MeOH (60 mL), and concentrated. The resulting crude residue was partitioned between satd. aq. NaHCO₃ (50 mL) and EtOAc (50 mL). The aqueous layer was extracted with EtOAc (3×75 mL). The combined organic layers were dried, filtered, and concentrated. The title compound was used without further purification (1.2 g, 77%). MS (ESI): mass calcd. for C₈H₁₂N₂O₂S, 200.0; m/z found, 201.1 [M+H]⁺.

### Intermediate 18: 4-(Propane-2-sulfonyl)-phenylamine

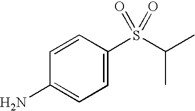

To a mixture of 1-fluoro-4-nitro-benzene (1.0 g, 7.1 mmol) and Cs₂CO₃ (4.6 g, 14.1 mmol) in DMF (25 mL) was added propane-2-thiol (600 mg, 7.78 mmol). After 12 h, the reaction mixture was concentrated and the crude residue was purified by FCC to afford 1-isopropylsulfanyl-4-nitro-benzene as a colorless solid (1.1 g, 78%). MS (ESI): mass calcd. for C₉H₁₁NO₂S, 197.0; m/z found, 198.1 [M+H]⁺. To a mixture of 1-isopropylsulfanyl-4-nitro-benzene (1.0 g, 5.1 mmol), ammonium formate (1.9 g, 30 mmol), and MeOH (20 mL) was added Pd/C (10% by weight, 539 mg, 0.510 mmol). The resulting mixture was heated to 60° C. After 48 h, the reaction was cooled, filtered through a pad of diatomaceous earth, eluting with MeOH (60 mL), and concentrated. The resulting crude residue was partitioned between satd. aq. NaHCO₃ (50 mL) and EtOAc (50 mL). The aqueous layer was extracted with EtOAc (3×75 mL). The combined organic layers were dried, filtered, and concentrated. The residue was purified directly by FCC to afford a red solid (540 mg, 64%). MS (ESI): mass calcd. for C₉H₁₃NS, 167.1; m/z found, 168.1 [M+H]⁺.

### Intermediate 19: 2-(4-Amino-phenyl)-2-methyl-propionitrile

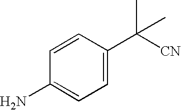

The title compound was prepared analogously to methods reported in Hicks, T. et al., J. Med. Chem., 1979, 22, 1460-1464.

### Intermediate 20: 2-(4-Amino-phenyl)-2-methyl-propionic acid methyl ester

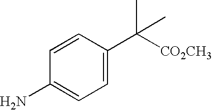

To a solution of 2-methyl-2-(4-nitrophenyl)-propionic acid (1.00 g, 4.76 mmol) in 10% MeOH/benzene (20 mL) was added dropwise (trimethylsilyl)-diazomethane (2.0 M in hexanes, 3.5 mL, 7.0 mmol). The reaction mixture was stirred at rt until evolution of N₂ ceased (<5 min) and then concentrated. The crude residue was purified (FCC) to give 2-methyl-2-(4-nitrophenyl)-propionic acid methyl ester (937.6 mg, 88%). To a solution of 2-methyl-2-(4-nitrophenyl)-propionic acid methyl ester (932 mg, 4.16 mmol) and ammonium formate (1.58 g, 25.1 mmol) in MeOH (30 mL) was added Pd/C (10%, 441.2 mg, 0.414 mmol). The reaction mixture was heated to 40° C. After 2 h, the mixture was filtered through a plug of diatomaceous earth, eluting with MeOH (30 mL). The filtrate was concentrated and the residue was diluted with satd. aq. NaHCO₃ (30 mL) and extracted with CH₂Cl₂ (3×30 mL). The combined organic layers were dried and concentrated to provide the title compound, which was used without further purification. MS (ESI): mass calcd. for C₁₁H₁₅NO₂, 193.1; m/z found, 194.1 [M+H]⁺.

### Intermediate 21: N-(4-Amino-phenyl)-dimethanesulfonamide

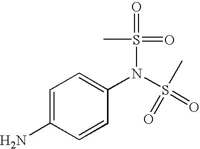

To a 0° C. solution of 4-nitro-phenylamine (1.5 g, 10.9 mmol) and iPr₂NEt (5.6 mL, 32.4 mmol) in CH₂Cl₂ (30 mL) was added methanesulfonyl chloride (1.25 mL, 16.3 mmol) dropwise. The reaction mixture was stirred at 0° C. for 1 h, and then allowed to warm to rt. The resulting mixture was diluted with satd. aq. NaHCO₃ (30 mL) and extracted with CH₂Cl₂ (3×30 mL). The combined organic layers were dried and concentrated to provide N-(4-nitro-phenyl)-dimethanesulfonamide (2.11 g, 66%). MS (ESI): mass calcd. for C₈H₁₀N₂O₆S₂, 216.0; m/z found, 217.1 [M+H]⁺.

To a solution of N-(4-nitro-phenyl)-dimethanesulfonamide (1.0 g, 3.4 mmol) and ammonium formate (1.3 g, 20 mmol) in MeOH (20 mL) was added Pd/C (10%, 362 mg, 0.34 mmol). The reaction mixture was heated to 40° C. After 2 h, the mixture was filtered through a plug of diatomaceous earth, eluting with MeOH (30 mL). The filtrate was concentrated and the residue was diluted with satd. aq. NaHCO₃ (30 mL) and extracted with CH₂Cl₂ (3×30 mL). The combined organic layers were dried and concentrated to provide the title compound, which was used without further purification. MS (ESI): mass calcd. for C₈H₁₂N₂O₄S₂, 264.0; m/z found, 265.1 [M+H]⁺.

### Intermediate 22: 2-Isothiocyanato-3-methyl-pyridine

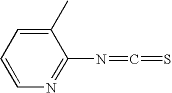

To a 0° C. solution of 3-methyl-pyridin-2-ylamine (3.2 g, 29 mmol) and iPr₂NEt (7.6 g, 59 mmol) in CH₂Cl₂ was added thiophosgene (3.4 g, 29 mmol). After 1 h, the solution was allowed to warm to rt and stirred for 72 h. The resulting solution was concentrated and the crude residue was purified by FCC to afford a colorless solid (2 g, 46%). MS (ESI): mass calcd. for C₇H₆N₂S, 150.0; m/z found, 151.0 [M+H]⁺.

Intermediates 23-40 were prepared using methods analogous to those described for Intermediate 1. In some cases, DBU (2 equiv.) was used in place of Cs₂CO₃.

### Intermediate 23: 3,5-Dichloro-4-(7-chloro-thiazolo[5,4-d]pyrimidin-2-ylamino)-benzonitrile

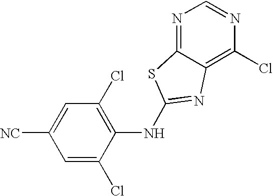

MS (ESI): mass calcd. for C₁₂H₄Cl₃N₅S, 354.9; m/z found, 355.9 [M+H]⁺ 0.1H NMR ((CD₃)₂SO): δ 8.68 (s, 1H), 8.32 (s, 2H).

### Intermediate 24: (7-Chloro-thiazolo[5,4-d]pyrimidin-2-yl)-(3-methyl-pyridin-2-yl)-amine

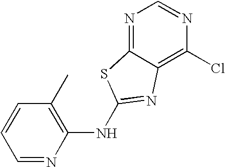

MS (ESI): mass calcd. for C₁₁H₈ClN₅S, 277.0; m/z found, 278.1 [M+H]⁺. ¹H NMR ((CD₃)₂SO): δ 11.57 (s, 1H), 8.72 (s, 1H), 8.27 (dd, J=4.84, 1.05 Hz, 1H), 7.67 (d, J=6.67 Hz, 1H), 7.08 (dd, J=7.33, 4.92 Hz, 1H), 2.40 (s, ³H).

### Intermediate 25: (7-Chloro-5-methyl-thiazolo[5,4-d]pyrimidin-2-yl)-(3-methyl-pyridin-2-yl)-amine

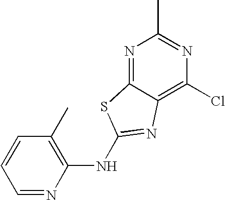

MS (ESI): mass calcd. for C₁₂H₁₀ClN₅S, 291.0; m/z found, 292.0 [M+H]⁺. ¹H NMR ((CD₃)₂SO): δ 11.40 (s, 1H), 8.25-8.21 (m, 1H), 7.65-7.62 (m, 1H), 7.04 (dd, J=7.32, 4.93 Hz, 1H), 2.64 (s, ³H), 2.38 (s, 3H).

### Intermediate 26: (7-Chloro-thiazolo[5,4-d]pyrimidin-2-yl)-(3,5-dichloro-pyridin-4-yl)-amine

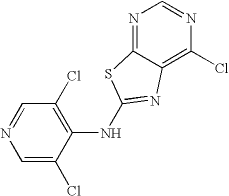

MS (ESI): mass calcd. for C₁₀H₄Cl₃N₅S, 330.9; m/z found, 332.0 [M+H]⁺. ¹H NMR ((CD₃)₂SO): δ 11.5 (brs, 1H), 8.78 (s, 2H), 8.69 (s, 1H).

### Intermediate 27: (7-Chloro-5-methyl-thiazolo[5,4-d]pyrimidin-2-yl)-(2-nitro-phenyl)-amine

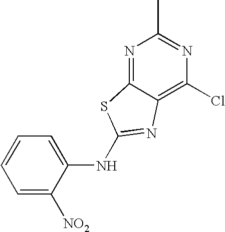

MS (ESI): mass calcd. for C₁₂H₈ClN₅O₂S, 321.0; m/z found, 322.0 [M+H]⁺. ¹H NMR (CDCl₃): δ 11.00 (s, 1H), 9.12 (dd, J=8.58, 1.19 Hz, 1H), 8.34 (dd, J=8.47, 1.55 Hz, 1H), 7.81 (ddd, J=8.70, 7.25, 1.60 Hz, 1H), 7.25 (ddd, J=8.48, 7.25, 1.26 Hz, 1H), 2.80 (s, 3H).

### Intermediate 28: Benzo[1,2,5]thiadiazol-4-yl-(7-chloro-5-methyl-thiazolo[5,4-d]pyrimidin-2-yl)-amine

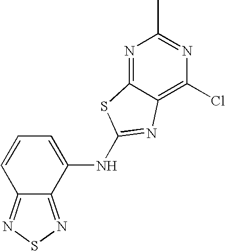

MS (ESI): mass calcd. for C₁₂H₇ClN₆S₂, 333.9; m/z found, 335 [M+H]⁺. ¹H NMR ((CD₃)₂SO): δ 11.86 (s, 1H), 8.79 (dd, J=6.86, 1.47 Hz, 1H), 7.85-7.75 (m, 2H), 2.65 (s, 3H).

### Intermediate 29: (7-Chloro-5-methyl-thiazolo[5,4-d]pyrimidin-2-yl)-(2-methylsulfanyl-phenyl)-amine

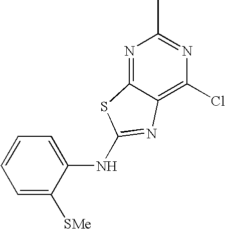

MS (ESI): mass calcd. for C₁₃H₁₁ClN₄S₂, 322.0; m/z found, 323.1 [M+H]⁺. ¹H NMR ((CD₃)₂SO): δ 10.53 (s, 1H), 7.64 (dd, J=7.79, 0.95 Hz, 1H), 7.42 (dd, J=7.93, 1.35 Hz, 1H), 7.38-7.34 (m, 1H), 7.28 (dt, J=7.60, 1.47 Hz, 1H), 2.59 (s, 3H), 2.45 (s, 3H).

### Intermediate 30: (7-Chloro-thiazolo[5,4-d]pyrimidin-2-yl)-(2-methylsulfanyl-phenyl)-amine

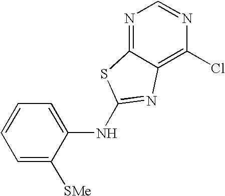

MS (ESI): mass calcd. for C₁₂H₉ClN₄S₂, 308.0; m/z found, 309.1 [M+H]⁺. ¹H NMR ((CD₃)₂SO): δ 10.70 (s, 1H), 8.59 (s, 1H), 7.64 (d, J=7.81 Hz, 1H), 7.46-7.41 (m, 1H), 7.40-7.35 (m, 1H), 7.32-7.27 (m, 1H), 2.46 (s, 3H).

### Intermediate 31: (7-Chloro-5-methyl-thiazolo[5,4-d]pyrimidin-2-yl)-(2-chloro-phenyl)-amine

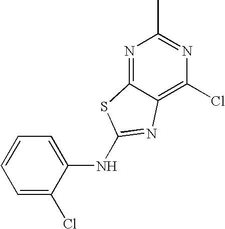

MS (ESI): mass calcd. for C₁₂H₈Cl₂N₄S, 309.9; m/z found, 311.1 [M+H]⁺. ¹H NMR (CDCl₃): δ 10.63 (s, 1H), 8.20 (dd, J=8.14, 1.25 Hz, 1H), 7.61-7.51 (m, 1H), 7.43 (dt, J=8.25, 1.46 Hz, 1H), 7.24 (dt, J=7.72, 1.53 Hz, 1H), 2.60 (s, 3H).

### Intermediate 32: (7-Chloro-5-methylsulfanyl-thiazolo[5,4-d]pyrimidin-2-yl)-(2,6-dichloro-phenyl)-amine

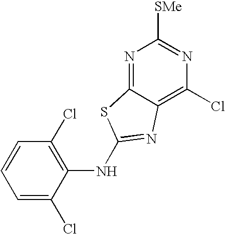

MS (ESI): mass calcd. for C₁₂H₇Cl₃N₄S₂, 375.9; m/z found, 377.2 [M+H]⁺. ¹H NMR ((CD₃)₂SO): δ 10.92 (s, 1H), 7.67 (d, J=8.15 Hz, 2H), 7.47 (t, J=8.16 Hz, 1H), 2.54 (s, 3H).

### Intermediate 33: (7-Chloro-5-methyl-thiazolo[5,4-d]pyrimidin-2-yl)-(3,5-dimethyl-isoxazol-4-yl)-amine

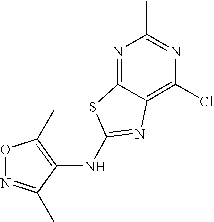

MS (ESI): mass calcd. for C₁₁H₁₀ClN₅OS, 295.0; m/z found, 296.0 [M+H]⁺. ¹H NMR ((CD₃)₂SO): δ 10.29 (s, 1H), 2.60 (s, 3H), 2.37 (s, 3H), 2.18 (s, 3H).

### Intermediate 34: (7-Chloro-thiazolo[5,4-d]pyrimidin-2-yl)-(3,5-dimethyl-isoxazol-4-yl)-amine

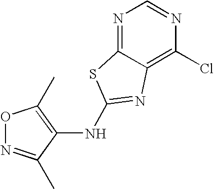

MS (ESI): mass calcd. for C₁₀H₈ClN₅OS, 281.0; m/z found, 282.3 [M+H]⁺.

### Intermediate 35: 3-(7-Chloro-thiazolo[5,4-d]pyrimidin-2-ylamino)-4-methyl-thiophene-2-carboxylic acid methyl ester

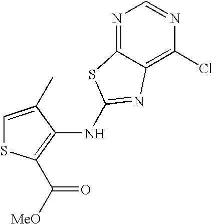

MS (ESI): mass calcd. for C₁₂H₉ClN₄O₂S₂, 339.9; m/z found, 341.2 [M+H]⁺. ¹H NMR ((CD₃)₂SO): δ 10.88 (s, 1H), 8.61 (s, 1H), 7.69 (d, J=0.93 Hz, 1H), 3.73 (s, 3H), 2.21-2.07 (m, 3H).

### Intermediate 36: (7-Chloro-5-methyl-thiazolo[5,4-d]pyrimidin-2-yl)-(2,6-dimethyl-phenyl)-amine

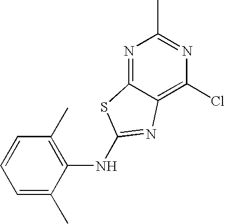

MS (ESI): mass calcd. for C₁₄H₁₃ClN₄S, 304.1; m/z found, 305.2 [M+H]⁺. ¹H NMR ((CD₃)₂SO): δ 7.29-7.16 (m, 3H), 2.64-2.58 (m, 3H), 2.30 (s, 6H).

### Intermediate 37: (7-Chloro-thiazolo[5,4-d]pyrimidin-2-yl)-(2-trifluoromethyl-phenyl)-amine

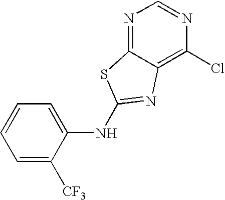

MS (ESI): mass calcd. for C₁₂H₆ClF₃N₄S, 330.0; m/z found, 301.1 [M+H]⁺. ¹H NMR (CDCl₃): δ 8.65 (s, 1H), 8.02 (d, J=8.16 Hz, 1H), 7.76 (d, J=7.92 Hz, 1H), 7.70 (t, J=7.79 Hz, 2H), 7.43 (t, J=7.69 Hz, 1H).

### Intermediate 38: (7-Chloro-thiazolo[5,4-d]pyrimidin-2-yl)-cyclohexyl-amine

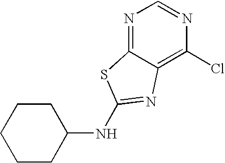

MS (ESI): mass calcd. for C₁₁H₁₃ClN₄S, 268.1; m/z found, 269.1 [M+H]⁺. ¹H NMR ((CD₃)₂SO): δ 8.89 (bs, 1H), 8.49 (s, 1H), 3.78 (bs, 1H), 2.04-1.93 (m, 2H), 1.81-1.65 (m, 2H), 1.63-1.53 (m, 1H), 1.46-1.11 (m, 5H).

### Intermediate 39: (1R,2R)-2-Benzyloxy-cyclohexyl)-(7-chloro-thiazolo[5,4-d]pyrimidin-2-yl)-amine

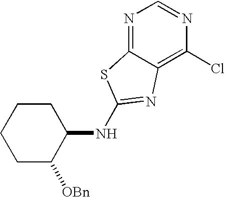

MS (ESI): mass calcd. for C₁₈H₁₉ClN₄OS, 374.1; m/z found, 375.1 [M+H]⁺. ¹H NMR ((CD₃)₂SO): δ 8.99 (bs, 1H), 8.48 (s, 1H), 7.39-6.96 (m, 5H), 4.63 (d, J=11.98 Hz, 1H), 4.48 (d, J=11.97 Hz, 1H), 3.51-3.41 (m, 1H), 2.26-1.90 (m, 2H), 1.81-1.55 (m, 2H), 1.54-1.16 (m, 5H).

### Intermediate 40: Adamantan-2-yl-(7-chloro-thiazolo[5,4-d]pyrimidin-2-yl)-amine

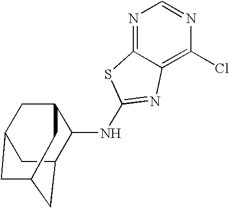

MS (ESI): mass calcd. for C₁₅H₁₇ClN₄S, 320.1; m/z found, 321.1 [M+H]⁺. ¹H NMR ((CD₃)₂SO): δ 8.92 (m, 1H), 8.46 (m, 1H), 4.09 (m, 1H), 2.20-1.95 (m, 4H), 1.92-1.76 (m, 6H), 1.77-1.68 (m, 2H), 1.65-1.48 (m, 2H).

### Intermediate 41: (7-Chloro-thiazolo[5,4-d]pyrimidin-2-yl)-(2-methanesulfonyl-phenyl)-amine

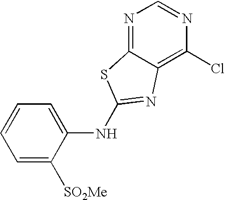

To a solution of (7-chloro-thiazolo[5,4-d]pyrimidin-2-yl)-(2-methylsulfanyl-phenyl)-amine (514 mg, 1.7 mmol) in CH₂Cl₂ (25 mL) was added m-CPBA (77%; 815 mg, 3.8 mmol). After 2 h, the solution was diluted with satd. aq. NaHCO₃ (25 mL) and extracted with CH₂Cl₂ (3×25 mL). The combined organic layers were dried, concentrated, and the residue was purified by FCC to afford a colorless solid (490 mg, 86%). MS (ESI): mass calcd. for C₁₂H₉ClN₄O₂S₂, 339.9; m/z found, 341.0 [M+H]⁺. ¹H NMR (CDCl₃): δ 8.78-8.75 (m, 1H), 8.70 (s, 1H), 7.99 (dd, J=7.97, 1.57 Hz, 1H), 7.82-7.74 (m, 1H), 7.39-7.30 (m, 1H), 3.14 (s, 3H).

### Intermediate 42: (7-Chloro-5-methyl-thiazolo[5,4-d]pyrimidin-2-yl)-(2-methanesulfonyl-phenyl)-amine

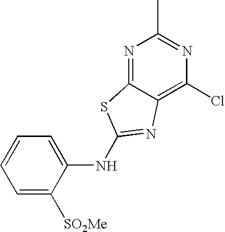

The title compound was prepared using methods analogous to those described for Intermediate 41. MS (ESI): mass calcd. for C₁₃H₁₁ClN₄O₂S₂, 354.0; m/z found, 355.0 [M+H]⁺. ¹H NMR ((CD₃)₂SO): δ 10.52 (s, 1H), 8.09-7.99 (m, 2H), 7.87-7.80 (m, 1H), 7.59-7.54 (m, 1H), 3.29 (s, 3H), 2.61 (s, 3H).

### Example 1

### N²-(2,6-Dichloro-phenyl)-N⁷-(4-trifluoromethyl-phenyl)-thiazolo[5,4-d]pyrimidine-2,7-diamine

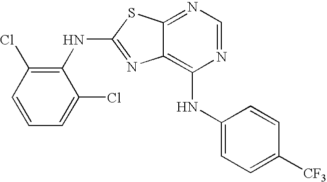

A mixture of (7-chloro-thiazolo[5,4-d]pyrimidin-2-yl)-(2,6-dichloro-phenyl)-amine (100 mg, 0.30 mmol), 4-trifluoromethyl-phenylamine (48 mg, 0.30 mmol), and p-toluenesulfonic acid (114 mg, 0.60 mmol) in toluene (3 mL) was heated at 125° C. After 2 h, the mixture was cooled and concentrated to give a crude residue, which was purified by FCC to afford a colorless solid (100 mg, 73%). MS (ESI): mass calcd. for C₁₈H₁₀Cl₂F₃N₅S, 455.0; m/z found, 456.3 [M+H]⁺. ¹H NMR (CDCl₃): δ 8.51 (s, 1H), 7.92 (d, J=8.5 Hz, 2H), 7.63-7.61 (m, 3H), 7.50 (d, J=8.1 Hz, 2H), 7.32 (t, J=8.1 Hz, 1H), 6.96 (br s, 1H).

The compounds in Examples 2 through 52 were prepared using methods similar to that described in Example 1, with the appropriate substituent changes in reactant materials.

### Example 2

### N²-(2,6-Dichloro-phenyl)-N⁷-(6-trifluoromethyl-pyridin-3-yl)-thiazolo[5,4-d]pyrimidine-2,7-diamine

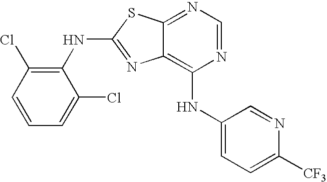

MS (ESI): mass calcd. for C₁₇H₉Cl₂F₃N₆S, 456.0; m/z found, 457.3 [M+H]⁺. ¹H NMR (CD₃OD): δ 9.04 (d, J=2.0 Hz, 1H), 8.61 (dd, J=8.8, 2.0, 1H), 8.43 (s, 1H), 7.77 (d, J=8.8, 1H), 7.60 (d, J=8.1 Hz, 2H), 7.42 (app dd, J=8.6, 8.1 Hz, 1H).

### Example 3

### N⁷-(4-tert-Butyl-phenyl)-N²-(2,6-dichloro-phenyl)-thiazolo[5,4-d]pyrimidine-2,7-diamine

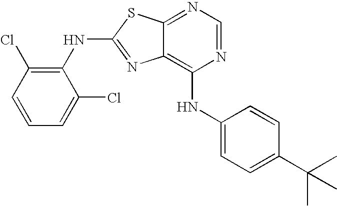

MS (ESI): mass calcd. for C₂₁H₁₉Cl₂N₅S, 443.1; m/z found, 444.4 [M+H]⁺. ¹H NMR (CD₃OD): δ 8.26 (s, 1H), 7.59-7.53 (m, 4H), 7.42-7.38 (m, 3H), 1.32 (s, 9H).

### Example 4

### N⁷-(3-Chloro-4-trifluoromethyl-phenyl)-N²-(2,6-dichloro-phenyl)-thiazolo[5,4-d]pyrimidine-2,7-diamine

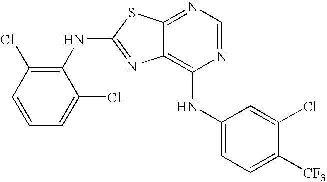

MS (ESI): mass calcd. for C₁₈H₉Cl₃F₃N₅S, 488.9; m/z found, 490.2 [M+H]⁺. ¹H NMR (CD₃OD): δ 8.43 (s, 1H), 8.28 (d, J=1.6 Hz, 1H), 7.80 (dd, J=8.7, 1.4 Hz, 1H), 7.67 (d, J=8.7 Hz, 1H), 7.60 (d, J=8.2 Hz, 2H), 7.42 (t, J=8.2, 1H).

### Example 5

### N²-(2,6-Dichloro-phenyl)-5-methyl-N⁷-(4-trifluoromethyl-phenyl)-thiazolo[5,4-d]pyrimidine-2,7-diamine

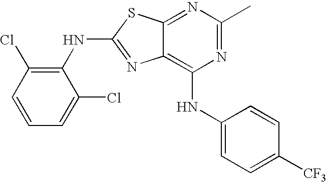

MS (ESI): mass calcd. for C₁₉H₁₂Cl₂F₃N₅S, 469.0; m/z found, 470.4 [M+H]⁺. ¹H NMR (CD₃OD): δ 7.99 (d, J=8.3 Hz, 2H), 7.60-7.56 (m, 4H), 7.40 (dd, J=8.6, 7.6 Hz, 1H), 2.62 (s, 3H).

### Example 6

### N²-(2,6-Dichloro-phenyl)-5-methyl-N⁷-(6-trifluoromethyl-pyridin-3-yl)-thiazolo[5,4-d]pyrimidine-2,7-diamine

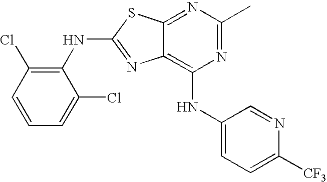

MS (ESI): mass calcd. for C₁₈H₁₁Cl₂F₃N₆S, 470.0; m/z found, 471.4 [M+H]⁺. ¹H NMR (CDCl₃): δ 9.05 (d, J=2.5 Hz, 1H), 8.64 (dd, J=8.6, 2.3 Hz, 1H), 7.96 (br s, 1H), 7.74 (d, J=8.6 Hz, 1H), 7.55-7.53 (m, 2H), 7.37 (dd, J=8.6, 7.6 Hz, 2H), 2.72 (s, 3H).

### Example 7

### N⁷-(4-tert-Butyl-phenyl)-N²-(2,6-dichloro-phenyl)-5-methyl-thiazolo[5,4-d]pyrimidine-2,7-diamine

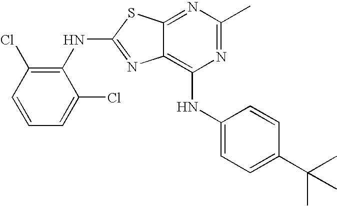

MS (ESI): mass calcd. for C₂₂H₂₁Cl₂N₅S, 457.1; m/z found, 458.4 [M+H]⁺. ¹H NMR (CDCl₃): δ 7.68 (br d, J=7.9 Hz, 2H), 7.49 (d, J=7.9 Hz, 2H), 7.40-7.38 (m, 2H), 7.30 (t, J=8.2 Hz, 1H), 2.67 (s, 3H), 1.34 (s, 9H).

### Example 8

### N⁷-(4-tert-Butyl-cyclohexyl)-N²-(2,6-dichloro-phenyl)-5-methyl-thiazolo[5,4-d]pyrimidine-2,7-diamine

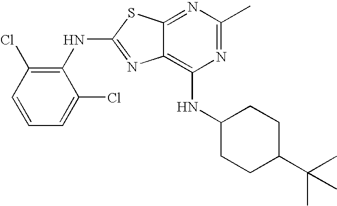

The title compound was obtained as a mixture of cis and trans isomers. MS (ESI): mass calcd. for C₂₂H₂₇Cl₂N₅S, 463.1; m/z found, 464.5 [M+H]⁺.

### Example 9

### N²-(2,6-Dichloro-phenyl)-5,N⁷-dimethyl-N⁷-(4-trifluoromethyl-phenyl)-thiazolo[5,4-d]pyrimidine-2,7-diamine

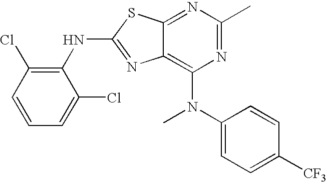

MS (ESI): mass calcd. for C₂₀H₁₄Cl₂F₃N₅S, 483.0; m/z found, 484.4 [M+H]⁺. ¹H NMR (CDCl₃): δ 7.46 (d, J=8.7 Hz, 2H), 7.31 (d, J=7.9 Hz, 2H), 7.19 (d, J=8.7 Hz, 2H), 7.15 (t, J=7.9 Hz, 1H), 3.67 (s, 3H), 2.64 (s, 3H).

### Example 10

### N²-(2,6-Dimethyl-phenyl)-N⁷-(4-trifluoromethyl-phenyl)thiazolo[5,4,d]pyrimidine-2,7-diamine

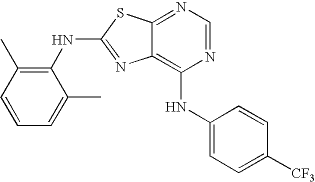

MS (ESI): mass calcd. for C₂₀H₁₆F₃N₅S, 415.1; m/z found, 416.4 [M+H]⁺. ¹H NMR (CD₃OD): δ 8.34 (s, 1H), 8.00 (d, J=8.5 Hz, 2H), 7.60 (d, J=8.5 Hz, 2H), 7.24-7.22 (m, 3H), 2.32 (s, 6H).

### Example 11

### N²-(2,6-Dimethyl-phenyl)-N⁷-(6-trifluoromethyl-pyridin-3-yl)-thiazolo[5,4-d]pyrimidine-2,7-diamine

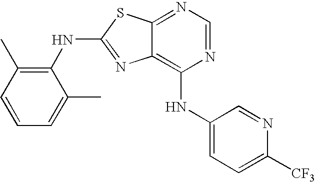

MS (ESI): mass calcd. for C₁₉H₁₅F₃N₆S, 416.1; m/z found, 417.4 [M+H]⁺. ¹H NMR (CD₃OD): δ 9.03 (br s, 1H), 8.64 (d, J=8.1 Hz, 1H), 8.38 (s, 1H), 7.76 (d, J=8.6 Hz, 1H), 7.27-7.21 (m, 3H), 2.32 (s, 6H).

### Example 12

### N⁷-(4-tert-Butyl-phenyl)-N²-(2,6-dimethyl-phenyl)-thiazolo[5,4-d]pyrimidine-2,7-diamine

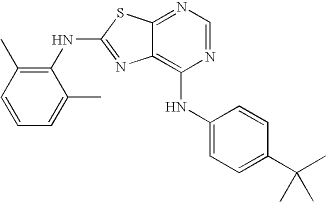

MS (ESI): mass calcd. for C₂₃H₂₅N₅S, 403.2; m/z found, 404.5 [M+H]⁺. ¹H NMR (CD₃OD): δ 8.23 (s, 1H), 7.59-7.57 (m, 2H), 7.41-7.39 (m, 2H), 7.25-7.21 (m, 3H), 2.32 (s, 6H), 1.33 (s, 9H).

### Example 13

### N⁷-(3-Chloro-4-trifluoromethyl-phenyl)-N²-(2,6-dimethyl-phenyl)-thiazolo[5,4-d]pyrimidine-2,7-diamine

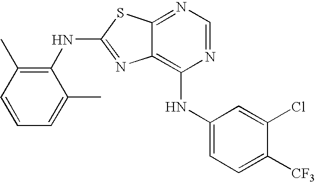

MS (ESI): mass calcd. for C₂₀H₁₅ClF₃N₅S, 449.0; m/z found, 450.4 [M+H]⁺. ¹H NMR (CD₃OD): δ 8.42 (s, 1H), 8.26 (s, 1H), 7.81-7.80 (m, 1H), 7.69 (d, J=8.8 Hz, 1H), 7.25-7.22 (m, 3H), 2.32 (s, 6H).

### Example 14

### N²-(2-Chloro-6-methyl-phenyl)-N⁷-(4-trifluoromethyl-phenyl)-thiazolo[5,4-d]pyrimidine-2,7-diamine

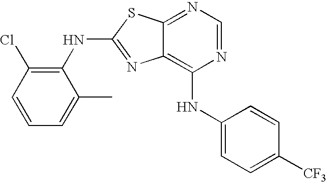

MS (ESI): mass calcd. for C₁₉H₁₃ClF₃N₅S, 435.0; m/z found, 436.4 [M+H]⁺. ¹H NMR (CDCl₃): δ 8.52 (s, 1H), 7.94 (d, J=8.5 Hz, 2H), 7.87 (br s, 1H), 7.64 (d, J=8.5 Hz, 2H), 7.42 (dd, J=7.4, 2.2 Hz, 1H), 7.33-7.29 (m, 2H), 2.41 (s, 3H).

### Example 15

### N²-(2-Chloro-6-methyl-phenyl)-N⁷-(6-trifluoromethyl-pyridin-3-yl)-thiazolo[5,4-d]pyrimidine-2,7-diamine

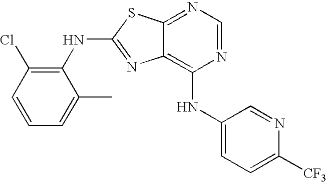

MS (ESI): mass calcd. for C₁₈H₁₂ClF₃N₆S, 436.0; m/z found, 437.4 [M+H]⁺. ¹H NMR (CDCl₃): δ 8.88 (d, J=2.5 Hz, 1H), 8.75 (dd, J=8.5, 2.5 Hz, 1H), 8.51 (s, 1H), 7.78 (br s, 1H), 7.71 (d, J=8.5 Hz, 1H), 7.43-7.41 (m, 1H), 7.33-7.30 (m, 2H), 2.41 (s, 3H).

### Example 16

### N²-(2-Chloro-phenyl)-N⁷-(4-trifluoromethyl-phenyl)-thiazolo[5,4-d]pyrimidine-2,7-diamine

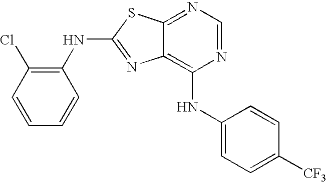

MS (ESI): mass calcd. for C₁₈H₁₁ClF₃N₅S, 421.0; m/z found, 422.4 [M+H]⁺. ¹H NMR (CDCl₃): δ 8.57 (s, 1H), 8.25 (dd, J=8.3, 1.5 Hz, 1H), 7.99 (d, J=8.3 Hz, 1H), 7.71-7.67 (m, 4H), 7.53 (dd, J=8.1, 1.5 Hz, 1H), 7.46-7.41 (m, 1H), 7.20-7.15 (m, 1H).

### Example 17

### N²-o-Tolyl-N⁷-(4-trifluoromethyl-phenyl)-thiazolo[5,4-d]pyrimidine-2,7-diamine

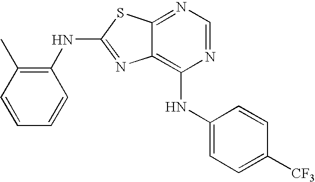

MS (ESI): mass calcd. for C₁₉H₁₄F₃N₅S, 401.1; m/z found, 402.5 [M+H]⁺. ¹H NMR (CDCl₃): δ 8.49 (s, 1H), 7.93 (d, J=8.7 Hz, 2H), 7.64-7.61 (m, 4H), 7.33-7.30 (m, 2H), 7.25-7.23 (m, 1H), 7.09 (br s, 1H), 2.37 (s, 3H).

### Example 18

### N²-(2-Chloro-6-trifluoromethyl-phenyl)-N⁷-(4-trifluoromethyl-phenyl)-thiazolo[5,4-d]pyrimidine-2,7-diamine

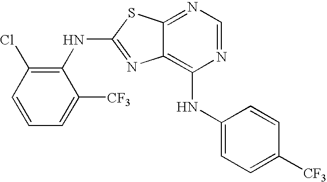

MS (ESI): mass calcd. for C₁₉H₁₀F₆N₅S, 489.0; m/z found, 490.4 [M+H]⁺. ¹H NMR (CDCl₃): δ 8.53 (s, 1H), 7.91-7.89 (m, 3H), 7.82 (d, J=7.9 Hz, 1H), 7.77 (d, J=7.9 Hz, 1H), 7.63 (d, J=8.5 Hz, 2H), 7.57 (t, J=7.9 Hz, 1H).

### Example 19

### N²-(2-Chloro-6-trifluoromethyl-phenyl)-N⁷-(6-trifluoromethyl-pyridin-3-yl)-thiazolo[5,4-d]pyrimidine-2,7-diamine

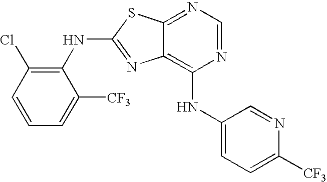

MS (ESI): mass calcd. for C₁₈H₉F₆N₆S, 490.0; m/z found, 491.4 [M+H]⁺. ¹H NMR (CDCl₃): δ 8.86 (d, J=2.5 Hz, 1H), 8.75 (dd, J=8.5, 2.5 Hz, 1H), 8.53 (s, 1H), 7.83 (d, J=7.9 Hz, 1H), 7.77-7.75 (m, 2H), 7.71 (d, J=8.5 Hz, 1H), 7.57 (t, J=7.9 Hz, 1H).

### Example 20

### N²-Phenyl-N⁷-(4-trifluoromethyl-phenyl)-thiazolo[5,4-d]pyrimidine-2,7-diamine

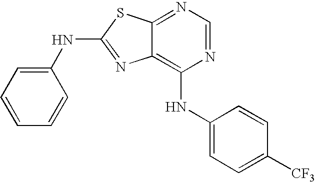

MS (ESI): mass calcd. for C₁₈H₁₂F₃N₅S, 387.1; m/z found, 388.4 [M+H]⁺. ¹H NMR (CD₃OD): δ 8.36 (s, 1H), 8.06 (d, J=8.5 Hz, 2H), 7.77-7.75 (m, 2H), 7.64-7.62 (m, 2H), 7.41-7.39 (m, 2H), 7.13-7.11 (m, 1H).

### Example 21

### N²-Phenyl-N⁷-(6-trifluoromethyl-pyridin-3-yl)-thiazolo[5,4-d]pyrimidine-2,7-diamine

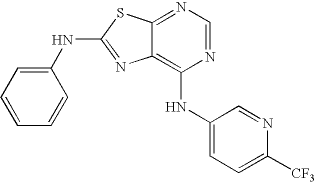

MS (ESI): mass calcd. for C₁₇H₁₁F₃N₆S, 388.1; m/z found, 389.4 [M+H]⁺. ¹H NMR (CD₃OD): δ 9.13 (d, J=2.5 Hz, 1H), 8.67 (dd, J=8.8, 2.5 Hz, 1H), 8.40 (s, 1H), 7.78 (d, J=8.5 Hz, 3H), 7.41-7.38 (m, 2H), 7.13-7.09 (m, 1H).

### Example 22

### N²,N⁷-Bis-(4-trifluoromethyl-phenyl)-thiazolo[5,4-d]pyrimidine-2,7-diamine

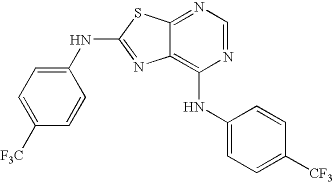

MS (ESI): mass calcd. for C₁₉H₁₁F₆N₅S, 455.1; m/z found, 456.4 [M+H]⁺. ¹H NMR (CDCl₃): δ 8.59 (s, 1H), 8.50 (br s, 1H), 7.91 (d, J=8.5 Hz, 2H), 7.70 (app d, J=8.5 Hz, 4H), 7.63 (d, J=8.5 Hz, 2H).

### Example 23

### N²-(2,6-Dichloro-phenyl)-5,N²-dimethyl-N⁷-(4-trifluoromethyl-phenyl)-thiazolo[5,4-d]pyrimidine-2,7-diamine

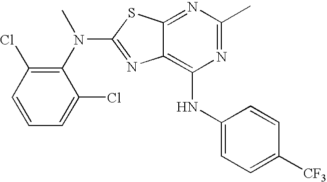

MS (ESI): mass calcd. for C₂₀H₁₄Cl₂F₃N₅S, 483.0; m/z found, 484.4 [M+H]⁺. ¹H NMR (CDCl₃): δ 7.88 (br s, 2H), 7.63 (br d, J=8.6 Hz, 2H), 7.52 (d, J=7.8 Hz, 2H), 7.40 (m, 1H), 3.49 (s, 3H), 2.70 (s, 3H).

### Example 24

### N²-(3,5-Dimethyl-isoxazol-4-yl)-N⁷-(6-trifluoromethyl-pyridin-3-yl)-thiazolo[5,4-d]pyrimidine-2,7-diamine

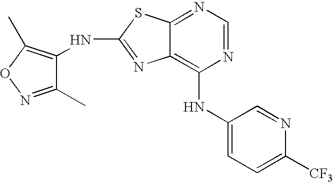

MS (ESI): mass calcd. for C₁₆H₁₂F₃N₇OS, 407.1; m/z found, 408.4 [M+H]⁺. ¹H NMR (CD₃OD): δ 9.09 (d, J=2.3 Hz, 1H), 8.66 (dd, J=8.3, 2.3 Hz, 1H), 8.42 (s, 1H), 7.78 (d, J=8.8 Hz, 1H), 2.41 (s, 3H), 2.25 (s, 3H).

### Example 25

### N²-(3,5-Dimethyl-isoxazol-4-yl)-N⁷-(4-trifluoromethyl-phenyl)-thiazolo[5,4-d]pyrimidine-2,7-diamine

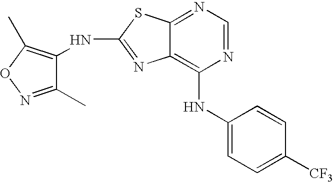

MS (ESI): mass calcd. for C₁₇H₁₃F₃N₆OS, 406.1; m/z found, 407.3 [M+H]⁺. ¹H NMR (CD₃OD): δ 8.37 (s, 1H), 8.03 (d, J=8.6 Hz, 2H), 7.60 (d, J=8.6 Hz, 2H), 2.41 (s, 3H), 2.24 (s, 3H).

### Example 26

### 5-Methyl-N²-(5-methyl-3-phenyl-isoxazol-4-yl)-N⁷-(6-trifluoromethyl-pyridin-3-yl)-thiazolo[5,4-d]pyrimidine-2,7-diamine

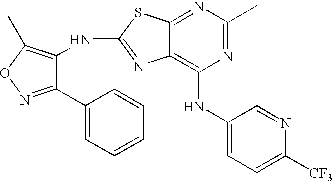

MS (ESI): mass calcd. for C₂₂H₁₆F₃N₇OS, 483.1; m/z found, 484.5 [M+H]⁺. ¹H NMR (CD₃OD): δ 9.10 (d, J=2.5 Hz, 1H), 8.61 (dd, J=8.5, 2.5 Hz, 1H), 7.77-7.39 (m, 3H), 7.45-7.43 (m, 3H), 2.58 (s, 3H), 2.49 (s, 3H).

### Example 27

### 5-Methyl-N²-(5-methyl-3-phenyl-isoxazol-4-yl)-N⁷-(4-trifluoromethyl-phenyl)-thiazolo[5,4-d]pyrimidine-2,7-diamine

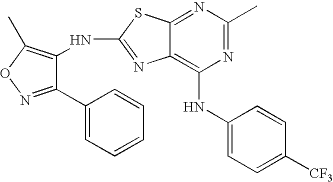

MS (ESI): mass calcd. for C₂₃H₁₇F₃N₆OS, 482.1; m/z found, 483.5 [M+H]⁺. ¹H NMR (CD₃OD): δ 8.01 (d, J=8.5 Hz, 2H), 7.76-7.74 (m, 2H), 7.61 (d, J=8.5 Hz, 2H), 7.45-7.43 (m, 3H), 2.56 (s, 3H), 2.49 (s, 3H).

### Example 28

### N²-(2,6-Dimethyl-phenyl)-N⁷-(4-trifluoromethoxy-phenyl)-thiazolo[5,4-d]pyrimidine-2,7-diamine

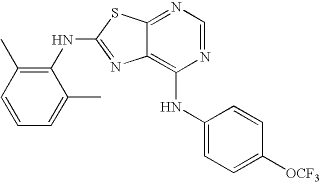

MS (ESI): mass calcd. for C₂₀H₁₆F₃N₅OS, 431.10; m/z found, 432.5 [M+H]⁺. ¹H NMR (CD₃OD): δ 8.28 (s, 1H), 7.85 (d, J=9.3 Hz, 2H), 7.25-7.20 (m, 5H), 2.32 (s, 6H).

### Example 29

### 5-[2-(2,6-Dimethyl-phenylamino)-thiazolo[5,4-d]pyrimidin-7-ylamino]-pyridine-2-carbonitrile

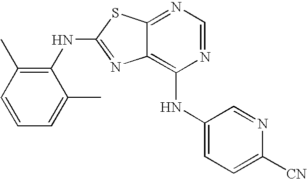

MS (ESI): mass calcd. for C₁₉H₁₅N₇S, 373.11; m/z found, 374.4 [M+H]⁺. ¹H NMR (CDCl₃): δ 9.01-8.98 (m, 1H), 8.72-8.68 (m, 1H), 8.56 (s, 1H), 7.72 (d, J=8.5 Hz, 1H), 7.31 (dd, J=8.2 Hz, 6.8, 1H), 7.22 (d, J=7.4 Hz, 2H), 2.33 (s, 6H).

### Example 30

### N⁷-(3-Chloro-4-trifluoromethyl-phenyl)-N²-(2,6-dichloro-phenyl)-5-methyl-thiazolo[5,4-d]pyrimidine-2,7-diamine

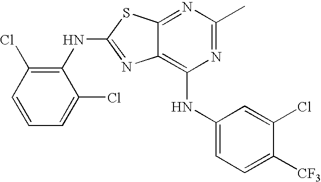

MS (ESI): mass calcd. for C₁₉H₁₁Cl₃F₃N₅S, 502.98; m/z found, 506.2 [M+H]⁺. ¹H NMR (CDCl₃): δ 8.11 (d, J=1.6 Hz, 1H), 7.76-7.72 (m, 1H), 7.64 (d, J=8.7 Hz, 1H), 7.49 (d, J=8.2 Hz, 2H), 7.34-7.31 (m, 1H), 2.70 (s, 3H).

### Example 31

### 4-[2-(2,6-Dimethyl-phenylamino)-thiazolo[5,4-d]pyrimidin-7-ylamino]-benzonitrile

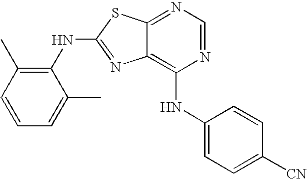

MS (ESI): mass calcd. for C₂₀H₁₆N₆S, 372.12; m/z found, 373.5 [M+H]⁺. ¹H NMR (CD₃OD): δ 8.36 (s, 1H), 8.03 (d, J=8.6 Hz, 2H), 7.65 (d, J=8.8 Hz, 2H), 7.26-7.21 (m, 3H), 2.32 (s, 6H).

### Example 32

### 2-{4-[2-(2,6-Dimethyl-phenylamino)-thiazolo[5,4-d]pyrimidin-7-ylamino]-phenyl}-2-methyl-propionitrile

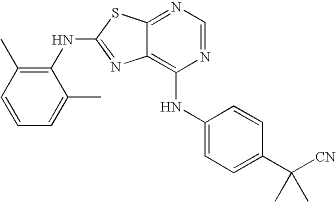

MS (ESI): mass calcd. for C₂₃H₂₂N₆S, 414.16; m/z found, 415.5 [M+H]⁺. ¹H NMR (CD₃OD): δ 8.27 (s, 1H), 7.80 (d, J=8.7 Hz, 2H), 7.50-7.41 (m, 2H), 7.24-7.21 (m, 3H), 2.32 (s, 6H), 1.71 (s, 6H).

### Example 33

### N²-(2,6-Dimethyl-phenyl)-N⁷-(4-methoxy-phenyl)-thiazolo[5,4-d]pyrimidine-2,7-diamine

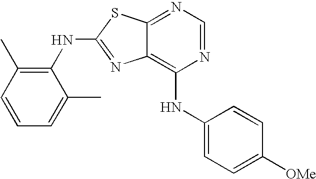

MS (ESI): mass calcd. for C₂₀H₁₉N₅OS, 377.13; m/z found, 378.4 [M+H]⁺. ¹H NMR (CDCl₃): δ 8.39 (s, 1H), 7.64-7.61 (m, 2H), 7.27-7.24 (m, 1H), 7.21 (d, J=7.4 Hz, 2H), 6.96-6.92 (m, 2H), 3.82 (s, 3H), 2.34 (s, 6H).

### Example 34

### N⁷-(3,4-Dichloro-phenyl)-N²-(2,6-dimethyl-phenyl)-thiazolo[5,4-d]pyrimidine-2,7-diamine

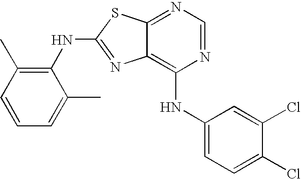

MS (ESI): mass calcd. for C₁₉H₁₅Cl₂N₅S, 415.04; m/z found, 416.2 [M+H]⁺. ¹H NMR (CD₃OD): δ 8.31 (s, 1H), 8.13 (d, J=2.5 Hz, 1H), 7.59-7.57 (m, 1H), 7.37 (d, 8.8, 1H), 7.23-7.18 (m, 3H), 2.31 (s, 6H).

### Example 35

### N²-(2,6-Dimethyl-phenyl)-N⁷-p-tolyl-thiazolo[5,4-d]pyrimidine-2,7-diamine

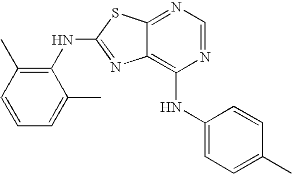

MS (ESI): mass calcd. for C₂₀H₁₉N₅S, 361.14; m/z found, 362.3 [M+H]⁺. ¹H NMR (CDCl₃): δ 8.42 (s, 1H), 7.64-7.61 (m, 2H), 7.28-7.24 (m, 1H), 7.22-7.18 (m, 4H), 2.40-2.34 (m, 9H).

### Example 36

### N²-(2,6-Dimethyl-phenyl)-N⁷-(2-trifluoromethyl-phenyl)-thiazolo[5,4-d]pyrimidine-2,7-diamine

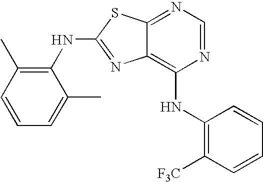

MS (ESI): mass calcd. for C₂₀H₁₆F₃N₅S, 415.11; m/z found, 416.3 [M+H]⁺. ¹H NMR (CDCl₃): δ 8.41 (s, 1H), 8.34 (d, J=7.9 Hz, 1H), 7.92 (s, 1H), 7.70 (d, J=7.9 Hz, 1H), 7.62 (t, J=7.9 Hz, 1H), 7.29-7.24 (m, 1H), 7.21 (d, J=7.9 Hz, 2H), 2.36 (s, 6H).

### Example 37

### 4-[2-(2,6-Dimethyl-phenylamino)-thiazolo[5,4-d]pyrimidin-7-ylamino]-benzoic acid methyl ester

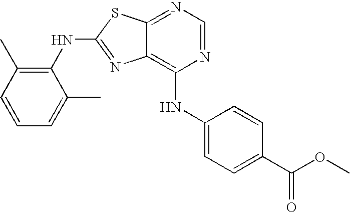

MS (ESI): mass calcd. for C₂₁H₁₉N₅O₂S, 405.13; m/z found, 406.3 [M+H]⁺. ¹H NMR (CDCl₃): δ 8.48 (s, 1H), 8.07-8.04 (m, 2H), 7.91-7.88 (m, 2H), 7.70 (s, 1H), 7.29-7.88 (m, 1H), 7.20 (d, J=7.4 Hz, 2H), 6.89 (s, 1H), 3.91 (s, 3H), 2.34 (s, 6H).

### Example 38

### 4-{[2-(2,6-Dimethyl-phenylamino)-thiazolo[5,4-d]pyrimidin-7-ylamino]-methyl}-2-methoxy-phenol

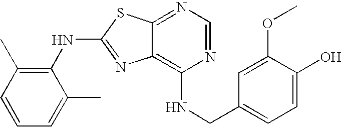

MS (ESI): mass calcd. for C₂₁H₂₁N₅O₂S, 407.14; m/z found, 408.4 [M+H]⁺. ¹H NMR (CDCl₃): δ 8.36 (s, 1H), 7.28-7.24 (m, 1H), 7.18 (d, J=7.6 Hz, 2H), 6.98-6.92 (m, 1H), 6.91-6.86 (m, 2H), 4.78-4.76 (m, 2H), 3.89 (s, 3H), 2.31 (s, 6H).

### Example 39

### N⁷-(3,4-Dichloro-benzyl)-N²-(2,6-dimethyl-phenyl)-thiazolo[5,4-d]pyrimidine-2,7-diamine

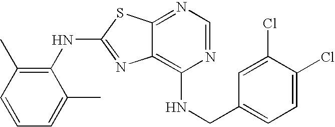

MS (ESI): mass calcd. for C₂₀H₁₇Cl₂N₅S, 429.06; m/z found, 430.3 [M+H]⁺. ¹H NMR (CDCl₃): δ 8.32 (s, 1H), 7.48-7.46 (m, 1H), 7.41 (d, J=8.2 Hz, 1H), 7.28-7.25 (m, 1H), 7.25-7.21 (m, 1H), 7.18 (d, J=7.6 Hz, 2H), 4.78-4.76 (m, 2H), 2.32 (s, 6H).

### Example 40

### N²-(2,6-Dimethyl-phenyl)-N⁷-(4-trifluoromethylsulfanyl-phenyl)-thiazolo[5,4-d]pyrimidine-2,7-diamine

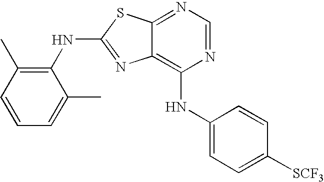

MS (ESI): mass calcd. for C₂₀H₁₆F₃N₅S₂, 447.08; m/z found, 448.3 [M+H]⁺. ¹H NMR (CDCl₃): δ 8.48 (s, 1H), 7.93-7.89 (m, 2H), 7.75 (s, 1H), 7.68-7.64 (m, 2H), 7.22 (d, J=7.7 Hz, 2H), 2.34 (s, 6H).

### Example 41

### N²-(2,6-Dimethyl-phenyl)-N⁷-indan-2-yl-thiazolo[5,4-d]pyrimidine-2,7-diamine

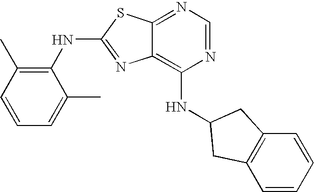

MS (ESI): mass calcd. for C₂₂H₂₁N₅S, 387.15; m/z found, 388.4 [M+H]⁺. ¹H NMR ((CD₃)₂CO): δ 8.33 (s, 1H), 7.28-7.08 (m, 7H), 5.10-5.09 (m, 1H), 3.41-3.35 (dd, J=15.6, 7.4 Hz, 2H), 3.11-3.05 (dd, J=15.9, 6.3 Hz, 2H), 2.30 (s, 6H).

### Example 42

### N²-(2,6-Dimethyl-phenyl)-N⁷-(3-trifluoromethyl-phenyl)-thiazolo[5,4-d]pyrimidine-2,7-diamine

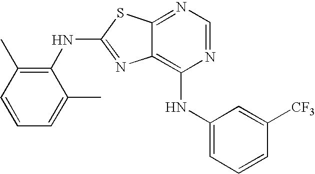

MS (ESI): mass calcd. for C₂₀H₁₆F₃N₅S, 415.11; m/z found, 416.3 [M+H]⁺. ¹H NMR (CD₃OD): δ 8.33 (s, 1H), 8.25 (s, 1H), 7.98 (d, J=8.2 Hz, 1H), 7.49 (t, J=8.2 Hz, 1H), 7.32 (d, J=7.6 Hz, 1H), 7.25-7.21 (m, 3H), 2.32 (s, 6H).

### Example 43

### N⁷-Benzyl-N²-(2,6-dimethyl-phenyl)-thiazolo[5,4-d]pyrimidine-2,7-diamine

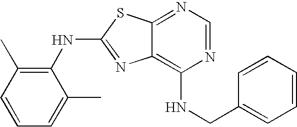

MS (ESI): mass calcd. for C₂₀H₁₉N₅S, 361.14; m/z found, 362.3 [M+H]⁺. ¹H NMR (CDCl₃): δ 8.42 (s, 1H), 7.44-7.24 (m, 7H), 7.20 (d, J=7.6 Hz, 2H), 6.69 (s, 1H), 4.90 (s, 2H), 2.30 (s, 6H).

### Example 44

### 4-[2-(2,6-Dimethyl-phenylamino)-thiazolo[5,4-d]pyrimidin-7-ylamino]-benzenesulfonamide

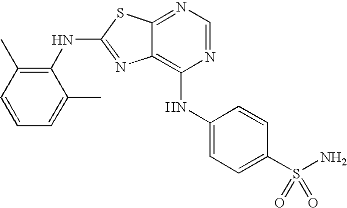

MS (ESI): mass calcd. for C₁₉H₁₈N₆O₂S₂, 426.0; m/z found, 427.4 [M+H]⁺.

### Example 45

### N²-(2,6-Dimethyl-phenyl)-N⁷-(4-ethyl-phenyl)-thiazolo[5,4-d]pyrimidine-2,7-diamine

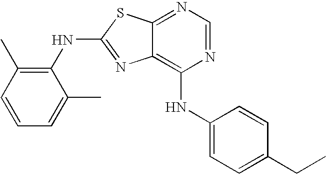

MS (ESI): mass calcd. for C₂₁H₂₁N₅S, 375.15; m/z found, 376.1 [M+H]⁺.

### Example 46

### N²-(2,6-Dimethyl-phenyl)-N⁷-(4-isopropyl-phenyl)-thiazolo[5,4-d]pyrimidine-2,7-diamine

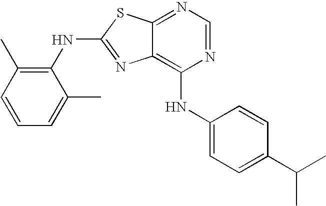

MS (ESI): mass calcd. for C₂₂H₂₃N₅S, 389.17; m/z found, 390.1 [M+H]⁺.

### Example 47

### N²-(2,6-Dimethyl-phenyl)-N⁷-(5-methyl-furan-2-ylmethyl)-thiazolo[5,4-d]pyrimidine-2,7-diamine

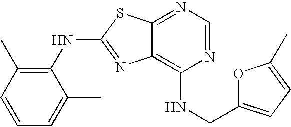

MS (ESI): mass calcd. for C₁₉H₁₉N₅OS, 365.13; m/z found, 366.1 [M+H]⁺.

### Example 48

### 4-Methyl-3-[7-(4-trifluoromethyl-phenylamino)-thiazolo[5,4-d]pyrimidin-2-ylamino]-thiophene-2-carboxylic acid methyl ester

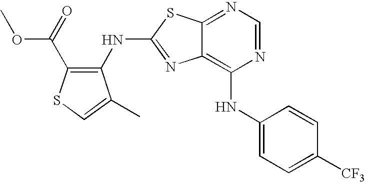

MS (ESI): mass calcd. for C₁₉H₁₄F₃N₅O₂S₂, 465.0; m/z found, 466.4 [M+H]⁺. ¹H NMR (CD₃OD): δ 8.37 (s, 1H), 8.01 (br d, J=7.4 Hz, 2H), 7.61 (br d, J=7.4 Hz, 2H), 7.52 (s, 1H), 3.81 (s, 3H), 2.21 (s, 3H).

### Example 49

### 4-Methyl-3-[7-(6-trifluoromethyl-pyridin-3-ylamino)-thiazolo[5,4-d]pyrimidin-2-ylamino]-thiophene-2-carboxylic acid methyl ester

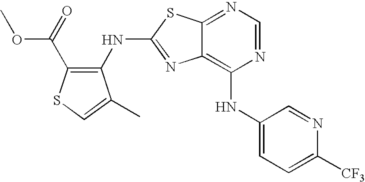

MS (ESI): mass calcd. for C₁₈H₁₃F₃N₆O₂S₂, 466.0; m/z found, 467.4 [M+H]⁺. ¹H NMR (CD₃OD): δ 8.96 (d, J=2.5 Hz, 1H), 8.55-8.52 (m, 1H), 8.31 (s, 1H), 7.67 (d, J=8.6 Hz, 1H), 7.42-4.40 (m, 1H), 3.70 (s, 3H), 2.11 (s, 3H).

### Example 50

### N⁷-(3-Chloro-4-trifluoromethyl-phenyl)-N²-(3,5-dimethyl-isoxazol-4-yl)thiazolo[5,4d]pyrimidine-2,7-diamine

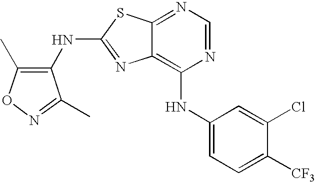

MS (ESI): mass calcd. for C₁₇H₁₂ClF₃N₆OS, 440.0; m/z found, 441.4 [M+H]⁺. ¹H NMR (CD₃OD): δ 8.42 (s, 1H), 8.33 (br d, J=2.2 Hz, 1H), 7.88-7.86 (m, 1H), 7.69 (d, J=8.8 Hz, 1H), 2.41 (s, 3H), 2.24 (s, 3H).

### Example 51

### N⁷-(4-tert-Butyl-phenyl)-N²-(3,5-dimethyl-isoxazol-4-yl)-thiazolo[5,4-d]pyrimidine-2,7-diamine

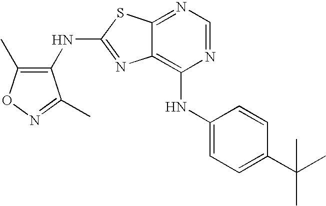

MS (ESI): mass calcd. for C₂₀H₂₂N₆OS, 394.1; m/z found, 395.5 [M+H]⁺. ¹H NMR (CD₃OD): δ 8.24 (s, 1H), 7.60 (d, J=8.8 Hz, 2H), 7.41 (d, J=8.8 Hz, 2H), 2.40 (s, 3H), 2.24 (s, 3H), 1.34 (s, 9H).

### Example 52

### N²-(2,6-Dichloro-phenyl)-5-methylsulfanyl-N⁷-(4-trifluoromethyl-phenyl)-thiazolo[5,4-d]pyrimidine-2,7-diamine

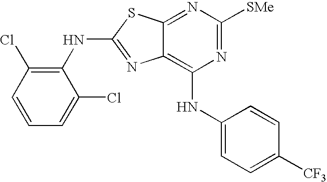

MS (ESI): mass calcd. for C₁₉H₁₂Cl₂F₃N₅S₂, 500.9; m/z found, 502.3 [M+H]⁺. ¹H NMR (CD₃OD): δ 7.96 (d, J=8.5 Hz, 2H), 7.59-7.57 (m, 4H), 7.40 (t, J=8.5 Hz, 1H), 2.57 (s, 3H).

### Example 53

### N²-(2,6-Dichloro-phenyl)-5-methanesulfonyl-N⁷-(4-trifluoromethyl-phenyl)-thiazolo[5,4-d]pyrimidine-2,7-diamine

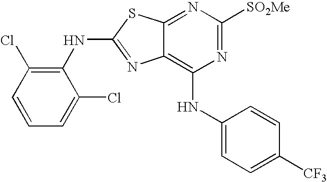

To a solution of N²-(2,6-dichloro-phenyl)-5-methylsulfanyl-N⁷-(4-trifluoromethyl-phenyl)-thiazolo[5,4-d]pyrimidine-2,7-diamine (Example 52; 724 mg, 1.45 mmol) in CH₂Cl₂ (20 mL) was added m-CPBA (77%; 700 mg, 2.2 mmol). After 5 h, the mixture was diluted with satd. aq. NaHCO₃ (50 mL) and extracted with CH₂Cl₂ (3×). The combined organic layers were dried and concentrated, and the residue was purified directly by FCC to afford a colorless solid (350 mg, 45%). MS (ESI): mass calcd. for C₁₉H₁₂Cl₂F₃N₅O₂S₂, 532.9; m/z found, 534.3 [M+H]⁺. ¹H NMR (CD₃OD): δ 8.01 (d, J=8.5 Hz, 2H), 7.65-7.60 (m, 4H), 7.46-7.42 (m, 1H), 3.32 (s, 3H).

Alternative Preparation: To a solution of N²-(2,6-dichloro-phenyl)-5-methylsulfanyl-N⁷-(4-trifluoromethyl-phenyl)-thiazolo[5,4-d]pyrimidine-2,7-diamine (Example 52; 2.3 g, 4.7 mmol) in THF (16 mL) and MeOH (16 mL) was added OXONE™ (12.6 g, 20.6 mmol) in H₂O (16 mL). After 30 h, the mixture was concentrated and the crude residue was partitioned between saturated aqueous NaHCO₃ (50 mL) and EtOAc (50 mL). The aqueous layer was extracted with EtOAc (3×75 mL). The combined organic layers were dried (MgSO₄), filtered, and concentrated. The residue was purified directly by FCC to afford a colorless solid (1.7 g, 69%).

### Example 54

### N²-(2,6-Dichloro-phenyl)-5-piperidin-1-yl-N⁷-(4-trifluoromethyl-phenyl)-thiazolo[5,4-d]pyrimidine-2,7-diamine

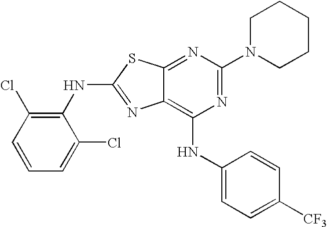

A mixture of N²-(2,6-dichloro-phenyl)-5-methanesulfonyl-N⁷-(4-trifluoromethyl-phenyl)-thiazolo[5,4-d]pyrimidine-2,7-diamine (Example 53; 82 mg, 0.15 mmol) and piperidine (20 mg, 0.23 mmol) in n-butanol or t-amyl alcohol (2 mL) were heated to 130° C. in a sealed tube. After 24 h, the reaction mixture was cooled and purified directly by preparative reverse-phase HPLC to afford a colorless solid (50 mg, 60%). MS (ESI): mass calcd. for C₂₃H₁₉Cl₂F₃N₆S, 538.1; m/z found, 539.4 [M+H]⁺. ¹H NMR (CD₃OD): δ 7.87 (d, J=8.5 Hz, 2H), 7.62 (d, J=8.5 Hz, 2H), 7.58 (d, J=8.2 Hz, 2H), 7.39 (t, J=8.2 Hz, 1H), 3.76 (m, 4H), 1.73-1.67 (m, 6H).

### Example 55

### N²-(2,6-Dichloro-phenyl)-5-methoxy-N⁷-(4-trifluoromethyl-phenyl)-thiazolo[5,4d]pyrimidine-2,7-diamine

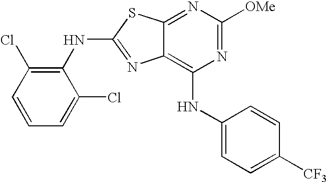

A solution of N²-(2,6-dichloro-phenyl)-5-methanesulfonyl-N⁷-(4-trifluoromethyl-phenyl)-thiazolo[5,4-d]pyrimidine-2,7-diamine (Example 53; 87 mg, 0.16 mmol) and NaOMe (44 mg, 0.82 mmol) in MeOH (1.5 mL) was heated at 80° C. in a sealed tube. After 12 h, the mixture was cooled, acidified with HOAc (3 drops), and directly purified using preparative reverse-phase HPLC to afford the title compound (30 mg, 38%). MS (ESI): mass calcd. for C₁₉H₁₂Cl₂F₃N₅OS, 485.0; m/z found, 486.3 [M+H]⁺.

Alternative Preparation: To a solution of N²-(2,6-dichloro-phenyl)-5-methanesulfonyl-N⁷-(4-trifluoromethyl-phenyl)-thiazolo[5,4-d]pyrimidine-2,7-diamine (Example 53; 25 mg, 0.05 mmol) and MeOH (0.5 mL) was added NH₃ (7 N in MeOH; 5 mL). The solution was heated to 80° C. in a sealed tube. After 12 h, the mixture was cooled and directly purified using preparative reverse-phase HPLC to afford the title compound (2 mg, 9%). MS (ESI): mass calcd. for C₁₉H₁₂Cl₂F₃N₅OS, 485.0; m/z found, 486.0 [M+H]⁺. ¹H NMR (CDCl₃): δ 7.91 (d, J=8.45 Hz, 2H), 7.67 (s, 1H), 7.62 (d, J=8.54 Hz, 2H), 7.50 (d, J=8.15 Hz, 2H), 7.33-7.29 (m, 1H), 4.03 (s, 3H).

### Example 56

### N²-(2,6-Dichloro-phenyl)-N⁵,N⁵-dimethyl-N⁷-(4-trifluoromethyl-phenyl)-thiazolo[5,4-d]pyrimidine-2,5,7-triamine

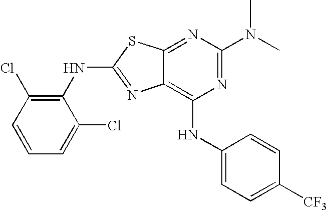

The title compound was prepared using methods analogous to those described in the preceding examples. MS (ESI): mass calcd. for C₂₀H₁₅Cl₂F₃N₆S, 498.0; m/z found, 499.0 [M+H]⁺. ¹H NMR (CD₃OD): δ 7.85 (d, J=8.59 Hz, 2H), 7.55 (d, J=8.70 Hz, 2H), 7.51 (d, J=8.15 Hz, 2H), 7.35-7.28 (m, 1H), 3.14 (s, 6H).

### Example 57

### 5-Azepan-1-yl-N²-(2,6-dichloro-phenyl)-N⁷-(4-trifluoromethyl-phenyl)-thiazolo[5,4-d]pyrimidine-2,7-diamine

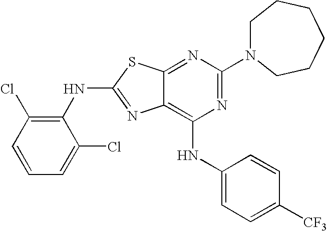

The title compound may be prepared using methods analogous to those described in the preceding examples.

The compounds in Examples 58-59 were prepared using methods analogous to those described in the preceding examples.

### Example 58

### N²-(2,6-Dichloro-phenyl)-5-pyrrolidin-1-yl-N⁷-(4-trifluoromethyl-phenyl)-thiazolo[5,4-d]pyrimidine-2,7-diamine

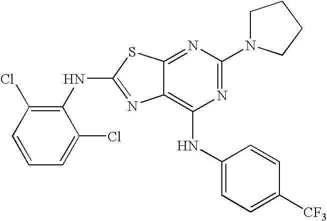

MS (ESI): mass calcd. for C₂₂H₁₇Cl₂F₃N₆S, 524.1; m/z found, 525.1 [M+H]⁺. ¹H NMR (CD₃OD): δ 7.94 (d, J=8.59 Hz, 2H), 7.58 (d, J=8.69 Hz, 2H), 7.54 (d, J=8.07 Hz, 2H), 7.37-7.32 (m, 1H), 3.60-3.54 (m, 4H), 2.06-1.99 (m, 4H).

### Example 59

### 5-Azetidin-1-yl-N²-(2,6-dichloro-phenyl)-N⁷-(4-trifluoromethyl-phenyl)-thiazolo[5,4-d]pyrimidine-2,7-diamine

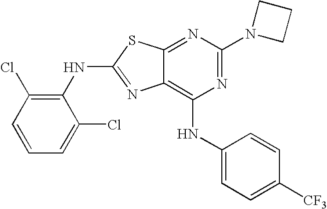

MS (ESI): mass calcd. for C₂₁H₁₅Cl₂F₃N₆S, 510.0; m/z found, 511.0 [M+H]⁺. ¹H NMR (CD₃OD): δ 7.89 (d, J=8.54 Hz, 2H), 7.58-7.48 (m, 4H), 7.35-7.30 (m, 1H), 4.16 (t, J=7.56, 7.56 Hz, 4H), 2.43-2.34 (m, 2H).

The compounds in Examples 60-61 may be prepared using methods analogous to those described in the preceding examples.

### Example 60

### N²-(2,6-Bis-methanesulfonyl-phenyl)-5-methyl-N⁷-(4-trifluoromethyl-phenyl)-thiazolo[5,4-d]pyrimidine-2,7-diamine

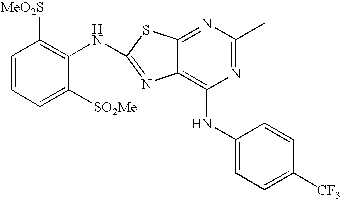

### Example 61

### N²-(2,6-Dichloro-phenyl)-N⁵-(2-methoxy-ethyl)-N⁷-(4-trifluoromethyl-phenyl)-thiazolo[5,4-d]pyrimidine-2,5,7-triamine

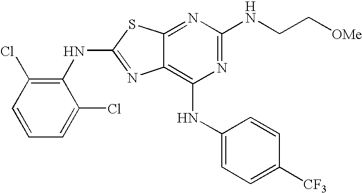

The compounds in Examples 62-64 were prepared using methods analogous to those described in the preceding examples.

### Example 62

### N⁵-Cyclopropylmethyl-N²-(2,6-dichloro-phenyl)-N⁷-(4-trifluoromethyl-phenyl)-thiazolo[5,4-d]pyrimidine-2,5,7-triamine

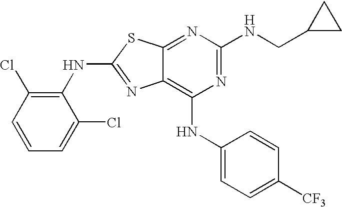

MS (ESI): mass calcd. for C₂₂H₁₇Cl₂F₃N₆S, 524.1; m/z found, 525.1 [M+H]⁺. ¹H NMR ((CD₃)₂SO) δ 9.79-9.67 (m, 1H), 9.31-9.13 (m, 1H), 8.10 (d, J=8.70 Hz, 2H), 7.64-7.56 (m, 4H), 7.41-7.36 (m, 1H), 3.14 (d, J=6.71 Hz, 2H), 1.12-1.02 (m, 1H), 0.48-0.36 (m, 2H), 0.24-0.19 (m, 2H).

### Example 63

### N²-(2,6-Dichloro-phenyl)-N⁵-(2-methoxy-ethyl)-N⁵-methyl-N⁷-(4-trifluoromethyl-phenyl)-thiazolo[5,4-d]pyrimidine-2,5,7-triamine

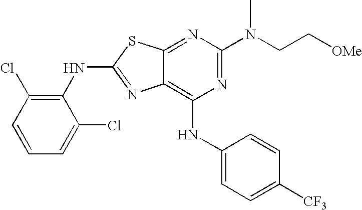

MS (ESI): mass calcd. for C₂₂H₁₉Cl₂F₃N₆OS, 542.1; m/z found, 543.1 [M+H]⁺. ¹H NMR (CD₃OD): δ 7.86-7.82 (m, 2H), 7.58-7.46 (m, 4H), 7.34-7.29 (m, 1H), 4.79-4.77 (m, 3H), 3.78-3.73 (m, 2H), 3.60-3.53 (m, 2H), 3.16 (s, 3H).

### Example 64

### N²-(2,6-Dichloro-phenyl)-5-morpholin-4-yl-N⁷-(4-trifluoromethyl-phenyl)-thiazolo[5,4-d]pyrimidine-2,7-diamine

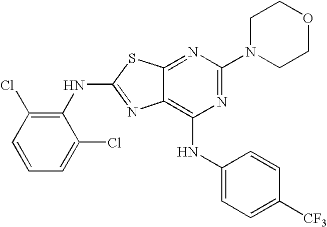

MS (ESI): mass calcd. for C₂₂H₁₇Cl₂F₃N₆OS, 540.1; m/z found, 541.1 [M+H]⁺. ¹H NMR ((CD₃)₂SO): δ 9.84 (s, 1H), 9.34 (s, 1H), 7.99 (d, J=8.61 Hz, 2H), 7.66-7.55 (m, 4H), 7.39 (t, J=8.15, 8.15 Hz, 1H), 3.71-3.56 (m, 8H).

The compounds in Examples 65-68 may be prepared using methods analogous to those described in the preceding examples.

### Example 65

### N²-(2,6-Dichloro-phenyl)-N⁷-(5-trifluoromethyl-pyridin-2-yl)-thiazolo[5,4-d]pyrimidine-2,7-diamine

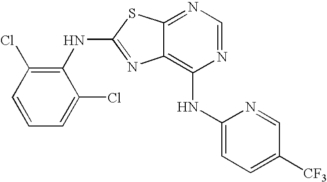

### Example 66

### N²-(2,6-Dichloro-phenyl)-5-methyl-N⁷-(5-trifluoromethyl-pyridin-2-yl)-thiazolo[5,4-d]pyrimidine-2,7-diamine

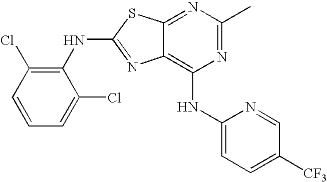

### Example 67

### N²-(2,6-Dichloro-phenyl)-5-phenoxy-N⁷-(4-trifluoromethyl-phenyl)-thiazolo[5,4-d]pyrimidine-2,7-diamine

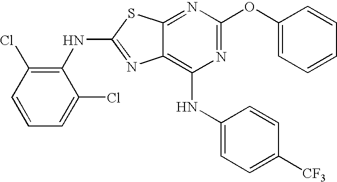

### Example 68

### N²-(2,6-Dichloro-phenyl)-N⁵-phenyl-N⁷-(4-trifluoromethyl-phenyl)-thiazolo[5,4-d]pyrimidine-2,5,7-triamine

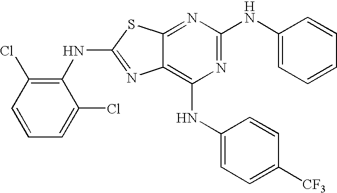

### Example 69

### N²-(2,6-Dichloro-phenyl)-5-(4-isopropyl-piperazin-1-yl)-N⁷-(4-trifluoromethyl-phenyl)-thiazolo[5,4-d]pyrimidine-2,7-diamine

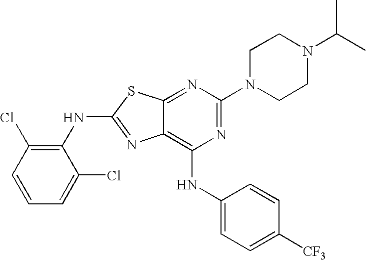

The title compound was prepared using methods analogous to those described in the preceding examples. MS (ESI): mass calcd. for C₂₅H₂₄Cl₂F₃N₇S, 581.1; m/z found, 582.1 [M+H]⁺. ¹H NMR (CD₃OD): δ 7.57 (d, J=8.55 Hz, 2H), 7.37-7.25 (m, 4H), 7.13-7.08 (m, 1H), 4.66-4.52 (m, 2H), 3.39-3.21 (m, 3H), 3.06-2.84 (m, 4H), 1.12 (d, J=6.65 Hz, 6H).

### Example 70

### N²-(2,6-Dichloro-phenyl)-5-phenyl-N⁷-(4-trifluoromethyl-phenyl)-thiazolo[5,4-d]pyrimidine-2,7-diamine

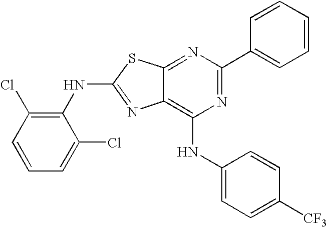

To a mixture of N²-(2,6-dichloro-phenyl)-5-methylsulfanyl-N⁷-(4-trifluoromethyl-phenyl)-thiazolo[5,4-d]pyrimidine-2,7-diamine (Example 52; 58 mg, 0.12 mmol), tri-2-furylphosphine (4.3 mg, 0.02 mmol), copper (I)-thiophene-2-carboxylate (29 mg, 0.15 mmol), and phenyl boronic acid (16 mg, 0.13 mmol) in THF (2 mL) was added tris(dibenzylideneacetone)dipalladium(0) (4.2 mg, 0.005 mmol). The mixture was heated to 50° C. for 24 h under N₂. The reaction mixture was cooled and filtered through a pad of diatomaceous earth, eluting with MeOH (30 mL). The filtrate was concentrated and the crude residue was purified using preparative reverse-phase HPLC to afford the title compound a colorless solid (15 mg, 24%). MS (ESI): mass calcd. for C₂₄H₁₄Cl₂F₃N₅S, 531.1; m/z found, 532.1 [M+H]⁺. ¹H NMR (CD₃OD): δ 9.04-9.00 (m, 2H), 8.78-8.73 (m, 2H), 8.35-8.31 (m, 2H), 8.30-8.26 (m, 2H), 8.19-8.07 (m, 4H).

### Example 71

### N²-(2,6-Dichloro-phenyl)-5-isopropyl-N⁷-(4-trifluoromethyl-phenyl)-thiazolo[5,4-d]pyrimidine-2,7-diamine

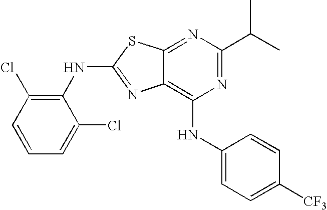

The title compound may be prepared using methods analogous to those described in the preceding examples.

### Example 72

### N²-(3,5-Dichloro-pyridin-4-yl)-N⁷-(4-trifluoromethyl-phenyl)-thiazolo[5,4-d]pyrimidine-2,7-diamine

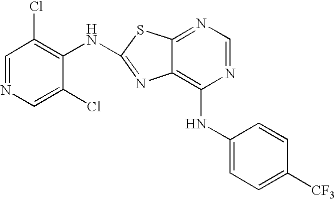

The title compound was prepared using methods analogous to those described in the preceding examples. MS (ESI): mass calcd. for C₁₇H₉Cl₂F₃N₆S, 455.9; m/z found, 457.1 [M+H]⁺. ¹H NMR (CD₃OD): δ 8.66-8.65 (m, 2H), 8.44 (s, 1H), 8.00 (d, J=8.59 Hz, 2H), 7.61 (d, J=8.66 Hz, 2H).

The compounds in Examples 73-258 were prepared using methods analogous to those described in the preceding examples, with exceptions where noted.

### Example 73

### N²-(2,6-Dichloro-phenyl)-5-methyl-N⁷-[4-(pyrrolidine-1-sulfonyl)-phenyl]-thiazolo[5,4-d]pyrimidine-2,7-diamine

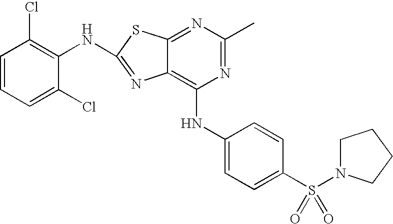

MS (ESI): mass calcd. for C₂₂H₂₀Cl₂N₆O₂S₂, 534.0; m/z found, 535.0 [M+H]⁺. ¹H NMR ((CD₃)₂SO): δ 10.24 (s, 1H), 9.54 (s, 1H), 8.16-8.11 (m, 2H), 7.70 (d, J=8.76 Hz, 2H), 7.65 (d, J=8.15 Hz, 2H), 7.44 (t, J=8.18 Hz, 1H), 3.15-3.10 (m, 4H), 2.52 (s, 3H), 1.67-1.63 (m, 4H).

### Example 74

### 2-{4-[2-(2,6-Dichloro-phenylamino)-5-methyl-thiazolo[5,4-d]pyrimidin-7-ylamino]-phenyl}-propan-2-ol

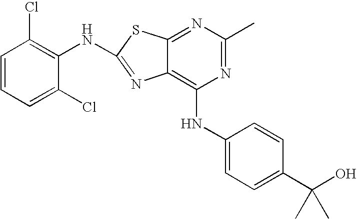

MS (ESI): mass calcd. for C₂₁H₁₉C₂N₅OS, 459.1; m/z found, 460.1 [M+H]⁺. ¹H NMR (CD₃OD): δ 7.72 (d, J=8.64 Hz, 2H), 7.59 (d, J=8.14 Hz, 2H), 7.45 (d, J=8.64 Hz, 2H), 7.42-7.38 (m, 1H), 2.55 (s, 3H), 1.54 (s, 6H).

### Example 75

### 4-[2-(2,6-Dichloro-phenylamino)-thiazolo[5,4-d]pyrimidin-7-ylamino]-N,N-dimethyl-benzenesulfonamide

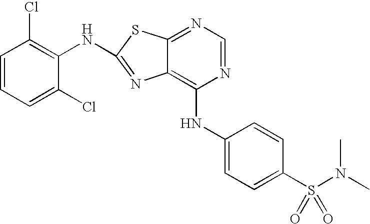

MS (ESI): mass calcd. for C₁₉H₁₆Cl₂N₆O₂S₂, 494.0; m/z found, 495.0 [M+H]⁺. ¹H NMR ((CD₃)₂SO): δ 10.39 (s, 1H), 9.66 (s, 1H), 8.41 (s, 1H), 8.12 (d, J=8.82 Hz, 2H), 7.66 (d, J=8.38 Hz, 4H), 7.48-7.43 (m, 1H), 2.59 (s, 6H).

### Example 76

### N²-(2,6-Dichloro-phenyl)-N⁷-[4-(pyrrolidine-1-sulfonyl)-phenyl]-thiazolo[5,4-d]pyrimidine-2,7-diamine

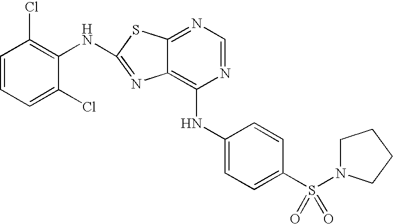

MS (ESI): mass calcd. for C₂₁H₁₈Cl₂N₆O₂S₂, 520.0; m/z found, 521.0 [M+H]⁺. ¹H NMR ((CD₃)₂SO): δ 10.39 (s, 1H), 9.64 (s, 1H), 8.41 (s, 1H), 8.10 (d, J=8.81 Hz, 2H), 7.72 (d, J=8.81 Hz, 2H), 7.66 (d, J=8.16 Hz, 2H), 7.48-7.42 (m, 1H), 3.15-3.10 (m, 4H), 1.67-1.62 (m, 4H).

### Example 77

### N²-(2,6-Dichloro-phenyl)-5-methyl-N⁷-(4-trifluoromethanesulfonyl-phenyl)-thiazolo[5,4-d]pyrimidine-2,7-diamine

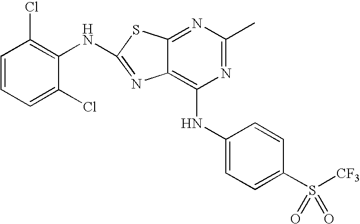

MS (ESI): mass calcd. for C₁₉H₁₂Cl₂F₃N₅O₂S₂, 532.9; m/z found, 534.0 [M+H]⁺. ¹H NMR ((CD₃)₂SO): δ 10.34 (s, 1H), 10.04 (s, 1H), 8.30 (d, J=9.08 Hz, 2H), 7.98 (d, J=9.01 Hz, 2H), 7.65 (d, J=8.16 Hz, 2H), 7.46-7.41 (m, 1H), 2.56 (s, 3H).

### Example 78

### N²-(2,6-Dichloro-phenyl)-N⁷-(4-methanesulfonyl-phenyl)-5-methyl-thiazolo[5,4-d]pyrimidine-2,7-diamine

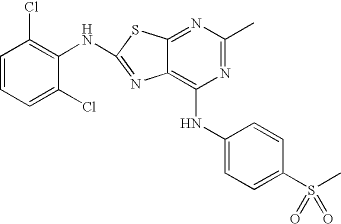

MS (ESI): mass calcd. for C₁₉H₁₅Cl₂N₅O₂S₂, 479.0; m/z found, 480.1 [M+Z]⁺. ¹H NMR ((CD₃)₂SO): δ 10.25 (s, 1H), 9.59 (s, 1H), 8.11 (d, J=8.86 Hz, 2H), 7.81 (d, J=8.85 Hz, 2H), 7.65 (d, J=8.16 Hz, 2H), 7.46-7.41 (m, 1H), 3.17 (s, 3H), 2.52 (s, 3H).

### Example 79

### N²-(2,6-Dichloro-phenyl)-N⁵-isobutyl-N⁷-(4-trifluoromethyl-phenyl)-thiazolo[5,4-d]pyrimidine-2,5,7-triamine

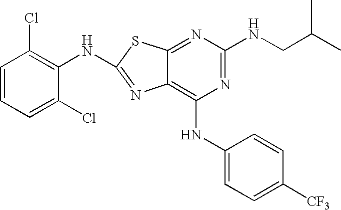

MS (ESI): mass calcd. for C₂₂H₁₉Cl₂F₃N₆S, 526.1; m/z found, 527.1 [M+H]⁺. ¹H NMR ((CD₃)₂SO): δ 9.71 (s, 1H), 9.21 (s, 1H), 8.10 (d, J=8.75 Hz, 2H), 7.64-7.55 (m, 4H), 7.41-7.36 (m, 1H), 3.07 (d, J=6.94 Hz, 2H), 1.92-1.83 (m, 1H), 0.90 (d, J=6.68 Hz, 6H).

### Example 80

### N²-(2,6-Dichloro-phenyl)-N⁷-[4-(morpholine-4-sulfonyl)-phenyl]-thiazolo[5,4-d]pyrimidine-2,7-diamine

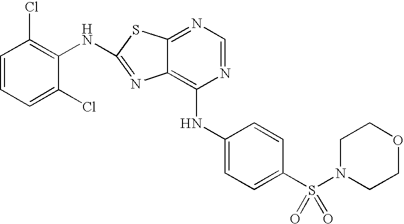

MS (ESI): mass calcd. for C₂₁H₁₈Cl₂N₆O₃S₂, 536.0; m/z found, 537.0 [M+H]⁺. ¹H NMR ((CD₃)₂SO): δ 10.40 (s, 1H), 9.70 (s, 1H), 8.42 (s, 1H), 8.13 (d, J=8.81 Hz, 2H), 7.68-7.63 (m, 4H), 7.48-7.43 (m, 1H), 3.66-3.60 (m, 4H), 2.87-2.82 (m, 4H).

### Example 81

### 4-[2-(2,6-Dichloro-phenylamino)-5-methyl-thiazolo[5,4-d]pyrimidin-7-ylamino]-N,N-dimethyl-benzenesulfonamide

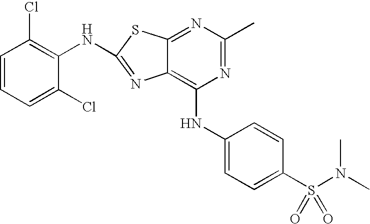

MS (ESI): mass calcd. for C₂₀H₁₈Cl₂N₆O₂S₂, 508.0; m/z found, 509.0 [M+H]⁺. ¹H NMR ((CD₃)₂SO): δ 10.24 (s, 1H), 9.57 (s, 1H), 8.15 (d, J=8.82 Hz, 2H), 7.67-7.62 (m, 4H), 7.46-7.41 (m, 1H), 2.59 (s, 6H), 2.52 (s, 3H).

### Example 82

### N²-(2,6-Dichloro-phenyl)-N⁷-(3-fluoro-4-methanesulfonyl-phenyl)-thiazolo[5,4-d]pyrimidine-2,7-diamine

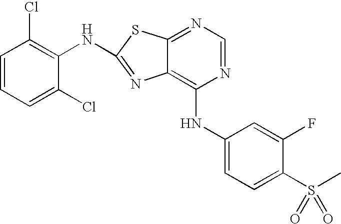

MS (ESI): mass calcd. for C₁₈H₁₂Cl₂FN₅O₂S₂, 482.9; m/z found, 484.0 [M+H]⁺. ¹H NMR ((CD₃)₂SO): δ 10.41 (s, 1H), 9.88 (s, 1H), 8.47 (s, 1H), 8.14 (dd, J=13.64, 1.95 Hz, 1H), 7.92-7.89 (m, 1H), 7.77-7.71 (m, 1H), 7.66 (d, J=8.16 Hz, 2H), 7.49-7.44 (m, 1H), 3.27 (s, 3H).

### Example 83

### N⁷-[4-(Pyrrolidine-1-sulfonyl)-phenyl]-N²-o-tolyl-thiazolo[5,4-d]pyrimidine-2,7-diamine

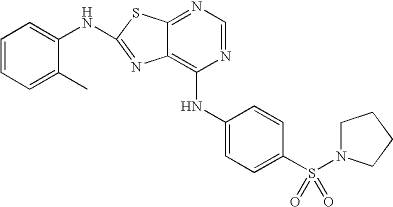

MS (ESI): mass calcd. for C₂₂H₂₂N₆O₂S₂, 466.1; m/z found, 467.1 [M+H]⁺. ¹H NMR ((CD₃)₂SO): δ 9.86 (s, 1H), 9.57 (s, 1H), 8.41 (s, 1H), 8.15-8.11 (m, 2H), 8.03 (d, J=7.84 Hz, 1H), 7.77-7.73 (m, 2H), 7.31-7.26 (m, 2H), 7.16-7.12 (m, 1H), 3.16-3.11 (m, 4H), 2.32 (s, 3H), 1.67-1.63 (m, 4H).

### Example 84

### N²-(2,6-Dichloro-phenyl)-N⁷-(4-isopropyl-phenyl)-5-methyl-thiazolo[5,4-d]pyrimidine-2,7-diamine

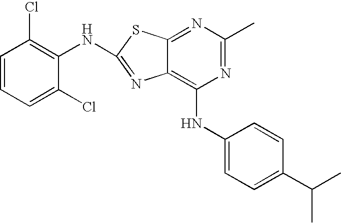

MS (ESI): mass calcd. for C₂₁H₁₉Cl₂N₅S, 443.0; m/z found, 444.1 [M+H]⁺. ¹H NMR ((CD₃)₂SO): δ 10.16 (s, 1H), 8.98 (s, 1H), 7.69 (d, J=8.57 Hz, 2H), 7.64 (d, J=8.15 Hz, 2H), 7.45-7.39 (m, 1H), 7.16 (d, J=8.55 Hz, 2H), 2.88-2.81 (m, 1H), 2.44 (s, 3H), 1.20 (d, J=6.91 Hz, 6H).

### Example 85

### 4-[2-(2,6-Dimethyl-phenylamino)-5-methyl-thiazolo[5,4-d]pyrimidin-7-ylamino]-N,N-dimethyl-benzenesulfonamide

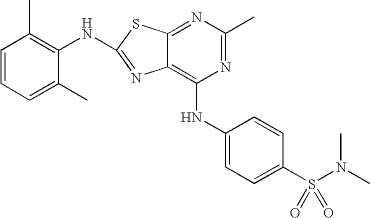

MS (ESI): mass calcd. for C₂₂H₂₄N₆O₂S₂, 468.1; m/z found, 469.1 [M+H]⁺. ¹H NMR ((CD₃)₂SO): δ 9.49 (s, 1H), 9.46 (s, 1H), 8.10 (d, J=8.83 Hz, 2H), 7.57 (d, J=8.86 Hz, 2H), 7.13 (s, 3H), 2.51 (s, 6H), 2.42 (s, 3H), 2.17 (s, 6H).

### Example 86

### 1-{4-[2-(2,6-Dichloro-phenylamino)-5-methyl-thiazolo[5,4-d]pyrimidin-7-ylamino]-phenyl}-ethanone

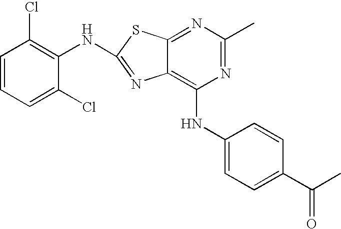

MS (ESI): mass calcd. for C₂₀H₁₅C₂N₅OS, 443.0; m/z found, 444.1 [M+H]⁺. ¹H NMR (CDCl₃): δ 8.01 (d, J=8.76 Hz, 2H), 7.92 (d, J=8.72 Hz, 2H), 7.51 (d, J=8.13 Hz, 2H), 7.36-7.31 (m, 1H), 2.72 (s, 3H), 2.62 (s, 3H).

### Example 87

### N²-(2,6-Dichloro-phenyl)-N⁷-(4-methanesulfonyl-phenyl)-thiazolo[5,4-d]pyrimidine-2,7-diamine

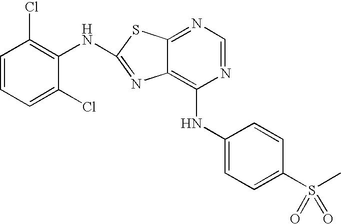

MS (ESI): mass calcd. for C₁₈H₁₃Cl₂N₅O₂S₂, 464.9; m/z found, 466.0 [M+H]⁺. ¹H NMR ((CD₃)₂SO): δ 10.39 (s, 1H), 9.67 (s, 1H), 8.41 (s, 1H), 8.10 (d, J=8.87 Hz, 2H), 7.82 (d, J=8.87 Hz, 2H), 7.66 (d, J=8.15 Hz, 2H), 7.48-7.43 (m, 1H), 3.17 (s, 3H).

### Example 88

### N²-(2,6-Dichloro-phenyl)-N⁷-[4-(4-methyl-piperazine-1-sulfonyl)-phenyl]-thiazolo[5,4-d]pyrimidine-2,7-diamine

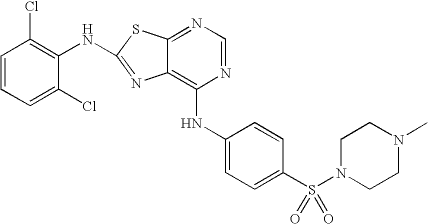

MS (ESI): mass calcd. for C₂₂H₂₁Cl₂N₇O₂S₂, 549.0; m/z found, 550.1 [M+H]⁺. ¹H NMR ((CD₃)₂SO): δ 10.38 (s, 1H), 9.72 (s, 1H), 8.42 (s, 1H), 8.16 (d, J=8.90 Hz, 2H), 7.71 (d, J=8.90 Hz, 2H), 7.66 (d, J=8.15 Hz, 2H), 7.49-7.43 (m, 1H), 3.84-3.64 (m, 4H), 3.28-3.06 (m, 4H), 2.79 (s, 3H).

### Example 89

### (racemic)-N²-(2,6-Dichloro-phenyl)-5-(2-isopropyl-pyrrolidin-1-yl)-N⁷-(4-trifluoromethyl-phenyl)-thiazolo[5,4-d]pyrimidine-2,7-diamine

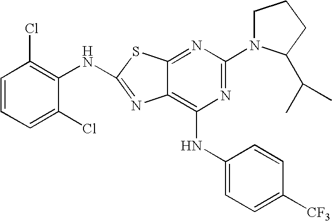

MS (ESI): mass calcd. for C₂₅H₂₃Cl₂F₃N₆S, 566.1; m/z found, 567.1 [M+H]⁺. ¹H NMR ((CD₃)₂SO): δ 9.75 (s, 1H), 9.22 (s, 1H), 8.09 (d, J=8.61 Hz, 2H), 7.64-7.57 (m, 4H), 7.42-7.36 (m, 1H), 4.05-4.00, (m, 2H), 3.67-3.44 (m, 1H), 2.46-2.31 (m, 1H), 1.93-1.79 (m, 4H), 0.88 (d, J=6.95 Hz, 3H), 0.76 (d, J=6.87 Hz, 3H).

### Example 90

### N²-(2,6-Dimethyl-phenyl)-5-methyl-N⁷-(4-trifluoromethanesulfonyl-phenyl)-thiazolo[5,4-d]pyrimidine-2,7-diamine

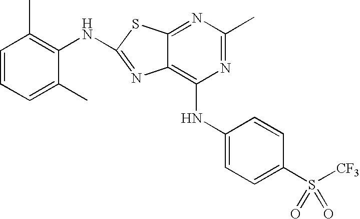

MS (ESI): mass calcd. for C₂₁H₁₈F₃N₅O₂S₂, 493.1; m/z found, 494.1 [M+H]⁺. ¹H NMR ((CD₃)₂SO): δ 10.03 (s, 1H), 9.67 (s, 1H), 8.35 (d, J=9.00 Hz, 2H), 7.99 (d, J=8.99 Hz, 2H), 7.23-7.21 (m, 3H), 2.54 (s, 3H), 2.26 (s, 6H).

### Example 91

### N²-(2,6-Dimethyl-phenyl)-N⁷-[4-(morpholine-4-sulfonyl)-phenyl]-thiazolo[5,4-d]pyrimidine-2,7-diamine

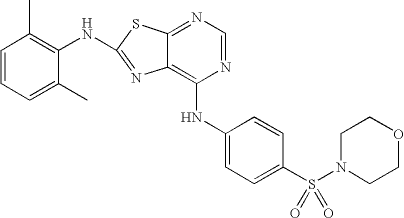

MS (ESI): mass calcd. for C₂₃H₂₄N₆O₃S₂, 496.1; m/z found, 497.2 [M+H]⁺. ¹H NMR ((CD₃)₂SO): δ 9.73 (s, 1H), 9.69 (s, 1H), 8.37 (s, 1H), 8.17 (d, J=8.82 Hz, 2H), 7.66 (d, J=8.87 Hz, 2H), 7.24-7.21 (m, 3H), 3.65-3.61 (m, 4H), 2.88-2.82 (m, 4H), 2.27 (s, 6H).

### Example 92

### N²-(2,6-Dimethyl-phenyl)-5-methyl-N⁷-[4-(pyrrolidine-1-sulfonyl)-phenyl]-thiazolo[5,4-d]pyrimidine-2,7-diamine

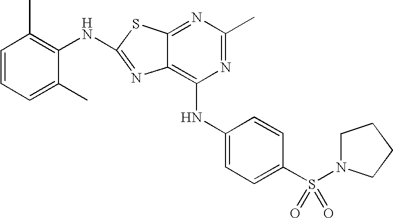

MS (ESI): mass calcd. for C₂₄H₂₆N₆O₂S₂, 494.1; m/z found, 495.2 [M+H]⁺. ¹H NMR ((CD₃)₂SO): δ 9.49 (s, 1H), 9.44 (s, 1H), 8.08 (d, J=8.84 Hz, 2H), 7.62 (d, J=8.85 Hz, 2H), 7.14-7.11 (m, 3H), 3.07-3.01 (m, 4H), 2.17 (s, 6H), 1.59-1.54 (m, 4H).

### Example 93

### N²-(2,6-Dichloro-phenyl)-5-methyl-N⁷-[4-(propane-2-sulfonyl)-phenyl]-thiazolo[5,4-d]pyrimidine-2,7-diamine

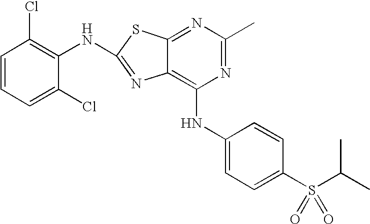

MS (ESI): mass cald. for C₂₁H₁₉Cl₂N₅O₂S₂, 507.0; m/z found, 508.1 [M+H]⁺. ¹H NMR ((CD₃)₂SO): δ 10.27 (s, 1H), 9.63 (s, 1H), 8.15 (d, J=8.82 Hz, 2H), 7.72 (d, J=8.82 Hz, 2H), 7.65 (d, J=8.16 Hz, 2H), 7.46-7.41 (m, 1H), 3.37-3.31 (m, 1H), 2.53 (s, 3H), 1.15 (d, J=6.80 Hz, 6H).

### Example 94

### N²-(2,6-Dichloro-phenyl)-5-methyl-N⁷-(4-methylsulfanyl-phenyl)-thiazolo[5,4-d]pyrimidine-2,7-diamine

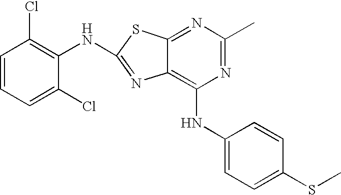

MS (ESI): mass calcd. for C₁₉H₁₅Cl₂N₅S₂, 447.0; m/z found, 448.1 [M+H]⁺. ¹H NMR ((CD₃)₂SO): δ 10.16 (s, 1H), 9.09 (s, 1H), 7.78 (d, J=8.76 Hz, 2H), 7.64 (d, J=8.15 Hz, 2H), 7.46-7.40 (m, 1H), 7.22 (d, J=8.75 Hz, 2H), 2.45 (s, 6H).

### Example 95

### N²-(2,6-Dimethyl-phenyl)-N⁷-(4-methanesulfonyl-phenyl)-5-methyl-thiazolo[5,4-d]pyrimidine-2,7-diamine

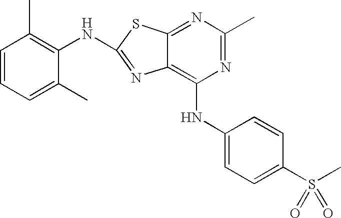

MS (ESI): mass calcd. for C₂₁H₂₁N₅O₂S₂, 439.1; m/z found, 440.2 [M+H]⁺. ¹H NMR (CDCl₃): δ 8.07 (d, J=8.83 Hz, 2H), 7.97-7.93 (m, 1H), 7.88 (s, 1H), 7.30-7.26 (m, 1H), 7.22 (d, J=7.53 Hz, 2H), 3.08 (s, 3H), 2.69 (s, 3H), 2.36 (s, 6H).

### Example 96

### 4-[2-(2,6-Dichloro-phenylamino)-5-methyl-thiazolo[5,4-d]pyrimidin-7-ylamino]-benzonitrile

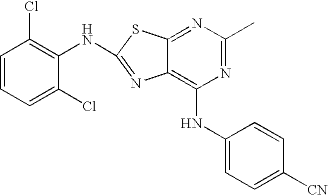

MS (ESI): mass calcd. for C₁₉H₁₂Cl₂N₆S, 426.0; m/z found, 427.1 [M+H]⁺. ¹H NMR ((CD₃)₂SO): δ 10.25 (s, 1H), 9.61 (s, 1H), 8.10 (d, J=8.84 Hz, 2H), 7.72 (d, J=8.81 Hz, 2H), 7.65 (d, J=8.14 Hz, 2H), 7.48-7.41 (m, 1H), 2.52 (s, 3H).

### Example 97

### N²-(2,6-Dimethyl-phenyl)-N⁷-(3-fluoro-4-methanesulfonyl-phenyl)-thiazolo[5,4-d]pyrimidine-2,7-diamine

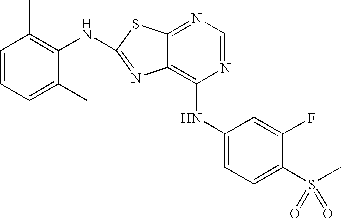

MS (ESI): mass calcd. for C₂₀H₁₈FN₅O₂S₂, 443.1; m/z found, 444.1 [M+H]⁺. ¹H NMR ((CD₃)₂SO): δ 9.87 (s, 1H), 9.74 (s, 1H), 8.42 (s, 1H), 8.16 (dd, J=13.61, 1.79 Hz, 1H), 7.94 (dd, J=8.75, 1.72 Hz, 1H), 7.79-7.71 (m, 1H), 7.26-7.20 (m, 3H), 3.27 (s, 3H), 2.27 (s, 6H).

### Example 98

### 4-[2-(2,6-Dimethyl-phenylamino)-thiazolo[5,4-d]pyrimidin-7-ylamino]-N,N-dimethyl-benzenesulfonamide

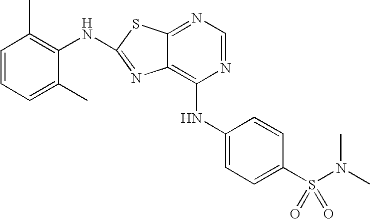

MS (ESI): mass calcd. for C₂₁H₂₂N₆O₂S₂, 454.1; m/z found, 455.1 [M+H]⁺. ¹H NMR ((CD₃)₂SO): δ 9.63 (s, 1H), 9.55 (s, 1H), 8.28 (s, 1H), 8.06 (d, J=8.78 Hz, 2H), 7.58 (d, J=8.85 Hz, 2H), 7.15-7.13 (m, 3H), 2.51 (s, 6H), 2.18 (s, 6H).

### Example 99

### N²-(2,6-Dimethyl-phenyl)-N⁷-(4-trifluoromethanesulfonyl-phenyl)-thiazolo[5,4-d]pyrimidine-2,7-diamine

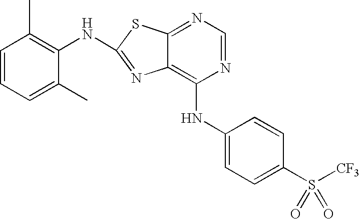

MS (ESI): mass calcd. for C₂₀H₁₆F₃N₅O₂S₂, 479.1; m/z found, 480.1 [M+H]⁺. ¹H NMR ((CD₃)₂SO): δ 10.11 (s, 1H), 9.81 (s, 1H), 8.44 (s, 1H), 8.33 (d, J=8.94 Hz, 2H), 8.01 (d, J=8.99 Hz, 2H), 7.27-7.19 (m, 3H), 2.27 (s, 6H).

### Example 100

### N²-(2,6-Dichloro-phenyl)-N⁵-(3-morpholin-4-yl-propyl)-N⁷-(4-trifluoromethyl-phenyl)-thiazolo[5,4-d]pyrimidine-2,5,7-triamine

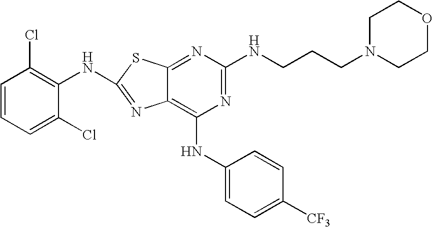

MS (ESI): mass calcd. for C₂₅H₂₄Cl₂F₃N₇OS, 597.1; m/z found, 598.1 [M+H]J. ¹H NMR (CDCl₃): δ 9.57 (s, 1H), 8.38 (s, 1H), 7.81 (d, J=8.48 Hz, 2H), 7.64 (d, J=8.58 Hz, 2H), 7.50 (d, J=8.14 Hz, 2H), 7.37-7.32 (m, 1H), 4.07-3.98 (m, 2H), 3.98-3.89 (m, 2H), 3.65-3.54 (m, 4H), 3.50 (s, 1H), 3.35-3.27 (m, 2H), 3.04-2.91 (m, 2H), 2.32-2.20 (m, 2H).

### Example 101

### N²-(2,6-Dichloro-phenyl)-N⁵-isopropyl-N⁷-(4-trifluoromethyl-phenyl)-thiazolo[5,4-d]pyrimidine-2,5,7-triamine

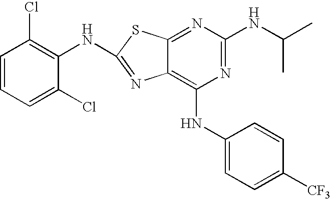

MS (ESI): mass calcd. for C₂₁H₁₇Cl₂F₃N₆S, 512.0; m/z found, 513.1 [M+H]⁺. ¹H NMR (CDCl₃): δ 8.18-8.01 (m, 1H), 7.89 (d, J=8.51 Hz, 2H), 7.69 (d, J=8.57 Hz, 2H), 7.51 (d, J=8.14 Hz, 2H), 7.38-7.33 (m, 1H), 4.21-4.14 (m, 1H), 1.36 (d, J=6.56 Hz, 6H).

### Example 102

### N²-(2,6-Dimethyl-phenyl)-5-methyl-N⁷-[4-(Propane-2-sulfonyl)-phenyl]-thiazolo[5,4-d]pyrimidine-2,7-diamine

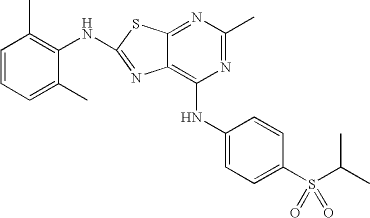

MS (ESI): mass calcd. for C₂₃H₂₅N₅O₂S₂, 467.1; m/z found, 468.2 [M+H]⁺. ¹H NMR ((CD₃)₂SO): δ 9.60 (s, 2H), 8.19 (d, J=8.87 Hz, 2H), 7.73 (d, J=8.89 Hz, 2H), 7.23-7.20 (m, 3H), 3.39-3.28 (m, 1H), 2.51 (s, 3H), 2.26 (s, 6H), 1.16 (d, J=6.81 Hz, 6H).

### Example 103

### N²-(2,6-Dichloro-phenyl)-N⁷-(4-isopropylsulfanyl-phenyl)-5-methyl-thiazolo[5,4-d]pyrimidine-2,7-diamine

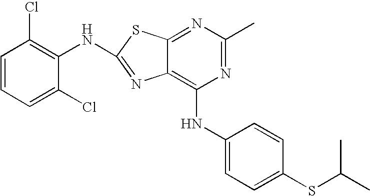

MS (ESI): mass calcd. for C₂₁H₁₉Cl₂N₅S₂, 475.0; m/z found, 476.1 [M+H]⁺. ¹H NMR (CD₃OD): δ 7.73 (d, J=8.69 Hz, 2H), 7.58 (d, J=8.10 Hz, 2H), 7.44-7.36 (m, 3H), 2.60 (s, 3H), 1.27 (d, J=6.67 Hz, 6H).

### Example 104

### N²-(2,6-Dimethyl-phenyl)-N⁷-[4-(pyrrolidine-1-sulfonyl)-phenyl]-thiazolo[5,4-d]pyrimidine-2,7-diamine

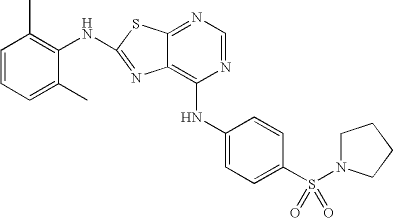

MS (ESI): mass calcd. for C₂₃H₂₄N₆O₂S₂, 480.1; m/z found, 481.2 [M+H]⁺. ¹H NMR ((CD₃)₂SO): δ 9.63 (s, 1H), 9.53 (s, 1H), 8.27 (s, 1H), 8.04 (d, J=8.80 Hz, 2H), 7.67-7.61 (m, 1H), 7.15-7.12 (m, 3H), 3.07-3.01 (m, 4H), 2.18 (s, 6H), 1.59-1.54 (m, 4H).

### Example 105

### (racemic)-N²-(2,6-Dichloro-phenyl)-5-(2-methyl-pyrrolidin-1-yl)-N⁷-(4-trifluoromethyl-phenyl)-thiazolo[5,4-d]pyrimidine-2,7-diamine

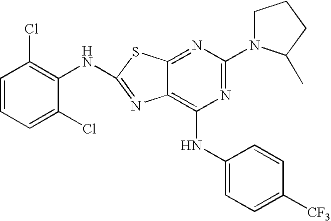

MS (ESI): mass calcd. for C₂₃H₁₉Cl₂F₃N₆S, 538.1; m/z found, 539.1 [M+H]⁺. ¹H NMR (CD₃OD): δ 8.02 (d, J=8.57 Hz, 2H), 7.61-7.54 (m, 4H), 7.42-7.35 (m, 1H), 4.38-4.28 (m, 1H), 3.74-3.63 (m, 1H), 3.60-3.50 (m, 1H), 2.18-2.08 (m, 2H), 2.06-1.97 (m, 1H), 1.83-1.71 (m, 1H), 1.31 (d, J=6.31 Hz, 3H).

### Example 106

### N²-(2,6-Dimethyl-phenyl)-N⁷-(4-isopropylsulfanyl-phenyl)-5-methyl-thiazolo[5,4-d]pyrimidine-2,7-diamine

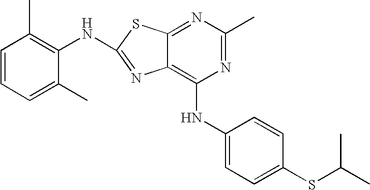

MS (ESI): mass calcd. for C₂₃H₂₅N₅S₂, 435.1; m/z found, 436.2 [M+H]⁺. ¹H NMR ((CD₃)₂SO): δ 9.53 (s, 1H), 9.13 (s, 1H), 7.88 (d, J=8.67 Hz, 2H), 7.34 (d, J=8.70 Hz, 2H), 7.22-7.20 (m, 3H), 3.39-3.31 (m, 1H), 2.46 (s, 3H), 2.26 (s, 6H), 1.21 (d, J=6.66 Hz, 6H).

### Example 107

### N²-(2,6-Dichloro-phenyl)-5-methyl-N⁷-(1,4,4-trimethyl-1,2,3,4-tetrahydro-quinolin-7-yl)-thiazolo[5,4-d]pyrimidine-2,7-diamine

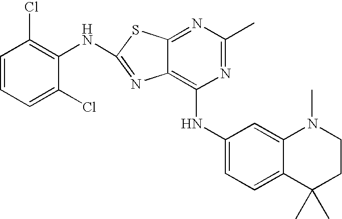

MS (ESI): mass calcd. for C₂₄H₂₄Cl₂N₆S, 498.1; m/z found, 499.2 [M+H]⁺. ¹H NMR (CDCl₃): δ 7.48 (s, 1H), 7.45 (d, J=8.19 Hz, 2H), 7.32-7.28 (m, 1H), 7.27-7.25 (m, 1H), 7.24-7.21 (m, 1H), 3.46-3.40 (m, 2H), 3.07 (s, 3H), 2.67 (s, 3H), 1.92-1.87 (m, 2H), 1.32 (s, 6H).

### Example 108

### N²-(2,6-Dimethyl-phenyl)-N⁷-(3-fluoro-4-trifluoromethyl-phenyl)-thiazolo[5,4-d]pyrimidine-2,7-diamine

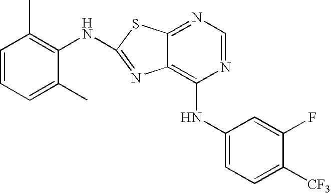

MS (ESI): mass calcd. for C₂₀H₁₅F₄N₅S, 433.1; m/z found, 434.2 [M+H]⁺. ¹H NMR (CDCl₃): δ 8.43 (s, 1H), 8.07-8.02 (m, 1H), 7.84 (s, 1H), 7.47 (t, J=8.26 Hz, 1H), 7.39-7.35 (m, 1H), 7.23-7.18 (m, 1H), 7.16-7.12 (m, 2H), 2.27 (s, 6H).

### Example 109

### N²-(2-Chloro-phenyl)-N⁷-[4-(pyrrolidine-1-sulfonyl)-phenyl]-thiazolo[5,4-d]pyrimidine-2,7-diamine

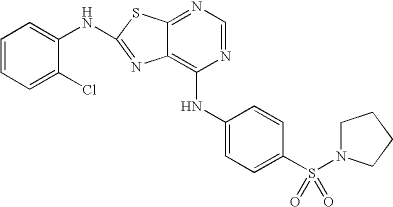

MS (ESI): mass calcd. for C₂₁H₁₉ClN₆O₂S₂, 486.1; m/z found, 487.1 [M+H]⁺. ¹H NMR ((CD₃)₂SO): δ 10.25 (s, 1H), 9.64 (s, 1H), 8.60-8.56 (m, 1H), 8.46 (s, 1H), 8.13 (d, J=8.87 Hz, 2H), 7.77 (d, J=8.85 Hz, 2H), 7.55 (dd, J=8.00, 1.45 Hz, 1H), 7.45-7.40 (m, 1H), 7.22-7.16 (m, 1H), 3.18-3.10 (m, 4H), 1.69-1.60 (m, 4H).

### Example 110

### N²-(2,6-Dimethyl-phenyl)-N⁷-[4-(4-methyl-piperazine-1-sulfonyl)-phenyl]-thiazolo[5,4-d]pyrimidine-2,7-diamine

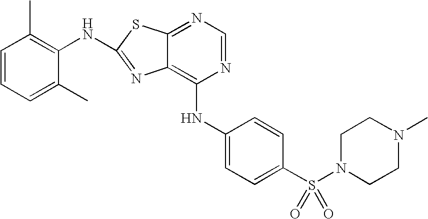

MS (ESI): mass calcd. for C₂₄H₂₇N₇O₂S₂, 509.1; m/z found, 510.2 [M+H]⁺. ¹H NMR ((CD₃)₂SO): δ 9.76-9.66 (m, 2H), 8.37 (s, 1H), 8.20 (d, J=8.89 Hz, 2H), 7.72 (d, J=8.89 Hz, 2H), 7.25-7.21 (m, 3H), 4.16-3.56 (m, 4H), 3.53-2.99 (m, 4H), 2.80 (s, 3H), 2.27 (s, 6H).

### Example 111

### N²-(2,6-Dichloro-phenyl)-5-methyl-N⁷-(4-trifluoromethoxy-phenyl)-thiazolo[5,4-d]pyrimidine-2,7-diamine

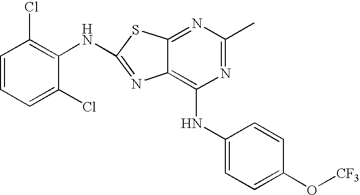

MS (ESI): mass calcd. for C₁₉H₁₂Cl₂F₃N₅OS, 485.0; m/z found 486.1 [M+H]⁺. ¹H NMR ((CD₃)₂SO): δ 10.18 (s, 1H), 9.29 (s, 1H), 7.95 (d, J=9.13 Hz, 2H), 7.64 (d, J=8.13 Hz, 2H), 7.47-7.39 (m, 1H), 7.29 (d, J=8.62 Hz, 2H), 2.47 (s, 3H).

### Example 112

### N²-(2,6-Dimethyl-phenyl)-NT-(4-isopropylsulfanyl-phenyl)-thiazolo[5,4-d]pyrimidine-2,7-diamine

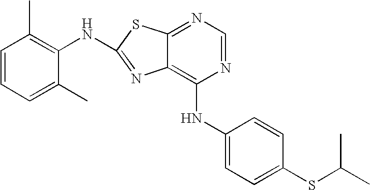

MS (ESI): mass calcd. for C₂₂H₂₃N₅S₂, 421.1; m/z found, 422.2 [M+H]⁺. ¹H NMR ((CD₃)₂SO): δ 9.65 (s, 1H), 9.18 (s, 1H), 8.28 (s, 1H), 7.84 (d, J=8.64 Hz, 2H), 7.35 (d, J=8.69 Hz, 2H), 7.22 (s, 3H), 3.40-3.30 (m, 1H), 2.27 (s, 6H), 1.21 (d, J=6.66 Hz, 6H).

### Example 113

### 4-[2-(2-Chloro-phenylamino)-thiazolo[5,4-d]pyrimidin-7-ylamino]-N,N-dimethyl-benzenesulfonamide

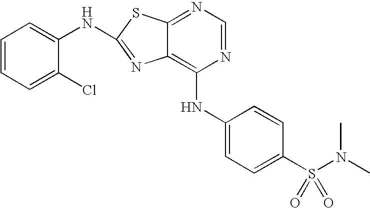

MS (ESI): mass calcd. for C₁₉H₁₇ClN₆O₂S₂, 460.0; m/z found, 461.1 [M+H]⁺. ¹H NMR ((CD₃)₂SO): δ 10.26 (s, 1H), 9.67 (s, 1H), 8.59-8.56 (m, 1H), 8.46 (s, 1H), 8.15 (d, J=8.83 Hz, 2H), 7.71 (d, J=8.83 Hz, 2H), 7.57-7.53 (m, 1H), 7.45-7.40 (m, 1H), 7.22-7.16 (m, 1H), 2.60 (s, 6H).

### Example 114

### N²-(2,6-Dimethyl-phenyl)-5-methyl-N⁷-(4-methylsulfanyl-phenyl)-thiazolo[5,4-d]pyrimidine-2,7-diamine

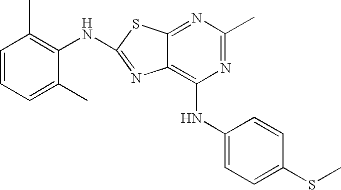

MS (ESI): mass calcd. for C₂₁H₂₁N₅S₂, 407.1; m/z found, 408.2 [M+H]⁺. ¹H NMR ((CD₃)₂SO): δ 9.53 (s, 1H), 9.11 (s, 1H), 7.83 (d, J=8.72 Hz, 2H), 7.24 (d, J=8.72 Hz, 2H), 7.22-7.20 (m, 3H), 2.46 (s, 3H), 2.44 (s, 3H), 2.26 (s, 6H).

### Example 115

### N²-(2,6-Dimethyl-phenyl)-N⁷-(4-methanesulfonyl-phenyl)-thiazolo[5,4-d]pyrimidine-2,7-diamine

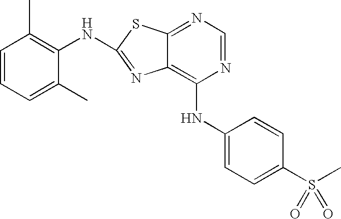

MS (ESI): mass calcd. for C₂₀H₁₉N₅O₂S₂, 425.1; m/z found, 426.1 [M+H]⁺. ¹H NMR ((CD₃)₂SO): δ 9.73 (s, 1H), 9.65 (s, 1H), 8.37 (s, 1H), 8.14 (d, J=8.79 Hz, 2H), 7.83 (d, J=8.85 Hz, 2H), 7.27-7.19 (m, 3H), 3.17 (s, 3H), 2.27 (s, 6H).

### Example 116

### N⁷-(4-Methanesulfonyl-phenyl)-N²-o-tolyl-thiazolo[5,4-d]pyrimidine-2,7-diamine

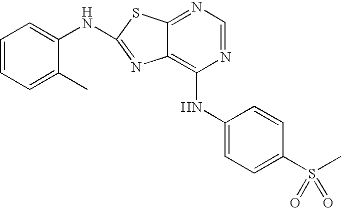

MS (ESI): mass calcd. for C₁₉H₁₇N₅O₂S₂, 411.1; m/z found, 412.1 [M+H]⁺. ¹H NMR ((CD₃)₂SO): δ 9.86 (s, 1H), 9.60 (s, 1H), 8.42 (s, 1H), 8.14 (d, J=8.90 Hz, 2H), 8.02 (d, J=7.97 Hz, 1H), 7.86 (d, J=8.88 Hz, 2H), 7.32-7.26 (m, 2H), 7.18-7.12 (m, 1H), 3.18 (s, 3H), 2.32 (s, 3H).

### Example 117

### (racemic)-N²-(2,6-Dichloro-phenyl)-N⁷-(4-methanesulfonyl-phenyl)-5-(2-methyl-pyrrolidin-1-yl)-thiazolo[5,4-d]pyrimidine-2,7-diamine

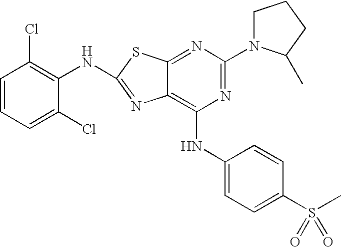

MS (ESI): mass calcd. for C₂₃H₂₂Cl₂N₆O₂S₂, 548.1; m/z found, 549.1 [M+H]⁺. ¹H NMR (CD₃OD): δ 8.03 (d, J=8.93 Hz, 2H), 7.90 (d, J=8.94 Hz, 2H), 7.60 (d, J=8.13 Hz, 2H), 7.45-7.39 (m, 1H), 4.43-4.30 (m, 1H), 3.78-3.66 (m, 1H), 3.60-3.51 (m, 1H), 3.12 (s, 3H), 2.28-2.15 (m, 2H), 2.15-2.07 (m, 1H), 1.89-1.80 (m, 1H), 1.32 (d, J=6.41 Hz, 3H).

### Example 118

### (racemic)-N⁷-(3-Chloro-4-trifluoromethyl-phenyl)-N²-(2,6-dichloro-phenyl)-5-(2-isopropyl-pyrrolidin-1-yl)-thiazolo[5,4-d]pyrimidine-2,7-diamine

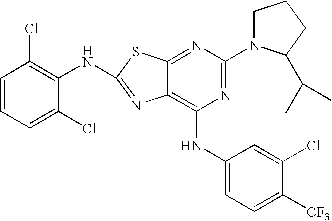

MS (ESI): mass calcd. for C₂₅H₂₂Cl₃F₃N₆S, 600.0; m/z found, 601.1 [M+H]⁺. ¹H NMR (CD₃OD): δ 8.21 (s, 1H), 7.78-7.68 (m, 2H), 7.59 (d, J=8.14 Hz, 2H), 7.44-7.38 (m, 1H), 4.28-4.17 (m, 1H), 3.73-3.60 (m, 2H), 2.49-2.30 (m, 1H), 2.23-1.97 (m, 4H), 0.96 (d, J=6.94 Hz, 3H), 0.88 (d, J=6.84 Hz, 3H).

### Example 119

### N⁷-(6-Chloro-pyridin-3-yl)-N²-(2,6-dimethyl-phenyl)-thiazolo[5,4-d]pyrimidine-2,7-diamine

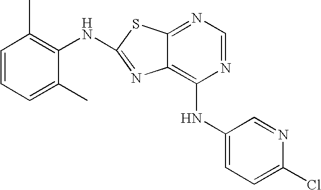

MS (ESI): mass calcd. for C₁₈H₁₅ClN₆S, 382.1; m/z found, 383.0 [M+H]⁺. ¹H NMR ((CD₃)₂SO): δ 9.67 (s, 1H), 9.53 (s, 1H), 8.83 (s, 1H), 8.33 (dd, J=8.66, 2.61 Hz, 1H), 8.29 (s, 1H), 7.44 (d, J=8.70 Hz, 1H), 7.23-7.17 (m, 3H), 2.25 (s, 6H).

### Example 120

### N²-(2,6-Dimethyl-phenyl)-N⁷-(4-methylsulfanyl-phenyl)-thiazolo[5,4-d]pyrimidine-2,7-diamine

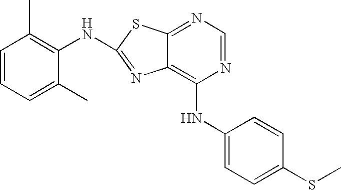

MS (ESI): mass calcd. for C₂₀H₁₉N₅S₂, 393.1; m/z found, 394.1 [M+H]⁺. ¹H NMR (CDCl₃): δ 8.41 (s, 1H), 7.89 (s, 1H), 7.66 (d, J=8.67 Hz, 2H), 7.26 (d, J=8.66 Hz, 2H), 7.24-7.20 (m, 1H), 7.14 (d, J=7.69 Hz, 2H), 2.43 (s, 3H), 2.27 (s, 6H).

### Example 121

### N²-(2,6-Dimethyl-phenyl)-N⁷-(3-fluoro-4-trifluoromethyl-phenyl)-5-methyl-thiazolo[5,4-d]pyrimidine-2,7-diamine

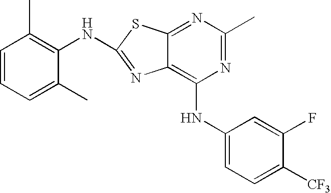

MS (ESI): mass calcd. for C₂₁H₁₇F₄N₅S, 447.1; m/z found, 448.2 [M+H]⁺. ¹H NMR (CDCl₃): δ 8.05 (d, J=13.11 Hz, 1H), 7.66 (s, 1H), 7.47 (t, J=8.30 Hz, 1H), 7.34 (d, J=8.70 Hz, 1H), 7.19 (d, J=15.01 Hz, 2H), 7.13 (d, J=7.47 Hz, 2H), 2.61 (s, 3H), 2.26 (s, 6H).

### Example 122

### N²-(2,6-Dimethyl-phenyl)-N⁷-[4-(Propane-2-sulfonyl)-phenyl]-thiazolo[5,4-d]pyrimidine-2,7-diamine

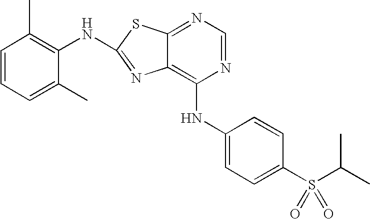

To a solution of N²-(2,6-dimethyl-phenyl)-N⁷-(4-isopropylsulfanyl-phenyl)-thiazolo[5,4-d]pyrimidine-2,7-diamine (Example 112; 100 mg, 0.21 mmol) and CH₂Cl₂ was added m-CPBA (73 mg, 0.42 mmol). After 12 h, the solution was partitioned between satd. aq. NaHCO₃ (10 mL) and CH₂Cl₂ (10 mL). The aqueous layer was extracted with CH₂Cl₂ (3×10 mL). The combined organic layers were dried, filtered, and concentrated. The residue was purified directly using preparative reverse-phase HPLC to afford the title compound (47 mg, 49%). MS (ESI): mass calcd. for C₂₂H₂₃N₅O₂S₂, 453.1; m/z found, 454.2 [M+H]⁺. ¹H NMR ((CD₃)₂SO): δ 9.73 (s, 1H), 9.68 (s, 1H), 8.38 (s, 1H), 8.16 (d, J=8.80 Hz, 2H), 7.75 (d, J=8.85 Hz, 2H), 7.24-7.20 (m, 3H), 3.39-3.29 (m, 1H), 2.27 (s, 6H), 1.16 (d, J=6.81 Hz, 6H).

### Example 123

### N⁷-(4-Bromo-phenyl)-N²-(2,6-dimethyl-phenyl)-thiazolo[5,4-d]pyrimidine-2,7-diamine

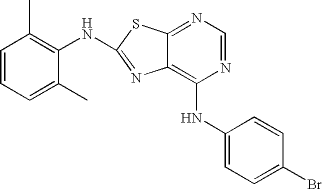

MS (ESI): mass calcd. for C₁₉H₁₆BrN₅S, 426.0; m/z found, 426.1 [M+H]⁺. ¹H NMR (CDCl₃): δ 8.36 (s, 1H), 7.61 (d, J=8.85 Hz, 2H), 7.43-7.38 (m, 2H), 7.22-7.16 (m, 1H), 7.13 (d, J=7.60 Hz, 2H), 2.27 (s, 6H).

### Example 124

### N⁷-(3-Chloro-4-methylsulfanyl-phenyl)-N²-(2,6-dichloro-phenyl)-thiazolo[5,4-d]pyrimidine-2,7-diamine

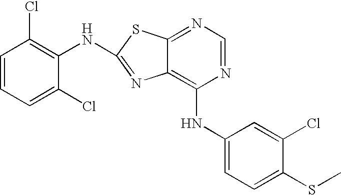

MS (ESI): mass calcd. for C₁₈H₁₂Cl₃N₅S₂, 466.9; m/z found, 468.0 [M+H]⁺. ¹H NMR ((CD₃)₂SO): δ 10.31 (s, 1H), 9.32 (s, 1H), 8.35 (s, 1H), 8.07 (d, J=2.29 Hz, 1H), 7.81 (dd, J=8.75, 2.30 Hz, 1H), 7.66 (d, J=8.15 Hz, 2H), 7.48-7.42 (m, 1H), 7.26 (d, J=8.82 Hz, 1H), 2.47 (s, 3H).

### Example 125

### N²-(2,6-Dimethyl-phenyl)-N⁷-(4-isopropyl-phenyl)-5-methyl-thiazolo[5,4-d]pyrimidine-2,7-diamine

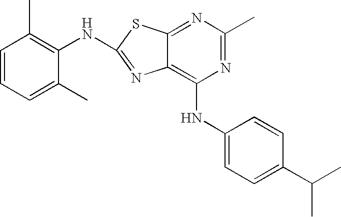

MS (ESI): mass calcd. for C₂₃H₂₅N₅S, 403.2; m/z found, 404.3 [M+H]⁺. ¹H NMR (CDCl₃): δ 7.62 (d, J=8.19 Hz, 2H), 7.23-7.16 (m, 3H), 7.12 (d, J=7.62 Hz, 2H), 2.90-2.82 (m, 1H), 2.61 (s, 3H), 2.26 (s, 6H), 1.20 (d, J=6.91 Hz, 6H).

### Example 126

### N²-(2-Chloro-phenyl)-N⁷-(4-methanesulfonyl-phenyl)-thiazolo[5,4-d]pyrimidine-2,7-diamine

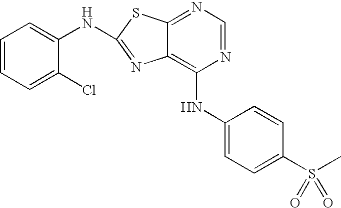

MS (ESI): mass calcd. for C₁₈H₁₄ClN₅O₂S₂, 431.0; m/z found, 432.1 [M+H]⁺. ¹H NMR ((CD₃)₂SO): δ 10.26 (s, 1H), 9.68 (s, 1H), 8.56 (dd, J=8.23, 1.41 Hz, 1H), 8.46 (s, 1H), 8.15 (d, J=8.89 Hz, 2H), 7.88 (d, J=8.88 Hz, 2H), 7.55 (dd, J=7.99, 1.45 Hz, 1H), 7.45-7.41 (m, 1H), 7.23-7.16 (m, 1H), 3.18 (s, 3H).

### Example 127

### 4-[2-(2,6-Dichloro-phenylamino)-5-methylsulfanyl-thiazolo[5,4-d]pyrimidin-7-ylamino]-N,N-dimethyl-benzenesulfonamide

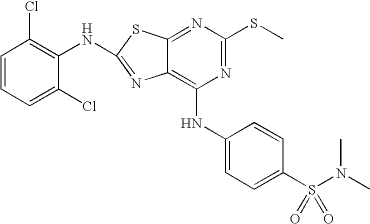

MS (ESI): mass-calcd. for C₂₀H₁₈Cl₂N₆O₂S₃, 540.0; m/z found, 541.0 [M+H]⁺. ¹H NMR (CDCl₃): δ 8.01 (d, J=8.78 Hz, 2H), 7.78 (d, J=8.77 Hz, 2H), 7.51 (d, J=8.17 Hz, 2H), 7.38-7.32 (m, 1H), 2.74 (s, 6H), 2.63 (s, 3H).

### Example 128

### 1-{4-[2-(2,6-Dimethyl-phenylamino)-thiazolo[5,4-d]pyrimidin-7-ylamino]-phenyl}-ethanone

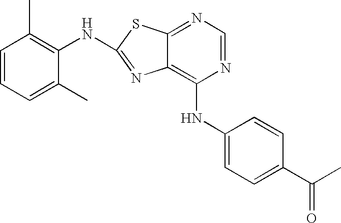

MS (ESI): mass clacd. for C₂₁H₁₉N₅OS, 389.1; m/z found, 390.1 [M+H]⁺. ¹H NMR ((CD₃)₂SO): δ 9.72 (s, 1H), 9.53 (s, 1H), 8.36 (s, 1H), 8.05 (d, J=8.80 Hz, 2H), 7.92 (d, J=8.83 Hz, 2H), 7.24-7.21 (m, 3H), 2.53 (s, 3H), 2.27 (s, 6H).

### Example 129

### N²-(2,6-Dichloro-phenyl)-N⁷-(4-methanesulfonyl-phenyl)-5-piperidin-1-yl-thiazolo[5,4-d]pyrimidine-2,7-diamine

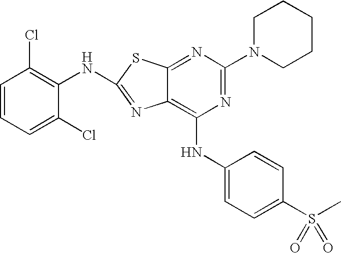

MS (ESI): mass calcd. for C₂₃H₂₂Cl₂N₆O₂S₂, 548.0; m/z found, 549.1 [M+H]⁺. ¹H NMR ((CD₃)₂SO): δ 9.78 (s, 1H), 9.35 (s, 1H), 8.03 (d, J=8.86 Hz, 2H), 7.80 (d, J=8.85 Hz, 2H), 7.61 (d, J=8.11 Hz, 2H), 7.43-7.33 (m, 1H), 3.76-3.60 (m, 4H), 3.16 (s, 3H), 1.68-1.58 (m, 2H), 1.57-1.50 (m, 4H).

### Example 130

### (racemic)-N⁷-(3-Chloro-4-trifluoromethyl-phenyl)-N²-(2,6-dichloro-phenyl)-5-(2-methyl-pyrrolidin-1-yl)-thiazolo[5,4-d]pyrimidine-2,7-diamine

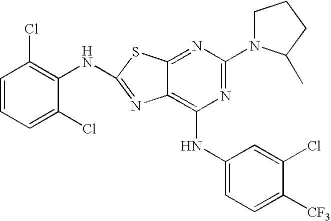

MS (ESI): mass calcd. for C₂₃H₁₈Cl₃F₃N₆S, 572.0; m/z found, 573.0 [M+H]⁺. ¹H NMR (CD₃OD): δ 8.49 (s, 1H), 7.69-7.61 (m, 2H), 7.59 (d, J=8.17 Hz, 2H), 7.44-7.37 (m, 1H), 4.44-4.28 (m, 1H), 3.80-3.63 (m, 1H), 3.58-3.48 (m, 1H), 2.31-2.01 (m, 3H), 1.88-1.78 (m, 1H), 1.34 (d, J=6.37 Hz, 3H).

### Example 131

### N⁷-(3-Chloro-4-trifluoromethylsulfanyl-phenyl)-N²-(2,6-dimethyl-phenyl)-5-methyl-thiazolo[5,4-d]pyrimidine-2,7-diamine

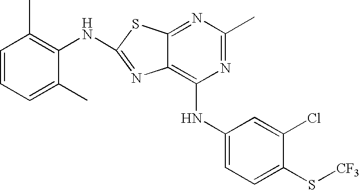

MS (ESI): mass calcd. for C₂₁H₁₇ClF₃N₅S₂, 495.0; m/z found, 496.1 [M+H]⁺. ¹H NMR ((CD₃)₂SO): δ 9.65 (s, 1H), 9.58 (s, 1H), 8.43-8.38 (m, 1H), 8.07 (dd, J=8.71, 2.33 Hz, 1H), 7.77 (d, J=8.67 Hz, 1H), 7.26-7.17 (m, 3H), 2.51 (s, 3H), 2.26 (s, 6H).

### Example 132

### N⁷-(4-Chloro-phenyl)-N²-(2,6-dimethyl-phenyl)-thiazolo[5,4-d]pyrimidine-2,7-diamine

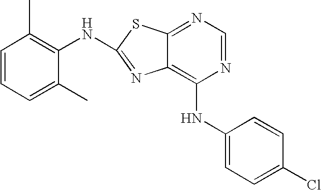

MS (ESI): mass calcd. for C₁₉H₁₆ClN₅S, 381.1; m/z found, 382.2 [M+H]⁺. ¹H NMR (CDCl₃): δ 8.35 (s, 1H), 7.66 (d, J=8.87 Hz, 2H), 7.51 (s, 1H), 7.26 (d, J=8.86 Hz, 2H), 7.21-7.17 (m, 1H), 7.13 (d, J=7.36 Hz, 2H), 2.27 (s, 6H).

### Example 133

### N²-(2,6-Dimethyl-phenyl)-N⁷-(3-fluoro-4-methyl-phenyl)-5-methyl-thiazolo[5,4-d]pyrimidine-2,7-diamine

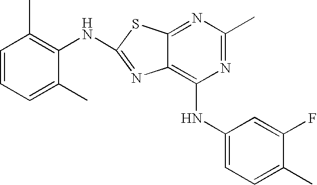

MS (ESI): mass calcd. for C₂₁H₂₀FN₅S, 393.1; m/z found, 394.2 [M+H]⁺. ¹H NMR ((CD₃)₂SO): δ 9.43 (s, 1H), 9.07 (s, 1H), 7.74 (dd, J=12.75, 1.77 Hz, 1H), 7.48 (dd, J=8.28, 1.88 Hz, 1H), 7.12 (s, 3H), 7.11-7.06 (m, 1H), 2.38 (s, 3H), 2.17 (s, 6H), 2.10 (s, 3H).

### Example 134

### N²-(2,6-Dichloro-phenyl)-N⁷-[4-(piperazine-1-sulfonyl)-phenyl]-thiazolo[5,4-d]pyrimidine-2,7-diamine

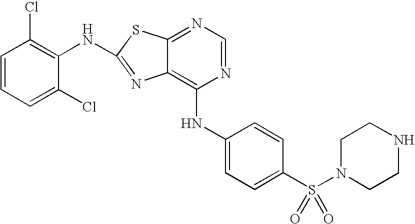

MS (ESI): mass calcd. for C₂₁H₁₉C₂N₇O₂S₂, 535.0; m/z found, 536.1 [M+H]⁺. ¹H NMR ((CD₃)₂SO): δ 10.40 (s, 1H), 9.76 (s, 1H), 8.63-8.55 (m, 2H), 8.42 (s, 1H), 8.16 (d, J=8.86 Hz, 2H), 7.75-7.63 (m, 4H), 7.51-7.42 (m, 1H), 3.27-3.14 (m, 4H), 3.12-3.04 (m, 4H).

### Example 135

### N⁷-(3-Chloro-4-trifluoromethylsulfanyl-phenyl)-N²-(2,6-dichloro-phenyl)-5-methyl-thiazolo[5,4-d]pyrimidine-2,7-diamine

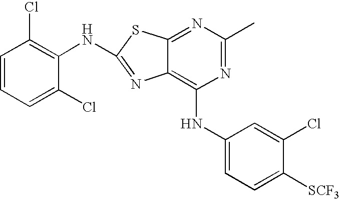

MS (ESI): mass calcd. for C₁₉H₁₁Cl₃F₃N₅S₂, 534.9, m/z found, 536.0 [M+H]⁺. ¹H NMR ((CD₃)₂SO): δ 10.25 (s, 1H), 9.63 (s, 1H), 8.37 (d, J=2.36 Hz, 1H), 8.05 (dd, J=8.67, 2.02 Hz, 1H), 7.76 (d, J=8.68 Hz, 1H), 7.65 (d, J=8.16 Hz, 2H), 7.47-7.41 (m, 1H), 2.53 (s, 3H).

### Example 136

### (racemic)-N⁷-(3-Chloro-4-trifluoromethyl-phenyl)-N²-(2,6-dichloro-phenyl)-5-(2-methyl-piperidin-1-yl)-thiazolo[5,4-d]pyrimidine-2,7-diamine

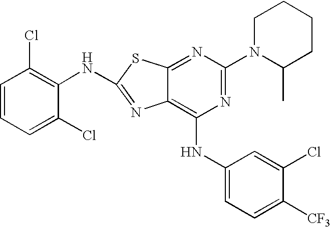

MS (ESI): mass calcd. for C₂₄H₂₀Cl₃F₃N₆S, 586.0; m/z found, 587.0 [M+H]⁺. ¹H NMR (CD₃OD): δ 8.38 (s, 1H), 7.67 (d, J=8.76 Hz, 2H), 7.60-7.54 (m, 2H), 7.44-7.36 (m, 1H), 4.50-4.34 (m, 2H), 3.23-3.00 (m, 2H), 1.94-1.76 (m, 2H), 1.75-1.64 (m, 2H), 1.63-1.48 (m, 1H), 1.30 (d, J=6.86 Hz, 3H).

### Example 137

### N²-(2,6-Dimethyl-phenyl)-N⁷-(4-iodo-phenyl)-thiazolo[5,4-d]pyrimidine-2,7-diamine

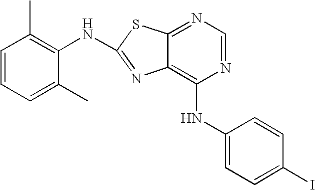

MS (ESI): mass calcd. for C₁₉H₁₆IN₅S, 473.0; m/z found, 474.1 [M+H]⁺. ¹H NMR ((CD₃)₂SO): δ 9.65 (s, 1H), 9.23 (s, 1H), 8.28 (s, 1H), 7.72 (d, J=8.82 Hz, 2H), 7.63 (d, J=8.83 Hz, 2H), 7.25-7.19 (m, 3H), 2.26 (s, 6H).

### Example 138

### N²-(2,6-Dimethyl-phenyl)-5-methyl-N⁷-p-tolyl-thiazolo[5,4-d]pyrimidine-2,7-diamine

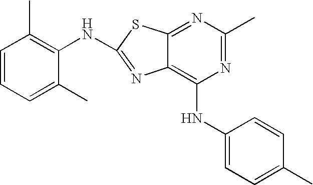

MS (ESI): mass calcd. for C₂₁H₂₁N₅S, 375.1; m/z found, 376.2 [M+H]⁺. ¹H NMR (CDCl₃): δ 7.58 (d, J=8.10 Hz, 2H), 7.21-7.15 (m, 1H), 7.13-7.10 (m, 4H), 2.58 (s, 3H), 2.28 (s, 3H), 2.26 (s, 6H).

### Example 139

### N²-(2,6-Dichloro-phenyl)-5-methyl-N⁷-(1-methyl-1,2,3,4-tetrahydro-quinolin-7-yl)-thiazolo[5,4-d]pyrimidine-2,7-diamine

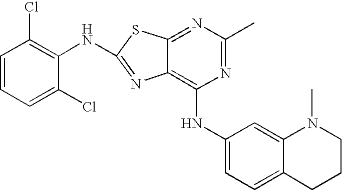

MS (ESI): mass calcd. for C₂₂H₂₀Cl₂N₆S, 470.0; m/z found, 471.1 [M+H]⁺. ¹H NMR (CDCl₃): δ 7.47 (d, J=8.21 Hz, 2H), 7.34-7.28 (m, 1H), 7.26-7.23 (m, 1H), 7.04-6.95 (m, 2H), 3.39-3.27 (m, 2H), 2.98 (s, 3H), 2.81-2.76 (m, 2H), 2.68 (s, 3H), 2.06-1.99 (m, 2H).

### Example 140

### (racemic)-1-{4-[2-(2,6-Dimethyl-phenylamino)-thiazolo[5,4-d]pyrimidin-7-ylamino]-phenyl}-ethanol

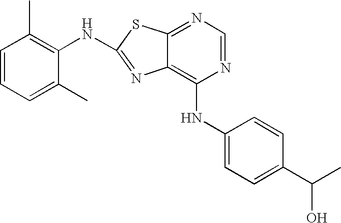

MS (ESI): mass calcd. for C₂₁H₂₁N₅OS, 391.1; m/z found, 392.2 [M+H]⁺. ¹H NMR (CDCl₃): δ 8.43 (s, 1H), 7.74 (d, J=8.27 Hz, 2H), 7.59 (s, 1H), 7.41 (d, J=8.31 Hz, 2H), 7.28-7.25 (m, 1H), 7.21 (d, J=7.56 Hz, 2H), 4.98-4.89 (m, 1H), 2.36 (s, 6H), 1.53 (d, J=6.47 Hz, 3H).

### Example 141

### N²-(2,6-Dimethyl-phenyl)-5-methyl-N⁷-phenyl-thiazolo[5,4-d]pyrimidine-2,7-diamine

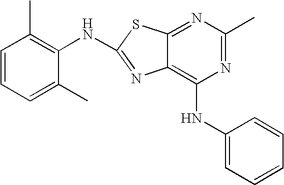

MS (ESI): mass calcd. for C₂₀H₁₉N₅S, 361.1; m/z found, 362.2 [M+H]⁺. ¹H NMR ((CD₃)₂SO): δ 9.54 (s, 1H), 9.01 (s, 1H), 7.86 (d, J=8.29 Hz, 2H), 7.34-7.26 (m, 2H), 7.23-7.18 (m, 3H), 7.05-6.98 (m, 1H), 2.45 (s, 3H), 2.26 (s, 6H).

### Example 142

### 2-Chloro-4-[2-(2,6-dimethyl-phenylamino)-thiazolo[5,4-d]pyrimidin-7-ylamino]-benzonitrile

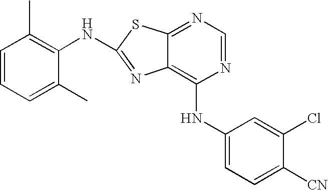

MS (ESI): mass calcd. for C₂₀H₁₅ClN₆S, 406.0; m/z found, 407.0 [M+H]⁺. ¹H NMR ((CD₃)₂SO): δ 10.59 (s, 1H), 10.41 (s, 1H), 9.08 (s, 1H), 9.07-9.05 (m, 1H), 8.72 (dd, J=8.75, 1.92 Hz, 1H), 8.52 (d, J.=8.72 Hz, 1H), 7.94-7.82 (m, 3H), 2.92 (s, 6H).

### Example 143

### (racemic)-N²-(2,6-Dimethyl-phenyl)-N⁷-(4-methanesulfinyl-phenyl)-5-methyl-thiazolo[5,4-d]pyrimidine-2,7-diamine

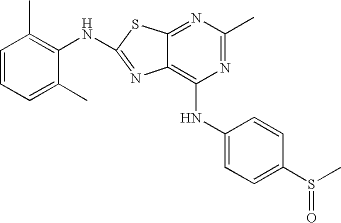

MS (ESI): mass calcd. for C₂₁H₂₁N₅OS₂, 423.1; m/z found, 424.2 [M+H]⁺. ¹H NMR (CDCl₃): δ 8.63 (s, 1H), 8.06 (d, J=8.56 Hz, 2H), 7.73 (d, J=8.61 Hz, 2H), 7.34-7.29 (m, 1H), 7.23 (d, J=7.60 Hz, 2H), 2.84 (s, 3H), 2.72 (s, 3H), 2.35 (s, 6H).

### Example 144

### N²-(2,6-Dimethyl-phenyl)-N⁷-(4-fluoro-phenyl)-thiazolo[5,4-d]pyrimidine-2,7-diamine

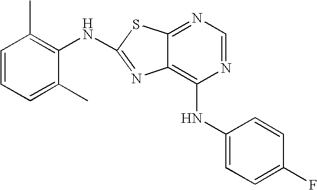

MS (ESI): mass calcd. for C₁₉H₁₆FN₅S, 365.1; m/z found, 366.2 [M+H]⁺. ¹H NMR ((CD₃)₂SO): δ 9.62 (s, 1H), 9.16 (s, 1H), 8.24 (s, 1H), 7.88-7.77 (m, 2H), 7.25-7.18 (m, 3H), 7.18-7.11 (m, 2H), 2.27 (s, 6H).

### Example 145

### 4-[2-(2,6-Dimethyl-phenylamino)-thiazolo[5,4-d]pyrimidin-7-ylamino]-N,N-dimethyl-benzamide

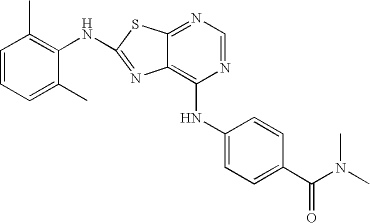

MS (ESI): mass calcd. for C₂₂H₂₂N₆OS, 418.1; m/z found, 419.2 [M+H]⁺. ¹H NMR ((CD₃)₂SO): δ 9.67 (s, 1H), 9.31 (s, 1H), 8.31 (s, 1H), 7.93 (d, J=8.55 Hz, 2H), 7.38 (d, J=8.62 Hz, 2H), 7.22 (s, 3H), 2.97 (s, 6H), 2.27 (s, 6H).

### Example 146

### (racemic)-{4-[2-(2,6-Dichloro-phenylamino)-7-(4-trifluoromethyl-phenylamino)-thiazolo[5,4-d]pyrimidin-5-yl]-morpholin-2-yl}-methanol

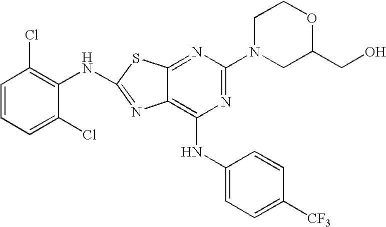

MS (ESI): mass calcd. for C₂₃H₁₉Cl₂F₃N₆O₂S, 570.0; m/z found, 571.1 [M+H]⁺. ¹H NMR (CD₃OD): δ 7.90 (d, J=8.52 Hz, 2H), 7.64-7.56 (m, 4H), 7.43-7.36 (m, 1H), 4.56-4.47 (m, 1H), 4.39-4.33 (m, 1H), 4.07-4.00 (m, 1H), 3.73-3.56 (m, 4H), 3.18-3.07 (m, 1H), 2.92-2.84 (m, 1H).

### Example 147

### N²-(2,6-Dimethyl-phenyl)-N⁷-phenyl-thiazolo[5,4-d]pyrimidine-2,7-diamine

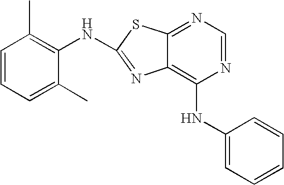

MS (ESI): mass calcd. for C₁₉H₁₇N₅S, 347.1; m/z found, 348.2 [M+H]⁺. ¹H NMR ((CD₃)₂SO): δ 9.64 (s, 1H), 9.03 (s, 1H), 8.26 (s, 1H), 7.83 (d, J=7.91 Hz, 2H), 7.35-7.27 (m, 2H), 7.22 (app s, 3H), 7.06-7.00 (m, 1H), 2.27 (s, 6H).

### Example 148

### N²-(2,6-Dimethyl-phenyl)-N⁷-(3-fluoro-4-methyl-phenyl)-thiazolo[5,4-d]pyrimidine-2,7-diamine

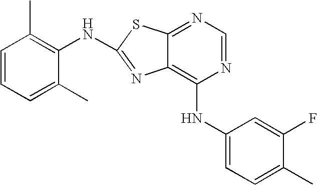

MS (ESI): mass calcd. for C₂₀H₁₈FN₅S, 379.1; m/z found, 380.2 [M+H]⁺. ¹H NMR (CD₃OD): δ 9.54 (s, 1H), 9.11 (s, 1H), 8.20 (s, 1H), 7.77-7.68 (m, 1H), 7.50-7.44 (m, 1H), 7.13 (app s, 3H), 7.12-7.06 (m, 1H), 2.17 (s, 6H), 2.10 (s, 3H).

### Example 149

### 4-[2-(2,6-Dimethyl-phenylamino)-thiazolo[5,4-d]pyrimidin-7-ylamino]-2-trifluoromethyl-benzonitrile

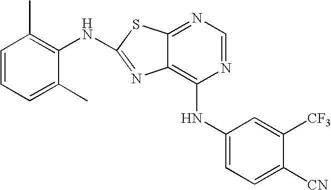

MS (ESI): mass calcd. for C₂₁H₁₅F₃N₆S, 440.1; m/z found, 441.0 [M+H]⁺. ¹H NMR ((CD₃)₂SO): δ 10.77 (s, 1H), 10.44 (s, 1H), 9.27 (s, 1H), 9.12-9.08 (m, 1H), 9.08 (s, 1H), 8.71 (d, J=8.68 Hz, 1H), 7.88 (app s, 3H), 2.92 (s, 6H).

### Example 150

### N⁷-(2,3-Dihydro-benzo[1,4]dioxin-6-yl)-N²-(2,6-dimethyl-phenyl)-thiazolo[5,4-d]pyrimidine-2,7-diamine

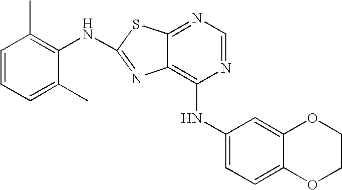

MS (ESI): mass calcd. for C₂₁H₁₉N₅O₂S, 405.1; m/z found, 406.1 [M+H]⁺. ¹H NMR (CDCl₃): δ 8.38 (s, 1H), 7.79 (s, 1H), 7.40 (d, J=2.56 Hz, 1H), 7.24-7.19 (m, 1H), 7.14 (d, J=7.60 Hz, 2H), 7.01 (dd, J=8.68, 2.56 Hz, 1H), 6.82 (d, J=8.69 Hz, 1H), 4.26-4.16 (m, 4H), 2.27 (s, 6H).

### Example 151

### N²-(2,6-Dimethyl-phenyl)-N⁷-[4-(Piperazine-1-sulfonyl)-phenyl]-thiazolo[5,4-d]pyrimidine-2,7-diamine

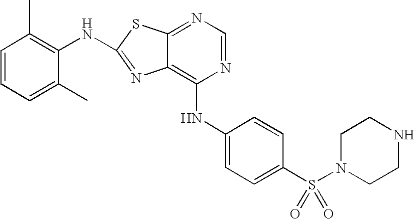

MS (ESI): mass calcd. for C₂₃H₂₅N₇O₂S₂, 495.1; m/z found, 496.2 [M+H]⁺. ¹H NMR ((CD₃)₂SO) as mono trifluoroacetic acid salt: δ 9.80-9.64 (m, 2H), 8.65-8.49 (m, 2H), 8.37 (s, 1H), 8.20 (d, J=8.78 Hz, 2H), 7.75-7.68 (m, 2H), 7.29-7.17 (m, 3H), 3.27-3.15 (m, 4H), 3.12-3.06 (m, 4H), 2.27 (s, 6H).

### Example 152

### N-{4-[2-(2,6-Dichloro-phenylamino)-5-methyl-thiazolo[5,4-d]pyrimidin-7-ylamino]-phenyl}-N-methyl-methanesulfonamide

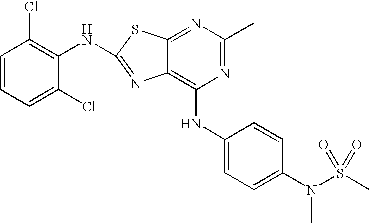

MS (ESI): mass calcd. for C₂₀H₁₈Cl₂N₆O₂S₂, 508.0; m/z found, 509.1 [M+H]⁺. ¹H NMR ((CD₃)₂SO): δ 10.17 (s, 1H), 9.18 (s, 1H), 7.87 (d, J=8.93 Hz, 2H), 7.67-7.62 (m, 1H), 7.47-7.40 (m, 1H), 7.32 (d, J=8.91 Hz, 2H), 3.21 (s, 3H), 2.93 (s, 3H), 2.47 (s, 3H).

### Example 153

### N²-(2,6-Dichloro-phenyl)-N⁵-[3-(4-methyl-piperazin-1-yl)-propyl]-N⁷-(4-trifluoromethyl-phenyl)-thiazolo[5,4-d]pyrimidine-2,5,7-diamine

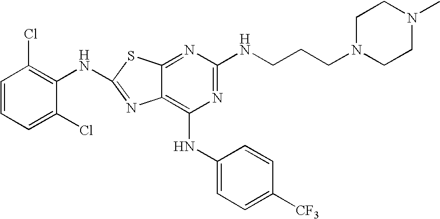

MS (ESI): mass calcd. for C₂₆H₂₇Cl₂F₃N₈S, 610.1; m/z found, 611.1 [M+H]⁺. ¹H NMR (CD₃OD): δ 7.98 (d, J=8.67 Hz, 2H), 7.63 (d, J=8.62 Hz, 2H), 7.60-7.57 (m, 2H), 7.43-7.37 (m, 1H), 3.55-3.48 (m, 2H), 3.29-3.20 (m, 4H), 3.14-2.95 (m, 4H), 2.92-2.86 (m, 2H), 2.81 (s, 3H), 2.01-1.92 (m, 2H).

### Example 154

### (racemic)-N²-(2,6-Dimethyl-phenyl)-N⁷-[4-(tetrahydro-furan-3-yloxy)-phenyl]-thiazolo[5,4-d]pyrimidine-2,7-diamine

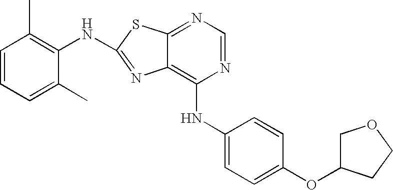

MS (ESI): mass calcd. for C₂₃H₂₃N₅O₂S, 433.1; m/z found, 434.2 [M+H]⁺. ¹H NMR (CDCl₃): δ 8.48 (s, 1H), 7.69-7.65 (m, 2H), 7.36-7.29 (m, 1H), 7.23 (d, J=7.64 Hz, 2H), 6.94 (d, J=9.00 Hz, 2H), 5.00-4.92 (m, 2H), 4.11-3.98 (m, 2H), 3.97-3.91 (m, 1H), 2.36 (s, 6H), 2.30-2.15 (m, 2H).

### Example 155

### (racemic)-{4-[7-(3-Chloro-4-trifluoromethyl-phenylamino)-2-(2,6-dichloro-phenylamino)-thiazolo[5,4-d]pyrimidin-5-yl]-morpholin-2-yl}-methanol

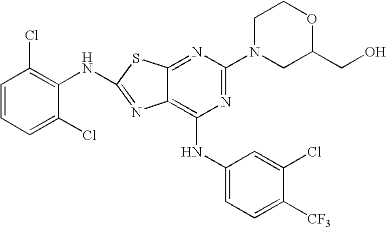

MS (ESI): mass calcd. for C₂₃H₁₈Cl₃F₃N₆O₂S, 604.0; m/z found, 605.0 [M+H]⁺. ¹H NMR (CD₃OD): δ 8.31-8.25 (m, 2H), 7.71-7.63 (m, 2H), 7.59 (d, J=8.16 Hz, 2H), 7.43-7.37 (m, 1H), 4.55-4.47 (m, 1H), 4.41-4.35 (m, 1H), 4.08-4.01 (m, 1H), 3.72-3.58 (m, 4H), 3.17-3.07 (m, 1H), 2.94-2.84 (m, 1H).

### Example 156

### Cyclopentyl-{4-[2-(2,6-dichloro-phenylamino)-5-methyl-thiazolo[5,4-d]pyrimidin-7-ylamino]-phenyl}-methanone

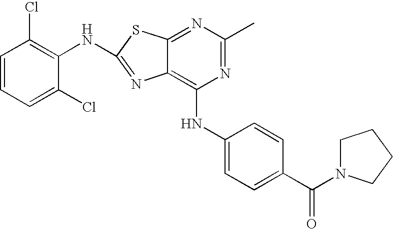

MS (ESI): mass calcd. for C₂₄H₂₀Cl₂N₆OS, 498.0; m/z found, 499.1 [M+H]⁺. ¹H NMR ((CD₃)₂SO): δ 10.19 (s, 1H), 9.23 (s, 1H), 7.91 (d, J=8.65 Hz, 2H), 7.65 (d, J=8.14 Hz, 2H), 7.48 (d, J=8.62 Hz, 2H), 7.46-7.40 (m, 1H), 2.49 (s, 3H), 1.92-1.76 (m, 4H).

### Example 157

### 4-[2-(2,6-Dichloro-phenylamino)-5-methyl-thiazolo[5,4-d]pyrimidin-7-ylamino]-N,N-dimethyl-benzamide

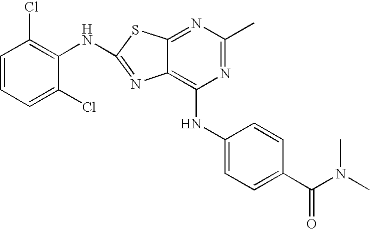

MS (ESI): mass calcd. for C₂₁H₁₈Cl₂N₆OS, 472.0; m/z found, 473.1 [M+H]⁺. ¹H NMR (CDCl₃): δ 7.78 (d, J=8.02 Hz, 2H), 7.52-7.47 (m, 4H), 7.36-7.31 (m, 1H), 3.26-2.97 (m, 6H), 2.80 (s, 3H).

### Example 158

### 2-[2-(2,6-Dimethyl-phenylamino)-thiazolo[5,4-d]pyrimidin-7-ylamino]-5-methyl-phenol

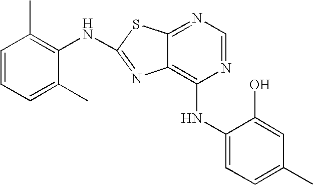

MS (ESI): mass calcd. for C₂₀H₁₉N₅OS, 377.1; m/z found, 378.1 [M+H]⁺. ¹H NMR (CD₃OD): δ 8.19 (s, 1H), 7.63-7.45 (m, 1H), 7.26-7.17 (m, 3H), 6.82-6.78 (m, 1H), 6.77-6:72 (m, 1H), 2.33 (s, 6H), 2.30 (s, 3H).

### Example 159

### N²-(2,6-Dimethyl-phenyl)-N⁷-(2-methyl-4-trifluoromethyl-phenyl)-thiazolo[5,4-d]pyrimidine-2,7-diamine

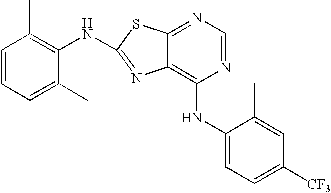

MS (ESI): mass calcd. for C₂₁H₁₈F₃N₅S, 429.1; m/z found, 430.2 [M+H]⁺. ¹H NMR (CD₃OD): δ 8.23 (s, 1H), 7.96-7.83 (m, 1H), 7.63-7.57 (m, 1H), 7.56-7.51 (m, 1H), 7.25-7.18 (m, 3H), 2.35 (s, 3H), 2.33 (s, 6H).

### Example 160

### 5-[2-(2,6-Dimethyl-phenylamino)-thiazolo[5,4-d]pyrimidin-7-ylamino]-2-methyl-phenol

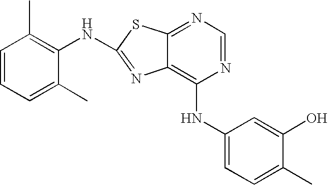

MS (ESI): mass calcd. for C₂₀H₁₉N₅OS, 377.1; m/z found, 378.1 [M+H]⁺. ¹H NMR (CD₃OD): δ 8.90 (s, 1H), 7.95-7.87 (m, 4H), 7.77-7.70 (m, 1H), 7.64-7.59 (m, 1H), 3.00 (s, 6H), 2.84 (s, 3H).

### Example 161

### N-{4-[2-(2,6-Dimethyl-phenylamino)-thiazolo[5,4-d]pyrimidin-7-ylamino]-phenyl}-N-methyl-methanesulfonamide

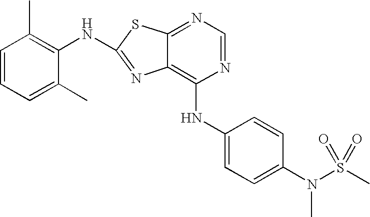

MS (ESI): mass calcd. for C₂₁H₂₂N₆O₂S₂, 454.1; m/z found, 455.2 [M+H]⁺. ¹H NMR ((CD₃)₂SO): δ 9.65 (s, 1H), 9.24 (s, 1H), 8.27 (s, 1H), 7.86 (d, J=8.86 Hz, 2H), 7.38-7.30 (m, 1H), 7.24-7.20 (m, 3H), 3.22 (s, 3H), 2.94 (s, 3H), 2.27 (s, 6H).

### Example 162

### N⁷-(3-Chloro-4-trifluoromethyl-phenyl)-N²-(2,6-dichloro-phenyl)-5-piperazin-1-yl-thiazolo[5,4-d]pyrimidine-2,7-diamine

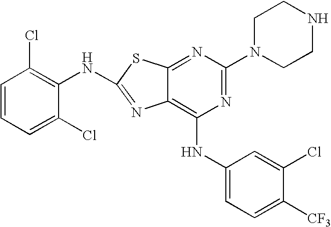

MS (ESI): mass calcd. for C₂₂H₁₇Cl₃F₃N₇S, 573.0; m/z found, 574.0 [M+H]⁺. ¹H NMR ((CD₃)₂SO) as mono trifluoroacetic acid salt: δ 9.92 (s, 1H), 9.65 (s, 1H), 8.91-8.70 (m, 2H), 8.23-8.12 (m, 1H), 7.97-7.90 (m, 1H), 7.78-7.72 (m, 1H), 7.62 (d, J=8.13 Hz, 2H), 7.45-7.37 (m, 1H), 3.93-3.82 (m, 4H), 3.26-3.12 (m, 4H).

### Example 163

### N²-(2,6-Dichloro-phenyl)-5-piperazin-1-yl-N⁷-(4-trifluoromethyl-phenyl)-thiazolo[5,4-d]pyrimidine-2,7-diamine

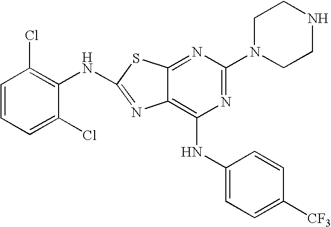

MS (ESI): mass calcd. for C₂₂H₁₈Cl₂F₃N₇S, 539.0; m/z found, 540.1 [M+H]⁺. ¹H NMR ((CD₃)₂SO) as mono trifluoroacetic acid salt: δ 9.88 (s, 1H), 9.41. (s, 1H), 8.84-8.72 (m, 2H), 7.97 (d, J=8.57 Hz, 2H), 7.69-7.59 (m, 4H), 7.46-7.36 (m, 1H), 3.91-3.80 (m, 4H), 3.26-3.13 (m, 4H).

### Example 164

### N-{4-[2-(2,6-Dimethyl-phenylamino)-5-methyl-thiazolo[5,4-d]pyrimidin-7-ylamino]-phenyl}-N-methyl-methanesulfonamide

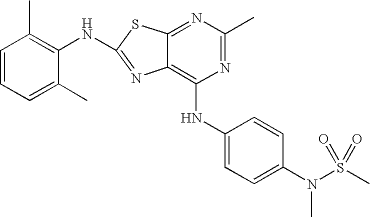

MS (ESI): mass calcd. for C₂₂H₂₄N₆O₂S₂, 468.1; m/z found, 469.2 [M+H]⁺. ¹H NMR ((CD₃)₂SO): δ 7.87 (d, J=8.76 Hz, 2H), 7.45-7.39 (m, 2H), 7.32-7.27 (m, 1H), 7.22 (d, J=7.62 Hz, 2H), 3.36 (s, 3H), 2.90 (s, 3H), 2.69 (s, 3H), 2.34 (s, 6H).

### Example 165

### N²-(2,6-Dimethyl-phenyl)-N⁷-(3,4-dimethyl-phenyl)-thiazolo[5,4-d]pyrimidine-2,7-diamine

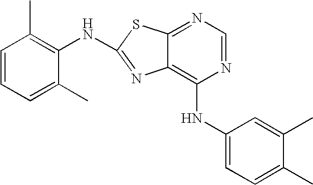

MS (ESI): mass calcd. for C₂₁H₂₁N₅S, 375.1; m/z found, 376.1 [M+H]⁺. ¹H NMR ((CD₃)₂SO): δ 9.66 (s, 1H), 8.86 (s, 1H), 8.29-8.20 (m, 1H), 7.62-7.46 (m, 2H), 7.22 (s, 3H), 7.06 (d, J=8.18 Hz, 1H), 2.26 (s, 6H), 2.19 (d, J=11.87 Hz, 6H).

### Example 166

### N²-(2,6-Dichloro-phenyl)-5-methyl-N⁷-pyridin-3-yl-thiazolo[5,4-d]pyrimidine-2,7-diamine

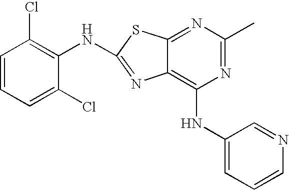

MS (ESI): mass calcd. for C₁₇H₁₂Cl₂N₆S, 402.3; m/z found, 403.1 [M+H]⁺. ¹H NMR (CD₃OD): δ 8.96 (d, J=2.54 Hz, 1H), 8.44-8.31 (m, 1H), 8.22-8.17 (m, 1H), 7.57 (d, J=8.12 Hz, 2H), 7.42-7.35 (m, 2H), 2.59 (s, 3H).

### Example 167

### N⁷-(2-Chloro-4-trifluoromethyl-phenyl)-N²-(2,6-dimethyl-phenyl)-thiazolo[5,4-d]pyrimidine-2,7-diamine

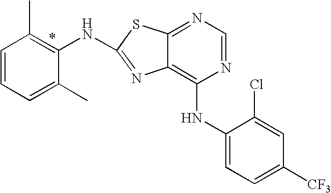

MS (ESI): mass calcd. for C₂₁H₁₈F₃N₅S, 449.0; m/z found, 450.0 [M+H]⁺. ¹H NMR ((CD₃)₂SO): δ 10.13 (s, 1H), 8.74-8.63 (m, 1H), 8.51 (s, 1H), 8.36 (s, 1H), 7.96-7.92 (m, 1H), 7.79-7.74 (m, 1H), 7.24-7.16 (m, 3H), 2.25 (s, 6H).

### Example 168

### N²-(2,6-Dimethyl-phenyl)-N⁷-(4-methoxy-3-trifluoromethyl-phenyl)-thiazolo[5,4-d]pyrimidine-2,7-diamine

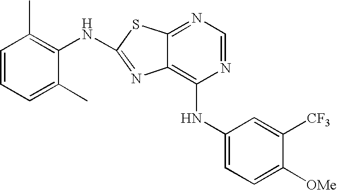

MS (ESI): mass calcd. for C₂₁H₁₈F₃N₅OS, 445.1; m/z found, 446.1 [M+H]⁺. ¹H NMR ((CD₃)₂SO): δ 9.62 (s, 1H), 9.24 (s, 1H), 8.23 (s, 1H), 8.15-8.07 (m, 1H), 8.03-7.99 (m, 1H), 7.26-7.14 (m, 4H), 3.85 (s, 3H), 2.25 (s, 6H).

### Example 169

### 4-[2-(2,6-Dimethyl-phenylamino)-thiazolo[5,4-d]pyrimidin-7-ylamino]-benzamide

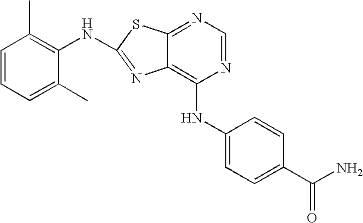

MS (ESI): mass calcd. for C₂₀H₁₈N₆OS, 390.1; m/z found, 391.1 [M+H]⁺. ¹H NMR (CDCl₃): δ 8.47 (s, 1H), 8.04 (s, 1H), 7.89 (d, J=8.76 Hz, 2H), 7.80 (d, J=8.76 Hz, 2H), 7.27-7.18 (m, 1H), 7.17-7.13 (m, 2H), 6.04 (s, 1H), 2.28 (s, 6H).

### Example 170

### N²-(2,6-Dichloro-phenyl)-5-methyl-N⁷-phenyl-thiazolo[5,4-d]pyrimidine-2,7-diamine

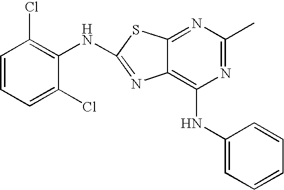

MS (ESI): mass calcd. for C₁₈H₁₃Cl₂N₅S, 401.0; m/z found, 402.1 [M+H]⁺. ¹H NMR ((CD₃)₂SO): δ 10.16 (s, 1H), 9.01 (s, 1H), 7.82 (d, J=8.36 Hz, 2H), 7.64 (d, J=8:11 Hz, 2H), 7.47-7.38 (m, 1H), 7.33-7.25 (m, 2H), 7.07-6.96 (m, 1H), 2.46 (s, 3H).

### Example 171

### N²-(2,6-Dimethyl-phenyl)-5-methyl-N⁷-pyridin-3-yl-thiazolo[5,4-d]pyrimidine-2,7-diamine

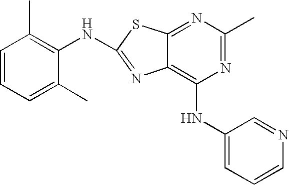

MS (ESI): mass calcd. for C₁₉H₁₈N₆S, 362.1; m/z found, 363.2 [M+H]⁺. ¹H NMR (CD₃OD): δ 9.02-8.91 (m, 1H), 8.42-8.33 (m, 1H), 8.22-8.16 (m, 1H), 7.44-7.33 (m, 1H), 7.27-7.19 (m, 3H), 2.57 (s, 3H), 2.33 (s, 6H).

### Example 172

### 4-[2-(2,6-Dimethyl-phenylamino)-thiazolo[5,4-d]pyrimidin-7-ylamino]-benzoic acid

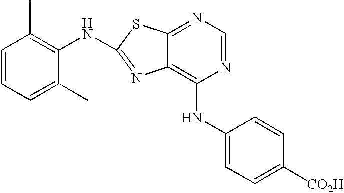

MS (ESI): mass calcd. for C₂₀H₁₇N₅O₂S, 391.1; m/z found, 392.2 [M+H]⁺. ¹H NMR ((CD₃)₂SO): δ 9.75-9.64 (m, 1H), 9.49-9.38 (m, 1H), 8.33 (s, 1H), 8.00 (d, J=8.78 Hz, 2H), 7.86 (d, J=8.82 Hz, 2H), 7.23-7.19 (m, 3H), 2.25 (s, 6H).

### Example 173

### N-{4-[2-(2,6-Dichloro-phenylamino)-5-methyl-thiazolo[5,4-d]pyrimidin-7-ylamino]-phenyl}-dimethanesulfonamide

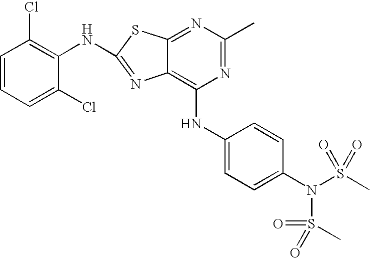

MS (ESI): mass calcd. for C₂₀H₁₈C₂N₆O₄S₃, 571.9; m/z found, 573.0 [M+H]⁺. ¹H NMR ((CD₃)₂SO): δ 10.21 (s, 1H), 9.38 (s, 1H), 8.02-7.90 (m, 2H), 7.65 (d, J=8.13 Hz, 2H), 7.48-7.37 (m, 3H), 3.52 (s, 6H), 2.50 (s, 3H).

### Example 174

### N-{4-[2-(2,6-Dimethyl-phenylamino)-5-methyl-thiazolo[5,4-d]pyrimidin-7-ylamino]-phenyl}-methanesulfonamide

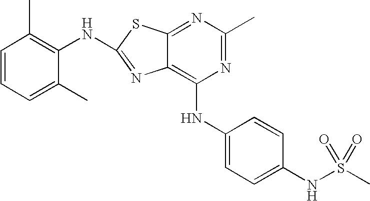

MS (ESI): mass calcd. for C₂₁H₂₂N₆O₂S₂, 454.1; m/z found, 455.2 [M+H]⁺. ¹H NMR ((CD₃)₂SO): δ 9.47-9.39 (m, 2H), 8.95 (s, 1H), 7.77-7.68 (m, 2H), 7.12 (s, 3H), 7.08-7.05 (m, 2H), 2.86 (s, 3H), 2.35 (s, 3H), 2.17 (s, 6H).

### Example 175

### N-{4-[2-(2,6-Dimethyl-phenylamino)-thiazolo[5,4-d]pyrimidin-7-ylamino]-phenyl}-methanesulfonamide

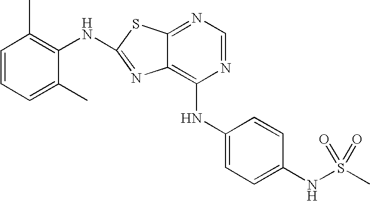

MS (ESI): mass calcd. for C₂₀H₂₀N₆O₂S₂, 440.1; m/z found, 441.2 [M+H]⁺. ¹H NMR (CDCl₃): δ 8.42 (s, 1H), 8.04 (s, 1H), 7.75-7.69 (m, 2H), 7.26-7.17 (m, 4H), 7.14 (d, J=7.62 Hz, 2H), 6.55 (s, 1H), 2.95 (s, 3H), 2.27 (s, 6H).

### Example 176

### N²-(2,6-Dimethyl-phenyl)-N⁷-(5-trifluoromethyl-pyridin-2-yl)-thiazolo[5,4-d]pyrimidine-2,7-diamine

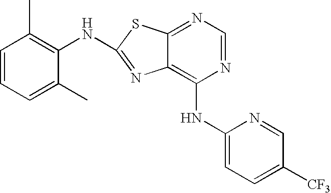

To a sealed tube under N₂ was added (7-chloro-thiazolo[5,4-d]pyrimidin-2-yl)-(2,6-dimethyl-phenyl)-amine (72.0 mg, 0.26 mmol), 5-trifluoromethyl-pyridin-2-ylamine (50 mg, 0.2 mmol), Pd₂(dba)₂ (10 mg, 0.01 mmol), 2-(di-t-butylphosphino)biphenyl (13 mg, 0.04 mmol), sodium t-butoxide (29 mg, 0.31 mmol) and freshly distilled toluene (2 mL). The resulting mixture was heated to 90° C. After 24 h, the mixture was filtered through a plug of diatomaceous earth, eluting with EtOAc (20 mL). The filtrate was concentrated and the crude residue was purified by reverse phase HPLC to provide the title compound (7.2 mg, 6.5%). MS (ESI): mass calcd. for C₁₉H₁₅F₃N₆S, 416.1; m/z found, 417.0 [M+H]⁺. ¹H NMR (CD₃OD): δ 9.04-8.96 (m, 1H), 8.93-8.81 (m, 1H), 8.46 (dd, J=8.81, 2.26 Hz, 1H), 8.21-8.09 (m, 1H), 7.25-7.05 (m, 3H), 2.32 (s, 6H).

### Example 177

### (racemic)-N²-(2,6-Dichloro-phenyl)-5-(2-isopropyl-pyrrolidin-1-yl)-N⁷-(4-methanesulfonyl-phenyl)-thiazolo[5,4-d]pyrimidine-2,7-diamine

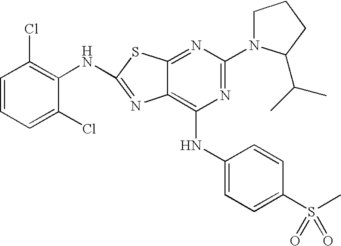

MS (ESI): mass calcd. for C₂₅H₂₆Cl₂N₆O₂S₂, 576.1, m/z found, 577.1 [M+H]⁺. ¹H NMR ((CD₃)₂SO): δ 8.06-7.98 (m, 2H), 7.91-7.85 (m, 2H), 7.62-7.56 (m, 2H), 7.45-7.37 (m, 1H), 4.31-4.11 (m, 1H), 3.73-3.57 (m, 2H), 3.11 (s, 3H), 2.50-2.34 (m, 1H), 2.21-1.97 (m, 4H), 0.98 (d, J=6.94 Hz, 3H), 0.87 (d, J=6.81 Hz, 3H).

### Example 178

### N²-(2,6-Dichloro-phenyl)-N⁷-(4-methanesulfonyl-phenyl)-5-morpholin-4-yl-thiazolo[5,4-d]pyrimidine-2,7-diamine

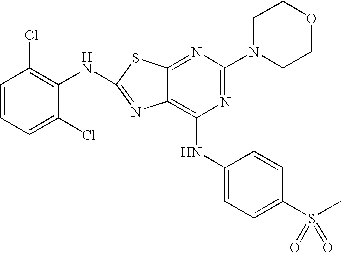

MS (ESI): mass calcd. for C₂₂H₂₀Cl₂N₆O₃S₂, 550.0; m/z found, 551.0 [M+H]⁺. ¹H NMR ((CD₃)₂SO): δ 9.83 (s, 1H), 9.41 (s, 1H), 8.04 (d, J=8.85 Hz, 2H), 7.81 (d, J=8.84 Hz, 2H), 7.61 (d, J=8.12 Hz, 2H), 7.44-7.33 (m, 1H), 3.70-3.64 (m, 4H), 3.64-3.59 (m, 4H), 3.15 (s, 3H).

### Example 179

### (racemic)-N²-(2,6-Dichloro-phenyl)-N⁷-(4-methanesulfonyl-phenyl)-5-(2-methyl-piperidin-1-yl)-thiazolo[5,4-d]pyrimidine-2,7-diamine

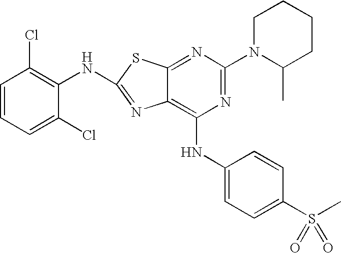

MS (ESI): mass calcd. for C₂₄H₂₄Cl₂N₆O₂S₂, 562.1; m/z found, 563.1 [M+H]⁺. ¹H NMR (CD₃OD): δ 7.91-7.85 (m, 2H), 7.84-7.79 (m, 2H), 7.50 (d, J=8.05 Hz, 2H), 7.34-7.28 (m, 1H), 4.32-4.21 (m, 1H), 3.03 (s, 3H), 1.81-1.67 (m, 4H), 1.68-1.55 (m, 2H), 1.57-1.37 (m, 2H), 1.21 (d, J=6.88 Hz, 3H).

### Example 180

### N²-(2,6-Dichloro-phenyl)-N⁵-(2-piperidin-1-yl-ethyl)-N⁷-(4-trifluoromethyl-phenyl)-thiazolo[5,4-d]pyrimidine-2,5,7-triamine

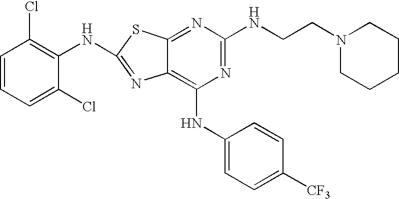

MS (ESI): mass calcd. for C₂₅H₂₄Cl₂F₃N₇S, 581.1; m/z found, 582.1 [M+H]⁺. ¹H NMR (CD₃OD): δ 7.88 (d, J=8.51 Hz, 2H), 7.58 (d, J=8.64 Hz, 2H), 7.53 (d, J=8.08 Hz, 2H), 7.37-7.30 (m, 1H), 3.80-3.72 (m, 2H), 3.63-3.51 (m, 2H), 3.30-3.27 (m, 2H), 2.97-2.80 (m, 2H), 1.93-1.61 (m, 4H), 1.57-1.35 (m, 2H).

### Example 181

### N²-(2,6-Dichloro-phenyl)-N⁵-(2-methylamino-ethyl)-N⁷-(4-trifluoromethyl-phenyl)-thiazolo[5,4-d]pyrimidine-2,5,7-triamine

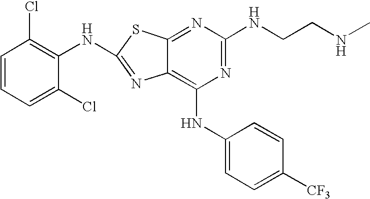

MS (ESI): mass calcd. for C₂₁H₁₈Cl₂F₃N₇S, 541.1; m/z found, 542.1 [M+H]⁺. ¹H NMR (CD₃OD): δ 7.95 (d, J=8.58 Hz, 2H), 7.61 (d, J=8.77 Hz, 2H), 7.58 (d, J=8.12 Hz, 2H), 7.43-7.37 (m, 1H), 3.99-3.93 (m, 2H), 3.26 (s, 3H), 2.72 (s, 3H).

### Example 182

### N²-(2,6-Dichloro-phenyl)-N⁵-(2-dimethhylamino-ethyl)-methyl-N⁷-(4-trifluoromethyl-phenyl)-thiazolo[5,4-d]pyrimidine-2,5,7-triamine

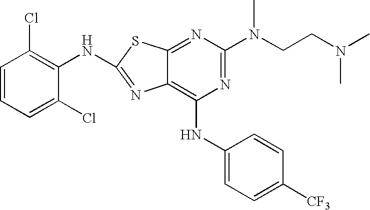

MS (ESI): mass calcd. for C₂₃H₂₂Cl₂F₃N₇S, 555.1; m/z found, 556.1 [M+H]⁺. ¹H NMR (CD₃OD): δ 7.89 (d, J=8.48 Hz, 2H), 7.61 (d, J=8.63 Hz, 2H), 7.58-7.55 (m, 2H), 7.41-7.35 (m, 1H), 4.06-3.99 (m, 2H), 3.47-3.38 (m, 2H), 3.23 (s, 3H), 2.92 (s, 6H).

### Example 183

### (3R)-N²-(2,6-Dichloro-phenyl)-5-(3-methylamino-pyrrolidin-1-yl)-N⁷-(4-trifluoromethyl-phenyl)-thiazolo[5,4-d]pyrimidine-2,7-diamine

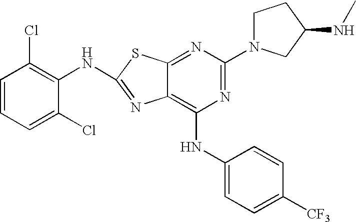

MS (ESI): mass calcd. for C₂₃H₂₀Cl₂F₃N₇S, 553.0; m/z found, 554.1 [M+H]⁺. ¹H NMR ((CD₃)₂SO) as mono trifluoroacetic acid salt: δ 9.79 (s, 1H), 9.31 (s, 1H), 8.82-8.62 (m, 2H), 8.12 (d, J=8.55 Hz, 2H), 7.64-7.59 (m, 4H), 7.43-7.36 (m, 1H), 3.92-3.76 (m, 2H), 3.74-3.63 (m, 2H), 3.61-3.53 (m, 1H), 2.68-2.63 (m, 3H), 2.40-2.31 (m, 2H).

### Example 184

### N⁵-Cyclopropylmethyl-N²-(2,6-dichloro-phenyl)-N⁷-(4-methanesulfonyl-phenyl)-thiazolo[5,4-d]pyrimidine-2,5,7-triamine

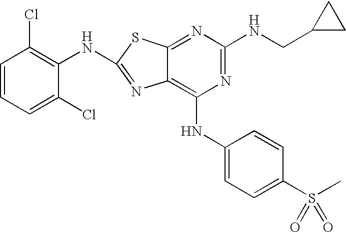

MS (ESI): mass calcd. for C₂₂H₂₀Cl₂N₆O₂S₂, 534.0; m/z found, 535.0 [M+H]⁺. ¹H NMR ((CD₃)₂SO): δ 9.75 (s, 1H), 9.34 (s, 1H), 8.16 (d, J=8.90 Hz, 2H), 7.78 (d, J=8.92 Hz, 2H), 7.61 (d, J=8.14 Hz, 2H), 7.43-7.35 (m, 1H), 3.16 (s, 3H), 3.19-3.13 (m, 2H), 1.14-1.03 (m, 1H), 0.50-0.35 (m, 2H), 0.25-0.19 (m, 2H).

### Example 185

### N²-(2,6-Dichloro-phenyl)-5-methyl-N⁷-(6-methylsulfanyl-pyridin-3-yl)-thiazolo[5,4-d]pyrimidine-2,7-diamine

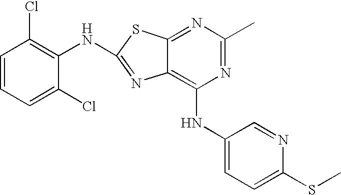

MS (ESI): mass calcd. for C₁₈H₁₄Cl₂N₆S2, 433.9; m/z found, 435.0 [M+H]⁺. ¹H NMR ((CD₃)₂SO): δ 10.29 (s, 1H), 9.33 (s, 1H), 8.81 (d, J=2.53 Hz, 1H), 8.30 (s, 1H), 8.07 (dd, J=8.72, 2.63 Hz, 1H), 7.65 (d, J=8.14 Hz, 2H), 7.47-7.40 (m, 1H), 7.29-7.23 (m, 1H), 2.51 (s, 3H).

### Example 186

### (racemic)-2-[2-(2,6-Dichloro-phenylamino)-7-(4-trifluoromethyl-phenylamino)-thiazolo[5,4-d]pyrimidin-5-ylamino]-propan-1-ol

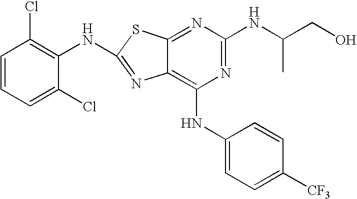

MS (ESI): mass calcd. for C₂₁H₁₇Cl₂F₃N₆OS, 528.0; m/z found, 529.0 [M+H]⁺. ¹H NMR (CD₃OD): δ 7.96 (d, J=8.47 Hz, 2H), 7.66 (d, J=8.66 Hz, 2H), 7.60-7.56 (m, 2H), 7.43-7.37 (m, 1H), 4.15-4.04 (m, 1H), 3.68-3.59 (m, 2H), 1.37-1.21 (m, 3H).

### Example 187

### N²-(2,6-Dichloro-phenyl)-5-(4-methyl-piperazin-1-yl)-N⁷-(4-trifluoromethyl-phenyl)-thiazolo[5,4-d]pyrimidine-2,7-diamine

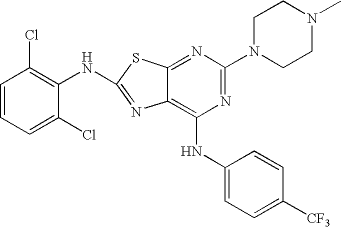

MS (ESI): mass calcd. for C₂₃H₂₀Cl₂F₃N₇S, 553.1; m/z found, 554.1 [M+H]⁺. ¹H NMR (CD₃OD): δ 7.57 (d, J=8.62 Hz, 2H), 7.35-7.25 (m, 4H), 7.13-7.08 (m, 1H), 4.58-4.49 (m, 4H), 3.39-3.19 (m, 2H), 2.93-2.80 (m, 2H), 2.67 (s, 3H).

### Example 188

### N²-(2,6-Dichloro-phenyl)-N⁵,N⁵-diethyl-N⁷-(4-trifluoromethyl-phenyl)-thiazolo[5,4-d]pyrimidine-2,5,7-triamine

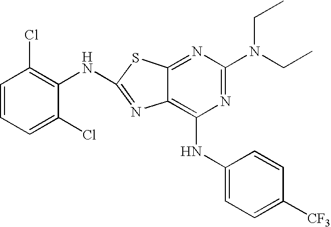

MS (ESI): mass calcd. for C₂₂H₁₉Cl₂F₃N₆S, 526.1; m/z found, 527.1 [M+H]⁺. ¹H NMR (CD₃OD): δ 7.85 (d, J=8.71 Hz, 2H), 7.57-7.47 (m, 4H), 7.34-7.28 (m, 1H), 3.57 (q, J=7.10 Hz, 4H), 1.18 (t, J=7.06 Hz, 6H).

### Example 189

### 5-Butoxy-N²-(2,6-dichloro-phenyl)-N⁷-(4-trifluoromethyl-phenyl)-thiazolo[5,4-d]pyrimidine-2,7-diamine

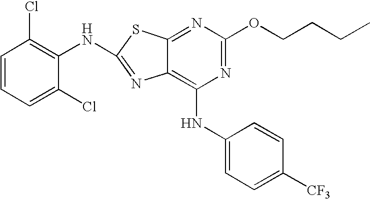

MS (ESI): mass calcd. for C₂₂H₁₈Cl₂F₃N₅OS, 527.1; m/z found, 528.1 [M+H]⁺. ¹H NMR (CD₃OD): δ 8.01 (d, J=8.48 Hz, 2H), 7.71-7.62 (m, 4H), 7.49-7.44 (m, 1H), 4.45 (t, J=6.58, 6.58 Hz, 2H), 1.91-1.82 (m, 2H), 1.65-1.51 (m, 2H), 1.06 (t, J=7.41, 7.41 Hz, 3H).

### Example 190

### N²-(2,6-Dichloro-phenyl)-5-(4-methyl-piperidin-1-yl)-N²-(4-trifluoromethyl-phenyl)-thiazolo[5,4-d]pyrimidine-2,7-diamine

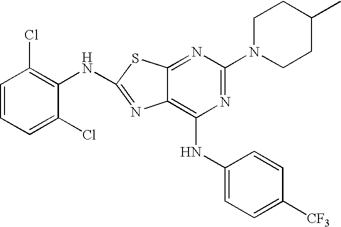

MS (ESI): mass calcd. for C₂₄H₂₁Cl₂F₃N₆S, 552.1; m/z found, 553.1 [M+H]⁺. ¹H NMR (CD₃OD): δ 7.76 (d, J=8.53 Hz, 2H), 7.53 (d, J=8.62 Hz, 2H), 7.50-7.46 (m, 2H), 7.31-7.26 (m, 1H), 4.36 (d, J=13.32 Hz, 2H), 2.95 (dt, J=13.29, 13.18, 2.25 Hz, 2H), 1.78-1.53 (m, 3H), 1.22-1.05 (m, 2H), 0.89 (d, J=6.45 Hz, 3H).

### Example 191

### (racemic)-N²-(2,6-Dichloro-phenyl)-5-(2-methyl-piperidin-1-yl)-N⁷-(4-trifluoromethyl-phenyl)-thiazolo[5,4-d]pyrimidine-2,7-diamine

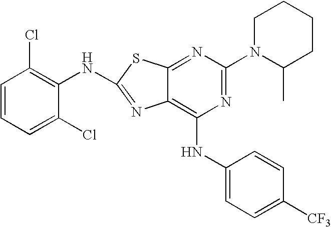

MS (ESI): mass calcd. for C₂₄H₂₁Cl₂F₃N₆S, 552.1; m/z found, 553.1 [M+H]⁺. ¹H NMR (CDCl₃): δ 8.11 (s, 1H), 7.85 (d, J=8.49 Hz, 2H), 7.65 (d, J=8.51 Hz, 2H), 7.50 (d, J=7.83 Hz, 2H), 7.35 (dd, J=8.68, 7.58 Hz, 1H), 4.94-4.84 (m, 1H), 4.51-4.38 (m, 1H), 3.11-3.01 (m, 1H), 1.88-1.42 (m, 6H), 1.25 (d, J=6.90 Hz, 3H)

### Example 192

### (3S)-N²-(2,6-Dichloro-phenyl)-5-(3-methyl-morpholin-4-yl)-N⁷-(4-trifluoromethyl-phenyl)-thiazolo[5,4-d]pyrimidine-2,7-diamine

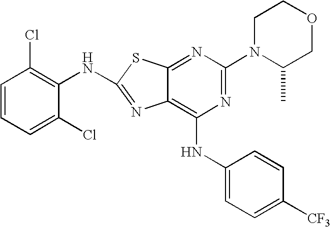

MS (ESI): mass calcd. for C₂₃H₁₉Cl₂F₃N₆OS, 554.1; m/z found, 555.1 [M+H]⁺. ¹H NMR (CD₃OD): δ 7.89 (d, J=8.97 Hz, 2H), 7.66-7.54 (m, 4H), 7.42-7.38 (m, 1H), 4.57-4.49 (m, 1H), 4.20-4.07 (m, 1H), 4.05-3.96 (m, 1H), 3.86-3.70 (m, 2H), 3.64-3.53 (m, 1H), 3.40-3.34 (m, 1H), 1.35 (d, J=6.80 Hz, 3H).

### Example 193

### (2S)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine

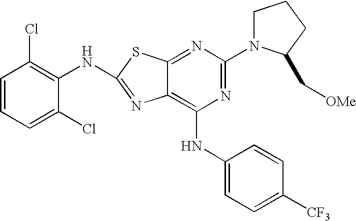

MS (ESI): mass calcd. for C₂₄H₂₁Cl₂F₃N₆OS, 568.1; m/z found, 569.1 [M+H]⁺. ¹H NMR (CD₃OD): δ 7.95 (d, J=8.56 Hz, 2H), 7.64-7.54 (m, 4H), 7.39 (t, J=8.15, 8.15 Hz, 1H), 4.33 (s, 1H), 3.69-3.56 (m, 2H), 3.57-3.45 (m, 1H), 3.39 (t, J=8.28, 8.28 Hz, 1H), 3.31 (s, 3H), 2.25-1.93 (m, 4H).

### Example 194

### (2R)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine

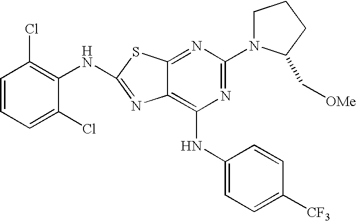

MS (ESI): mass calcd. for C₂₄H₂₁Cl₂F₃N₆OS, 568.1; m/z found, 569.1 [M+H]⁺. ¹H NMR (CD₃OD): δ 7.95 (d, J=8.56 Hz, 2H), 7.64-7.54 (m, 4H), 7.39 (t, J=8.15, 8.15 Hz, 1H), 4.33 (s, 1H), 3.69-3.56 (m, 2H), 3.57-3.45 (m, 1H), 3.39 (t, J=8.28, 8.28 Hz, 1H), 3.31 (s, 3H), 2.25-1.93 (m, 4H).

### Example 195

### 5-Methyl-N²-(2-methylsulfanyl-phenyl)-N⁷-(4-trifluoromethyl-phenyl)-thiazolo[5,4-d]pyrimidine-2,7-diamine

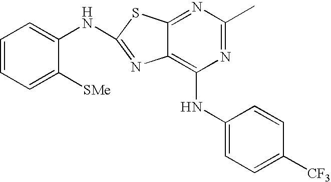

MS (ESI): mass calcd. for C₂₀H₁₆F₃N₅S₂, 447.1; m/z found, 448.1 [M+H]⁺. ¹H NMR ((CD₃)₂SO): δ 9.82 (s, 1H), 9.45 (s, 1H), 8.11 (d, J=8.58 Hz, 2H), 7.89-7.84 (m, 1H), 7.66 (d, J=8.69 Hz, 2H), 7.43-7.39 (m, 1H), 7.30-7.25 (m, 2H), 2.52 (s, 3H), 2.45 (s, 3H).

### Example 196

### N²-(2-Methylsulfanyl-phenyl)-N⁷-(4-trifluoromethyl-phenyl)-thiazolo[5,4-d]pyrimidine-2,7-diamine

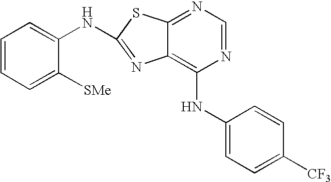

MS (ESI): mass calcd. for C₁₉H₁₄F₃N₅S₂, 433.1; m/z found, 434.1 [M+H]⁺. ¹H NMR ((CD₃)₂SO): δ 9.95 (s, 1H), 9.53 (s, 1H), 8.39 (s, 1H), 8.10 (d, J=8.60 Hz, 2H), 7.85 (dd, J=7.24, 1.99 Hz, 1H), 7.67 (d, J=8.71 Hz, 2H), 7.43-7.41 (m, 1H), 7.33-7.27 (m, 2H), 2.45 (s, 3H).

### Example 197

### N²-(2-Methanesulfonyl-phenyl)-5-methyl-N⁷-(4-trifluoromethylphenyl)-thiazolo[5,4-d]pyrimidine-2,7-diamine

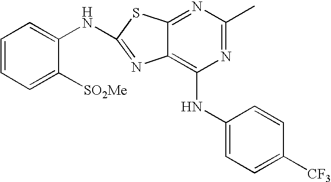

MS (ESI): mass calcd. for C₂₀H₁₆F₃N₅O₂S₂, 479.1; m/z found, 480.1 [M+H]⁺. ¹H NMR ((CD₃)₂SO): δ 9.90 (s, 1H), 9.63 (s, 1H), 8.46-8.39 (m, 1H), 8.13 (d, J=8.47 Hz, 2H), 7.95 (dd, J=7.95, 1.54 Hz, 1H), 7.83-7.75 (m, 1H), 7.68 (d, J=8.70 Hz, 2H), 7.43 (t, J=7.41 Hz, 1H), 3.32 (s, 3H), 2.54 (s, 3H).

### Example 198

### N²-(2-Methanesulfonyl-phenyl)-N⁷-(4-methanesulfonyl-phenyl)-thiazolo[5,4-d]pyrimidine-2,7-diamine

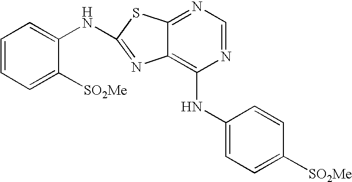

MS (ESI): mass calcd. for C₁₉H₁₇N₅O₄S₃, 475.0; m/z found, 476.1 [M+H]⁺. ¹H NMR ((CD₃)₂SO): δ 10.03 (s, 1H), 9.80 (s, 1H), 8.48 (s, 1H), 8.37 (d, J=7.91 Hz, 1H), 8.17-8.13 (m, 2H), 7.98 (dd, J=7.96, 1.47 Hz, 1H), 7.89-7.84 (m, 2H), 7.84-7.79 (m, 1H), 7.52-7.43 (m, 1H), 3.32 (s, 3H), 3.18 (s, 3H).

### Example 199

### N²-(2-Methanesulfonyl-phenyl)-N⁷-(6-trifluoromethyl-pyridin-3-yl)-thiazolo[5,4-d]pyrimidine-2,7-diamine

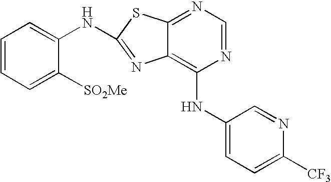

MS (ESI): mass calcd. for C₁₈H₁₃F₃N₆O₂S₂, 466.0; m/z found, 467.1 [M+H]⁺. ¹H NMR ((CD₃)₂SO): δ 10.06 (s, 1H), 9.97 (s, 1H), 9.18 (d, J=2.30 Hz, 1H), 8.60 (dd, J=8.60, 2.28 Hz, 1H), 8.48 (s, 1H), 8.31 (d, J=8.05 Hz, 1H), 7.98 (dd, J=7.96, 1.44 Hz, 1H), 7.88 (d, J=8.69 Hz, 1H), 7.84-7.79 (m, 1H), 7.48 (t, J=7.60 Hz, 1H), 3.32 (s, 3H).

### Example 200

### N²-(2-Methanesulfonyl-phenyl)-N⁷-(4-trifluoromethanesulfonyl-phenyl)-thiazolo[5,4-d]pyrimidine-2,7-diamine

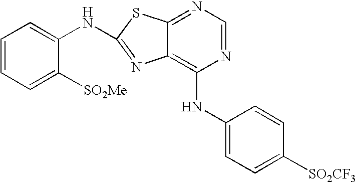

MS (ESI): mass calcd. for C₁₉H₁₄F₃N₅O₄S₃, 529.0; m/z found, 530.1 [M+H]⁺. ¹H NMR (CDCl₃): δ 8.65 (s, 1H), 8.41 (dd, J=8.34, 0.83 Hz, 1H), 8.23-8.19 (m, 2H), 8.08-8.03 (m, 3H), 7.79 (ddd, J=8.43, 7.46, 1.60 Hz, 1H), 7.42-7.34 (m, 1H), 3.16 (s, 3H).

### Example 201

### N²-(2-Methanesulfonyl-phenyl)-N⁷-(4-trifluoromethyl-phenyl)-thiazolo[5,4-d]pyrimidine-2,7-diamine

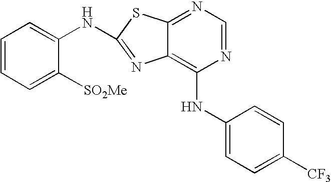

MS (ESI): mass calcd. for C₁₉H₁₄F₃N₅O₂S₂, 465.1; m/z found, 465.1 [M+H]⁺. ¹H NMR (CDCl₃): δ 8.58 (s, 1H), 8.43 (d, J=8.33 Hz, 1H), 8.03 (dd, J=7.96, 1.52 Hz, 1H), 7.96 (d, J=8.50 Hz, 2H), 7.82 (s, 1H), 7.79-7.74 (m, 1H), 7.67 (d, J=8.57 Hz, 2H), 7.37-7.33 (m, 1H), 3.16 (s, 3H).

### Example 202

### N²-(2-Chloro-phenyl)-5-methyl-N⁷-(4-trifluoromethyl-phenyl)-thiazolo[5,4-d]pyrimidine-2,7-diamine

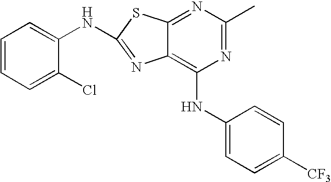

MS (ESI): mass calcd. for C₁₉H₁₃ClF₃N₅S, 435.1; m/z found, 436.1 [M+H]⁺. ¹H NMR (CDCl₃): δ 8.07-8.02 (m, 1H), 7.91-7.86 (m, 2H), 7.68-7.63 (m, 2H), 7.49-7.43 (m, 1H), 7.35-7.28 (m, 1H), 7.16-7.08 (m, 1H), 2.73 (s, 3H).

### Example 203

### N²-(2-Chloro-phenyl)-N⁷-(4-trifluoromethanesulfonyl-phenyl)-thiazolo[5,4-d]pyrimidine-2,7-diamine

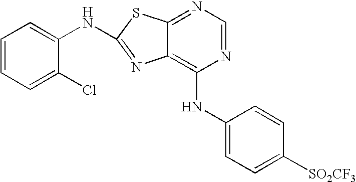

MS (ESI): mass calcd. for C₁₈H₁₁ClF₃N₅O₂S₂, 484.9; m/z found, 486.1 [M+H]⁺. ¹H NMR (CDCl₃): δ 8.64 (s, 1H), 8.22-8.18 (m, 3H), 8.08-8.03 (m, 3H), 7.54 (dd, J=8.05, 1.41 Hz, 1H), 7.46-7.41 (m, 1H), 7.27-7.22 (m, 1H).

### Example 204

### N²-(2,6-Dichloro-phenyl)-N⁷-phenyl-thiazolo[5,4-d]pyrimidine-2,7-diamine

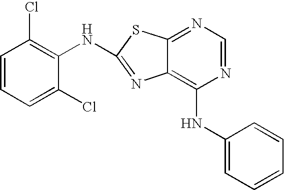

MS (ESI): mass calcd. for C₁₇H₁₁Cl₂N₅S, 387.0; m/z found, 388.1 [M+H]⁺. ¹H NMR (CDCl₃): δ 8.47 (s, 1H), 7.77-7.73 (m, 2H), 7.63 (s, 1H), 7.50 (d, J=8.02 Hz, 2H), 7.42-7.28 (m, 3H), 7.26 (s, 1H), 7.15-7.08 (m, 1H).

### Example 205

### N²-Benzo[1,2,5]thiadiazol-4-yl-5-methyl-N⁷-(4-trifluoromethyl-phenyl)-thiazolo[5,4-d]pyrimidine-2,7-diamine

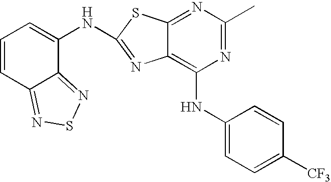

MS (ESI): mass calcd. for C₁₉H₁₂F₃N₇S₂, 459.0; m/z found, 460.1 [M+H]⁺. ¹H NMR ((CD₃)₂SO): δ 11.50 (s, 1H), 9.65 (s, 1H), 9.08 (dd, J=5.84, 2.57 Hz, 1H), 8.20 (d, J=8.53 Hz, 2H), 7.76-7.72 (m, 4H), 2.56 (s, 3H).

### Example 206

### 5-Methyl-N²-(2-nitro-phenyl)-N⁷-(4-trifluoromethyl-phenyl)-thiazolo[5,4-d]pyrimidine-2,7-diamine

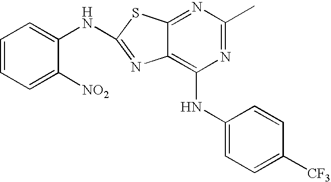

MS (ESI): mass calcd. for C₁₉H₁₃F₃N₆O₂S, 446.1; m/z found, 447.1 [M+H]⁺. ¹H NMR (CD₃OD): δ 8.83-8.64 (m, 1H), 8.25-8.19 (m, 1H), 8.11-8.06 (m, 2H), 7.84-7.77 (m, 1H), 7.67 (d, J=8.83 Hz, 2H), 7.34-7.29 (m, 1H), 2.64 (s, 3H).

### Example 207

### 3-[7-(3-Chloro-4-trifluoromethyl-phenylamino)-thiazolo[5,4-d]pyrimidin-2-ylamino]-4-methyl-thiophene-2-carboxylic acid methyl ester

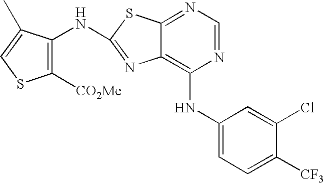

MS (ESI): mass calcd. for C₁₉H₁₃ClF₃N₅O₂S₂, 499.0; m/z found, 500.1 [M+H]⁺. ¹H NMR (CD₃OD): δ 8.36 (s, 1H), 8.23 (d, J=1.91 Hz, 1H), 7.81-7.75 (m, 1H), 7.60 (d, J=8.80 Hz, 1H), 7.46-7.45 (m, 1H), 3.73 (s, 3H), 2.13-2.13 (m, 3H).

### Example 208

### N²-(3,5-Dimethyl-isoxazol-4-yl)-5-methyl-N⁷-(4-trifluoromethyl-phenyl)thiazolo[5,4-d]pyrimidine-2,7-diamine

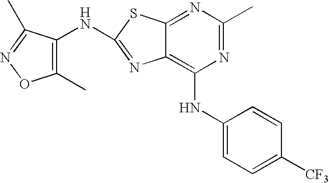

MS (ESI): mass calcd. for C₁₈H₁₅F₃N₆OS, 420.1; m/z found, 421.2 [M+H]⁺. ¹H NMR (CD₃OD): δ 8.02 (d, J=8.53 Hz, 2H), 7.62 (d, J=8.61 Hz, 2H), 2.59 (s, 3H), 2.41 (s, 3H), 2.24 (s, 3H).

### Example 209

### N⁷-(4-tert-Butyl-phenyl)-N²-(3,5-dimethyl-isoxazol-4-yl)-5-methyl-thiazolo[5,4-d]pyrimidine-2,7-diamine

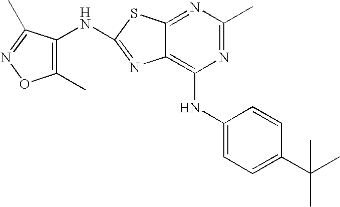

MS (ESI): mass calcd. for C₂₁H₂₄N₆OS, 408.2; m/z found, 409.2 [M+H]⁺. ¹H NMR (CD₃OD): δ 7.64-7.61 (m, 2H), 7.43-7.39 (m, 2H), 2.56 (s, 3H), 2.40 (s, 3H), 2.23 (s, 3H), 1.33 (s, 9H).

### Example 210

### N²-(3-Methyl-pyridin-2-yl)-N⁷-[4-(pyrrolidine-1-sulfonyl)-phenyl]-thiazolo[5,4-d]pyrimidine-2,7-diamine

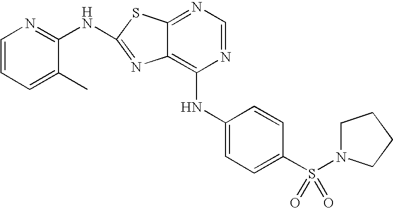

MS (ESI): mass calcd. for C₂₁H₂₁N₇O₂S₂, 467.1; m/z found, 468.2 [M+H]⁺. ¹H NMR (CD₃OD): δ 8.61 (s, 1H), 8.35-8.31 (m, 1H), 8.21-8.16 (m, 2H), 8.01-7.96 (m, 1H), 7.92-7.87 (m, 2H), 7.32-7.26 (m, 1H), 3.31-3.26 (m, 4H), 2.54 (s, 3H), 1.81-1.77 (m, 4H).

### Example 211

### 5-Methyl-N²-(3-methyl-pyridin-2-yl)-N⁷-(4-trifluoromethyl-phenyl)-thiazolo[5,4-d]pyrimidine-2,7-diamine

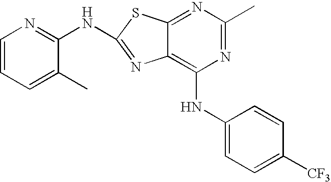

MS (ESI): mass calcd. for C₁₉H₁₅F₃N₆S, 416.1; m/z found, 417.2 [M+H]⁺. ¹H NMR (CD₃OD): δ 8.04-8.02 (m, 1H), 7.88 (d, J=8.48 Hz, 2H), 7.53 (d, J=7.18 Hz, 1H), 7.48 (d, J=8.59 Hz, 2H), 6.88 (dd, J=7.27, 5.30 Hz, 1H), 2.46 (s, 3H), 2.23 (s, 3H).

### Example 212

### N,N-Dimethyl-4-[5-methyl-2-(3-methyl-pyridin-2-ylamino)-thiazolo[5,4-d]pyrimidin-7-ylamino]-benzenesulfonamide

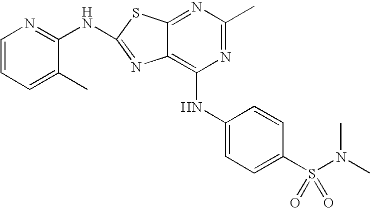

MS (ESI): mass calcd. for C₂₀H₂₁N₇O₂S₂, 455.1; m/z found, 456.2 [M+H]⁺. ¹H NMR (CD₃OD): δ 8.06-8.03 (m, 1H), 7.99-7.95 (m, 2H), 7.64-7.54 (m, 3H), 6.90 (dd, J=7.27, 5.35 Hz, 1H), 2.51 (s, 6H), 2.48 (s, 3H), 2.24 (s, 3H).

### Example 213

### N²-(3-Methyl-pyridin-2-yl)-N⁷-(4-trifluoromethyl-phenyl)-thiazolo[5,4-d]pyrimidine-2,7-diamine

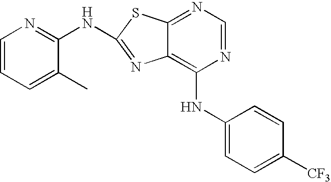

MS (ESI): mass calcd. for C₁₈H₁₃F₃N₆S, 402.1; m/z found, 403.2 [M+H]⁺. ¹H NMR (CD₃OD): δ 8.54 (s, 1H), 8.30 (d, J=4.43 Hz, 1H), 8.12 (d, J=8.47 Hz, 2H), 7.89-7.82 (m, 1H), 7.71 (d, J=8.56 Hz, 2H), 7.23-7.13 (m, 1H), 2.50 (s, 3H).

### Example 214

### N²-(3,5-Dichloro-pyridin-4-yl)-N⁷-[4-(pyrrolidine-1-sulfonyl)-phenyl]thiazolo[5,4-d]pyrimidine-2,7-diamine

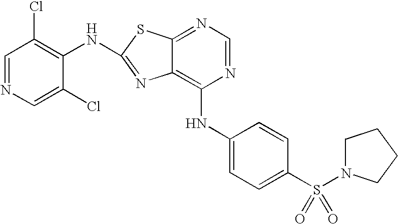

MS (ESI): mass calcd. for C₂₀H₁₇Cl₂N₇O₂S₂, 521.0; m/z found, 522.1 [M+H]⁺. ¹H NMR (CD₃OD): δ 8.67 (s, 2H), 8.47 (s, 1H), 8.11-8.03 (m, 2H), 7.80-7.78 (m, 2H), 3.26-3.22 (m, 4H), 1.78-1.74 (m, 4H).

### Example 215

### N²-(2,6-Dichloro-phenyl)-N⁷-(3-fluoro-4-trifluoromethyl-phenyl)-thiazolo[5,4-d]pyrimidine-2,7-diamine

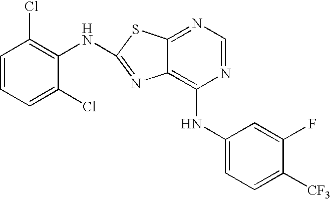

MS (ESI): mass calcd. for C₁₈H₉Cl₂F₄N₅S, 472.9; m/z found, 474.1 [M+H]⁺. ¹H NMR (CD₃OD): δ 8.45 (s, 1H), 8.17 (d, J=14.09 Hz, 1H), 7.64-7.54 (m, 4H), 7.47-7.41 (m, 1H).

### Example 216

### N²-(2-Chlorophenyl)-N⁷-[4-(morpholin-4-ylsulfonyl)phenyl][1,3]thiazolo[5,4-d]pyrimidine-2,7-diamine

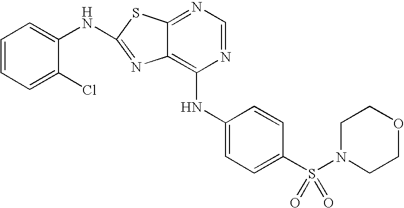

MS (ESI): mass calcd. for C₂₁H₁₉ClN₆O₃S₂, 502.1; m/z found, 503.1 [M+H]⁺. ¹H NMR ((CD₃)₂SO): δ 10.29-10.15 (m, 1H), 9.69-9.63 (m, 1H), 8.57-8.53 (m, 1H), 8.46 (s, 1H), 8.16 (d, J=8.90 Hz, 2H), 7.70 (d, J=8.86 Hz, 2H), 7.55 (dd, J=7.99, 1.45 Hz, 1H), 7.45-7.40 (m, 1H), 7.22-7.17 (m, 1H), 3.72-3.59 (m, 4H), 2.90-2.84 (m, 4H).

### Example 217

### N²-(2-Methylphenyl)-N⁷-[4-(morpholin-4-ylsulfonyl)phenyl][1,3]thiazolo[5,4-d]pyrimidine-2,7-diamine

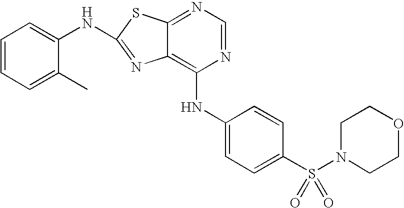

MS (ESI): mass calcd. for C₂₂H₂₂N₆O₃S₂, 482.1; m/z found, 483.1 [M+H]⁺. ¹H NMR ((CD₃)₂SO): δ 9.85 (s, 1H), 9.59 (s, 1H), 8.42 (s, 1H), 8.16 (d, J=8.89 Hz, 2H), 8.00 (d, J=7.82 Hz, 1H), 7.68 (d, J=8.88 Hz, 2H), 7.29 (t, J=7.71 Hz, 2H), 7.15 (dd, J=11.12, 3.72 Hz, 1H), 3.65-3.61 (m, 4H), 2.89-2.84 (m, 4H), 2.32 (s, 3H).

### Example 218

### N²-(2-Methylphenyl)-N⁷-[6-(trifluoromethyl)pyridin-3-yl][1,3]thiazolo[5,4-d]pyrimidine-2,7-diamine

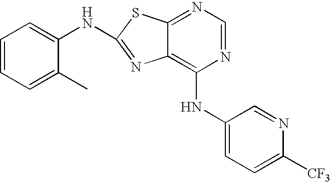

MS (ESI): mass calcd. for C₁₈H₁₃F₃N₆S, 402.1; m/z found, 403.1 [M+H]⁺. ¹H NMR ((CD₃)₂SO): δ 9.86 (s, 1H), 9.76 (s, 1H), 9.17 (d, J=2.39 Hz, 1H), 8.58 (dd, J=8.62, 2.35 Hz, 1H), 8.41 (s, 1H), 7.96 (d, J=7.94 Hz, 1H), 7.86 (d, J=8.66 Hz, 1H), 7.34-7.23 (m, 2H), 7.18-7.12 (m, 1H), 2.32 (s, 3H).

### Example 219

### N²-[2-(Trifluoromethyl)phenyl]-N⁷-[6-(trifluoromethyl)pyridin-3-yl][1,3]thiazolo[5,4-d]pyrimidine-2,7-diamine

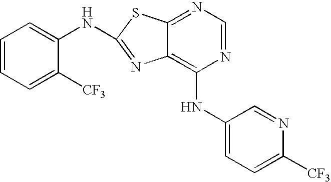

MS (ESI): mass calcd. for C₁₈H₁₀F₆N₆S, 456.1; m/z found, 457.1 [M+H]⁺. ¹H NMR ((CD₃)₂SO): δ 9.35 (s, 1H), 8.98 (s, 1H), 8.36 (d, J=1.60 Hz, 1H), 7.78 (dd, J=8.61, 2.27 Hz, 1H), 7.66 (s, 1H), 7.29 (d, J=8.07 Hz, 1H), 7.09-6.97 (m, 3H), 6.78-6.65 (m, 1H).

### Example 220

### N²-(2-Chlorophenyl)-N⁷-[6-(trifluoromethyl)pyridin-3-yl][1,3]thiazolo[5,4-d]pyrimidine-2,7-diamine

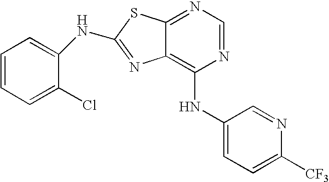

MS (ESI): mass calcd. for C₁₇H₁₀ClF₃N₆S, 422.0; m/z found, 423.1 [M+H]⁺. ¹H NMR ((CD₃)₂SO): 610.25 (s, 1H), 9.81 (s, 1H), 9.18 (d, J=2.06 Hz, 1H), 8.60-8.55 (m, 1H), 8.50-8.44 (m, 2H), 7.87 (d, J=8.64 Hz, 1H), 7.55 (dd, J=7.96, 1.41 Hz, 1H), 7.46-7.38 (m, 1H), 7.24-7.17 (m, 1H).

### Example 221

### N²-(3,5-Dimethylisoxazol-4-yl)-N⁷-[4-(morpholin-4-ylsulfonyl)phenyl][1,3]thiazolo[5,4-d]pyrimidine-2,7-diamine

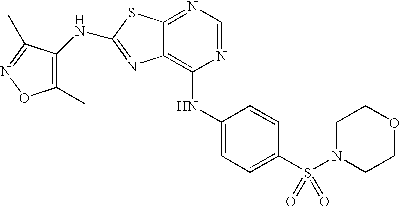

MS (ESI): mass calcd. for C₂₀H₂₁N₇O₄S₂, 487.1; m/z found, 488.1 [M+H]⁺. ¹H NMR ((CD₃)₂SO): δ 9.74 (s, 1H), 9.61 (s, 1H), 8.41 (s, 1H), 8.17 (d, J=8.89 Hz, 2H), 7.67 (d, J=8.85 Hz, 2H), 3.65-3.61 (m, 4H), 2.89-2.84 (m, 4H), 2.38 (s, 3H), 2.20 (s, 3H).

### Example 222

### Methyl 2-[4-({2-[(3,5-dimethylisoxazol-4-yl)amino][1,3]thiazolo[5,4-d]pyrimidin-7-yl}amino)phenyl]-2-methylpropanoate

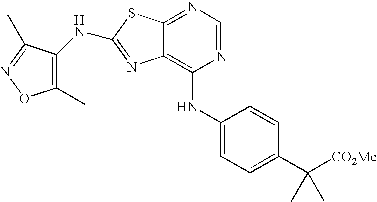

MS (ESI): mass calcd. for C₂₁H₂₂N₆O₃S, 438.1; m/z found, 439.2 [M+H]⁺. ¹H NMR ((CD₃)₂SO): δ 9.64 (s, 1H), 9.04 (s, 1H), 8.27 (s, 1H), 7.73 (d, J=8.74 Hz, 2H), 7.25 (d, J=8.73 Hz, 2H), 3.59 (s, 3H), 2.37 (s, 3H), 2.19 (s, 3H), 1.50 (s, 6H).

### Example 223

### 2-[4-({2-[(3,5-Dimethylisoxazol-4-yl)amino][1,3]thiazolo[5,4-d]pyrimidin-7-yl}amino)phenyl]-2-methylpropanenitrile

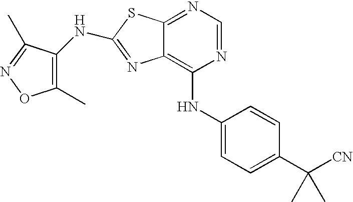

MS (ESI): mass calcd. for C₂₀H₁₉N₇OS, 405.1; m/z found, 406.2 [M+H]⁺. ¹H NMR ((CD₃)₂SO): δ 9.66 (s, 1H), 9.16 (s, 1H), 8.30 (d, J=1.06 Hz, 1H), 7.87-7.82 (m, 2H), 7.47-7.42 (m, 2H), 2.37 (s, 3H), 2.19 (d, J=1.06 Hz, 3H), 1.69-1.68 (m, 6H).

### Example 224

### N²-(3,5-Dimethylisoxazol-4-yl)-N⁷-[4-(methylsulfonyl)phenyl][1,3]thiazolo[5,4-d]pyrimidine-2,7-diamine

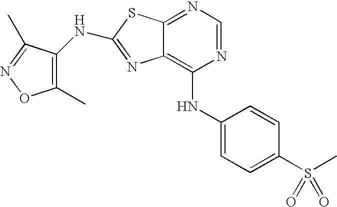

MS (ESI): mass calcd. for C₁₇H₁₆N₆O₃S₂, 416.1; m/z found, 417.1 [M+H]⁺. ¹H NMR ((CD₃)₂SO): δ 9.74 (s, 1H), 9.59 (s, 1H), 8.41 (d, J=7.63 Hz, 1H), 8.16-8.11 (m, 2H), 7.89-7.80 (m, 2H), 3.20-3.13 (m, 3H), 2.42-2.33 (m, 3H), 2.23-2.13 (m, 3H).

### Example 225

### N²-[2-(Trifluoromethyl)phenyl]-N⁷-[4-(trifluoromethyl)phenyl][1,3]thiazolo[5,4-d]pyrimidine-2,7-diamine

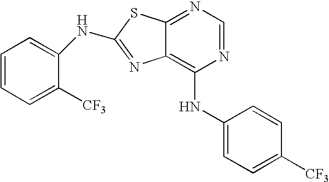

MS (ESI): mass calcd. for C₁₉H₁₁F₆N₅S, 455.1; m/z found, 456.1 [M+H]⁺. ¹H NMR ((CD₃)₂SO): δ 10.08 (s, 1H), 9.46 (s, 1H), 8.41 (s, 1H), 8.09 (m, 3H), 7.85-7.72 (m, 2H), 7.66 (d, J=8.69 Hz, 2H), 7.48 (t, J=7.57 Hz, 1H).

### Example 226

### N⁷-[4-(Methylsulfonyl)phenyl]-N²-[2-(trifluoromethyl)phenyl][1,3]thiazolo[5,4-d]pyrimidine-2,7-diamine

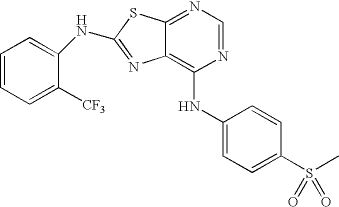

MS (ESI): mass calcd. for C₁₉H₁₄F₃N₅O₂S₂, 465.1; m/z found, 466.1 [M+H]⁺. ¹H NMR ((CD₃)₂SO): δ 10.11 (s, 1H), 9.57 (s, 1H), 8.43 (s, 1H), 8.13-8.07 (m, 3H), 7.93-7.72 (m, 4H), 7.48 (t, J=7.66 Hz, 1H), 3.16 (s, 3H).

### Example 227

### 4-({2-[(2,6-Dichlorophenyl)amino][1,3]thiazolo[5,4-d]pyrimidin-7-yl}amino)benzene-1,2-diol

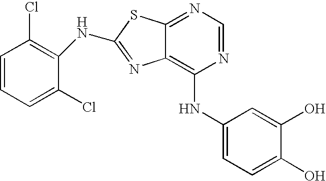

MS (ESI): mass calcd. for C₁₇H₁₁Cl₂N₅O₂S, 419.0; m/z found, 420 [M+H]⁺. ¹H NMR ((CD₃)₂SO): δ 10.22 (bs, 1H), 8.67 (s, 2H), 8.22 (s, 1H), 7.70-7.61 (m, 3H), 7.49-7.35 (m, 1H), 7.20 (d, J=2.50 Hz, 1H), 6.91 (dd, J=8.53, 2.53 Hz, 1H), 6.65 (d, J=8.49 Hz, 1H).

### Example 228

### 2-[4-({2-[(2,6-Dichlorophenyl)amino][1,3]thiazolo[5,4-d]pyrimidin-7-yl}amino)phenyl]-2-methylpropanenitrile

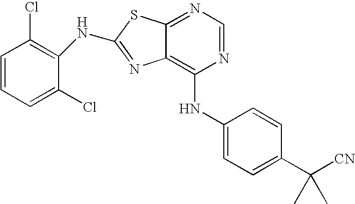

MS (ESI): mass calcd. for C₂₁H₁₆Cl₂N₆S, 454.1; m/z found, 455.1 [M+H]⁺. ¹H NMR ((CD₃)₂SO): δ 10.27 (bs, 1H), 9.18 (s, 1H), 8.30 (s, 1H), 7.82 (dd, J=9.21, 2.42 Hz, 1H), 7.65 (d, J=8.12 Hz, 2H), 7.47-7.40 (m, 3H), 2.69-2.65 (m, 1H), 1.68 (s, 6H).

### Example 229

### Methyl 2-[4-({2-[(2,6-dichlorophenyl)amino][1,3]thiazolo[5,4-d]pyrimidin-7-yl}amino)phenyl]-2-methylpropanoate

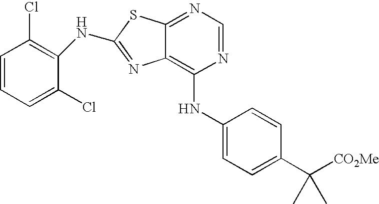

A solution of (7-chloro-thiazolo[5,4-d]pyrimidin-2-yl)-(2,6-dichloro-phenyl)-amine (166 mg, 0.50 mmol), 2-(4-amino-phenyl)-2-methyl-propionic acid methyl ester (97 mg, 0.50 mmol), and HCl (4 N in dioxane; 0.28 mL, 1.10 mmol) in 95% IPA in H₂O (2 mL) was heated to 90° C. After 18 h, the mixture was cooled, concentrated, and purified using preparative reverse-phase HPLC to afford the following compound(s) as colorless solids: methyl 2-[4-({2-[(2,6-dichlorophenyl)amino][1,3]thiazolo[5,4-d]pyrimidin-7-yl}amino)phenyl]-2-methylpropanoate (67 mg, 28%); 2-[4-({2-[(2,6-dichlorophenyl)amino][1,3]thiazolo[5,4-d]pyrimidin-7-yl}amino)phenyl]-2-methylpropanoic acid (Example 230; 23 mg, 10%); and 1-methylethyl 2-[4-({2-[(2,6-dichlorophenyl)amino][1,3]thiazolo[5,4-d]pyrimidin-7-yl}amino)phenyl]-2-methylpropanoate (Example 231; 25 mg, 10%). Analytical data for methyl 2-[4-({2-[(2,6-dichlorophenyl)amino][1,3]thiazolo[5,4-d]pyrimidin-7-yl}amino)phenyl]-2-methylpropanoate: MS (ESI): mass calcd. for C₂₂H₁₉Cl₂N₅O₂S, 487.1; m/z found, 488.1 [M+H]⁺. ¹H NMR ((CD₃)₂SO): δ 10.26 (bs, 2H), 9.07 (s, 1H), 8.27 (s, 1H), 7.74-7.68 (m, 2H), 7.65 (d, J=8.13 Hz, 2H), 7.49-7.40 (m, 1H), 7.23 (d, J=8.72 Hz, 2H), 3.59 (s, 3H), 1.50 (s, 6H).

### Example 230

### 2-[4-({2-[(2,6-Dichlorophenyl)amino][1,3]thiazolo[5,4-d]pyrimidin-7-yl}amino)phenyl]-2-methylpropanoic acid

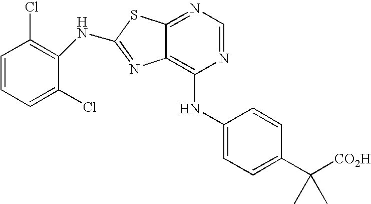

MS (ESI): mass calcd. for C₂₁H₁₇Cl₂N₅O₂S, 473.0; m/z found, 474.1 [M+H]⁺. ¹H NMR ((CD₃)₂SO): δ 10.25 (bs, 1H), 9.04 (s, 1H), 8.27 (s, 1H), 7.72-7.67 (m, 2H), 7.65 (d, J=8.14 Hz, 1H), 7.51-7.38 (m, 2H), 7.30-7.25 (m, 2H), 1.47 (s, 6H).

### Example 231

### 1-Methylethyl 2-[4-({2-[(2,6-dichlorophenyl)amino][1,3]thiazolo[5,4-d]pyrimidin-7-yl}amino)phenyl]-2-methylpropanoate

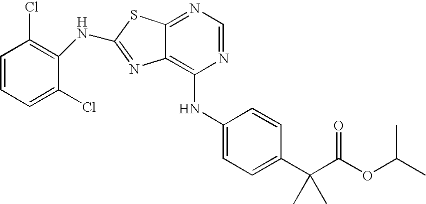

MS (ESI): mass calcd. for C₂₄H₂₃Cl₂N₅O₂S, 515.1; m/z found, 516.1 [M+H]⁺. ¹H NMR ((CD₃)₂SO): δ 10.26 (bs, 1H), 9.05 (s, 1H), 8.28 (s, 1H), 7.75-7.68 (m, 2H), 7.65 (d, J=8.14 Hz, 2H), 7.50-7.38 (m, 1H), 7.27-7.18 (m, 2H), 4.89 (td, J=12.51, 6.26 Hz, 1H), 1.47 (s, 6H), 1.12 (d, J=6.25 Hz, 6H).

### Example 232

### N²-Cyclohexyl-N⁷-[4-(trifluoromethyl)phenyl][1,3]thiazolo[5,4-d]pyrimidine-2,7-diamine

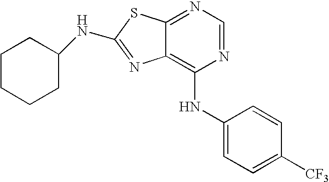

MS (ESI): mass calcd. for C₁₈H₁₈F₃N₅S, 393.1; m/z found, 394.2 [M+H]⁺ 0.1H NMR ((CD₃)₂SO): δ 9.21 (s, 1H), 8.36-8.28 (m, 2H), 8.11 (d, J=8.57 Hz, 2H), 7.66 (d, J=8.71 Hz, 2H), 4.04-3.88 (m, 1H), 2.09-1.92 (m, 2H), 1.82-1.68 (m, 2H), 1.66-1.54 (m, 1H), 1.47-1.13 (m, 5H).

### Example 233

### N²-Cyclohexyl-N⁷-[6-(trifluoromethyl)pyridin-3-yl][1,3]thiazolo[5,4-d]pyrimidine-2,7-diamine

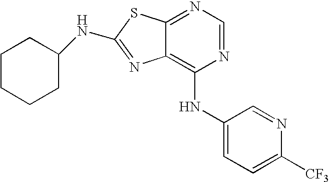

MS (ESI): mass calcd. for C₁₇H₁₇F₃N₆S, 394.1; m/z found, 395.1 [M+H]⁺. ¹H NMR ((CD₃)₂SO): δ 9.52 (s, 1H), 9.17 (d, J=2.38 Hz, 1H), 8.65-8.51 (m, 1H), 8.37 (d, J=7.90 Hz, 1H), 8.33 (s, 1H), 7.85 (d, J=8.71 Hz, 1H), 4.07-3.87 (m, 1H), 2.10-1.90 (m, 2H), 1.82-1.67 (m, 2H), 1.67-1.54 (m, 1H), 1.46-1.13 (m, 5H).

### Example 234

### 3,5-Dichloro-4-[7-(4-trifluoromethyl-phenylamino)-thiazolo[5,4-d]pyrimidin-2-ylamino]-benzonitrile

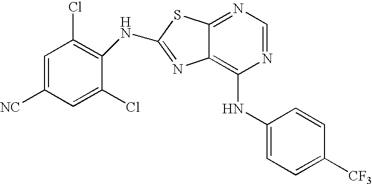

A mixture of 3,5-dichloro-4-(7-chloro-thiazolo[5,4-d]pyrimidin-2-ylamino)-benzonitrile (470 mg, 1.32 mmol), 4-trifluoromethyl-phenylamine (212 mg, 1.32 mmol), and p-toluenesulfonic acid (504 mg, 2.65 mmol) in toluene (12 mL) was heated to 125° C. After 2 h, the mixture was cooled and concentrated to afford a crude residue which was purified by FCC to afford the title compound (400 mg, 63%) and 3,5-dichloro-4-[7-(4-trifluoromethyl-phenylamino)-thiazolo[5,4-d]pyrimidin-2-ylamino]-benzamide (Example 235; 50 mg, 8%). Analytical data for the title compound: MS (ESI): mass calcd. for C₁₉H₉Cl₂F₃N₆S, 479.9; m/z found, 481.0 [M+H]⁺. ¹H NMR ((CD₃)₂SO): δ 10.75 (s, 1H), 9.55 (s, 1H), 8.42 (s, 1H), 8.27 (s, 2H), 8.04 (d, J=8.70 Hz, 2H), 7.64 (d, J=8.19 Hz, 2H).

### Example 235

### 3,5-Dichloro-4-[7-(4-trifluoromethyl-phenylamino)-thiazolo[5,4-d]pyrimidin-2-ylamino]-benzamide

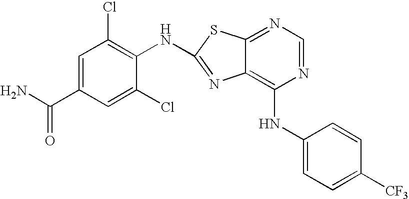

MS (ESI): mass calcd. for C₁₉H₁₁Cl₂F₃N₆OS, 498.0; m/z found, 499.0 [M+H]⁺. ¹H NMR (CDCl₃): δ 8.54 (s, 1H), 7.95-7.91 (m, 4H), 7.70-7.60 (m, 3H).

The compounds in Examples 236-237 were prepared using methods analogous to those described in Examples 234-235.

### Example 236

### 3,5-Dichloro-4-[7-(4-methanesulfonyl-phenylamino)-thiazolo[5,4-d]pyrimidin-2-ylamino]-benzonitrile

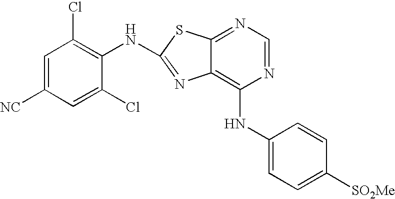

MS (ESI): mass calcd. for C₁₉H₁₂Cl₂N₆O₂S₂, 489.9; m/z found, 491.0 [M+H]⁺. ¹H NMR ((CD₃)₂SO): δ 10.78-10.72 (m, 1H), 9.72-9.47 (m, 1H), 8.45 (s, 1H), 8.26 (s, 2H), 8.08 (d, J=8.46 Hz, 2H), 7.83 (d, J=8.44 Hz, 2H), 3.16 (s, 3H).

### Example 237

### 3,5-Dichloro-4-[7-(4-methanesulfonyl-phenylamino)-thiazolo[5,4-d]pyrimidin-2-ylamino]-benzamide

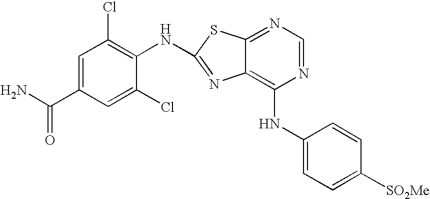

MS (ESI): mass calcd. for C₁₉H₁₄Cl₂N₆O₃S₂, 507.9; m/z found, 509.0 [M+H]⁺. ¹H NMR ((CD₃)₂SO): δ 10.55 (s, 1H), 9.63 (d, J=0.70 Hz, 1H), 8.43 (s, 1H), 8.22 (s, 1H), 8.13-8.07 (m, 4H), 7.82 (d, J=8.68 Hz, 2H), 7.70 (s, 1H), 3.16 (s, 3H).

### Example 238

### N²-(2,6-Dichloro-4-morpholin-4-ylmethyl-phenyl)-N⁷-(4-trifluoromethyl-phenyl)-thiazolo[5,4-d]pyrimidine-2,7-diamine

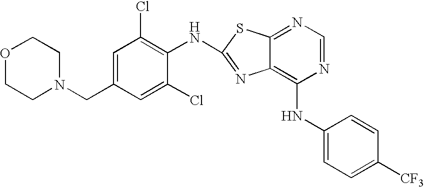

To a −20° C. solution of 3,5-dichloro-4-[7-(4-trifluoromethyl-phenylamino)-thiazolo[5,4-d]pyrimidin-2-ylamino]-benzonitrile (202 mg, 0.42 mmol) in THF (10 mL) was added diisobutylaluminum hydride (1.5 M in toluene, 0.8 mL, 1.3 mmol). After 1 h, the solution was warmed to rt over 3 h, at which time the solution was diluted with 30% aq. sodium potassium tartrate (10 mL). The resulting mixture was stirred for 1 h and then extracted with EtOAc (3×20 mL). The combined organic layers were dried and concentrated to give 3,5-dichloro-4-[7-(4-trifluoromethyl-phenylamino)-thiazolo[5,4-d]pyrimidin-2-ylamino]-benzaldehyde (195 mg), which was used immediately. MS (ESI): mass calcd. for C₁₉H₁₀Cl₂F₃N₅OS, 482.9; m/z found, 484.0 [M+H]⁺.

To a solution of crude aldehyde (100 mg, 0.21 mmol), morpholine (23 mg, 0.26 mmol) in CH₂Cl₂ (5 mL) was added sodium triacetoxyborohydride (88 mg, 0.41 mmol). After 5 h, the mixture was diluted with satd. aq. NaHCO₃ (10 mL) and extracted with CH₂Cl₂ (3×10 mL). The combined organic layers were dried, concentrated, and purified using preparative reverse-phase HPLC to afford the title compound (30 mg, 26%). MS (ESI): mass calcd. for C₂₃H₁₉Cl₂F₃N₆OS, 554.1; m/z found, 555.1 [M+H]⁺. ¹H NMR (CD₃OD): δ 8.43 (s, 1H), 7.99 (d, J=8.73 Hz, 2H), 7.82 (s, 2H), 7.62 (d, J=8.75 Hz, 2H), 4.45 (s, 2H), 4.20-3.77 (m, 4H), 3.44-3.34 (m, 4H).

### Example 239

### N²-(4-Azetidin-1-ylmethyl-2,6-dichloro-phenyl)-N⁷-(4-trifluoromethy-phenyl)-thiazolo[5,4-d]pyrimidine-2,7-diamine

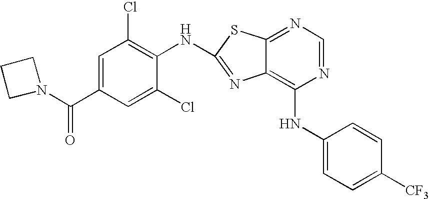

To a solution of 3,5-dichloro-4-[7-(4-trifluoromethyl-phenylamino)-thiazolo[5,4-d]pyrimidin-2-ylamino]-benzaldehyde (100 mg, 0.21 mmol) and azetidine (15 mg, 0.26 mmol) in CH₂Cl₂ (5 mL) was added sodium triacetoxyborohydride (178 mg, 0.84 mmol). After 5 h, the mixture was diluted with satd. aq. NaHCO₃ (10 mL) and extracted with CH₂Cl₂ (3×10 mL). The combined organic layers were dried, concentrated, and purified using preparative reverse-phase HPLC to afford the title compound (38 mg, 34%). MS (ESI): mass calcd. for C₂₂H₁₇Cl₂F₃N₆S, 524.1; m/z found, 525.1 [M+H]⁺. ¹H NMR (CD₃OD): δ 8.42 (s, 1H), 7.99 (d, J=8.55 Hz, 2H), 7.75 (s, 2H), 7.62 (d, J=8.66 Hz, 2H), 4.46 (s, 2H), 4.29-4.22 (m, 4H), 2.70-2.49 (m, 2H).

### Example 240

### N²-(4-Aminomethyl-2,6-dichloro-phenyl)-N⁷-(4-trifluoromethyl-phenyl)-thiazolo[5,4-d]pyrimidine-2,7-diamine

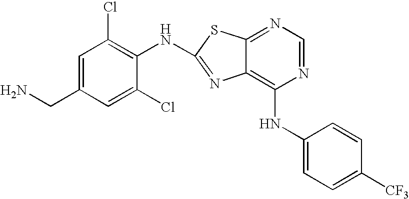

To a solution of 3,5-dichloro-4-[7-(4-trifluoromethyl-phenylamino)-thiazolo[5,4-d]pyrimidin-2-ylamino]-benzonitrile (32 mg, 0.07 mmol) in THF (2 mL) was added LiAlH₄ (5 mg, 0.13 mmol). After 2 h, the mixture was diluted with 30% aq. sodium potassium tartrate (5 mL). The resulting mixture was stirred for 1 h and then extracted with EtOAc (3×10 mL). The combined organic layers were dried, concentrated, and purified using preparative reverse-phase HPLC to afford the title compound (24 mg, 75%). MS (ESI): mass calcd. for C₁₉H₁₃Cl₂F₃N₆S, 484.0; m/z found, 485.0 [M+H]⁺. ¹H NMR ((CD₃)₂SO): δ 10.46 (s, 1H), 9.58 (s, 1H), 8.40 (s, 1H), 8.27 (s, 2H), 8.07 (d, J=8.49 Hz, 2H), 7.77 (s, 2H), 7.66 (d, J=8.71 Hz, 2H), 4.16-4.10 (m, 2H).

### Example 241

### 3,5-Dichloro-4-[7-(4-trifluoromethyl-phenylamino)-thiazolo[5,4-d]pyrimidin-2-ylamino]-benzoic acid methyl ester

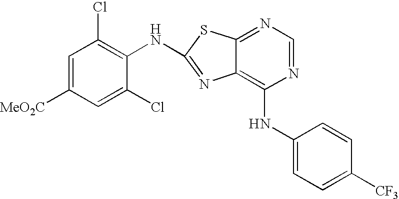

To solution of 3,5-dichloro-4-[7-(4-trifluoromethyl-phenylamino)-thiazolo[5,4-d]pyrimidin-2-ylamino]-benzonitrile (300 mg, 0.62 mmol) in MeOH (5 mL) was added concentrated H₂SO₄ (0.2 mL). The resulting mixture was heated to reflux with vigorous stirring. After 48 h, the solution was allowed to cool and was precipitated with H₂O (10 mL). The resulting solid was collected by vacuum filtration and washed with 10% MeOH—H₂O (25 mL) to provide tan solid. This material was further purified by preparative reverse-phase HPLC to afford the title compound (290 mg, 91%). MS (ESI): mass calcd. for C₂₀H₁₂Cl₂F₃N₅O₂S, 513.0; m/z found, 514.0 [M+H]⁺. ¹H NMR (CDCl₃): δ 8.56 (s, 1H), 8.17 (s, 2H), 7.93 (d, J=8.48 Hz, 2H), 7.64 (d, J=8.49 Hz, 2H), 4.00 (s, 3H).

### Example 242

### {3,5-Dichloro-4-[7-(4-trifluoromethyl-phenylamino)-thiazolo[5,4-d]pyrimidin-2-ylamino]-phenyl}-methanol

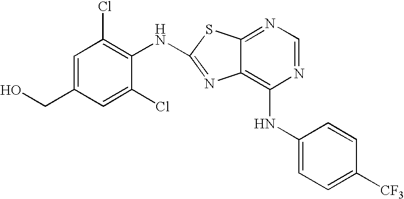

To a solution of 3,5-dichloro-4-[7-(4-trifluoromethyl-phenylamino)-thiazolo[5,4-d]pyrimidin-2-ylamino]-benzoic acid methyl ester (93 mg, 0.19 mmol) in THF (5 mL) was added diisobutylaluminum hydride (1.5 M in toluene, 0.6 mL, 0.96 mmol). After 2 h, the solution was diluted with 30% aq. sodium potassium tartrate (10 mL). The resulting mixture was stirred for 1 h and then extracted with EtOAc (3×10 mL). The combined organic layers were dried, concentrated and purified by preparative reverse-phase HPLC to afford the title compound (65 mg, 74%). MS (ESI): mass calcd. for C₁₉H₁₂Cl₂F₃N₅OS, 485.0; m/z found, 486.0 [M+H]⁺. ¹H NMR ((CD₃)₂SO): δ 10.30 (s, 1H), 9.58 (s, 1H), 8.38 (s, 1H), 8.07 (d, J=8.56 Hz, 2H), 7.65 (d, J=8.65 Hz, 2H), 7.57 (s, 2H), 4.56 (s, 2H).

### Example 243

### 3,5-Dichloro-4-[7-(4-trifluoromethyl-phenylamino)-thiazolo[5,4-d]pyrimidin-2-ylamino]-benzoic acid

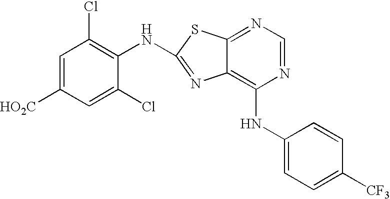

To a solution of 3,5-dichloro-4-[7-(4-trifluoromethyl-phenylamino)-thiazolo[5,4-d]pyrimidin-2-ylamino]-benzoic acid methyl ester (55 mg, 0.11 mmol) in THF (5 mL) and H₂O (10 mL) was added lithium hydroxide monohydrate (18 mg, 0.43 mmol). After 12 h, the solution was acidified with 4 drops of AcOH and concentrated. The resulting residue was purified by preparative reverse-phase HPLC to afford the title compound (38 mg, 72%). MS (ESI): mass calcd. for C₁₉H₁₀Cl₂F₃N₅O₂S, 498.9; m/z found, 500.0 [M+H]⁺. ¹H NMR ((CD₃)₂SO): δ 10.60 (s, 1H), 9.47 (s, 1H), 8.41 (s, 1H), 8.07-8.00 (m, 4H), 7.63 (d, J=8.42 Hz, 2H).

### Example 244

### N⁷-(4-tert-Butyl-phenyl)-N²-(2,6-dimethyl-phenyl)-5-methyl-thiazolo[5,4-d]pyrimidine-2,7-diamine

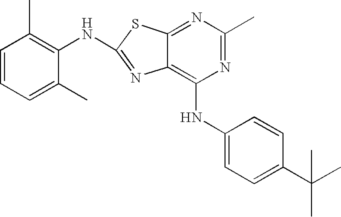

MS (ESI): mass calcd. for C₂₄H₂₇N₅S, 417.2; m/z found, 418.3 [M+H]⁺. ¹H NMR (CDCl₃): 7.63 (d, J=8.44 Hz, 2H), 7.35 (d, J=8.69 Hz, 2H), 7.23-7.17 (m, 1H), 7.12 (d, J=7.56 Hz, 2H), 2.61 (s, 3H), 2.26 (s, 6H), 1.27 (s, 9H).

### Example 245

### N²-(2,6-Dimethyl-phenyl)-5-methyl-N⁷-(4-trifluoromethyl-phenyl)-thiazolo[5,4-d]pyrimidine-2,7-diamine

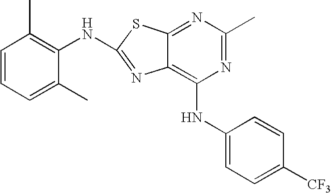

MS (ESI): mass calcd. for C₂₁H₁₈F₃N₅S, 429.1; m/z found, 430.2 [M+H]⁺. ¹H NMR (CDCl₃): 7.88 (d, J=8.54 Hz, 2H), 7.76 (s, 1H), 7.55 (d, J=8.61 Hz, 2H), 7.22-7.16 (m, 1H), 7.12 (d, J=7.46 Hz, 2H), 2.60 (s, 3H), 2.26 (s, 6H).

### Example 246

### N²-(2,6-Dimethyl-phenyl)-5-methyl-N⁷-(6-trifluoromethyl-pyridin-3-yl)-thiazolo[5,4-d]pyrimidine-2,7-diamine

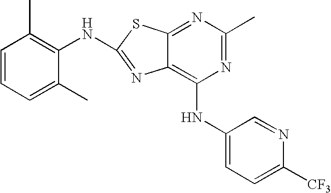

MS (ESI): mass calcd. for C₂₀H₁₇F₃N₆S, 430.1; m/z found, 431.2 [M+H]⁺. ¹H NMR (CDCl₃): 9.03 (d, J=2.39 Hz, 1H), 8.53-8.48 (m, 1H), 8.09 (s, 1H), 7.64 (d, J=8.62 Hz, 1H), 7.24-7.17 (m, 1H), 7.14 (d, J=7.56 Hz, 2H), 2.61 (s, 3H), 2.27 (s, 6H).

### Example 247

### N⁷-(3-Chloro-4-trifluoromethyl-phenyl)-N²-(2,6-dimethyl-phenyl)-5-methyl-thiazolo[5,4-d]pyrimidine-2,7-diamine

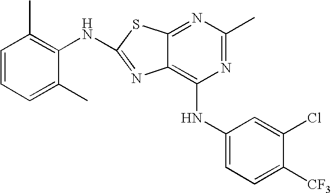

MS (ESI): mass calcd. for C₂₁H₁₇ClF₃N₅S, 463.1; m/z found, 464.2 [M+H]⁺. ¹H NMR (CDCl₃): 8.35 (s, 1H), 8.17 (s, 1H), 7.69-7.65 (m, 1H), 7.62 (d, J=8.69 Hz, 1H), 7.26-7.20 (m, 1H), 7.14 (d, J=7.58 Hz, 2H), 2.65 (s, 3H), 2.27 (s, 6H).

### Example 248

### N⁷-(4-tert-Butyl-cyclohexyl)-N²-(2,6-dimethyl-phenyl)-thiazolo[5,4-d]pyrimidine-2,7-diamine

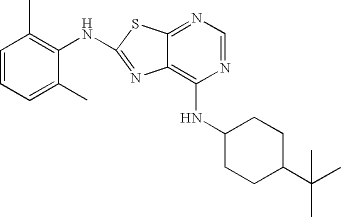

The title compound was isolated as a single diastereomer with undetermined relative stereochemistry. MS (ESI): mass calcd. for C₂₃H₃₁N₅S, 409.2; m/z found, 410.3 [M+H]⁺. ¹H NMR (CDCl₃): 9.55 (s, 1H), 8.15 (s, 1H), 7.22-7.15 (m, 3H), 2.23 (s, 6H), 1.97-1.86 (m, 2H), 1.82-1.69 (m, 1H), 1.51-1.26 (m, 2H), 1.21-1.02 (m, 3H), 1.03-0.92 (m, 1H), 0.85 (s, 9H).

### Example 249

### N⁷-(4-tert-Butyl-cyclohexyl)-N²-(2,6-dimethyl-phenyl)-5-methyl-thiazolo[5,4-d]pyrimidine-2,7-diamine

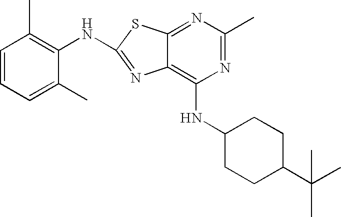

The title compound was isolated as 2:1 mixture of diastereomers. MS (ESI): mass calcd. for C₂₄H₃₃N₅S, 423.2; m/z found, 424.3 [M+H]⁺.

### Example 250

### (R)-1-[2-(2,6-Dichloro-phenylamino)-7-(4-trifluoromethyl-phenylamino)-thiazolo[5,4-d]pyrimidin-5-ylamino]-propan-2-ol

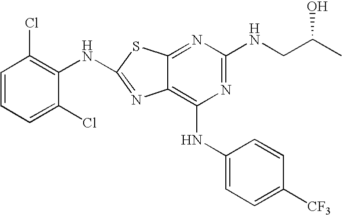

MS (ESI): mass calcd. for C₂₁H₁₇Cl₂F₃N₆OS, 528.0; m/z found, 529.0 [M+H]⁺. ¹H NMR ((CD₃)₂SO): 9.69 (s, 1H), 9.13 (s, 1H), 8.11 (d, J=8.52 Hz, 2H), 7.64-7.54 (m, 4H), 7.42-7.35 (m, 1H), 6.70-6.59 (m, 1H), 4.63 (d, J=4.68 Hz, 2H), 3.87-3.75 (m, 1H), 3.27-3.17 (m, 1H), 1.08 (d, J=6.22 Hz, 3H).

### Example 251

### 1-[2-(2,6-Dichloro-phenylamino)-7-(4-trifluoromethyl-phenylamino)-thiazolo[5,4-d]pyrimidin-5-ylamino]-2-methyl-propan-2-ol

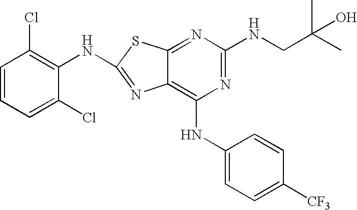

MS (ESI): mass calcd. for C₂₂H₁₉Cl₂F₃N₆OS, 542.0; m/z found, 543.1 [M+H]⁺. ¹H NMR ((CD₃)₂SO): 9.68 (s, 1H), 9.11 (s, 1H), 8.11 (d, J=8.35 Hz, 2H), 7.60 (d, J=8.11 Hz, 2H), 7.56 (d, J=8.64 Hz, 2H), 7.42-7.35 (m, 1H), 6.82-6.73 (m, 1H), 1.75-1.54 (m, 1H), 1.47-1.40 (m, 2H), 0.89 (d, J=6.63 Hz, 6H).

### Example 252

### (racemic)-{1-[2-(2,6-Dichloro-Phenylamino)-7-(4-trifluoromethyl-phenylamino)-thiazolo[5,4-d]pyrimidin-5-yl]-pyrrolidin-2-yl}-methanol

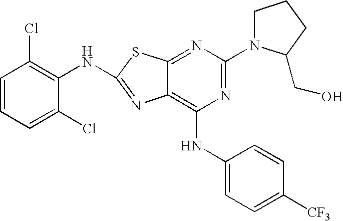

MS (ESI): mass calcd. for C₂₃H₁₉Cl₂F₃N₆OS, 554.0; m/z found, 555.1 [M+H]⁺. ¹H NMR (CD₃OD): 7.98 (d, J=8.53 Hz, 2H), 7.65 (d, J=8.63 Hz, 2H), 7.59 (d, J=8.15 Hz, 2H), 7.43-7.38 (m, 1H), 4.34-4.25 (m, 1H), 3.82-3.74 (m, 1H), 3.73-3.65 (m, 2H), 3.65-3.56 (m, 1H), 2.29-1.97 (m, 4H).

### Example 253

### N²-(2,6-Dichloro-phenyl)-N⁵-methyl-N⁵-(2-piperidin-1-yl-ethyl)-N⁷-(4-trifluoromethyl-phenyl)-thiazolo[5,4-d]pyrimidine-2,5,7-triamine

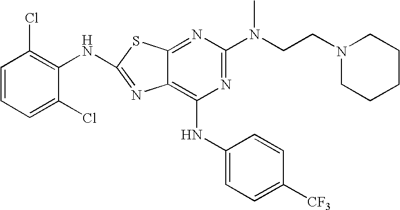

MS (ESI): mass calcd. for C₂₆H₂₆Cl₂F₃N₇S, 595.1; m/z found, 596.1 [M+H]⁺. ¹H NMR (CD₃OD): 7.90 (d, J=8.52 Hz, 2H), 7.62 (d, J=8.70 Hz, 2H), 7.59-7.56 (m, 2H), 7.42-7.36 (m, 1H), 4.05-3.97 (m, 2H), 3.67 (d, J=11.80 Hz, 2H), 3.38-3.33 (m, 2H), 3.24 (s, 3H), 3.00-2.87 (m, 2H), 1.92-1.75 (m, 3H), 1.75-1.62 (m, 2H), 1.55-1.40 (m, 1H).

### Example 254

### N²-(2,6-Dichloro-phenyl)-N⁷-(6-methanesulfonyl-pyridin-3-yl)-thiazolo[5,4-d]pyrimidine-2,7-diamine

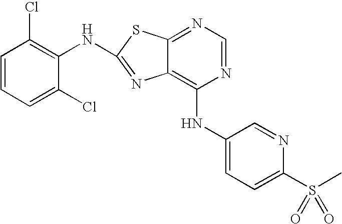

MS (ESI): mass calcd. for C₁₇H₁₂Cl₂N₆O₂S₂, 465.9; m/z found, 467.0 [M+H]⁺. ¹H NMR (CD₃OD): 9.10 (d, J=2.36 Hz, 1H), 8.71 (dd, J=8.70, 2.49 Hz, 1H), 8.46 (s, 1H), 8.05 (d, J=8.69 Hz, 1H), 7.62 (d, J=8.17 Hz, 2H), 7.47-7.42 (m, 1H), 3.21 (s, 3H).

### Example 255

### 2-{4-[2-(2,6-Dichloro-phenylamino)-thiazolo[5,4-d]pyrimidin-7-ylamino]-phenyl}-isobutyramide

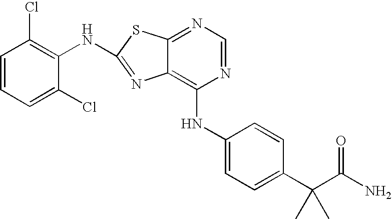

MS (ESI): mass calcd. for C₂₁H₁₈Cl₂N₆OS, 472.0; m/z found, 473.1 [M+H]⁺. ¹H NMR ((CD₃)₂SO): 10.30 (s, 1H), 9.12 (s, 1H), 8.28 (s, 1H), 7.71-7.62 (m, 4H), 7.48-7.41 (m, 1H), 7.26 (d, J=8.72 Hz, 2H), 6.90-6.84 (m, 2H), 1.42 (s, 6H).

### Example 256

### (racemic)-1-[2-(2,6-Dichloro-phenylamino)-7-(4-trifluoromethyl-phenylamino)-thiazolo[5,4-d]pyrimidin-5-ylamino]-propan-2-ol

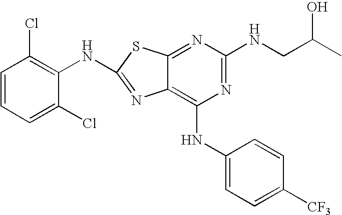

MS (ESI): mass calcd. for C₂₁H₁₇Cl₂F₃N₆OS, 528.0; m/z found, 529.1 [M+H]⁺. ¹H NMR ((CD₃)₂SO): 9.75 (s, 1H), 9.28 (s, 1H), 8.10 (d, J=8.29 Hz, 2H), 7.64-7.56 (m, 4H), 7.42-7.35 (m, 1H), 3.96-3.86 (m, 2H), 3.53-3.45 (m, 1H), 3.37-3.31 (m, 1H), 1.14 (d, J=6.61 Hz, 3H).

### Example 257

### (racemic)-3-[2-(2,6-Dichloro-phenylamino)-7-(4-trifluoromethyl-phenylamino)-thiazolo[5,4-d]pyrimidin-5-ylamino]-propane-1,2-diol

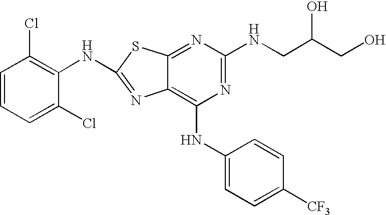

MS (ESI): mass calcd. for C₂₁H₁₇Cl₂F₃N₆O₂S, 544.0; m/z found, 545.1 [M+H]⁺. ¹H NMR ((CD₃)₂SO): 9.74 (s, 1H), 9.23 (s, 1H), 8.12 (d, J=8.63 Hz, 2H), 7.65-7.55 (m, 4H), 7.43-7.35 (m, 1H), 3.49-3.40 (m, 2H), 3.37 (d, J=5.49 Hz, 2H), 3.21-3.14 (m, 1H).

### Example 258

### N²-(2,6-Dichloro-phenyl)-N⁵-(2-pyrrolidin-1-yl-ethyl)-N⁷-(4-trifluoromethyl-phenyl)-thiazolo[5,4-d]pyrimidine-2,5,7-triamine

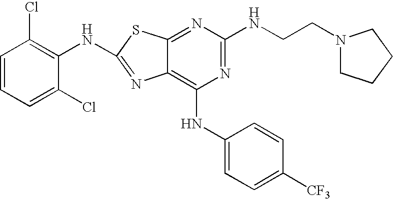

MS (ESI): mass calcd. for C₂₄H₂₂Cl₂F₃N₇S, 567.0; m/z found, 568.1 [M+H]⁺. ¹H NMR (CD₃OD): 7.91 (d, J=8.50 Hz, 2H), 7.64 (d, J=8.64 Hz, 2H), 7.57 (d, J=8.18 Hz, 2H), 7.41-7.36 (m, 1H), 3.82-3.77 (m, 2H), 3.74-3.62 (m, 2H), 3.48-3.42 (m, 2H), 3.14-3.00 (m, 2H), 2.19-1.91 (m, 4H).

### Biological Testing:

Functional assay: block of capsaicin-induced Ca²⁺ influx

### A. Human Assay

HEK293 cells were transfected with human TRPV1 cloned in pcDNA3.1zeo(+) using the Effectene non-liposomal lipid based transfection kit (Qiagen) (hTRPV1/HEK293). hTRPV1/HEK293 cells were routinely grown as monolayers under selection in zeocin (200 μg/mL; Invitrogen) in Dulbecco's Modified Eagle Medium (DMEM, Gibco BRL) supplemented with 10% fetal bovine serum, and penicillin/streptomycin (50 units/mL) in 5% CO₂ at 37° C. Cells were passaged frequently, every 3-5 days, to avoid overgrowth, depletion of essential medium components, or acidic medium exposure. Cells were passaged using a brief wash in 0.05% trypsin with 1 mM EDTA, followed by dissociation in divalent-free phosphate-buffered saline (Hyclone #SH30028.02). Dissociated cells were seeded onto poly-D-lysine coated black-walled 96-well plates (Biocoat; Becton Dickinson #354640) at about 40,000 cells per well and grown for approximately 1 day in culture medium to near confluency. The assay buffer was composed of 130 mM NaCl, 2 mM KCl, 2 mM MgCl₂, 10 mM HEPES, 5 mM glucose, and either 2 mM or 20 μM CaCl₂. On the day of the experiment, the culture medium was replaced with 2 mM calcium assay buffer using an automated plate washer (ELx405; Biotek, VT). The cells were incubated in 100 μL/well Fluo-3/AM (2 μM; TEFLabs #0116) with Pluronic F127 (100 μg/mL; Sigma #P2443) for 1 h at rt in the dark. After loading the cells, the dye solution was replaced with 50 μL/well of 20 μM calcium assay buffer using the ELx405 plate washer. Test compounds (50 μL/well) were added to the plate and incubated for 30 min. Intracellular Ca²⁺ levels were subsequently assayed using a Fluorometric Imaging Plate Reader (FLIPR™ instrument, Molecular Devices, CA) to simultaneously monitor Fluo-3 fluorescence in all wells (λ_{excitation}=488 nm, λₑₘᵢₛₛᵢₒₙ=540 nm) during challenge with agonist (capsaicin). The IC₅₀ values were determined. Cells were challenged with 150 nM capsaicin and the fluorescence counts were captured following agonist addition at a sampling rate of 0.33 Hz. The contents of the wells were mixed 3 times (40 μL mix volume) immediately after the additions were made. Concentration-dependence of block was determined by exposing each well of cells in duplicate rows of a 96-well plate to a serial dilution of test compound. The concentration series usually started at 10 μM with a three-fold serial decrement in concentration. The magnitude of the capsaicin response was determined by measuring the change in fluo3 fluorescence before and 100 seconds after the addition of the agonist. Data were analyzed using a non-linear regression program (Origin; OriginLab, MA).

### B. Rat Assay

This assay was performed similarly to the human assay described above, but using HEK293 cells transfected with rat TRPV1 (rTRPV1/HEK293). These cells had a geneticin selection marker and were grown in Dulbecco's Modified Eagle Medium (DMEM, Gibco BRL) supplemented with 10% fetal bovine serum, penicillin/streptomycin (50 units/mL), and 500 μg/mL geneticin in 5% CO₂ at 37° C.

Results for the compounds tested in these assays are presented in Table 1. IC₅₀ values shown are the average (mean) of the results obtained. Where activity is shown as greater than (>) a particular value, the value is the solubility limit of the compound in the assay medium. Compounds were tested in either the free base or trifluoroacetic acid salt form. Compounds marked with an asterisk were observed to act as agonists rather than antagonists.

TABLE 1

| | Human | Rat |
|---|---|---|
| Ex. | IC₅₀ (nM) | IC₅₀ (nM) |
| 1 | 12 | 3 |
| 2 | 14 | 4 |
| 3 | 14 | 1 |
| 4 | 15 | 8 |
| 5 | 3 | 2 |
| 6 | 5 | 1 |
| 7 | 5 | 1 |
| 8 | 1088 | 70 |
| 9 | >20000 | >20000 |
| 10 | 26 | 6 |
| 11 | 23 | 7 |
| 12 | 30 | 4 |
| 13 | 113 | 34 |
| 14 | 11 | 1 |
| 15 | 13 | 5 |
| 16 | 92 | 39 |
| 17 | 70 | 19 |
| 18 | 30 | 9 |
| 19 | 74 | 31 |
| 20 | >6670 | 2680 |
| 21 | 557 | 299 |
| 22 | >6670 | 1695 |
| 23 | 1140 | 319 |
| 24 | 2365 | 709 |
| 25 | 421 | 68 |
| 26* | 208 | 55 |
| 27* | 91 | 25 |
| 28 | 81 | 39 |
| 29 | 207 | 178 |
| 30 | 3 | 3 |
| 31 | 40 | 33 |
| 32 | 51 | 8 |
| 33 | 205 | 70 |
| 34 | >6670 | >6670 |
| 35 | 31 | 10 |
| 36 | >6670 | >6670 |
| 37 | 134 | 38 |
| 38 | >20000 | 7130 |
| 39 | >6670 | >6670 |
| 40 | 67 | 16 |
| 41 | >6670 | 2040 |
| 42 | 4980 | 236 |
| 43 | >6670 | >6670 |
| 44 | 14700 | 3625 |
| 45 | 70 | 37 |
| 46 | 8 | 5 |
| 47 | >6670 | >6670 |
| 48 | 207 | 202 |
| 49 | 375 | 431 |
| 50 | >6670 | >6670 |
| 51 | 223 | 34 |
| 52 | 226 | 154 |
| 53 | 196 | 93 |
| 54 | 63 | 33 |
| 55 | >20000 | >20000 |
| 56 | 32 | 44 |
| 58 | 15 | 48 |
| 59 | 63 | 22 |
| 62 | 20 | 4 |
| 63 | 46 | 26 |
| 64 | 41 | 17 |
| 69 | 185 | 154 |
| 70 | 225 | 83 |
| 72 | 740 | 627 |
| 73 | 1 | 1 |
| 74 | 1 | 0.05 |
| 75 | 1 | 1 |
| 76 | 1 | 1 |
| 77 | 1 | 2 |
| 78 | 2 | 1 |
| 79 | 2 | 1 |
| 80 | 2 | 1 |
| 81 | 2 | 2 |
| 82 | 5 | 1 |
| 83 | 3 | 2 |
| 84 | 4 | 2 |
| 85 | 4 | 3 |
| 86 | 5 | 3 |
| 87 | 6 | 4 |
| 88 | 7 | 4 |
| 89 | 7 | 2 |
| 90 | 7 | 15 |
| 91 | 7 | 1 |
| 92 | 8 | 2 |
| 93 | 8 | 10 |
| 94 | 8 | 3 |
| 95 | 10 | 7 |
| 96 | 12 | 9 |
| 97 | 12 | 32 |
| 98 | 15 | 7 |
| 99 | 17 | 4 |
| 100 | 17 | 6 |
| 101 | 18 | 12 |
| 102 | 18 | 19 |
| 103 | 20 | 33 |
| 104 | 22 | 7 |
| 105 | 24 | 17 |
| 106 | 25 | 26 |
| 107 | 25 | 2 |
| 108 | 26 | 14 |
| 109 | 28 | 18 |
| 110 | 26 | 20 |
| 111 | 36 | 16 |
| 112 | 36 | 56 |
| 113 | 38 | 17 |
| 114 | 39 | 13 |
| 115 | 44 | 4 |
| 116 | 45 | 11 |
| 117 | 50 | 7 |
| 118 | 56 | 18 |
| 119 | 56 | 51 |
| 120 | 64 | 15 |
| 121 | 82 | 25 |
| 122 | 87 | 129 |
| 123 | 97 | 59 |
| 124 | 98 | 40 |
| 125 | 123 | 19 |
| 126 | 129 | 30 |
| 127 | 138 | 59 |
| 128 | 138 | 121 |
| 129 | 149 | 23 |
| 129 | 149 | 23 |
| 130 | 170 | 87 |
| 131 | 171 | 140 |
| 132 | 172 | 73 |
| 133 | 175 | 107 |
| 134 | 202 | 78 |
| 135 | 203 | 112 |
| 136 | 205 | 25 |
| 137 | 225 | 48 |
| 138 | 271 | 128 |
| 139 | 280 | 78 |
| 140 | 324 | 185 |
| 141 | 350 | 223 |
| 142 | 394 | 231 |
| 143 | 423 | 279 |
| 144 | 431 | 221 |
| 145 | 452 | 442 |
| 146 | 646 | 319 |
| 147 | 723 | 354 |
| 148 | 735 | 126 |
| 149 | 737 | 257 |
| 150 | 937 | 204 |
| 151 | 1220 | 349 |
| 152 | 1220 | 205 |
| 153 | 1421 | 440 |
| 154 | 1515 | 1860 |
| 155 | 1536 | 414 |
| 156 | 1765 | 646 |
| 157 | 1925 | 1215 |
| 158 | 1974 | 313 |
| 159 | >2220 | 1455 |
| 160 | 2990 | >6670 |
| 161 | 3780 | 2290 |
| 162 | 4285 | 3075 |
| 163 | 5952 | 5136 |
| 164 | 6510 | 626 |
| 165 | >6670 | >6670 |
| 166 | >6670 | >6670 |
| 167 | >6670 | >6670 |
| 168 | >6670 | >6670 |
| 169 | >20000 | 17300 |
| 170 | >20000 | >20000 |
| 171 | >20000 | 5320 |
| 172 | >20000 | >20000 |
| 173 | >6670 | >6670 |
| 174 | >20000 | 7380 |
| 175 | >20000 | >20000 |
| 176 | >20000 | >20000 |
| 177 | 21 | 3 |
| 178 | 287 | 60 |
| 179 | 159 | 12 |
| 180 | 123 | 32 |
| 181 | 6440 | >20000 |
| 182 | 1320 | 1820 |
| 183 | 13600 | >20000 |
| 184 | 8 | 5 |
| 185 | 31 | 32 |
| 186 | 642 | 666 |
| 187 | 809 | 1325 |
| 188 | 36 | 40 |
| 189 | 19 | 8 |
| 190 | 45 | 26 |
| 191 | 14 | 56 |
| 192 | 63 | 19 |
| 193 | 35 | 36 |
| 194 | 133 | 125 |
| 195 | 419 | 319 |
| 196 | 64 | 37 |
| 197 | 312 | 287 |
| 198 | 9740 | >20000 |
| 199 | 2145 | 1080 |
| 200 | 386 | 874 |
| 201 | 257 | 244 |
| 202 | 57 | 58 |
| 203 | 48 | 76 |
| 204 | 213 | 226 |
| 205 | 16600 | 12500 |
| 206 | 2470 | 1710 |
| 207 | 758 | 345 |
| 208 | 401 | 398 |
| 209 | 269 | 162 |
| 210 | 4000 | 4000 |
| 211 | 6670 | 6670 |
| 212 | 6670 | 6670 |
| 213 | 6670 | 6670 |
| 214 | 907 | 240 |
| 215 | 15 | 8 |
| 216 | 24 | 15 |
| 217 | 8 | 4 |
| 218 | 55 | 24 |
| 219 | 104 | 76 |
| 220 | 43 | 38 |
| 221 | 1077 | 271 |
| 222 | 100 | 72 |
| 223 | 169 | 95 |
| 224 | 5530 | 1580 |
| 225 | 91 | 32 |
| 226 | 567 | 135 |
| 227 | >6670 | >6670 |
| 228 | 101 | 98 |
| 229 | 139 | 71 |
| 230 | 43 | 2 |
| 231 | 698 | 81 |
| 232 | >6670 | 3190 |
| 233 | 7075 | 7725 |
| 234 | 140 | 68 |
| 235 | 90 | 61 |
| 236 | 115 | 38 |
| 237 | 840 | 189 |
| 238 | 223 | 215 |
| 239 | 3425 | 3745 |
| 240 | 154 | 61 |
| 241 | 820 | 3195 |
| 242 | 1 | 1 |
| 243 | 4605 | 6540 |
| 244 | 12 | 3 |
| 245 | 6 | 5 |
| 246 | 18 | 22 |
| 247 | 35 | 19 |
| 248 | 1105 | 240 |
| 249 | 4370 | 1009 |
| 250 | 3.3 | 0.7 |
| 251 | 290 | 110 |

While the invention has been illustrated by reference to exemplary and preferred embodiments, it will be understood that the invention is intended not to be limited by the foregoing detailed description, but to be defined by the appended claims as properly construed under principles of patent law.

What is claimed is:

1. A composition of matter selected from the group consisting of:
(a) compounds of Formula (I): 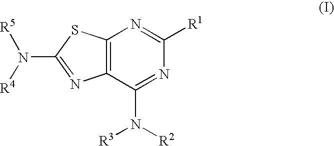
wherein:
R¹ is —H; —NR^{a}R^{b}; a —C₁₋₆alkyl, —OC₁₋₆alkyl, —S—C₁₋₆alkyl, or —SO₂—C₁₋₆alkyl group unsubstituted or substituted with an —OH, —OC₁₋₄alkyl, —NR^{e}R^{f}, or halo substituent; or a monocyclic cycloalkyl or phenyl group unsubstituted or substituted with a —C₁₋₆alkyl, —OH, —OC₁₋₁₄alkyl, —NR^{e}R^{f}, or halo substituent;
where R^{a} and R^{b} are each independently —H; —C₁₋₆alkyl; a —C₂₋₄alkyl group substituted with one or two —OH, —OC₁₋₄alkyl, —NR^{c}R^{d}, or halo substituents; or a saturated monocyclic cycloalkyl, —C₁alkyl-(saturated monocyclic cycloalkyl), saturated monocyclic heterocycloalkyl, —C₁ alkyl-(saturated monocyclic heterocycloalkyl), phenyl, or benzyl group unsubstituted or substituted with one, two, or three moieties independently selected from the group consisting of —C₁₋₆alkyl, —OH, —OC₁₋₄alkyl, —NR^{p}R^{q}, and halo substituents; or
R^{a} and R^{b} taken together with the nitrogen of attachment in —NR^{a}R^{b} form a saturated monocyclic heterocycloalkyl group unsubstituted or substituted with one, two, or three moieties independently selected from the group consisting of —C₁₋₆alkyl, —C₁₋₂alkyl-OH, —C₁₋₂alkyl-OC₁₋₂alkyl, —OH, —OC₁₋₄alkyl, —NR^{p}R^{q}, halo, —CO₂H, and benzyl substituents;
where R^{e} and R^{d} are each independently —H or —C₁₋₆alkyl; or R^{c} and R^{d} taken together with the nitrogen of attachment in —NR^{c}R^{d} form a saturated monocyclic heterocycloalkyl unsubstituted or substituted with methyl; and
R^{p} and R^{q} are each independently —H or —C₁₋₆alkyl; or R^{p} and R^{q} taken together with the nitrogen of attachment in —NR^{p}R^{q} form a saturated monocyclic heterocycloalkyl unsubstituted or substituted with methyl; and
R^{e} and R^{f} are each independently —H or —C₁₋₆alkyl; or R^{e} and R^{f} taken together with their nitrogen of attachment in —NR^{e}R^{f} form a saturated monocyclic heterocycloalkyl unsubstituted or substituted with methyl;
R² is —H or —C₁₋₆alkyl;
R³ is a monocyclic cycloalkyl, phenyl, benzyl, phenethyl, indanyl, quinolinyl, monocyclic five-membered heteroaryl, monocyclic six-membered heteroaryl, or —C₁alkyl-(monocyclic heteroaryl) group unsubstituted or substituted with one, two, or three R^{g} substituents;
where each R^{g} substituent is independently —C₁₋₆alkyl, —OH, —OC₁₋₆alkyl, —O-(saturated monocyclic heterocycloalkyl), phenoxy, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH; or
two adjacent R^{g} substituents taken together form —OC₁₋₂alkylO—, —C₂₋₆alkylO—, or —C₂₋₆alkylN(R)—;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl unsubstituted or substituted with methyl; and
each R^{j} is independently —H, —C₁₋₆alkyl, or —CF₃; or both R^{j} substituents taken together with the carbon to which they are attached form a monocyclic cycloalkyl ring;
R⁴ is —H or —C₁₋₆alkyl; and
R⁵ is a phenyl, monocyclic five-membered heteroaryl, or monocyclic six-membered heteroaryl group unsubstituted or substituted with one, two, or three R^{k} substituents;
where each R^{k} substituent is independently —C₁₋₆alkyl unsubstituted or substituted with one or two —OH groups, —C₁₋₂alkyl-N(R^{l})R^{m}, —OH, —OC₁₋₆alkyl, phenyl, phenoxy, —CN, —NO₂, —N(R^{l})R^{m}, —C(O)N(R^{l})R^{m}, —N(R^{l})C(O)R^{m}, —N(R^{l})SO₂C₁₋₆alkyl, —N(R^{l})SO₂CF₃, —C(O)C₁₋₆alkyl, —S(O)₀₋₂—C₁₋₆alkyl, —SO₂CF₃, —SO₂N(R^{l})R^{m}, —SCF₃, halo, —CF₃, —OCF₃, —CO₂H, or —CO₂C₁₋₆alkyl; or
two adjacent R^{k} substituents taken together form —OC₁₋₂alkylO— or ═N—S—N═;
where R^{l} and R^{m} are each independently —H or —C₁₋₆alkyl; or R^{l} and R^{m} taken together with their nitrogen of attachment in —NR^{l}R^{m} form a saturated monocyclic heterocycloalkyl unsubstituted or substituted with methyl; and
(b) pharmaceutically acceptable salts of the compounds of Formula (I), pharmaceutically acceptable prodrugs of the compounds of Formula (I), and pharmaceutically active metabolites of the compounds of Formula (I).

2. A composition of matter as defined in claim 1 selected from the group consisting of:
(a) the compounds of Formula (I) wherein R¹ is —H, methyl, —CH₂— (monocyclic cycloalkyl), or —NR^{a}R^{b};
where R^{a} and R^{b} are each independently —H; —C₁₋₆alkyl; a —C₂₋₃alkyl group substituted with an —OH, —OC₁₋₄alkyl, or —NR^{c}R^{d} substituent; or a saturated monocyclic cycloalkyl or —C₁₋₄alkyl-(saturated monocyclic cycloalkyl) group unsubstituted or substituted with a methyl, —OH, or —OC₁₋₄alkyl substituent; or
R^{a} and R^{b} taken together with the nitrogen of attachment in —NR^{a}R^{b} form a saturated monocyclic heterocycloalkyl group unsubstituted or substituted with a methyl, —OH, or —OC₁₋₄alkyl substituent; and
where R^{c} and R^{d} are each independently —H or —C₁₋₆alkyl; and
(b) pharmaceutically acceptable salts of said compounds.

3. A composition of matter as defined in claim 1 selected from the group consisting of:
(a) the compounds of Formula (I) wherein R^{g} is —C₁₋₄alkyl, methoxy, —CF₃, halo, —C(CH₃)₂CONH₂, 1-hydroxy-cyclopropyl, —SO₂CH₃, —SO₂CF₃, or —SO₂N(R^{h})R¹;
where R^{h} and Rⁱ are each independently —H or —C₁₋₆alkyl; and
(b) pharmaceutically acceptable salts of said compounds.

4. A composition of matter as defined in claim 1 selected from the group consisting of:
(a) the compounds of Formula (I) wherein each R^{k} substituent is independently —H, chloro, methyl, —CH₂OH, or —CH₂N(R^{l})R^{m}; where R^{l} and R^{m} are each independently —H or —C₁₋₆alkyl; and
(b) pharmaceutically acceptable salts of said compounds.

5. A composition of matter as defined in claim 1 selected from the group consisting of
(a) the compounds of Formula (I) wherein:
R¹ is —H; —NR^{a}R^{b}; a —C₁₋₆alkyl, —OC₁₋₆alkyl, —S—C₁₋₆alkyl, or —SO₂—C₁₋₆alkyl group unsubstituted or substituted with an —OH, —OC₁₋₄alkyl, —NR^{e}R^{f}, or halo substituent; or a monocyclic cycloalkyl or phenyl group unsubstituted or substituted with a —C₁₋₆alkyl, —OH, —OC₁₋₄alkyl, —NR^{e}R^{f}, or halo substituent;
where R^{a} and R^{b} are each independently —H; —C₁₋₆alkyl; a —C₂₋₃alkyl group substituted with an —OH, —OC₁₋₄alkyl, —NR^{c}R^{d}, or halo substituent; or a saturated monocyclic cycloalkyl, —C₁alkyl-(saturated monocyclic cycloalkyl), saturated monocyclic heterocycloalkyl, —C₁₋₄alkyl-(saturated monocyclic heterocycloalkyl), phenyl, or benzyl group unsubstituted or substituted with one, two, or three moieties independently selected from the group consisting of —C₁₋₆alkyl, —OH, —OC₁₋₄alkyl, —NR^{p}R^{q}, and halo substituents; or
R^{a} and R^{b} taken together with the nitrogen of attachment in —NR^{a}R^{b} form a saturated monocyclic heterocycloalkyl group unsubstituted or substituted with one, two, or three moieties independently selected from the group consisting of —C₁₋₆alkyl, —OH, —OC₁₋₄alkyl, —NR^{p}R^{q}, halo, —CO₂H, and benzyl substituents;
where R^{c} and R^{d} are each independently —H or —C₁₋₆alkyl; or R^{c} and R^{d} taken together with the nitrogen of attachment in —NR^{c}R^{d} form a saturated monocyclic heterocycloalkyl; and
R^{p} and R^{q} are each independently —H or —C₁₋₆alkyl; or R^{p} and R^{q} taken together with the nitrogen of attachment in —NR^{p}R^{q} form a saturated monocyclic heterocycloalkyl; and
R^{e} and R^{f} are each independently —H or —C₁₋₆alkyl; or R^{e} and R^{f} taken together with their nitrogen of attachment in —NR^{e}R^{f} form a saturated monocyclic heterocycloalkyl;
R² is —H or —C₁₋₆alkyl;
R³ is a monocyclic cycloalkyl, phenyl, benzyl, phenethyl, indanyl, monocyclic five-membered heteroaryl, monocyclic six-membered heteroaryl, or —C₁alkyl-(monocyclic heteroaryl) group unsubstituted or substituted with one, two, or three R^{g} substituents;
where each R^{g} substituent is —C₁₋₆alkyl, —OH, —OC₁₋₆alkyl, phenoxy, —CN, —NO₂, —N(R^{h})Rⁱ, —C(O)N(R^{h})Rⁱ, —N(R^{h})C(O)Rⁱ, —N(R^{h})SO₂C₁₋₆alkyl, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, or —C(R^{j})₂—OH; or
two adjacent R^{g} substituents taken together form —OC₁₋₂alkylO—, —C₂₋₆alkylO—, or —C₂₋₆alkylN(R^{h})—;
where R^{h} and Rⁱ are each independently —H or —C₁₋₆alkyl; and
each R^{j} is independently —H or —C₁₋₆alkyl;
R⁴ is —H or —C₁₋₆alkyl; and
R⁵ is a phenyl, monocyclic five-membered heteroaryl, or monocyclic six-membered heteroaryl group unsubstituted or substituted with one, two, or three R^{k} substituents;
where each R^{k} substituent is independently —C₁₋₆alkyl, —OH, —OC₁₋₆alkyl, phenyl, phenoxy, —CN, —NO₂, —N(R^{l})R^{m}, —C(O)N(R^{l})R^{m}, —N(R^{l})C(O)R^{m}, —N(R^{l})SO₂C₁₋₆alkyl, —N(R^{l})SO₂CF₃, —C(O)C₁₋₆alkyl, —S(O)₀₋₂—C₁₋₆alkyl, —SO₂CF₃, —SO₂N(R^{l})R^{m}, —SCF₃, halo, —CF₃, —OCF₃, —CO₂H, or —CO₂C₁₋₆alkyl; or
two adjacent R^{k} substituents taken together form —OC₁₋₂alkylO—;
where R^{l} and R^{m} are each independently —H or —C₁₋₆alkyl;
and (b) pharmaceutically acceptable salts of said compounds.

6. A composition of matter as defined in claim 5, wherein R¹ is —H or a methyl, ethyl, propyl, or isopropyl group unsubstituted or substituted with a —OH, —OC₁₋₄alkyl, —NR^{e}R^{f}, or halo substituent; or a cyclopropyl, cyclobutyl, cyclopentyl, or cyclohexyl group unsubstituted or substituted with a —C₁₋₆alkyl, —OH, —OC₁₋₄alkyl, —NR^{e}R^{f}, or halo substituent.

7. A composition of matter as defined in claim 5, wherein R¹ is —NR^{a}R^{b} or a methoxy, ethoxy, propyloxy, isopropyloxy, methanesulfanyl, ethanesulfanyl, propylsulfanyl, isopropylsulfanyl, methanesulfonyl, ethanesulfonyl, propylsulfonyl, or isopropylsulfonyl group unsubstituted or substituted with a —OH, —OC₁₋₄alkyl, —NR^{e}R^{f}, or halo substituent.

8. A composition of matter as defined in claim 5, wherein R¹ is —NR^{a}R^{b}, and R^{a} and R^{b} are each independently —H; methyl, ethyl, propyl, isopropyl, butyl, isobutyl, sec-butyl, tert-butyl, pentyl, isopentyl, or hexyl; an ethyl or propyl group substituted with an —OC₁₋₄alkyl or —NR^{c}R^{d} substituent; or a cyclopropyl, cyclobutyl, cyclopentyl, cyclohexyl, cycloheptyl, cyclopropylmethyl, cyclopentylmethyl, aziridinyl, pyrrolidinyl, tetrahydrofuranyl, piperidinyl, tetrahydropyranyl, piperazinyl, morpholinyl, thiomorpholinyl, 1,1-dioxo-1λ⁶-thiomorpholin-4-yl, or phenyl group unsubstituted or substituted with a —C₁₋₆alkyl, —OC₁₋₄alkyl, or halo substituent.

9. A composition of matter as defined in claim 5, wherein R¹ is —NR^{a}R^{b}, and R^{a} and R^{b} are each independently —H, methyl, methoxyethyl, cyclopropylmethyl, or phenyl.

10. A composition of matter as defined in claim 5, wherein R¹ is —NR^{a}R^{b}, and R^{a} and R^{b} taken together with the nitrogen of attachment form an azetidinyl, pyrrolidinyl, piperidinyl, 2-oxo-piperidin-1-yl, piperazinyl, oxo-piperazinyl, morpholinyl, thiomorpholinyl, 1,1-dioxo-1λ⁶-thiomorpholin-4-yl, 1,1-dioxo-1λ⁶-[1,2]thiazinan-2-yl, or azepanyl group unsubstituted or substituted with a —C₁₋₆alkyl, —OH, or —CO₂H substituent.

11. A composition of matter as defined in claim 5, wherein R¹ is —NR^{a}R^{b}, and at least one of R^{a} and R^{b} is a —C₂₋₃alkyl group substituted with an —OH, —OC₁₋₄alkyl, —NR^{c}R^{d}, or halo substituent, where R^{c} and R^{d} are each independently —H, methyl, or ethyl.

12. A composition of matter as defined in claim 5, wherein R² is H, and R⁴ is H.

13. A composition of matter as defined in claim 12, wherein R¹ is H.

14. A composition of matter as defined in claim 5, wherein R¹ is —H, methyl, isopropyl, methanesulfanyl, methanesulfonyl, methoxy, phenyl, phenoxy, dimethylamino, azetidinyl, pyrrolidinyl, piperidinyl, azepanyl, morpholinyl, 4-isopropyl-piperazin-1-yl, 2-methoxyethylamino, (2-methoxyethylamino)methylamino, cyclopropylmethylamino, or phenylamino.

15. A composition of matter as defined in claim 5, wherein R¹ is —H or methyl.

16. A composition of matter as defined in claim 5, wherein R² is —H or methyl.

17. A composition of matter as defined in claim 5, wherein R³ is a cyclopentyl, cyclohexyl, phenyl, indanyl, furanyl, thiophenyl, pyrrolyl, oxazolyl, thiazolyl, pyridyl, pyrimidinyl, or pyrazinyl group unsubstituted or substituted with one or two R^{g} substituents.

18. A composition of matter as defined in claim 5, wherein R³ is a phenyl or pyridyl group substituted with one or two R^{g} substituents.

19. A composition of matter as defined in claim 18, wherein each R^{g} substituent is independently methyl, isopropyl, tert-butyl, —OH, —OCH₃, phenoxy, —CN, —NO₂, —NH₂, —C(O)CH₃, —SO₂CF₃, —SO₂NH₂, —SCF₃, chloro, bromo, —CF₃, —OCF₃, —CO₂CH₃, —C(CH₃)₂—CN, or —C(CH₃)₂—OH; or two adjacent R^{g} substituents taken together form —OC₁₋₂alkylO—.

20. A composition of matter as defined in claim 18, wherein each R^{g} substituent is independently methyl, tert-butyl, —OH, —OCH₃, —CN, —SCF₃, chloro, —CF₃, —OCF₃, —CO₂CH₃, or —C(CH₃)₂—CN.

21. A composition of matter as defined in claim 18, wherein R¹ is —H or methyl; and R² is —H or methyl.

22. A composition of matter as defined in claim 21, wherein R⁴ is —H, methyl, or ethyl.

23. A composition of matter as defined in claim 22, wherein R⁵ is a phenyl, furanyl, thiophenyl, isoxazolyl, or pyridyl group substituted with one or two R^{k} substituents independently selected from the group consisting of methyl, ethyl, propyl, isopropyl, —OH, —OCH₃, phenyl, phenoxy, —CN, —NO₂, —NH₂, methylamino, dimethylamino, —NHSO₂CH₃, —C(O)CH₃, —SO₂NH₂, —SO₂CF₃, —SCF₃, chloro, bromo, —CF₃, —OCF₃, —CO₂H, and —CO₂CH₃.

24. A composition of matter as defined in claim 5, wherein R⁵ is a phenyl, furanyl, thiophenyl, isoxazolyl, or pyridyl group substituted with one or two R^{k} substituents.

25. A composition of matter as defined in claim 5, wherein R⁵ is a phenyl or pyridyl group ortho-substituted with one or two R^{k} substituents, where each R^{k} substituent is independently methyl, ethyl, propyl, isopropyl, —OH, —OCH₃, phenyl, phenoxy, —CN, —NO₂, —NH₂, methylamino, dimethylamino, —NHSO₂CH₃, —C(O)CH₃, —SO₂NH₂, —SO₂CF₃, —SCF₃, chloro, bromo, —CF₃, —OCF₃, —CO₂H, or —CO₂CH₃.

26. A composition of matter as defined in claim 25, wherein each R^{k} substituent is independently methyl, —CF₃, chloro, phenyl, —SO₂CH₃, or —CO₂CH₃.

27. A composition of matter as defined in claim 5, wherein R¹ is —H, methyl, isopropyl, methanesulfanyl, methanesulfonyl, methoxy, phenyl, phenoxy, dimethylamino, azetidinyl, pyrrolidinyl, piperidinyl, azepanyl, morpholinyl, 4-isopropyl-piperazin-1-yl, 2-methoxyethylamino, (2-methoxyethylamino)methylamino, cyclopropylmethylamino, or phenylamino; and R³ is a phenyl or pyridyl group substituted with one or two R^{g} substituents.

28. A composition of matter as defined in claim 27, wherein R⁵ is a phenyl, furanyl, thiophenyl, isoxazolyl, or pyridyl group substituted with one or two R^{k} substituents.

29. A composition of matter as defined in claim 28, wherein R⁵ is a phenyl or pyridyl group ortho-substituted with one or two R^{k} substituents.

30. A composition of matter selected from the group consisting of:
(a) compounds of Formula (I′): 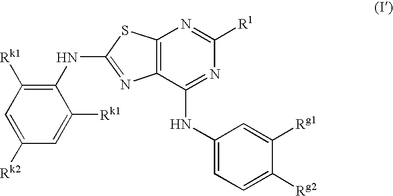
wherein:
R¹ is —H, methyl, —CH₂— (monocyclic cycloalkyl), or —NR^{a}R^{b};
where R^{a} and R^{b} are each independently —H; —C₁₋₆alkyl; a —C₂₋₃alkyl group substituted with an —OH, —OC₁₋₄alkyl, or —NR^{c}R^{d} substituent; or a saturated monocyclic cycloalkyl or —C₁alkyl-(saturated monocyclic cycloalkyl) group unsubstituted or substituted with a methyl, —OH, or —OC₁₋₄alkyl substituent; or
R^{a} and R^{b} taken together with the nitrogen of attachment in —NR^{a}R^{b} form a saturated monocyclic heterocycloalkyl group unsubstituted or substituted with a methyl, —OH, or —OC₁₋₄alkyl substituent;
where R^{c} and R^{d} are each independently —H or —C₁₋₆alkyl;
R^{g1} is —H or halo;
R^{g2} is —C₁₋₄alkyl, methoxy, —CF₃, —SO₂CH₃, —SO₂CF₃, or —SO₂N(R^{h})Rⁱ;
where R^{h} and Rⁱ are each independently —H or —C₁₋₆alkyl;
both R^{k1} are chloro or methyl; and
R^{k2} is —H, —CH₂OH, or —CH₂N(R^{l})R^{m};
where R^{l} and R^{m} are each independently —H or —C₁₋₆alkyl; and
(b) pharmaceutically acceptable salts of the compounds of Formula (I′), pharmaceutically acceptable prodrugs of the compounds of Formula (I′), and pharmaceutically active metabolites of the compounds of Formula (I′).

31. A composition of matter as defined in claim 30 selected from the group consisting of:
(a) the compounds of the Formula (I′) wherein R^{g1} is —H; and
(b) pharmaceutically acceptable salts of said compounds.

32. A composition of matter as defined in claim 30 selected from the group consisting of:
(a) the compounds of the Formula (I′) wherein R^{g2} is —CF₃; and
(b) pharmaceutically acceptable salts of said compounds.

33. A composition of matter as defined in claim 32, wherein both R^{k1} are chloro.

34. A composition of matter as defined in claim 1, selected from the group consisting of:
N²-(2,6-Dichloro-phenyl)-N⁷-(4-trifluoromethyl-phenyl)-thiazolo[5,4-d]pyrimidine-2,7-diamine;
N²-(2,6-Dichloro-phenyl)-N⁷-(6-trifluoromethyl-pyridin-3-yl)-thiazolo[5,4-d]pyrimidine-2,7-diamine;
N⁷-(4-tert-Butyl-phenyl)-N²-(2,6-dichloro-phenyl)-thiazolo[5,4-d]pyrimidine-2,7-diamine;
N⁷-(3-Chloro-4-trifluoromethyl-phenyl)-N²-(2,6-dichloro-phenyl)-thiazolo[5,4-d]pyrimidine-2,7-diamine;
N²-(2,6-Dichloro-phenyl)-5-methyl-N⁷-(4-trifluoromethyl-phenyl)-thiazolo[5,4-d]pyrimidine-2,7-diamine;
N²-(2,6-Dichloro-phenyl)-5-methyl-N⁷-(6-trifluoromethyl-pyridin-3-yl)-thiazolo[5,4-d]pyrimidine-2,7-diamine;
N⁷-(4-tert-Butyl-phenyl)-N²-(2,6-dichloro-phenyl)-5-methyl-thiazolo[5,4-d]pyrimidine-2,7-diamine;
N⁷-(4-tert-Butyl-cyclohexyl)-N²-(2,6-dichloro-phenyl)-5-methyl-thiazolo[5,4-d]pyrimidine-2,7-diamine;
N²-(2,6-Dichloro-phenyl)-5,N⁷-dimethyl-N⁷-(4-trifluoromethyl-phenyl)-thiazolo[5,4-d]pyrimidine-2,7-diamine;
N²-(2,6-Dimethyl-phenyl)-N⁷-(4-trifluoromethyl-phenyl)thiazolo[5,4,d]pyrimidine-2,7-diamine;
N²-(2,6-Dimethyl-phenyl)-N⁷-(6-trifluoromethyl-pyridin-3-yl)-thiazolo[5,4-d]pyrimidine-2,7-diamine;
N⁷-(4-tert-Butyl-phenyl)-N⁷-(2,6-dimethyl-phenyl)-thiazolo[5,4-d]pyrimidine-2,7-diamine;
N⁷-(3-Chloro-4-trifluoromethyl-phenyl)-N²-(2,6-dimethyl-phenyl)-thiazolo[5,4-d]pyrimidine-2,7-diamine;
N²-(2-Chloro-6-methyl-phenyl)-N⁷-(4-trifluoromethyl-phenyl)-thiazolo[5,4-d]pyrimidine-2,7-diamine;
N²-(2-Chloro-6-methyl-phenyl)-N⁷-(6-trifluoromethyl-pyridin-3-yl)-thiazolo[5,4-d]pyrimidine-2,7-diamine;
N²-(2-Chloro-phenyl)-N⁷-(4-trifluoromethyl-phenyl)-thiazolo[5,4-d]pyrimidine-2,7-diamine;
N²-o-Tolyl-N⁷-(4-trifluoromethyl-phenyl)-thiazolo[5,4-d]pyrimidine-2,7-diamine;
N²-(2-Chloro-6-trifluoromethyl-phenyl)-N⁷-(4-trifluoromethyl-phenyl)-thiazolo[5,4-d]pyrimidine-2,7-diamine;
N²-(2-Chloro-6-trifluoromethyl-phenyl)-N⁷-(6-trifluoromethyl-pyridin-3-yl)-thiazolo[5,4-d]pyrimidine-2,7-diamine;
N²-Phenyl-N⁷-(4-trifluoromethyl-phenyl)-thiazolo[5,4-d]pyrimidine-2,7-diamine;
N²-Phenyl-N⁷-(6-trifluoromethyl-pyridin-3-yl)-thiazolo[5,4-d]pyrimidine-2,7-diamine;
N²,N⁷-Bis-(4-trifluoromethyl-phenyl)-thiazolo[5,4-d]pyrimidine-2,7-diamine;
N²-(2,6-Dichloro-phenyl)-5,N²-dimethyl-N⁷-(4-trifluoromethyl-phenyl)-thiazolo[5,4-d]pyrimidine-2,7-diamine;
N²-(3,5-Dimethyl-isoxazol-4-yl)-N⁷-(6-trifluoromethyl-pyridin-3-yl)-thiazolo[5,4-d]pyrimidine-2,7-diamine;
N²-(3,5-Dimethyl-isoxazol-4-yl)-N⁷-(4-trifluoromethyl-phenyl)-thiazolo[5,4-d]pyrimidine-2,7-diamine;
5-Methyl-N²-(5-methyl-3-phenyl-isoxazol-4-yl)−/−(6-trifluoromethyl-pyridin-3-yl)-thiazolo[5,4-d]pyrimidine-2,7-diamine;
5-Methyl-N²-(5-methyl-3-phenyl-isoxazol-4-yl)-N⁷-(4-trifluoromethyl-phenyl)-thiazolo[5,4-d]pyrimidine-2,7-diamine;
N²-(2,6-Dimethyl-phenyl)-N⁷-(4-trifluoromethoxy-phenyl)-thiazolo[5,4-d]pyrimidine-2,7-diamine;
5-[2-(2,6-Dimethyl-phenylamino)-thiazolo[5,4-d]pyrimidin-7-ylamino]-pyridine-2-carbonitrile;
N⁷-(3-Chloro-4-trifluoromethyl-phenyl)-N²-(2,6-dichloro-phenyl)-5-methyl-thiazolo[5,4-d]pyrimidine-2,7-diamine;
4-[2-(2,6-Dimethyl-phenylamino)-thiazolo[5,4-d]pyrimidin-7-ylamino]-benzonitrile;
2-{4-[2-(2,6-Dimethyl-phenylamino)-thiazolo[5,4-d]pyrimidin-7-ylamino]-phenyl}-2-methyl-propionitrile;
N²-(2,6-Dimethyl-phenyl)-N⁷-(4-methoxy-phenyl)-thiazolo[5,4-d]pyrimidine-2,7-diamine;
N⁷-(3,4-Dichloro-phenyl)-N²-(2,6-dimethyl-phenyl)-thiazolo[5,4-d]pyrimidine-2,7-diamine;
N²-(2,6-Dimethyl-phenyl)-N⁷-p-tolyl-thiazolo[5,4-d]pyrimidine-2,7-diamine;
N²-(2,6-Dimethyl-phenyl)-N⁷-(2-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzoic acid methyl ester;
4-{[2-(2,6-Dimethyl-phenylamino)-thiazolo[5,4-d]pyrimidin-7-ylamino]-methyl}-2-methoxy-phenol;
N⁷-(3,4-Dichloro-benzyl)-N²-(2,6-dimethyl-phenyl)-thiazolo[5,4-d]pyrimidine-2,7-diamine;
N²-(2,6-Dimethyl-phenyl)-N⁷-(4-trifluoromethylsulfanyl-phenyl)-thiazolo[5,4-d]pyrimidine-2,7-diamine;
N²-(2,6-Dimethyl-phenyl)-N⁷-indan-2-yl-thiazolo[5,4-d]pyrimidine-2,7-diamine;
N²-(2,6-Dimethyl-phenyl)-N⁷-(3-trifluoromethyl-phenyl)-thiazolo[5,4-d]pyrimidine-2,7-diamine;
N²-Benzyl-N²-(2,6-dimethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzenesulfonamide;
N²-(2,6-Dimethyl-phenyl)-N⁷-(4-ethyl-phenyl)-thiazolo[5,4-d]pyrimidine-2,7-diamine;
N²-(2,6-Dimethyl-phenyl)-N⁷-(4-isopropyl-phenyl)-thiazolo[5,4-d]pyrimidine-2,7-diamine;
N²-(2,6-Dimethyl-phenyl)-N⁷-(5-methyl-furan-2-ylmethyl)-thiazolo[5,4-d]pyrimidine-2,7-diamine;
4-Methyl-3-[7-(4-trifluoromethyl-phenylamino)-thiazolo[5,4-d]pyrimidin-2-ylamino]-thiophene-2-carboxylic acid methyl ester;
4-Methyl-3-[7-(6-trifluoromethyl-pyridin-3-ylamino)-thiazolo[5,4-d]pyrimidin-2-ylamino]-thiophene-2-carboxylic acid methyl ester;
N⁷-(3-Chloro-4-trifluoromethyl-phenyl)-N²-(3,5-dimethyl-isoxazol-4-yl)thiazolo[5,4d]pyrimidine-2,7-diamine;
N⁷-(4-tert-Butyl-phenyl)-N²-(3,5-dimethyl-isoxazol-4-yl)-thiazolo[5,4-d]pyrimidine-2,7-diamine;
N²-(2,6-Dichloro-phenyl)-5-methylsulfanyl-N²-(4-trifluoromethyl-phenyl)-thiazolo[5,4-d]pyrimidine-2,7-diamine;
N²-(2,6-Dichloro-phenyl)-5-methanesulfonyl-N⁷-(4-trifluoromethy-phenyl)-thiazolo[5,4-d]pyrimidine-2,7-diamine;
N²-(2,6-Dichloro-phenyl)-5-piperidin-1-yl-N⁷-(4-trifluoromethyl-phenyl)-thiazolo[5,4-d]pyrimidine-2,7-diamine;
N²-(2,6-Dichloro-phenyl)-5-methoxy-N⁷-(4-trifluoromethyl-phenyl)-thiazolo[5,4d]pyrimidine-2,7-diamine;
N²-(2,6-Dichloro-phenyl)-N⁵,N⁵-dimethyl-N⁷-(4-trifluoromethyl-phenyl)-thiazolo[5,4-d]pyrimidine-2,5,7-triamine;
5-Azepan-1-yl-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-pyrrolidin-1-yl-N⁷-(4-trifluoromethyl-phenyl)-thiazolo[5,4-d]pyrimidine-2,7-diamine;
5-Azetidin-1-yl-N²-(2,6-dichloro-phenyl)-N⁷-(4-trifluoromethyl-phenyl)-thiazolo[5,4-d]pyrimidine-2,7-diamine;
N²-(2,6-Bis-methanesulfonyl-phenyl)-5-methyl-N⁷-(4-trifluoromethyl-phenyl)-thiazolo[5,4-d]pyrimidine-2,7-diamine;
N²-(2,6-Dichloro-phenyl)-N⁵-(2-methoxy-ethyl)-N⁷-(4-trifluoromethyl-phenyl)-thiazolo[5,4-d]pyrimidine-2,5,7-triamine;
N⁵-Cyclopropylmethyl-N²-(2,6-dichloro-phenyl)-N⁷-(4-trifluoromethyl-phenyl)-thiazolo[5,4-d]pyrimidine-2,5,7-triamine;
N²-(2,6-Dichloro-phenyl)-N⁵-(2-methoxy-ethyl)-N⁵-methyl-N⁷-(4-trifluoromethyl-phenyl)-thiazolo[5,4-d]pyrimidine-2,5,7-triamine;
N-(2,6-Dichloro-phenyl)-5-morpholin-4-yl-N⁷-(4-trifluoromethyl-phenyl)-thiazolo[5,4-d]pyrimidine-2,7-diamine;
N²-(2,6-Dichloro-phenyl)-N⁷-(5-trifluoromethyl-pyridin-2-yl)-thiazolo[5,4-d]pyrimidine-2,7-diamine;
N²-(2,6-Dichloro-phenyl)-5-methyl-N⁷-(5-trifluoromethyl-pyridin-2-yl)-thiazolo[5,4-d]pyrimidine-2,7-diamine;
N²-(2,6-Dichloro-phenyl)-5-phenoxy-N⁷-(4-trifluoromethyl-phenyl)-thiazolo[5,4-d]pyrimidine-2,7-diamine;
N²-(2,6-Dichloro-phenyl)-N⁵-phenyl-N⁷-(4-trifluoromethyl-phenyl)-thiazolo[5,4-d]pyrimidine-2,5,7-triamine;
N²-(2,6-Dichloro-phenyl)-5-(4-isopropyl-piperazin-1-yl)-N⁷-(4-trifluoromethyl-phenyl)-thiazolo[5,4-d]pyrimidine-2,7-diamine;
N²-(2,6-Dichloro-phenyl)-5-phenyl-N²-(4-trifluoromethyl-phenyl)-thiazolo[5,4-d]pyrimidine-2,7-diamine;
N²-(2,6-Dichloro-phenyl)-5-isopropyl-N⁷-(4-trifluoromethyl-phenyl)-thiazolo[5,4-d]pyrimidine-2,7-diamine; and
N²-(3,5-Dichloro-pyridin-4-yl)-N⁷-(4-trifluoromethyl-phenyl)-thiazolo[5,4-d]pyrimidine-2,7-diamine;
and pharmaceutically acceptable salts thereof.

35. A composition of matter as defined in claim 1 selected from the group consisting of:
N²-(2,6-Dichloro-phenyl)-5-methyl-N⁷-[4-(pyrrolidine-1-sulfonyl)-phenyl]-thiazolo[5,4-d]pyrimidine-2,7-diamine;
2-{4-[2-(2,6-Dichloro-phenylamino)-5-methyl-thiazolo[5,4-d]pyrimidin-7-ylamino]-phenyl}-propan-2-ol;
4-[2-(2,6-Dichloro-phenylamino)-thiazolo[5,4-d]pyrimidin-7-ylamino]-N,N-dimethyl-benzenesulfonamide;
N²-(2,6-Dichloro-phenyl)-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-(4-trifluoromethanesulfonyl-phenyl)-thiazolo[5,4-d]pyrimidine-2,7-diamine;
N²-(2,6-Dichloro-phenyl)-N⁷-(4-methanesulfonyl-phenyl)-5-methyl-thiazolo[5,4-d]pyrimidine-2,7-diamine;
N²-(2,6-Dichloro-phenyl)-N⁵-isobutyl-N⁷-(4-trifluoromethyl-phenyl)-thiazolo[5,4-d]pyrimidine-2,5,7-triamine;
N²-(2,6-Dichloro-phenyl)-N⁷-[4-(morpholine-4-sulfonyl)-phenyl]-thiazolo[5,4-d]pyrimidine-2,7-diamine;
4-[2-(2,6-Dichloro-phenylamino)-5-methyl-thiazolo[5,4-d]pyrimidin-7-ylamino]-N,N-dimethyl-benzenesulfonamide;
N²-(2,6-Dichloro-phenyl)-N⁷-(3-fluoro-4-methanesulfonyl-phenyl)-thiazolo[5,4-d]pyrimidine-2,7-diamine;
N⁷-[4-(Pyrrolidine-1-sulfonyl)-phenyl]-N²-o-tolyl-thiazolo[5,4-d]pyrimidine-2,7-diamine;
N²-(2,6-Dichloro-phenyl)-N⁷-(4-isopropyl-phenyl)-5-methyl-thiazolo[5,4-d]pyrimidine-2,7-diamine;
4-[2-(2,6-Dimethyl-phenylamino)-5-methyl-thiazolo[5,4-d]pyrimidin-7-ylamino]-N,N-dimethyl-benzenesulfonamide;
1-{4-[2-(2,6-Dichloro-phenylamino)-5-methyl-thiazolo[5,4-d]pyrimidin-7-ylamino]-phenyl}-ethanone;
N²-(2,6-Dichloro-phenyl)-N⁷-(4-methanesulfonyl-phenyl)-thiazolo[5,4-d]pyrimidine-2,7-diamine;
N²-(2,6-Dichloro-phenyl)-N⁷-[4-(4-methyl-piperazine-1-sulfonyl)-phenyl]-thiazolo[5,4-d]pyrimidine-2,7-diamine;
(racemic)-N²-(2,6-Dichloro-phenyl)-5-(2-isopropyl-pyrrolidin-1-yl)-N⁷-(4-trifluoromethyl-phenyl)-thiazolo[5,4-d]pyrimidine-2,7-diamine;
N²-(2,6-Dimethyl-phenyl)-5-methyl-N⁷-(4-trifluoromethanesulfonyl-phenyl)-thiazolo[5,4-d]pyrimidine-2,7-diamine;
N²-(2,6-Dimethyl-phenyl)-N⁷-[4-(morpholine-4-sulfonyl)-phenyl]-thiazolo[5,4-d]pyrimidine-2,7-diamine;
N²-(2,6-Dimethyl-phenyl)-5-methyl-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-[4-(propane-2-sulfonyl)-phenyl]-thiazolo[5,4-d]pyrimidine-2,7-diamine;
N²-(2,6-Dichloro-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-5-methyl-thiazolo[5,4-d]pyrimidine-2,7-diamine;
4-[2-(2,6-Dichloro-phenylamino)-5-methyl-thiazolo[5,4-d]pyrimidin-7-ylamino]-benzonitrile;
N²-(2,6-Dimethyl-phenyl)-N⁷-(3-fluoro-4-methanesulfonyl-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzenesulfonamide;
N²-(2,6-Dimethyl-phenyl)-N⁷-(4-trifluoromethanesulfonyl-phenyl)-thiazolo[5,4-d]pyrimidine-2,7-diamine;
N²-(2,6-Dichloro-phenyl)-N⁷-(3-morpholin-4-yl-propyl)-N⁷-(4-trifluoromethyl-phenyl)-thiazolo[5,4-d]pyrimidine-2,5,7-triamine;
N²-(2,6-Dichloro-phenyl)-N⁵-isopropyl-N⁷-(4-trifluoromethyl-phenyl)-thiazolo[5,4-d]pyrimidine-2,5,7-triamine;
N²-(2,6-Dimethyl-phenyl)-5-methyl-N⁷-[4-(propane-2-sulfonyl)-phenyl]-thiazolo[5,4-d]pyrimidine-2,7-diamine;
N²-(2,6-Dichloro-phenyl)-N⁷-(4-isopropylsulfanyl-phenyl)-5-methyl-thiazolo[5,4-d]pyrimidine-2,7-diamine;
N²-(2,6-Dimethyl-phenyl)-N⁷-[4-(pyrrolidine-1-sulfonyl)-phenyl]-thiazolo[5,4-d]pyrimidine-2,7-diamine;
(racemic)-N²-(2,6-Dichloro-phenyl)-5-(2-methyl-pyrrolidin-1-yl)-N⁷-(4-trifluoromethyl-phenyl)-thiazolo[5,4-d]pyrimidine-2,7-diamine;
N²-(2,6-Dimethyl-phenyl)-N⁷-(4-isopropylsulfanyl-phenyl)-5-methyl-thiazolo[5,4-d]pyrimidine-2,7-diamine;
N²-(2,6-Dichloro-phenyl)-5-methyl-N⁷-(1,4,4-trimethyl-1,2,3,4-tetrahydro-quinolin-7-yl)-thiazolo[5,4-d]pyrimidine-2,7-diamine;
N²-(2,6-Dimethyl-phenyl)-N⁷-(3-fluoro-4-trifluoromethyl-phenyl)-thiazolo[5,4-d]pyrimidine-2,7-diamine;
N²-(2-Chloro-phenyl)-N⁷-[4-(pyrrolidine-1-sulfonyl)-phenyl]-thiazolo[5,4-d]pyrimidine-2,7-diamine;
N²-(2,6-Dimethyl-phenyl)-N⁷-[4-(4-methyl-piperazine-1-sulfonyl)-phenyl]-thiazolo[5,4-d]pyrimidine-2,7-diamine;
N²-(2,6-Dichloro-phenyl)-5-methyl-N⁷-(4-trifluoromethoxy-phenyl)-thiazolo[5,4-d]pyrimidine-2,7-diamine;
N²-(2,6-Dimethyl-phenyl)-N⁷-(4-isopropylsulfanyl-phenyl)-thiazolo[5,4-d]pyrimidine-2,7-diamine;
4-[2-(2-Chloro-phenylamino)-thiazolo[5,4-d]pyrimidin-7-ylamino]-N,N-dimethyl-benzenesulfonamide;
N²-(2,6-Dimethyl-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-thiazolo[5,4-d]pyrimidine-2,7-diamine;
N⁷-(4-Methanesulfonyl-phenyl)-N²-o-tolyl-thiazolo[5,4-d]pyrimidine-2,7-diamine;
(racemic)-N²-(2,6-Dichloro-phenyl)-N⁷-(4-methanesulfonyl-phenyl)-5-(2-methyl-pyrrolidin-1-yl)-thiazolo[5,4-d]pyrimidine-2,7-diamine;
(racemic)-N⁷-(3-Chloro-4-trifluoromethyl-phenyl)-N²-(2,6-dichloro-phenyl)-5-(2-isopropyl-pyrrolidin-1-yl)-thiazolo[5,4-d]pyrimidine-2,7-diamine;
N⁷-(6-Chloro-pyridin-3-yl)-N²-(2,6-dimethyl-phenyl)-thiazolo[5,4-d]pyrimidine-2,7-diamine;
N²-(2,6-Dimethyl-phenyl)-N⁷-(4-methylsulfanyl-phenyl)-thiazolo[5,4-d]pyrimidine-2,7-diamine;
N²-(2,6-Dimethyl-phenyl)-N⁷-(3-fluoro-4-trifluoromethyl-phenyl)-5-methyl-thiazolo[5,4-d]pyrimidine-2,7-diamine;
N²-(2,6-Dimethyl-phenyl)-N⁷-[4-(propane-2-sulfonyl)-phenyl]-thiazolo[5,4-d]pyrimidine-2,7-diamine;
N⁷-(4-Bromo-phenyl)-N²-(2,6-dimethyl-phenyl)-thiazolo[5,4-d]pyrimidine-2,7-diamine;
N⁷-(3-Chloro-4-methylsulfanyl-phenyl)-N²-(2,6-dichloro-phenyl)-thiazolo[5,4-d]pyrimidine-2,7-diamine;
N²-(2,6-Dimethyl-phenyl)-N⁷-(4-isopropyl-phenyl)-5-methyl-thiazolo[5,4-d]pyrimidine-2,7-diamine;
N-(2-Chloro-phenyl)-N⁷-(4-methanesulfonyl-phenyl)-thiazolo[5,4-d]pyrimidine-2,7-diamine;
4-[2-(2,6-Dichloro-phenylamino)-5-methylsulfanyl-thiazolo[5,4-d]pyrimidin-7-ylamino]-N,N-dimethyl-benzenesulfonamide;
1-{4-[2-(2,6-Dimethyl-phenylamino)-thiazolo[5,4-d]pyrimidin-7-ylamino]-phenyl}-ethanone;
N²-(2,6-Dichloro-phenyl)-N⁷-(4-methanesulfonyl-phenyl)-5-piperidin-1-yl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-pyrrolidin-1-yl)-thiazolo[5,4-d]pyrimidine-2,7-diamine;
N⁷-(3-Chloro-4-trifluoromethylsulfanyl-phenyl)-N²-(2,6-dimethyl-phenyl)-5-methyl-thiazolo[5,4-d]pyrimidine-2,7-diamine;
N⁷-(4-Chloro-phenyl)-N²-(2,6-dimethyl-phenyl)-thiazolo[5,4-d]pyrimidine-2,7-diamine;
N²-(2,6-Dimethyl-phenyl)-N⁷-(3-fluoro-4-methyl-phenyl)-5-methyl-thiazolo[5,4-d]pyrimidine-2,7-diamine;
N²-(2,6-Dichloro-phenyl)-N⁷-[4-(piperazine-1-sulfonyl)-phenyl]-thiazolo[5,4-d]pyrimidine-2,7-diamine;
N⁷-(3-Chloro-4-trifluoromethylsulfanyl-phenyl)-N²-(2,6-dichloro-phenyl)-5-methyl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-piperidin-1-yl)-thiazolo[5,4-d]pyrimidine-2,7-diamine;
N²-(2,6-Dimethyl-phenyl)-N⁷-(4-iodo-phenyl)-thiazolo[5,4-d]pyrimidine-2,7-diamine;
N²-(2,6-Dimethyl-phenyl)-5-methyl-N⁷-p-tolyl-thiazolo[5,4-d]pyrimidine-2,7-diamine;
N²-(2,6-Dichloro-phenyl)-5-methyl-N⁷-(1-methyl-1,2,3,4-tetrahydro-quinolin-7-yl)-thiazolo[5,4-d]pyrimidine-2,7-diamine;
(racemic)-1-{4-[2-(2,6-Dimethyl-phenylamino)-thiazolo[5,4-d]pyrimidin-7-ylamino]-phenyl}-ethanol;
N²-(2,6-Dimethyl-phenyl)-5-methyl-N⁷-phenyl-thiazolo[5,4-d]pyrimidine-2,7-diamine;
2-Chloro-4-[2-(2,6-dimethyl-phenylamino)-thiazolo[5,4-d]pyrimidin-7-ylamino]-benzonitrile;
(racemic)-N²-(2,6-Dimethyl-phenyl)-N⁷-(4-methanesulfinyl-phenyl)-5-methyl-thiazolo[5,4-d]pyrimidine-2,7-diamine;
N²-(2,6-Dimethyl-phenyl)-N⁷-(4-fluoro-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzamide;
(racemic)-{4-[2-(2,6-Dichloro-phenylamino)-7-(4-trifluoromethyl-phenylamino)-thiazolo[5,4-d]pyrimidin-5-yl]-morpholin-2-yl}-methanol;
N²-(2,6-Dimethyl-phenyl)-N⁷-phenyl-thiazolo[5,4-d]pyrimidine-2,7-diamine;
N²-(2,6-Dimethyl-phenyl)-N⁷-(3-fluoro-4-methyl-phenyl)-thiazolo[5,4-d]pyrimidine-2,7-diamine;
4-[2-(2,6-Dimethyl-phenylamino)-thiazolo[5,4-d]pyrimidin-7-ylamino]-2-trifluoromethyl-benzonitrile;
N⁷-(2,3-Dihydro-benzo[1,4]dioxin-6-yl)-N²-(2,6-dimethyl-phenyl)-thiazolo[5,4-d]pyrimidine-2,7-diamine;
N²-(2,6-Dimethyl-phenyl)-N⁷-[4-(piperazine-1-sulfonyl)-phenyl]-thiazolo[5,4-d]pyrimidine-2,7-diamine;
N-{4-[2-(2,6-Dichloro-phenylamino)-5-methyl-thiazolo[5,4-d]pyrimidin-7-ylamino]-phenyl}-N-methyl-methanesulfonamide;
N²-(2,6-Dichloro-phenyl)-N⁵-[3-(4-methyl-piperazin-1-yl)-propyl]-N⁷-(4-trifluoromethyl-phenyl)-thiazolo[5,4-d]pyrimidine-2,5,7-diamine;
(racemic)-N²-(2,6-Dimethyl-phenyl)-N⁷-[4-(tetrahydro-furan-3-yloxy)-phenyl]-thiazolo[5,4-d]pyrimidine-2,7-diamine;
(racemic)-{4-[7-(3-Chloro-4-trifluoromethyl-phenylamino)-2-(2,6-dichloro-phenylamino)-thiazolo[5,4-d]pyrimidin-5-yl]-morpholin-2-yl}-methanol;
Cyclopentyl-{4-[2-(2,6-dichloro-phenylamino)-5-methyl-thiazolo[5,4-d]pyrimidin-7-ylamino]-phenyl}-methanone;
4-[2-(2,6-Dichloro-phenylamino)-5-methyl-thiazolo[5,4-d]pyrimidin-7-ylamino]-N,N-dimethyl-benzamide;
2-[2-(2,6-Dimethyl-phenylamino)-thiazolo[5,4-d]pyrimidin-7-ylamino]-5-methyl-phenol;
N²-(2,6-Dimethyl-phenyl)-N⁷-(2-methyl-4-trifluoromethyl-phenyl)-thiazolo[5,4-d]pyrimidine-2,7-diamine;
5-[2-(2,6-Dimethyl-phenylamino)-thiazolo[5,4-d]pyrimidin-7-ylamino]-2-methyl-phenol;
N-{4-[2-(2,6-Dimethyl-phenylamino)-thiazolo[5,4-d]pyrimidin-7-ylamino]-phenyl}-N-methyl-methanesulfonamide;
N⁷-(3-Chloro-4-trifluoromethyl-phenyl)-N²-(2,6-dichloro-phenyl)-5-piperazin-1-yl-thiazolo[5,4-d]pyrimidine-2,7-diamine;
N²-(2,6-Dichloro-phenyl)-5-piperazin-1-yl-N⁷-(4-trifluoromethyl-phenyl)-thiazolo[5,4-d]pyrimidine-2,7-diamine;
N-{4-[2-(2,6-Dimethyl-phenylamino)-5-methyl-thiazolo[5,4-d]pyrimidin-7-ylamino]-phenyl}-N-methyl-methanesulfonamide;
N²-(2,6-Dimethyl-phenyl)-N⁷-(3,4-dimethyl-phenyl)-thiazolo[5,4-d]pyrimidine-2,7-diamine;
N²-(2,6-Dichloro-phenyl)-5-methyl-N⁷-pyridin-3-yl-thiazolo[5,4-d]pyrimidine-2,7-diamine;
N⁷-(2-Chloro-4-trifluoromethyl-phenyl)-N²-(2,6-dimethyl-phenyl)-thiazolo[5,4-d]pyrimidine-2,7-diamine;
N²-(2,6-Dimethyl-phenyl)-N⁷-(4-methoxy-3-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzamide;
N²-(2,6-Dichloro-phenyl)-5-methyl-N⁷-phenyl-thiazolo[5,4-d]pyrimidine-2,7-diamine;
N²-(2,6-Dimethyl-phenyl)-5-methyl-N⁷-pyridin-3-yl-thiazolo[5,4-d]pyrimidine-2,7-diamine;
4-[2-(2,6-Dimethyl-phenylamino)-thiazolo[5,4-d]pyrimidin-7-ylamino]-benzoic acid;
N-{4-[2-(2,6-Dichloro-phenylamino)-5-methyl-thiazolo[5,4-d]pyrimidin-7-ylamino]-phenyl}-dimethanesulfonamide;
N-{4-[2-(2,6-Dimethyl-phenylamino)-5-methyl-thiazolo[5,4-d]pyrimidin-7-ylamino]-phenyl}-methanesulfonamide;
N-{4-[2-(2,6-Dimethyl-phenylamino)-thiazolo[5,4-d]pyrimidin-7-ylamino]-phenyl}-methanesulfonamide;
N²-(2,6-Dimethyl-phenyl)-N⁷-(5-trifluoromethyl-pyridin-2-yl)-thiazolo[5,4-d]pyrimidine-2,7-diamine;
(racemic)-N²-(2,6-Dichloro-phenyl)-5-(2-isopropyl-pyrrolidin-1-yl)-N⁷-(4-methanesulfonyl-phenyl)-thiazolo[5,4-d]pyrimidine-2,7-diamine;
N²-(2,6-Dichloro-phenyl)-N⁷-(4-methanesulfonyl-phenyl)-5-morpholin-4-yl-thiazolo[5,4-d]pyrimidine-2,7-diamine;
(racemic)-N²-(2,6-Dichloro-phenyl)-N⁷-(4-methanesulfonyl-phenyl)-5-(2-methyl-piperidin-1-yl)-thiazolo[5,4-d]pyrimidine-2,7-diamine;
N²-(2,6-Dichloro-phenyl)-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁵-(2-methylamino-ethyl)-N⁷-(4-trifluoromethyl-phenyl)-thiazolo[5,4-d]pyrimidine-2,5,7-triamine;
N²-(2,6-Dichloro-phenyl)-N⁵-(2-dimethylamino-ethyl)-N⁵-methyl-N⁷-(4-trifluoromethyl-phenyl)-thiazolo[5,4-d]pyrimidine-2,5,7-triamine;
(3R)-N²-(2,6-Dichloro-phenyl)-5-(3-methylamino-pyrrolidin-1-yl)-N⁷-(4-trifluoromethyl-phenyl)-thiazolo[5,4-d]pyrimidine-2,7-diamine;
N⁵-Cyclopropylmethyl-N²-(2,6-dichloro-phenyl)-N⁷-(4-methanesulfonyl-phenyl)-thiazolo[5,4-d]pyrimidine-2,5,7-triamine;
N²-(2,6-Dichloro-phenyl)-5-methyl-N⁷-(6-methylsulfanyl-pyridin-3-yl)-thiazolo[5,4-d]pyrimidine-2,7-diamine;
(racemic)-2-[2-(2,6-Dichloro-phenylamino)-7-(4-trifluoromethyl-phenylamino)-thiazolo[5,4-d]pyrimidin-5-ylamino]-propan-1-ol;
N²-(2,6-Dichloro-phenyl)-5-(4-methyl-piperazin-1-yl)-N⁷-(4-trifluoromethyl-phenyl)-thiazolo[5,4-d]pyrimidine-2,7-diamine;
N²-(2,6-Dichloro-phenyl)-N⁵,N⁵-diethyl-N⁷-(4-trifluoromethyl-phenyl)-thiazolo[5,4-d]pyrimidine-2,5,7-triamine;
5-Butoxy-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-(4-methyl-piperidin-1-yl)-N²-(4-trifluoromethyl-phenyl)-thiazolo[5,4-d]pyrimidine-2,7-diamine;
(racemic)-N²-(2,6-Dichloro-phenyl)-5-(2-methyl-piperidin-1-yl)-N⁷-(4-trifluoromethyl-phenyl)-thiazolo[5,4-d]pyrimidine-2,7-diamine;
(3S)-N²-(2,6-Dichloro-phenyl)-5-(3-methyl-morpholin-4-yl)-N⁷-(4-trifluoromethyl-phenyl)-thiazolo[5,4-d]pyrimidine-2,7-diamine;
(2S)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
(2R)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
5-Methyl-N²-(2-methylsulfanyl-phenyl)-N⁷-(4-trifluoromethyl-phenyl)-thiazolo[5,4-d]pyrimidine-2,7-diamine;
N²-(2-Methylsulfanyl-phenyl)-N⁷-(4-trifluoromethyl-phenyl)-thiazolo[5,4-d]pyrimidine-2,7-diamine;
N²-(2-Methanesulfonyl-phenyl)-5-methyl-N⁷-(4-trifluoromethylphenyl)-thiazolo[5,4-d]pyrimidine-2,7-diamine;
N²-(2-Methanesulfonyl-phenyl)-N⁷-(4-methanesulfonyl-phenyl)-thiazolo[5,4-d]pyrimidine-2,7-diamine;
N²-(2-Methanesulfonyl-phenyl)-N⁷-(6-trifluoromethyl-pyridin-3-yl)-thiazolo[5,4-d]pyrimidine-2,7-diamine;
N²-(2-Methanesulfonyl-phenyl)-N⁷-(4-trifluoromethanesulfonyl-phenyl)-thiazolo[5,4-d]pyrimidine-2,7-diamine;
N²-(2-Methanesulfonyl-phenyl)-N⁷-(4-trifluoromethyl-phenyl)-thiazolo[5,4-d]pyrimidine-2,7-diamine;
N²-(2-Chloro-phenyl)-5-methyl-N⁷-(4-trifluoromethyl-phenyl)-thiazolo[5,4-d]pyrimidine-2,7-diamine;
N²-(2-Chloro-phenyl)-N⁷-(4-trifluoromethanesulfonyl-phenyl)-thiazolo[5,4-d]pyrimidine-2,7-diamine;
N²-(2,6-Dichloro-phenyl)-N⁷-phenyl-thiazolo[5,4-d]pyrimidine-2,7-diamine;
N²-Benzo[1,2,5]thiadiazol-4-yl-5-methyl-N⁷-(4-trifluoromethyl-phenyl)-thiazolo[5,4-d]pyrimidine-2,7-diamine;
5-Methyl-N²-(2-nitro-phenyl)-N⁷-(4-trifluoromethyl-phenyl)-thiazolo[5,4-d]pyrimidine-2,7-diamine;
3-[7-(3-Chloro-4-trifluoromethyl-phenylamino)-thiazolo[5,4-d]pyrimidin-2-ylamino]-4-methyl-thiophene-2-carboxylic acid methyl ester;
N²-(3,5-Dimethyl-isoxazol-4-yl)-5-methyl-N⁷-(4-trifluoromethyl-phenyl)thiazolo[5,4-d]pyrimidine-2,7-diamine;
N⁷-(4-tert-Butyl-phenyl)-N²-(3,5-dimethyl-isoxazol-4-yl)-5-methyl-thiazolo[5,4-d]pyrimidine-2,7-diamine;
N²-(3-Methyl-pyridin-2-yl)-N⁷-[4-(pyrrolidine-1-sulfonyl)-phenyl]-thiazolo[5,4-d]pyrimidine-2,7-diamine;
5-Methyl-N²-(3-methyl-pyridin-2-yl)-N⁷-(4-trifluoromethyl-phenyl)-thiazolo[5,4-d]pyrimidine-2,7-diamine;
N,N-Dimethyl-4-[5-methyl-2-(3-methyl-pyridin-2-ylamino)-thiazolo[5,4-d]pyrimidin-7-ylamino]-benzenesulfonamide;
N²-(3-Methyl-pyridin-2-yl)-N⁷-(4-trifluoromethyl-phenyl)-thiazolo[5,4-d]pyrimidine-2,7-diamine;
N²-(3,5-Dichloro-pyridin-4-yl)-N⁷-[4-(pyrrolidine-1-sulfonyl)-phenyl]-thiazolo[5,4-d]pyrimidine-2,7-diamine;
N²-(2,6-Dichloro-phenyl)-N⁷-(3-fluoro-4-trifluoromethyl-phenyl)-thiazolo[5,4-d]pyrimidine-2,7-diamine;
N²-(2-Chlorophenyl)-N⁷-[4-(morpholin-4-ylsulfonyl)phenyl][1,3]thiazolo[5,4-d]pyrimidine-2,7-diamine;
N²-(2-Methylphenyl)-N⁷-[4-(morpholin-4-ylsulfonyl)phenyl][1,3]thiazolo[5,4-d]pyrimidine-2,7-diamine;
N²-(2-Methylphenyl)-N⁷-[6-(trifluoromethyl)pyridin-3-yl][1,3]thiazolo[5,4-d]pyrimidine-2,7-diamine;
N²-[2-(Trifluoromethyl)phenyl]-N⁷-[6-(trifluoromethyl)pyridin-3-yl][1,3]thiazolo[5,4-d]pyrimidine-2,7-diamine;
N²-(2-Chlorophenyl)-N⁷-[6-(trifluoromethyl)pyridin-3-yl][1,3]thiazolo[5,4-d]pyrimidine-2,7-diamine;
N²-(3,5-Dimethylisoxazol-4-yl)-N⁷-[4-(morpholin-4-ylsulfonyl)phenyl][1,3]thiazolo[5,4-d]pyrimidine-2,7-diamine;
Methyl 2-[4-({2-[(3,5-dimethylisoxazol-4-yl)amino][1,3]thiazolo[5,4-d]pyrimidin-7-yl}amino)phenyl]-2-methylpropanoate;
2-[4-({2-[(3,5-Dimethylisoxazol-4-yl)amino][1,3]thiazolo[5,4-d]pyrimidin-7-yl}amino)phenyl]-2-methylpropanenitrile;
N²-(3,5-Dimethylisoxazol-4-yl)-N⁷-[4-(methylsulfonyl)phenyl][1,3]thiazolo[5,4-d]pyrimidine-2,7-diamine;
N²-[2-(Trifluoromethyl)phenyl]-N⁷-[4-(trifluoromethyl)phenyl][1,3]thiazolo[5,4-d]pyrimidine-2,7-diamine;
N⁷-[4-(Methylsulfonyl)phenyl]-N²-[2-(trifluoromethyl)phenyl][1,3]thiazolo[5,4-d]pyrimidine-2,7-diamine;
4-({2-[(2,6-Dichlorophenyl)amino][1,3]thiazolo[5,4-d]pyrimidin-7-yl}amino)benzene-1,2-diol;
2-[4-({2-[(2,6-Dichlorophenyl)amino][1,3]thiazolo[5,4-d]pyrimidin-7-yl}amino)phenyl]-2-methylpropanenitrile;
Methyl 2-[4-({2-[(2,6-dichlorophenyl)amino][1,3]thiazolo[5,4-d]pyrimidin-7-yl}amino)phenyl]-2-methylpropanoate;
2-[4-({2-[(2,6-Dichlorophenyl)amino][1,3]thiazolo[5,4-d]pyrimidin-7-yl}amino)phenyl]-2-methylpropanoic acid;
1-Methylethyl 2-[4-({2-[(2,6-dichlorophenyl)amino][1,3]thiazolo[5,4-d]pyrimidin-7-yl}amino)phenyl]-2-methylpropanoate;
N²-Cyclohexyl-N⁷-[4-(trifluoromethyl)phenyl][1,3]thiazolo[5,4-d]pyrimidine-2,7-diamine;
N²-Cyclohexyl-N⁷-[6-(trifluoromethyl)pyridin-3-yl][1,3]thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzonitrile;
3,5-Dichloro-4-[7-(4-trifluoromethyl-phenylamino)-thiazolo[5,4-d]pyrimidin-2-ylamino]-benzamide;
3,5-Dichloro-4-[7-(4-methanesulfonyl-phenylamino)-thiazolo[5,4-d]pyrimidin-2-ylamino]-benzonitrile;
3,5-Dichloro-4-[7-(4-methanesulfonyl-phenylamino)-thiazolo[5,4-d]pyrimidin-2-ylamino]-benzamide;
N²-(2,6-Dichloro-4-morpholin-4-ylmethyl-phenyl)-N⁷-(4-trifluoromethyl-phenyl)-thiazolo[5,4-d]pyrimidine-2,7-diamine;
N²-(4-Azetidin-1-ylmethyl-2,6-dichloro-phenyl)-N⁷-(4-trifluoromethyl-phenyl)-thiazolo[5,4-d]pyrimidine-2,7-diamine;
N²-(4-Aminomethyl-2,6-dichloro-phenyl)-N⁷-(4-trifluoromethyl-phenyl)-thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzoic acid methyl ester;
{3,5-Dichloro-4-[7-(4-trifluoromethyl-phenylamino)-thiazolo[5,4-d]pyrimidin-2-ylamino]-phenyl}-methanol;
3,5-Dichloro-4-[7-(4-trifluoromethyl-phenylamino)-thiazolo[5,4-d]pyrimidin-2-ylamino]-benzoic acid;
N⁷-(4-tert-Butyl-phenyl)-N²-(2,6-dimethyl-phenyl)-5-methyl-thiazolo[5,4-d]pyrimidine-2,7-diamine;
N²-(2,6-Dimethyl-phenyl)-5-methyl-N⁷-(4-trifluoromethyl-phenyl)-thiazolo[5,4-d]pyrimidine-2,7-diamine;
N²-(2,6-Dimethyl-phenyl)-5-methyl-N⁷-(6-trifluoromethyl-pyridin-3-yl)-thiazolo[5,4-d]pyrimidine-2,7-diamine;
N⁷-(3-Chloro-4-trifluoromethyl-phenyl)-N⁷ (2,6-dimethyl-phenyl)-5-methyl-thiazolo[5,4-d]pyrimidine-2,7-diamine;
N⁷-(4-tert-Butyl-cyclohexyl)-N²-(2,6-dimethyl-phenyl)-thiazolo[5,4-d]pyrimidine-2,7-diamine;
N⁷-(4-tert-Butyl-cyclohexyl)-N²-(2,6-dimethyl-phenyl)-5-methyl-thiazolo[5,4-d]pyrimidine-2,7-diamine;
(R)-1-[2-(2,6-Dichloro-phenylamino)-7-(4-trifluoromethyl-phenylamino)-thiazolo[5,4-d]pyrimidin-5-ylamino]-propan-2-ol;
1-[2-(2,6-Dichloro-phenylamino)-7-(4-trifluoromethyl-phenylamino)-thiazolo[5,4-d]pyrimidin-5-ylamino]-2-methyl-propan-2-ol;
(racemic)-{1-[2-(2,6-Dichloro-phenylamino)-7-(4-trifluoromethyl-phenylamino)-thiazolo[5,4-d]pyrimidin-5-yl]-pyrrolidin-2-yl}-methanol;
N²-(2,6-Dichloro-phenyl)-N⁵-methyl-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁷-(6-methanesulfonyl-pyridin-3-yl)-thiazolo[5,4-d]pyrimidine-2,7-diamine;
2-{4-[2-(2,6-Dichloro-phenylamino)-thiazolo[5,4-d]pyrimidin-7-ylamino]-phenyl}-isobutyramide;
(racemic)-1-[2-(2,6-Dichloro-phenylamino)-7-(4-trifluoromethyl-phenylamino)-thiazolo[5,4-d]pyrimidin-5-ylamino]-propan-2-ol;
(racemic)-3-[2-(2,6-Dichloro-phenylamino)-7-(4-trifluoromethyl-phenylamino)-thiazolo[5,4-d]pyrimidin-5-ylamino]-propane-1,2-diol; and
N²-(2,6-Dichloro-phenyl)-N⁵-(2-pyrrolidin-1-yl-ethyl)-N⁷-(4-trifluoromethyl-phenyl)-thiazolo[5,4-d]pyrimidine-2,5,7-triamine;
and pharmaceutically acceptable salts thereof.

36. A pharmaceutical composition for treating a disease, disorder, or medical condition mediated by TRPV1 activity, comprising:
(a) an effective amount of at least one active agent selected from compounds of Formula (I) and pharmaceutically acceptable salts, pharmaceutically acceptable prodrugs, and pharmaceutically active metabolites of said compounds of Formula (I): 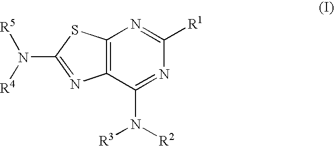
wherein:
R¹ is —H; —NR^{a}R^{b}; a —C₁₋₆alkyl, —OC₁₋₆alkyl, —S—C₁₋₆alkyl, or —SO₂—C₁₋₆alkyl group unsubstituted or substituted with an —OH, —OC₁₋₄alkyl, —NR^{e}R^{f}, or halo substituent;
or a monocyclic cycloalkyl or phenyl group unsubstituted or substituted with a —C₁₋₆alkyl, —OH, —OC₁₋₄alkyl, —NR^{e}R^{f}, or halo substituent;
where R^{a} and R^{b} are each independently —H; —C₁₋₆alkyl; a —C₂₋₄alkyl group substituted with one or two —OH, —OC₁₋₄alkyl, —NR^{c}R^{d}, or halo substituents; or a saturated monocyclic cycloalkyl, —C₁alkyl-(saturated monocyclic cycloalkyl), saturated monocyclic heterocycloalkyl, —C₁alkyl-(saturated monocyclic heterocycloalkyl), phenyl, or benzyl group unsubstituted or substituted with one, two, or three moieties independently selected from the group consisting of —C₁₋₆alkyl, —OH, —OC₁₋₄alkyl, —NR^{p}R^{q}, and halo substituents; or
R^{a} and R^{b} taken together with the nitrogen of attachment in —NR^{a}R^{b} form a saturated monocyclic heterocycloalkyl group unsubstituted or substituted with one, two, or three moieties independently selected from the group consisting of —C₁₋₆alkyl, —C₁₋₂alkyl-OH, —C₁₋₂alkyl-OC₁₋₂alkyl, —OH, —OC₁₋₄alkyl, —NR^{p}R^{q}, halo, —CO₂H, and benzyl substituents;
where R^{c} and R^{d} are each independently —H or —C₁₋₆alkyl; or R^{c} and R^{d} taken together with the nitrogen of attachment in —NR^{c}R^{d} form a saturated monocyclic heterocycloalkyl unsubstituted or substituted with methyl; and
R^{p} and R^{q} are each independently —H or —C₁₋₆alkyl; or R^{p} and R^{q} taken together with the nitrogen of attachment in —NR^{p}R^{q} form a saturated monocyclic heterocycloalkyl unsubstituted or substituted with methyl; and
R^{e} and R^{f} are each independently —H or —C₁₋₆alkyl; or R^{e} and R^{f} taken together with their nitrogen of attachment in —NR^{e}R^{f} form a saturated monocyclic heterocycloalkyl unsubstituted or substituted with methyl;
R² is —H or —C₁₋₆alkyl;
R³ is a monocyclic cycloalkyl, phenyl, benzyl, phenethyl, indanyl, quinolinyl, monocyclic five-membered heteroaryl, monocyclic six-membered heteroaryl, or —C₁alkyl-(monocyclic heteroaryl) group unsubstituted or substituted with one, two, or three R^{g} substituents;
where each R^{g} substituent is independently —C₁₋₆alkyl, —OH, —OC₁₋₆alkyl, —O-(saturated monocyclic heterocycloalkyl), phenoxy, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH; or
two adjacent R^{g} substituents taken together form —OC₁₋₂alkylO—, —C₂₋₆alkylO—, or —C₂₋₆alkylN(R^{h})—;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl unsubstituted or substituted with methyl; and
each R^{j} is independently —H, —C₁₋₆alkyl, or —CF₃; or both R^{j} substituents taken together with the carbon to which they are attached form a monocyclic cycloalkyl ring;
R⁴ is —H or —C₁₋₆alkyl; and
R⁵ is a phenyl, monocyclic five-membered heteroaryl, or monocyclic six-membered heteroaryl group unsubstituted or substituted with one, two, or three R^{k} substituents;
where each R^{k} substituent is independently —C₁₋₆alkyl unsubstituted or substituted with one or two —OH groups, —C₁₋₂alkyl-N(R^{l})R^{m}, —OH, —OC₁₋₆alkyl, phenyl, phenoxy, —CN, —NO₂, —N(R^{l})R^{m}, —C(O)N(R^{l})R^{m}, —N(R^{l})C(O)R^{m}, —N(R^{l})SO₂C₁₋₆alkyl, —N(R^{l})SO₂CF₃, —C(O)C₁₋₆alkyl, —S(O)₀₋₂—C₁₋₆alkyl, —SO₂CF₃, —SO₂N(R^{l})R^{m}, —SCF₃, halo, —CF₃, —OCF₃, —CO₂H, or —CO₂C₁₋₆alkyl; or
two adjacent R^{k} substituents taken together form —OC₁₋₂alkylO— or ═N—S—N═;
where R^{l}, and R^{m} are each independently —H or —C₁₋₆alkyl; or R^{l} and R^{m} taken together with their nitrogen of attachment in —NR^{l}R^{m} form a saturated monocyclic heterocycloalkyl unsubstituted or substituted with methyl;
and (b) a pharmaceutically acceptable excipient.

37. A pharmaceutical composition according to claim 36, wherein said active agent is selected from the group consisting of:
N²-(2,6-Dichloro-phenyl)-N⁷-(4-trifluoromethyl-phenyl)-thiazolo[5,4-d]pyrimidine-2,7-diamine;
N²-(2,6-Dichloro-phenyl)-N⁷-(6-trifluoromethyl-pyridin-3-yl)-thiazolo[5,4-d]pyrimidine-2,7-diamine;
N⁷-(4-tert-Butyl-phenyl)-N⁷-(2,6-dichloro-phenyl)-thiazolo[5,4-d]pyrimidine-2,7-diamine;
N⁸-(3-Chloro-4-trifluoromethyl-phenyl)-N²-(2,6-dichloro-phenyl)-thiazolo[5,4-d]pyrimidine-2,7-diamine;
N²-(2,6-Dichloro-phenyl)-5-methyl-N⁷-(4-trifluoromethyl-phenyl)-thiazolo[5,4-d]pyrimidine-2,7-diamine;
N²-(2,6-Dichloro-phenyl)-5-methyl-N⁷-(6-trifluoromethyl-pyridin-3-yl)-thiazolo[5,4-d]pyrimidine-2,7-diamine;
N⁷-(4-tert-Butyl-phenyl)-N²-(2,6-dichloro-phenyl)-5-methyl-thiazolo[5,4-d]pyrimidine-2,7-diamine;
N⁷-(4-tert-Butyl-cyclohexyl)-N²-(2,6-dichloro-phenyl)-5-methyl-thiazolo[5,4-d]pyrimidine-2,7-diamine;
N²-(2,6-Dichloro-phenyl)-5,N⁷-dimethyl-N⁷-(4-trifluoromethyl-phenyl)-thiazolo[5,4-d]pyrimidine-2,7-diamine;
N²-(2,6-Dimethyl-phenyl)-N⁷-(4-trifluoromethyl-phenyl)thiazolo[5,4,d]pyrimidine-2,7-diamine;
N²-(2,6-Dimethyl-phenyl)-N⁷-(6-trifluoromethyl-pyridin-3-yl)-thiazolo[5,4-d]pyrimidine-2,7-diamine;
N⁷-(4-tert-Butyl-phenyl)-N²-(2,6-dimethyl-phenyl)-thiazolo[5,4-d]pyrimidine-2,7-diamine;
N⁷-(3-Chloro-4-trifluoromethyl-phenyl)-N²-(2,6-dimethyl-phenyl)-thiazolo[5,4-d]pyrimidine-2,7-diamine;
N²-(2-Chloro-6-methyl-phenyl)-N⁷-(4-trifluoromethyl-phenyl)-thiazolo[5,4-d]pyrimidine-2,7-diamine;
N²-(2-Chloro-6-methyl-phenyl)-N⁷-(6-trifluoromethyl-pyridin-3-yl)-thiazolo[5,4-d]pyrimidine-2,7-diamine;
N²-(2-Chloro-phenyl)-N⁷-(4-trifluoromethyl-phenyl)-thiazolo[5,4-d]pyrimidine-2,7-diamine;
N²-o-Tolyl-N⁷-(4-trifluoromethyl-phenyl)-thiazolo[5,4-d]pyrimidine-2,7-diamine;
N²-(2-Chloro-6-trifluoromethyl-phenyl)-N⁷-(4-trifluoromethyl-phenyl)-thiazolo[5,4-d]pyrimidine-2,7-diamine;
N²-(2-Chloro-6-trifluoromethyl-phenyl)-N⁷-(6-trifluoromethyl-pyridin-3-yl)-thiazolo[5,4-d]pyrimidine-2,7-diamine;
N²-Phenyl-N⁷-(4-trifluoromethyl-phenyl)-thiazolo[5,4-d]pyrimidine-2,7-diamine;
N²-Phenyl-N⁷-(6-trifluoromethyl-pyridin-3-yl)-thiazolo[5,4-d]pyrimidine-2,7-diamine;
N²,N⁷-Bis-(4-trifluoromethyl-phenyl)-thiazolo[5,4-d]pyrimidine-2,7-diamine;
N²-(2,6-Dichloro-phenyl)-5,N²-dimethyl-N⁷-(4-trifluoromethyl-phenyl)-thiazolo[5,4-d]pyrimidine-2,7-diamine;
N²-(3,5-Dimethyl-isoxazol-4-yl)-N⁷-(6-trifluoromethyl-pyridin-3-yl)-thiazolo[5,4-d]pyrimidine-2,7-diamine;
N²-(3,5-Dimethyl-isoxazol-4-yl)-N⁷-(4-trifluoromethyl-phenyl)-thiazolo[5,4-d]pyrimidine-2,7-diamine;
5-Methyl-N²-(5-methyl-3-phenyl-isoxazol-4-yl)-N⁷-(6-trifluoromethyl-pyridin-3-yl)-thiazolo[5,4-d]pyrimidine-2,7-diamine;
5-Methyl-N²-(5-methyl-3-phenyl-isoxazol-4-yl)-N⁷-(4-trifluoromethyl-phenyl)-thiazolo[5,4-d]pyrimidine-2,7-diamine;
N²-(2,6-Dimethyl-phenyl)-N⁷-(4-trifluoromethoxy-phenyl)-thiazolo[5,4-d]pyrimidine-2,7-diamine;
5-[2-(2,6-Dimethyl-phenylamino)-thiazolo[5,4-d]pyrimidin-7-ylamino]-pyridine-2-carbonitrile;
N⁷-(3-Chloro-4-trifluoromethyl-phenyl)-N²-(2,6-dichloro-phenyl)-5-methyl-thiazolo[5,4-d]pyrimidine-2,7-diamine;
4-[2-(2,6-Dimethyl-phenylamino)-thiazolo[5,4-d]pyrimidin-7-ylamino]-benzonitrile;
2-(4-[2-(2,6-Dimethyl-phenylamino)-thiazolo[5,4-d]pyrimidin-7-ylamino]-phenyl)-2-methyl-propionitrile;
N²-(2,6-Dimethyl-phenyl)-N⁷-(4-methoxy-phenyl)-thiazolo[5,4-d]pyrimidine-2,7-diamine;
N⁷-(3,4-Dichloro-phenyl)-N²-(2,6-dimethyl-phenyl)-thiazolo[5,4-d]pyrimidine-2,7-diamine;
N²-(2,6-Dimethyl-phenyl)-N⁷-p-tolyl-thiazolo[5,4-d]pyrimidine-2,7-diamine;
N²-(2,6-Dimethyl-phenyl)-N⁷-(2-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzoic acid methyl ester;
4-{[2-(2,6-Dimethyl-phenylamino)-thiazolo[5,4-d]pyrimidin-7-ylamino]-methyl}-2-methoxy-phenol;
N⁷-(3,4-Dichloro-benzyl)-N²-(2,6-dimethyl-phenyl)-thiazolo[5,4-d]pyrimidine-2,7-diamine;
N²-(2,6-Dimethyl-phenyl)-N⁷-(4-trifluoromethylsulfanyl-phenyl)-thiazolo[5,4-d]pyrimidine-2,7-diamine;
N²-(2,6-Dimethyl-phenyl)-N⁷-indan-2-yl-thiazolo[5,4-d]pyrimidine-2,7-diamine;
N²-(2,6-Dimethyl-phenyl)-N⁷-(3-trifluoromethyl-phenyl)-thiazolo[5,4-d]pyrimidine-2,7-diamine;
N⁷-Benzyl-N²-(2,6-dimethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzenesulfonamide;
N²-(2,6-Dimethyl-phenyl)-N⁷-(4-ethyl-phenyl)-thiazolo[5,4-d]pyrimidine-2,7-diamine;
N²-(2,6-Dimethyl-phenyl)-N⁷-(4-isopropyl-phenyl)-thiazolo[5,4-d]pyrimidine-2,7-diamine;
N²-(2,6-Dimethyl-phenyl)-N⁷-(5-methyl-furan-2-ylmethyl)-thiazolo[5,4-d]pyrimidine-2,7-diamine;
4-Methyl-3-[7-(4-trifluoromethyl-phenylamino)-thiazolo[5,4-d]pyrimidin-2-ylamino]-thiophene-2-carboxylic acid methyl ester;
4-Methyl-3-[7-(6-trifluoromethyl-pyridin-3-ylamino)-thiazolo[5,4-d]pyrimidin-2-ylamino]-thiophene-2-carboxylic acid methyl ester;
N⁷-(3-Chloro-4-trifluoromethyl-phenyl)-N²-(3,5-dimethyl-isoxazol-4yl)thiazolo[5,4d]pyrimidine-2,7-diamine;
N⁷-(4-tert-Butyl-phenyl)-N²-(3,5-dimethyl-isoxazol-4-yl)-thiazolo[5,4-d]pyrimidine-2,7-diamine;
N²-(2,6-Dichloro-phenyl)-5-methylsulfanyl-N⁷-(4-trifluoromethyl-phenyl)-thiazolo[5,4-d]pyrimidine-2,7-diamine;
N²-(2,6-Dichloro-phenyl)-5-methanesulfonyl-N⁷-(4-trifluoromethyl-phenyl)-thiazolo[5,4-d]pyrimidine-2,7-diamine;
N²-(2,6-Dichloro-phenyl)-5-piperidin-1-yl-N⁷-(4-trifluoromethyl-phenyl)-thiazolo[5,4-d]pyrimidine-2,7-diamine;
N²-(2,6-Dichloro-phenyl)-5-methoxy-N⁷-(4-trifluoromethyl-phenyl)-thiazolo[5,4d]pyrimidine-2,7-diamine;
N²-(2,6-Dichloro-phenyl)-N⁵,N⁵-dimethyl-N⁷-(4-trifluoromethyl-phenyl)-thiazolo[5,4-d]pyrimidine-2,5,7-triamine;
5-Azepan-1-yl-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-pyrrolidin-1-yl-N⁷-(4-trifluoromethyl-phenyl)-thiazolo[5,4-d]pyrimidine-2,7-diamine;
5-Azetidin-1-yl-N²-(2,6-dichloro-phenyl)-N⁷-(4-trifluoromethyl-phenyl)-thiazolo[5,4-d]pyrimidine-2,7-diamine;
N²-(2,6-Bis-methanesulfonyl-phenyl)-5-methyl-N⁷-(4-trifluoromethyl-phenyl)-thiazolo[5,4-d]pyrimidine-2,7-diamine;
N²-(2,6-Dichloro-phenyl)-N⁵-(2-methoxy-ethyl)-N⁷-(4-trifluoromethyl-phenyl)-thiazolo[5,4-d]pyrimidine-2,5,7-triamine;
N⁵-Cyclopropylmethyl-N²-(2,6-dichloro-phenyl)-N⁷-(4-trifluoromethyl-phenyl)-thiazolo[5,4-d]pyrimidine-2,5,7-triamine;
N²-(2,6-Dichloro-phenyl)-N⁵-(2-methoxy-ethyl)-N⁵-methyl-N⁷-(4-trifluoromethyl-phenyl)-thiazolo[5,4-d]pyrimidine-2,5,7-triamine;
N²-(2,6-Dichloro-phenyl)-5-morpholin-4-yl-N⁷-(4-trifluoromethyl-phenyl)-thiazolo[5,4-d]pyrimidine-2,7-diamine;
N²-(2,6-Dichloro-phenyl)-N⁷-(5-trifluoromethyl-pyridin-2-yl)-thiazolo[5,4-d]pyrimidine-2,7-diamine;
N²-(2,6-Dichloro-phenyl)-5-methyl-N⁷-(5-trifluoromethyl-pyridin-2-yl)-thiazolo[5,4-d]pyrimidine-2,7-diamine;
N²-(2,6-Dichloro-phenyl)-5-phenoxy-N⁷-(4-trifluoromethyl-phenyl)-thiazolo[5,4-d]pyrimidine-2,7-diamine;
N²-(2,6-Dichloro-phenyl)-N⁷-phenyl-N⁷-(4-trifluoromethyl-phenyl)-thiazolo[5,4-d]pyrimidine-2,5,7-triamine;
N²-(2,6-Dichloro-phenyl)-5-(4-isopropyl-piperazin-1-yl)-N⁷-(4-trifluoromethyl-phenyl)-thiazolo[5,4-d]pyrimidine-2,7-diamine;
N²-(2,6-Dichloro-phenyl)-5-phenyl-N⁷-(4-trifluoromethyl-phenyl)-thiazolo[5,4-d]pyrimidine-2,7-diamine;
N²-(2,6-Dichloro-phenyl)-5-isopropyl-N⁷-(4-trifluoromethyl-phenyl)-thiazolo[5,4-d]pyrimidine-2,7-diamine; and
N²-(3,5-Dichloro-pyridin-4-yl)-N⁷-(4-trifluoromethyl-phenyl)-thiazolo[5,4-d]pyrimidine-2,7-diamine;
and pharmaceutically acceptable salts thereof.

38. A pharmaceutical composition according to claim 36, wherein said active agent is selected from the group consisting of:
N²-(2,6-Dichloro-phenyl)-5-methyl-N⁷-[4-(pyrrolidine-1-sulfonyl)-phenyl]-thiazolo[5,4-d]pyrimidine-2,7-diamine;
2-{4-[2-(2,6-Dichloro-phenylamino)-5-methyl-thiazolo[5,4-d]pyrimidin-7-ylamino]-phenyl}-propan-2-ol;
4-[2-(2,6-Dichloro-phenylamino)-thiazolo[5,4-d]pyrimidin-7-ylamino]-N,N-dimethyl-benzenesulfonamide;
N²-(2,6-Dichloro-phenyl)-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-(4-trifluoromethanesulfonyl-phenyl)-thiazolo[5,4-d]pyrimidine-2,7-diamine;
N²-(2,6-Dichloro-phenyl)-N⁷-(4-methanesulfonyl-phenyl)-5-methyl-thiazolo[5,4-d]pyrimidine-2,7-diamine;
N²-(2,6-Dichloro-phenyl)-N⁵-isobutyl-N⁷-(4-trifluoromethyl-phenyl)-thiazolo[5,4-d]pyrimidine-2,5,7-triamine;
N²-(2,6-Dichloro-phenyl)-N⁷-[4-(morpholine-4-sulfonyl)-phenyl]-thiazolo[5,4-d]pyrimidine-2,7-diamine;
4-[2-(2,6-Dichloro-phenylamino)-5-methyl-thiazolo[5,4-d]pyrimidin-7-ylamino]-N,N-dimethyl-benzenesulfonamide;
N²-(2,6-Dichloro-phenyl)-N⁷-(3-fluoro-4-methanesulfonyl-phenyl)-thiazolo[5,4-d]pyrimidine-2,7-diamine;
N⁷-[4-(Pyrrolidine-1-sulfonyl)-phenyl]-N²-o-tolyl-thiazolo[5,4-d]pyrimidine-2,7-diamine;
N²-(2,6-Dichloro-phenyl)-N⁷-(4-isopropyl-phenyl)-5-methyl-thiazolo[5,4-d]pyrimidine-2,7-diamine;
4-[2-(2,6-Dimethyl-phenylamino)-5-methyl-thiazolo[5,4-d]pyrimidin-7-ylamino]-N,N-dimethyl-benzenesulfonamide;
1-{4-[2-(2,6-Dichloro-phenylamino)-5-methyl-thiazolo[5,4-d]pyrimidin-7-ylamino]-phenyl}-ethanone;
N²-(2,6-Dichloro-phenyl)-N⁷-(4-methanesulfonyl-phenyl)-thiazolo[5,4-d]pyrimidine-2,7-diamine;
N²-(2,6-Dichloro-phenyl)-N⁷-[4-(4-methyl-piperazine-1-sulfonyl)-phenyl]-thiazolo[5,4-d]pyrimidine-2,7-diamine;
(racemic)-N²-(2,6-Dichloro-phenyl)-5-(2-isopropyl-pyrrolidin-1-yl)-N⁷-(4-trifluoromethyl-phenyl)-thiazolo[5,4-d]pyrimidine-2,7-diamine;
N²-(2,6-Dimethyl-phenyl)-5-methyl-N⁷-(4-trifluoromethanesulfonyl-phenyl)-thiazolo[5,4-d]pyrimidine-2,7-diamine;
N²-(2,6-Dimethyl-phenyl)-N⁷-[4-(morpholine-4-sulfonyl)-phenyl]-thiazolo[5,4-d]pyrimidine-2,7-diamine;
N²-(2,6-Dimethyl-phenyl)-5-methyl-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-[4-(propane-2-sulfonyl)-phenyl]-thiazolo[5,4-d]pyrimidine-2,7-diamine;
N²-(2,6-Dichloro-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-5-methyl-thiazolo[5,4-d]pyrimidine-2,7-diamine;
4-[2-(2,6-Dichloro-phenylamino)-5-methyl-thiazolo[5,4-d]pyrimidin-7-ylamino]-benzonitrile;
N²-(2,6-Dimethyl-phenyl)-N⁷-(3-fluoro-4-methanesulfonyl-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzenesulfonamide;
N²-(2,6-Dimethyl-phenyl)-N⁷-(4-trifluoromethanesulfonyl-phenyl)-thiazolo[5,4-d]pyrimidine-2,7-diamine;
N²-(2,6-Dichloro-phenyl)-N⁵-(3-morpholin-4-yl-propyl)-N⁷-(4-trifluoromethyl-phenyl)-thiazolo[5,4-d]pyrimidine-2,5,7-triamine;
N²-(2,6-Dichloro-phenyl)-N⁵-isopropyl-N⁷-(4-trifluoromethyl-phenyl)-thiazolo[5,4-d]pyrimidine-2,5,7-triamine;
N²-(2,6-Dimethyl-phenyl)-5-methyl-N⁷-[4-(propane-2-sulfonyl)-phenyl]-thiazolo[5,4-d]pyrimidine-2,7-diamine;
N²-(2,6-Dichloro-phenyl)-N⁷-(4-isopropylsulfanyl-phenyl)-5-methyl-thiazolo[5,4-d]pyrimidine-2,7-diamine;
N²-(2,6-Dimethyl-phenyl)-N⁷-[4-(pyrrolidine-1-sulfonyl)-phenyl]-thiazolo[5,4-d]pyrimidine-2,7-diamine;
(racemic)-N²-(2,6-Dichloro-phenyl)-5-(2-methyl-pyrrolidin-1-yl)-N⁷-(4-trifluoromethyl-phenyl)-thiazolo[5,4-d]pyrimidine-2,7-diamine;
N²-(2,6-Dimethyl-phenyl)-N⁷-(4-isopropylsulfanyl-phenyl)-5-methyl-thiazolo[5,4-d]pyrimidine-2,7-diamine;
N²-(2,6-Dichloro-phenyl)-5-methyl-N⁷-(1,4,4-trimethyl-1,2,3,4-tetrahydro-quinolin-7-yl)-thiazolo[5,4-d]pyrimidine-2,7-diamine;
N²-(2,6-Dimethyl-phenyl)-N⁷-(3-fluoro-4-trifluoromethyl-phenyl)-thiazolo[5,4-d]pyrimidine-2,7-diamine;
N²-(2-Chloro-phenyl)-N⁷-[4-(pyrrolidine-1-sulfonyl)-phenyl]-thiazolo[5,4-d]pyrimidine-2,7-diamine;
N²-(2,6-Dimethyl-phenyl)-N⁷-[4-(4-methyl-piperazine-1-sulfonyl)-phenyl]-thiazolo[5,4-d]pyrimidine-2,7-diamine;
N²-(2,6-Dichloro-phenyl)-5-methyl-N⁷-(4-trifluoromethoxy-phenyl)-thiazolo[5,4-d]pyrimidine-2,7-diamine;
N²-(2,6-Dimethyl-phenyl)-N⁷-(4-isopropylsulfanyl-phenyl)-thiazolo[5,4-d]pyrimidine-2,7-diamine;
4-[2-(2-Chloro-phenylamino)-thiazolo[5,4-d]pyrimidin-7-ylamino]-N,N-dimethyl-benzenesulfonamide;
N²-(2,6-Dimethyl-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-thiazolo[5,4-d]pyrimidine-2,7-diamine;
N⁷-(4-Methanesulfonyl-phenyl)-N²-o-tolyl-thiazolo[5,4-d]pyrimidine-2,7-diamine;
(racemic)-N²-(2,6-Dichloro-phenyl)-N⁷-(4-methanesulfonyl-phenyl)-5-(2-methyl-pyrrolidin-1-yl)-thiazolo[5,4-d]pyrimidine-2,7-diamine;
(racemic)-N⁷-(3-Chloro-4-trifluoromethyl-phenyl)-N²-(2,6-dichloro-phenyl)-5-(2-isopropyl-pyrrolidin-1-yl)-thiazolo[5,4-d]pyrimidine-2,7-diamine;
N⁷-(6-Chloro-pyridin-3-yl)-N²-(2,6-dimethyl-phenyl)-thiazolo[5,4-d]pyrimidine-2,7-diamine;
N²-(2,6-Dimethyl-phenyl)-N⁷-(4-methylsulfanyl-phenyl)-thiazolo[5,4-d]pyrimidine-2,7-diamine;
N²-(2,6-Dimethyl-phenyl)-N⁷-(3-fluoro-4-trifluoromethyl-phenyl)-5-methyl-thiazolo[5,4-d]pyrimidine-2,7-diamine;
N²-(2,6-Dimethyl-phenyl)-N⁷-[4-(propane-2-sulfonyl)-phenyl]-thiazolo[5,4-d]pyrimidine-2,7-diamine;
N²-(4-Bromo-phenyl)-N⁷-(2,6-dimethyl-phenyl)-thiazolo[5,4-d]pyrimidine-2,7-diamine;
N⁷-(3-Chloro-4-methylsulfanyl-phenyl)-N²-(2,6-dichloro-phenyl)-thiazolo[5,4-d]pyrimidine-2,7-diamine;
N²-(2,6-Dimethyl-phenyl)-N⁷-(4-isopropyl-phenyl)-5-methyl-thiazolo[5,4-d]pyrimidine-2,7-diamine;
N²-(2-Chloro-phenyl)-N⁷-(4-methanesulfonyl-phenyl)-thiazolo[5,4-d]pyrimidine-2,7-diamine;
4-[2-(2,6-Dichloro-phenylamino)-5-methylsulfanyl-thiazolo[5,4-d]pyrimidin-7-ylamino]-N,N-dimethyl-benzenesulfonamide;
1-{4-[2-(2,6-Dimethyl-phenylamino)-thiazolo[5,4-d]pyrimidin-7-ylamino]-phenyl}-ethanone;
N²-(2,6-Dichloro-phenyl)-N⁷-(4-methanesulfonyl-phenyl)-5-piperidin-1-yl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-pyrrolidin-1-yl)-thiazolo[5,4-d]pyrimidine-2,7-diamine;
N⁷-(3-Chloro-4-trifluoromethylsulfanyl-phenyl)-N²-(2,6-dimethyl-phenyl)-5-methyl-thiazolo[5,4-d]pyrimidine-2,7-diamine;
N⁷-(4-Chloro-phenyl)-N²-(2,6-dimethyl-phenyl)-thiazolo[5,4-d]pyrimidine-2,7-diamine;
N²-(2,6-Dimethyl-phenyl)-N⁷-(3-fluoro-4-methyl-phenyl)-5-methyl-thiazolo[5,4-d]pyrimidine-2,7-diamine;
N²-(2,6-Dichloro-phenyl)-N⁷-[4-(piperazine-1-sulfonyl)-phenyl]-thiazolo[5,4-d]pyrimidine-2,7-diamine;
N⁷-(3-Chloro-4-trifluoromethylsulfanyl-phenyl)-N²-(2,6-dichloro-phenyl)-5-methyl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-piperidin-1-yl)-thiazolo[5,4-d]pyrimidine-2,7-diamine;
N²-(2,6-Dimethyl-phenyl)-N⁷-(4-iodo-phenyl)-thiazolo[5,4-d]pyrimidine-2,7-diamine;
N²-(2,6-Dimethyl-phenyl)-5-methyl-N⁷-p-tolyl-thiazolo[5,4-d]pyrimidine-2,7-diamine;
N²-(2,6-Dichloro-phenyl)-5-methyl-N⁷-(1-methyl-1,2,3,4-tetrahydro-quinolin-7-yl)-thiazolo[5,4-d]pyrimidine-2,7-diamine;
(racemic)-1-{4-[2-(2,6-Dimethyl-phenylamino)-thiazolo[5,4-d]pyrimidin-7-ylamino]-phenyl}-ethanol;
N²-(2,6-Dimethyl-phenyl)-5-methyl-N⁷-phenyl-thiazolo[5,4-d]pyrimidine-2,7-diamine;
2-Chloro-4-[2-(2,6-dimethyl-phenylamino)-thiazolo[5,4-d]pyrimidin-7-ylamino]-benzonitrile;
(racemic)-N²-(2,6-Dimethyl-phenyl)-N⁷-(4-methanesulfinyl-phenyl)-5-methyl-thiazolo[5,4-d]pyrimidine-2,7-diamine;
N²-(2,6-Dimethyl-phenyl)-N⁷-(4-fluoro-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzamide;
(racemic)-{4-[2-(2,6-Dichloro-phenylamino)-7-(4-trifluoromethyl-phenylamino)-thiazolo[5,4-d]pyrimidin-5-yl]-morpholin-2-yl}-methanol;
N²-(2,6-Dimethyl-phenyl)-N⁷-phenyl-thiazolo[5,4-d]pyrimidine-2,7-diamine;
N²-(2,6-Dimethyl-phenyl)-N⁷-(3-fluoro-4-methyl-phenyl)-thiazolo[5,4-d]pyrimidine-2,7-diamine;
4-[2-(2,6-Dimethyl-phenylamino)-thiazolo[5,4-d]pyrimidin-7-ylamino]-2-trifluoromethyl-benzonitrile,
N⁷-(2,3-Dihydro-benzo[1,4]dioxin-6-yl)-N²-(2,6-dimethyl-phenyl)-thiazolo[5,4-d]pyrimidine-2,7-diamine;
N²-(2,6-Dimethyl-phenyl)-N⁷-[4-(piperazine-1-sulfonyl)-phenyl]-thiazolo[5,4-d]pyrimidine-2,7-diamine;
N-{4-[2-(2,6-Dichloro-phenylamino)-5-methyl-thiazolo[5,4-d]pyrimidin-7-ylamino]-phenyl}-N-methyl-methanesulfonamide;
N²-(2,6-Dichloro-phenyl)-N⁵-[3-(4-methyl-piperazin-1-yl)-propyl]-N⁷-(4-trifluoromethyl-phenyl)-thiazolo[5,4-d]pyrimidine-2,5,7-diamine;
(racemic)-N²-(2,6-Dimethyl-phenyl)-N⁷-[4-(tetrahydro-furan-3-yloxy)-phenyl]-thiazolo[5,4-d]pyrimidine-2,7-diamine;
(racemic)-{4-[7-(3-Chloro-4-trifluoromethyl-phenylamino)-2-(2,6-dichloro-phenylamino)-thiazolo[5,4-d]pyrimidin-5-yl]-morpholin-2-yl}-methanol;
Cyclopentyl-{4-[2-(2,6-dichloro-phenylamino)-5-methyl-thiazolo[5,4-d]pyrimidin-7-ylamino]-phenyl}-methanone;
4-[2-(2,6-Dichloro-phenylamino)-5-methyl-thiazolo[5,4-d]pyrimidin-7-ylamino]-N,N-dimethyl-benzamide;
2-[2-(2,6-Dimethyl-phenylamino)-thiazolo[5,4-d]pyrimidin-7-ylamino]-5-methyl-phenol;
N²-(2,6-Dimethyl-phenyl)-N⁷-(2-methyl-4-trifluoromethyl-phenyl)-thiazolo[5,4-d]pyrimidine-2,7-diamine;
5-[2-(2,6-Dimethyl-phenylamino)-thiazolo[5,4-d]pyrimidin-7-ylamino]-2-methyl-phenol;
N-{4-[2-(2,6-Dimethyl-phenylamino)-thiazolo[5,4-d]pyrimidin-7-ylamino]-phenyl}-N-methyl-methanesulfonamide;
N⁷-(3-Chloro-4-trifluoromethyl-phenyl)-N²-(2,6-dichloro-phenyl)-5-piperazin-1-yl-thiazolo[5,4-d]pyrimidine-2,7-diamine;
N²-(2,6-Dichloro-phenyl)-5-piperazin-1-yl-N⁷-(4-trifluoromethyl-phenyl)-thiazolo[5,4-d]pyrimidine-2,7-diamine;
N-{4-[2-(2,6-Dimethyl-phenylamino)-5-methyl-thiazolo[5,4-d]pyrimidin-7-ylamino]-phenyl}-N-methyl-methanesulfonamide;
N²-(2,6-Dimethyl-phenyl)-N⁷-(3,4-dimethyl-phenyl)-thiazolo[5,4-d]pyrimidine-2,7-diamine;
N²-(2,6-Dichloro-phenyl)-5-methyl-N⁷-pyridin-3-yl-thiazolo[5,4-d]pyrimidine-2,7-diamine;
N⁷-(2-Chloro-4-trifluoromethyl-phenyl)-N²-(2,6-dimethyl-phenyl)-thiazolo[5,4-d]pyrimidine-2,7-diamine;
N²-(2,6-Dimethyl-phenyl)-N⁷-(4-methoxy-3-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzamide;
N²-(2,6-Dichloro-phenyl)-5-methyl-N⁷-phenyl-thiazolo[5,4-d]pyrimidine-2,7-diamine;
N²-(2,6-Dimethyl-phenyl)-5-methyl-N⁷-pyridin-3-yl-thiazolo[5,4-d]pyrimidine-2,7-diamine;
4-[2-(2,6-Dimethyl-phenylamino)-thiazolo[5,4-d]pyrimidin-7-ylamino]-benzoic acid;
N-{4-[2-(2,6-Dichloro-phenylamino)-5-methyl-thiazolo[5,4-d]pyrimidin-7-ylamino]-phenyl}-dimethanesulfonamide;
N-{4-[2-(2,6-Dimethyl-phenylamino)-5-methyl-thiazolo[5,4-d]pyrimidin-7-ylamino]-phenyl}-methanesulfonamide;
N-{4-[2-(2,6-Dimethyl-phenylamino)-thiazolo[5,4-d]pyrimidin-7-ylamino]-phenyl}-methanesulfonamide;
N²-(2,6-Dimethyl-phenyl)-N⁷-(5-trifluoromethyl-pyridin-2-yl)-thiazolo[5,4-d]pyrimidine-2,7-diamine;
(racemic)-N²-(2,6-Dichloro-phenyl)-5-(2-isopropyl-pyrrolidin-1-yl)-N⁷-(4-methanesulfonyl-phenyl)-thiazolo[5,4-d]pyrimidine-2,7-diamine;
N²-(2,6-Dichloro-phenyl)-N⁷-(4-methanesulfonyl-phenyl)-5-morpholin-4-yl-thiazolo[5,4-d]pyrimidine-2,7-diamine;
(racemic)-N²-(2,6-Dichloro-phenyl)-N⁷-(4-methanesulfonyl-phenyl)-5-(2-methyl-piperidin-1-yl)-thiazolo[5,4-d]pyrimidine-2,7-diamine;
N²-(2,6-Dichloro-phenyl)-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁷-(2-methylamino-ethyl)-N (4-trifluoromethyl-phenyl)-thiazolo[5,4-d]pyrimidine-2,5,7-triamine;
N²-(2,6-Dichloro-phenyl)-N⁵-(2-dimethylamino-ethyl)-N⁵-methyl-N⁷-(4-trifluoromethyl-phenyl)-thiazolo[5,4-d]pyrimidine-2,5,7-triamine;
(3R)-N²-(2,6-Dichloro-phenyl)-5-(3-methylamino-pyrrolidin-1-yl)-N⁷-(4-trifluoromethyl-phenyl)-thiazolo[5,4-d]pyrimidine-2,7-diamine;
N⁵-Cyclopropylmethyl-N²-(2,6-dichloro-phenyl)-N⁷-(4-methanesulfonyl-phenyl)-thiazolo[5,4-d]pyrimidine-2,5,7-triamine;
N²-(2,6-Dichloro-phenyl)-5-methyl-N⁷-(6-methylsulfanyl-pyridin-3-yl)-thiazolo[5,4-d]pyrimidine-2,7-diamine;
(racemic)-2-[2-(2,6-Dichloro-phenylamino)-7-(4-trifluoromethyl-phenylamino)-thiazolo[5,4-d]pyrimidin-5-ylamino]-propan-1-ol;
N²-(2,6-Dichloro-phenyl)-5-(4-methyl-piperazin-1-yl)-N⁷-(4-trifluoromethyl-phenyl)-thiazolo[5,4-d]pyrimidine-2,7-diamine;
N²-(2,6-Dichloro-phenyl)-N⁵,N⁵-diethyl-N⁷-(4-trifluoromethyl-phenyl)-thiazolo[5,4-d]pyrimidine-2,5,7-triamine;
5-Butoxy-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-(4-methyl-piperidin-1-yl)-N²-(4-trifluoromethyl-phenyl)-thiazolo[5,4-d]pyrimidine-2,7-diamine;
(racemic)-N²-(2,6-Dichloro-phenyl)-5-(2-methyl-piperidin-1-yl)-N⁷-(4-trifluoromethyl-phenyl)-thiazolo[5,4-d]pyrimidine-2,7-diamine;
(3S)-N²-(2,6-Dichloro-phenyl)-5-(3-methyl-morpholin-4-yl)-N⁷-(4-trifluoromethyl-phenyl)-thiazolo[5,4-d]pyrimidine-2,7-diamine;
(2S)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
(2R)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
5-Methyl-N²-(2-methylsulfanyl-phenyl)-N⁷-(4-trifluoromethyl-phenyl)-thiazolo[5,4-d]pyrimidine-2,7-diamine;
N²-(2-Methylsulfanyl-phenyl)-N⁷-(4-trifluoromethyl-phenyl)-thiazolo[5,4-d]pyrimidine-2,7-diamine;
N²-(2-Methanesulfonyl-phenyl)-5-methyl-N⁷-(4-trifluoromethylphenyl)-thiazolo[5,4-d]pyrimidine-2,7-diamine;
N²-(2-Methanesulfonyl-phenyl)-N⁷-(4-methanesulfonyl-phenyl)-thiazolo[5,4-d]pyrimidine-2,7-diamine;
N²-(2-Methanesulfonyl-phenyl)-N⁷-(6-trifluoromethyl-pyridin-3-yl)-thiazolo[5,4-d]pyrimidine-2,7-diamine;
N²-(2-Methanesulfonyl-phenyl)-N⁷-(4-trifluoromethanesulfonyl-phenyl)-thiazolo[5,4-d]pyrimidine-2,7-diamine;
N²-(2-Methanesulfonyl-phenyl)-N⁷-(4-trifluoromethyl-phenyl)-thiazolo[5,4-d]pyrimidine-2,7-diamine;
N²-(2-Chloro-phenyl)-5-methyl-N⁷-(4-trifluoromethyl-phenyl)-thiazolo[5,4-d]pyrimidine-2,7-diamine;
N²-(2-Chloro-phenyl)-N⁷-(4-trifluoromethanesulfonyl-phenyl)-thiazolo[5,4-d]pyrimidine-2,7-diamine;
N²-(2,6-Dichloro-phenyl)-N⁷-phenyl-thiazolo[5,4-d]pyrimidine-2,7-diamine;
N²-Benzo[1,2,5]thiadiazol-4-yl-5-methyl-N⁷-(4-trifluoromethyl-phenyl)-thiazolo[5,4-d]pyrimidine-2,7-diamine;
5-Methyl-N (2-nitro-phenyl)-N (4-trifluoromethyl-phenyl)-thiazolo[5,4-d]pyrimidine-2,7-diamine;
3-[7-(3-Chloro-4-trifluoromethyl-phenylamino)-thiazolo[5,4-d]pyrimidin-2-ylamino]-4-methyl-thiophene-2-carboxylic acid methyl ester;
N²-(3,5-Dimethyl-isoxazol-4-yl)-5-methyl-N⁷-(4-trifluoromethyl-phenyl)thiazolo[5,4-d]pyrimidine-2,7-diamine;
N⁷-(4-tert-Butyl-phenyl)-N²-(3,5-dimethyl-isoxazol-4-yl)-5-methyl-thiazolo[5,4-d]pyrimidine-2,7-diamine;
N²-(3-Methyl-pyridin-2-yl)-N⁷-[4-(pyrrolidine-1-sulfonyl)-phenyl]-thiazolo[5,4-d]pyrimidine-2,7-diamine;
5-Methyl-N²-(3-methyl-pyridin-2-yl)-N⁷-(4-trifluoromethyl-phenyl)-thiazolo[5,4-d]pyrimidine-2,7-diamine;
N,N-Dimethyl-4-[5-methyl-2-(3-methyl-pyridin-2-ylamino)-thiazolo[5,4-d]pyrimidin-7-ylamino]-benzenesulfonamide;
N²-(3-Methyl-pyridin-2-yl)-N⁷-(4-trifluoromethyl-phenyl)-thiazolo[5,4-d]pyrimidine-2,7-diamine;
N²-(3,5-Dichloro-pyridin-4-yl)-N⁷-[4-(pyrrolidine-1-sulfonyl)-phenyl]-thiazolo[5,4-d]pyrimidine-2,7-diamine;
N²-(2,6-Dichloro-phenyl)-N⁷-(3-fluoro-4-trifluoromethyl-phenyl)-thiazolo[5,4-d]pyrimidine-2,7-diamine;
N²-(2-Chlorophenyl)-N⁷-[4-(morpholin-4-ylsulfonyl)phenyl][1,3]thiazolo[5,4-d]pyrimidine-2,7-diamine;
N²-(2-Methylphenyl)-N⁷-[4-(morpholin-4-ylsulfonyl)phenyl][1,3]thiazolo[5,4-d]pyrimidine-2,7-diamine;
N²-(2-Methylphenyl)-N⁷-[6-(trifluoromethyl)pyridin-3-yl][1,3]thiazolo[5,4-d]pyrimidine-2,7-diamine;
N²-[2-(Trifluoromethyl)phenyl]-N⁷-[6-(trifluoromethyl)pyridin-3-yl][1,3]thiazolo[5,4-d]pyrimidine-2,7-diamine;
N²-(2-Chlorophenyl)-N⁷-[6-(trifluoromethyl)pyridin-3-yl][1,3]thiazolo[5,4-d]pyrimidine-2,7-diamine;
N²-(3,5-Dimethylisoxazol-4-yl)-N⁷-[4-(morpholin-4-ylsulfonyl)phenyl][1,3]thiazolo[5,4-d]pyrimidine-2,7-diamine;
Methyl 2-[4-({2-[(3,5-dimethylisoxazol-4-yl)amino][1,3]thiazolo[5,4-d]pyrimidin-7-yl}amino)phenyl]-2-methylpropanoate;
2-[4-({2-[(3,5-Dimethylisoxazol-4-yl)amino][1,3]thiazolo[5,4-d]pyrimidin-7-yl}amino)phenyl]-2-methylpropanenitrile;
N²-(3,5-Dimethylisoxazol-4-yl)-N⁷-[4-(methylsulfonyl)phenyl][1,3]thiazolo[5,4-d]pyrimidine-2,7-diamine;
N²-[2-(Trifluoromethyl)phenyl]-N⁷-[4-(trifluoromethyl)phenyl][1,3]thiazolo[5,4-d]pyrimidine-2,7-diamine;
N⁷-[4-(Methylsulfonyl)phenyl]-N²-[2-(trifluoromethyl)phenyl][1,3]thiazolo[5,4-d]pyrimidine-2,7-diamine;
4-({2-[(2,6-Dichlorophenyl)amino][1,3]thiazolo[5,4-d]pyrimidin-7-yl}amino)benzene-1,2-diol;
2-[4-({2-[(2,6-Dichlorophenyl)amino][1,3]thiazolo[5,4-d]pyrimidin-7-yl}amino)phenyl]-2-methylpropanenitrile;
Methyl 2-[4-({2-[(2,6-dichlorophenyl)amino][1,3]thiazolo[5,4-d]pyrimidin-7-yl}amino)phenyl]-2-methylpropanoate;
2-[4-({2-[(2,6-Dichlorophenyl)amino][1,3]thiazolo[5,4-d]pyrimidin-7-yl}amino)phenyl]-2-methylpropanoic acid;
1-Methylethyl 2-[4-({2-[(2,6-dichlorophenyl)amino][1,3]thiazolo[5,4-d]pyrimidin-7-yl}amino)phenyl]-2-methylpropanoate;
N²-Cyclohexyl-N⁷-[4-(trifluoromethyl)phenyl][1,3]thiazolo[5,4-d]pyrimidine-2,7-diamine;
N²-Cyclohexyl-N⁷-[6-(trifluoromethyl)pyridin-3-yl][1,3]thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzonitrile;
3,5-Dichloro-4-[7-(4-trifluoromethyl-phenylamino)-thiazolo[5,4-d]pyrimidin-2-ylamino]-benzamide;
3,5-Dichloro-4-[7-(4-methanesulfonyl-phenylamino)-thiazolo[5,4-d]pyrimidin-2-ylamino]-benzonitrile;
3,5-Dichloro-4-[7-(4-methanesulfonyl-phenylamino)-thiazolo[5,4-d]pyrimidin-2-ylamino]-benzamide;
N²-(2,6-Dichloro-4-morpholin-4-ylmethyl-phenyl)-N⁷-(4-trifluoromethyl-phenyl)-thiazolo[5,4-d]pyrimidine-2,7-diamine;
N²-(4-Azetidin-1-ylmethyl-2,6-dichloro-phenyl)-N⁷-(4-trifluoromethyl-phenyl)-thiazolo[5,4-d]pyrimidine-2,7-diamine;
N²-(4-Aminomethyl-2,6-dichloro-phenyl)-N⁷-(4-trifluoromethyl-phenyl)-thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzoic acid methyl ester;
{3,5-Dichloro-4-[7-(4-trifluoromethyl-phenylamino)-thiazolo[5,4-d]pyrimidin-2-ylamino]-phenyl}-methanol;
3,5-Dichloro-4-[7-(4-trifluoromethyl-phenylamino)-thiazolo[5,4-d]pyrimidin-2-ylamino]-benzoic acid;
N⁷-(4-tert-Butyl-phenyl)-N²-(2,6-dimethyl-phenyl)-5-methyl-thiazolo[5,4-d]pyrimidine-2,7-diamine;
N²-(2,6-Dimethyl-phenyl)-5-methyl-N⁷-(4-trifluoromethyl-phenyl)-thiazolo[5,4-d]pyrimidine-2,7-diamine;
N²-(2,6-Dimethyl-phenyl)-5-methyl-N⁷-(6-trifluoromethyl-pyridin-3-yl)-thiazolo[5,4-d]pyrimidine-2,7-diamine;
N⁷-(3-Chloro-4-trifluoromethyl-phenyl)-N²-(2,6-dimethyl-phenyl)-5-methyl-thiazolo[5,4-d]pyrimidine-2,7-diamine;
N⁷-(4-tert-Butyl-cyclohexyl)-N²-(2,6-dimethyl-phenyl)-thiazolo[5,4-d]pyrimidine-2,7-diamine;
N⁷-(4-tert-Butyl-cyclohexyl)-N²-(2,6-dimethyl-phenyl)-5-methyl-thiazolo[5,4-d]pyrimidine-2,7-diamine;
(R)-1-[2-(2,6-Dichloro-phenylamino)-7-(4-trifluoromethyl-phenylamino)-thiazolo[5,4-d]pyrimidin-5-ylamino]-propan-2-ol;
1-[2-(2,6-Dichloro-phenylamino)-7-(4-trifluoromethyl-phenylamino)-thiazolo[5,4-d]pyrimidin-5-ylamino]-2-methyl-propan-2-ol;
(racemic)-{1-[2-(2,6-Dichloro-phenylamino)-7-(4-trifluoromethyl-phenylamino)-thiazolo[5,4-d]pyrimidin-5-yl]-pyrrolidin-2-yl}-methanol;
N²-(2,6-Dichloro-phenyl)-N⁵-methyl-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁷-(6-methanesulfonyl-pyridin-3-yl)-thiazolo[5,4-d]pyrimidine-2,7-diamine;
2-{4-[2-(2,6-Dichloro-phenylamino)-thiazolo[5,4-d]pyrimidin-7-ylamino]-phenyl}-isobutyramide;
(racemic)-1-[2-(2,6-Dichloro-phenylamino)-7-(4-trifluoromethyl-phenylamino)-thiazolo[5,4-d]pyrimidin-5-ylamino]-propan-2-ol;
(racemic)-3-[2-(2,6-Dichloro-phenylamino)-7-(4-trifluoromethyl-phenylamino)-thiazolo[5,4-d]pyrimidin-5-ylamino]-propane-1,2-diol; and
N²-(2,6-Dichloro-phenyl)-N⁵-(2-pyrrolidin-1-yl-ethyl)-N⁷-(4-trifluoromethyl-phenyl)-thiazolo[5,4-d]pyrimidine-2,5,7-triamine;
and pharmaceutically acceptable salts thereof.

39. A pharmaceutical composition according to claim 36, further comprising: an analgesic selected from the group consisting of opioids and non-steroidal anti-inflammatory drugs.

40. A pharmaceutical composition according to claim 36, further comprising: an active ingredient selected from the group consisting of aspirin, acetaminophen, opioids, ibuprofen, naproxen, COX-2 inhibitors, gabapentin, pregabalin, and tramadol.

41. A method of treating a subject suffering from or diagnosed with a disease, disorder, or condition mediated by TRPV1 activity, comprising administering to the subject an effective amount of at least one active agent selected from compounds of Formula (I) and pharmaceutically acceptable salts, pharmaceutically acceptable prodrugs, and pharmaceutically active metabolites of said compounds of Formula (I): 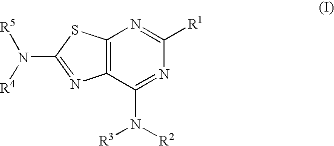
wherein:
R¹ is —H; —NR^{a}R^{b}; a —C₁₋₆alkyl, —OC₁₋₆alkyl, —S—C₁₋₆alkyl, or —SO₂—C₁₋₆alkyl group unsubstituted or substituted with an —OH, —OC₁₋₄alkyl, —NR^{e}R^{f}, or halo substituent;
or a monocyclic cycloalkyl or phenyl group unsubstituted or substituted with a —C₁₋₆alkyl, —OH, —OC₁₋₄alkyl, —NR^{e}R^{f}, or halo substituent;
where R^{a} and R^{b} are each independently —H; —C₁₋₆alkyl; a —C₂₋₄alkyl group substituted with one or two —OH, —OC₁₋₄alkyl, —NR^{c}R^{d}, or halo substituents; or a saturated monocyclic cycloalkyl, —C₁alkyl-(saturated monocyclic cycloalkyl), saturated monocyclic heterocycloalkyl, —C₁alkyl-(saturated monocyclic heterocycloalkyl), phenyl, or benzyl group unsubstituted or substituted with one, two, or three moieties independently selected from the group consisting of —C₁₋₆alkyl, —OH, —OC₁₋₄alkyl, —NR^{p}R^{q}, and halo substituents; or
R^{a} and R^{b} taken together with the nitrogen of attachment in —NR^{a}R^{b} form a saturated monocyclic heterocycloalkyl group unsubstituted or substituted with one, two, or three moieties independently selected from the group consisting of —C₁₋₆alkyl, —C₁₋₂alkyl-OH, —C₁₋₂alkyl-OC₁₋₂alkyl, —OH, —OC₁₋₄alkyl, —NR^{p}R^{q}, halo, —CO₂H, and benzyl substituents;
where R^{c} and R^{d} are each independently —H or —C₁₋₆alkyl; or R^{c} and R^{d} taken together with the nitrogen of attachment in —NR^{c}R^{d} form a saturated monocyclic heterocycloalkyl unsubstituted or substituted with methyl; and
R^{p} and R^{q} are each independently —H or —C₁₋₆alkyl; or R^{p} and R^{q} taken together with the nitrogen of attachment in —NR^{p}R^{q} form a saturated monocyclic heterocycloalkyl unsubstituted or substituted with methyl; and
R^{e} and R^{f} are each independently —H or —C₁₋₆alkyl; or R^{e} and R^{f} taken together with their nitrogen of attachment in —NR^{e}R^{f} form a saturated monocyclic heterocycloalkyl unsubstituted or substituted with methyl;
R² is —H or —C₁₋₆alkyl;
R³ is a monocyclic cycloalkyl, phenyl, benzyl, phenethyl, indanyl, quinolinyl, monocyclic five-membered heteroaryl, monocyclic six-membered heteroaryl, or —C₁alkyl-(monocyclic heteroaryl) group unsubstituted or substituted with one, two, or three R^{g} substituents;
where each R^{g} substituent is independently —C₁₋₆alkyl, —OH, —OC₁₋₆alkyl, —O— (saturated monocyclic heterocycloalkyl), phenoxy, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH; or
two adjacent R^{g} substituents taken together form —OC₁₋₂alkylO—, —C₂₋₆alkylO—, or —C₂₋₆alkylN(R^{h})—;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl unsubstituted or substituted with methyl; and
each R^{j} is independently —H, —C₁₋₆alkyl, or —CF₃; or both R^{j} substituents taken together with the carbon to which they are attached form a monocyclic cycloalkyl ring;
R⁴ is —H or —C₁₋₆alkyl; and
R⁵ is a phenyl, monocyclic five-membered heteroaryl, or monocyclic six-membered heteroaryl group unsubstituted or substituted with one, two, or three R^{k} substituents;
where each R^{k} substituent is independently —C₁₋₆alkyl unsubstituted or substituted with one or two —OH groups, —C₁₋₂alkyl-N(R^{l})R^{m}, —OH, —OC₁₋₁₆alkyl, phenyl, phenoxy, —CN, —NO₂, —N(R^{l})R^{m}, —C(O)N(R^{l})R^{m}, —N(R^{l})C(O)R^{m}, —N(R^{l})SO₂C₁₋₆alkyl, —N(R^{l})SO₂CF₃, —C(O)C₁₋₆alkyl, —S(O)₀₋₂—C₁₋₆alkyl, —SO₂CF₃, —SO₂N(R^{l})R^{m}, —SCF₃, halo, —CF₃, —OCF₃, —CO₂H, or —CO₂C₁₋₆alkyl; or
two adjacent R^{k} substituents taken together form —OC₁₋₂alkylO— or ═N—S—N═;
where R^{l} and R^{m} are each independently —H or —C₁₋₆alkyl; or R^{l} and R^{m} taken together with their nitrogen of attachment in —NR^{l}R^{m} form a saturated monocyclic heterocycloalkyl unsubstituted or substituted with methyl.

42. A method according to claim 41, wherein said active agent is selected from the group consisting of:
N²-(2,6-Dichloro-phenyl)-N⁷-(4-trifluoromethyl-phenyl)-thiazolo[5,4-d]pyrimidine-2,7-diamine;
N²-(2,6-Dichloro-phenyl)-N⁷-(6-trifluoromethyl-pyridin-3-yl)-thiazolo[5,4-d]pyrimidine-2,7-diamine;
N⁷-(4-tert-Butyl-phenyl)-N²-(2,6-dichloro-phenyl)-thiazolo[5,4-d]pyrimidine-2,7-diamine;
N⁷-(3-Chloro-4-trifluoromethyl-phenyl)-N²-(2,6-dichloro-phenyl)-thiazolo[5,4-d]pyrimidine-2,7-diamine;
N²-(2,6-Dichloro-phenyl)-5-methyl-N⁷-(4-trifluoromethyl-phenyl)-thiazolo[5,4-d]pyrimidine-2,7-diamine;
N²-(2,6-Dichloro-phenyl)-5-methyl-N⁷-(6-trifluoromethyl-pyridin-3-yl)-thiazolo[5,4-d]pyrimidine-2,7-diamine;
/7-(4-tert-Butyl-phenyl)-N²-(2,6-dichloro-phenyl)-5-methyl-thiazolo[5,4-d]pyrimidine-2,7-diamine;
N⁷-(4-tert-Butyl-cyclohexyl)-N²-(2,6-dichloro-phenyl)-5-methyl-thiazolo[5,4-d]pyrimidine-2,7-diamine;
N²-(2,6-Dichloro-phenyl)-5,N⁷-dimethyl-N⁷-(4-trifluoromethyl-phenyl)-thiazolo[5,4-d]pyrimidine-2,7-diamine;
N²-(2,6-Dimethyl-phenyl)-N⁷-(4-trifluoromethyl-phenyl)thiazolo[5,4,d]pyrimidine-2,7-diamine;
N²-(2,6-Dimethyl-phenyl)-N⁷-(6-trifluoromethyl-pyridin-3-yl)-thiazolo[5,4-d]pyrimidine-2,7-diamine;
N⁷-(4-tert-Butyl-phenyl)-N²-(2,6-dimethyl-phenyl)-thiazolo[5,4-d]pyrimidine-2,7-diamine;
N⁷-(3-Chloro-4-trifluoromethyl-phenyl)-N²-(2,6-dimethyl-phenyl)-thiazolo[5,4-d]pyrimidine-2,7-diamine;
N²-(2-Chloro-6-methyl-phenyl)-N⁷-(4-trifluoromethyl-phenyl)-thiazolo[5,4-d]pyrimidine-2,7-diamine;
N²-(2-Chloro-6-methyl-phenyl)-N⁷-(6-trifluoromethyl-pyridin-3-yl)-thiazolo[5,4-d]pyrimidine-2,7-diamine;
N²-(2-Chloro-phenyl)-N⁷-(4-trifluoromethyl-phenyl)-thiazolo[5,4-d]pyrimidine-2,7-diamine;
N²-o-Tolyl-N⁷-(4-trifluoromethyl-phenyl)-thiazolo[5,4-d]pyrimidine-2,7-diamine;
N²-(2-Chloro-6-trifluoromethyl-phenyl)-N⁷-(4-trifluoromethyl-phenyl)-thiazolo[5,4-d]pyrimidine-2,7-diamine;
N²-(2-Chloro-6-trifluoromethyl-phenyl)-N⁷-(6-trifluoromethyl-pyridin-3-yl)-thiazolo[5,4-d]pyrimidine-2,7-diamine;
N²-Phenyl-N⁷-(4-trifluoromethyl-phenyl)-thiazolo[5,4-d]pyrimidine-2,7-diamine;
N²-Phenyl-N⁷-(6-trifluoromethyl-pyridin-3-yl)-thiazolo[5,4-d]pyrimidine-2,7-diamine;
N²,N⁷-Bis-(4-trifluoromethyl-phenyl)-thiazolo[5,4-d]pyrimidine-2,7-diamine;
N²-(2,6-Dichloro-phenyl)-5,N²-dimethyl-N⁷-(4-trifluoromethyl-phenyl)-thiazolo[5,4-d]pyrimidine-2,7-diamine;
N²-(3,5-Dimethyl-isoxazol-4-yl)-N⁷-(6-trifluoromethyl-pyridin-3-yl)-thiazolo[5,4-d]pyrimidine-2,7-diamine;
N²-(3,5-Dimethyl-isoxazol-4-yl)-N⁷-(4-trifluoromethyl-phenyl)-thiazolo[5,4-d]pyrimidine-2,7-diamine;
5-Methyl-N²-(5-methyl-3-phenyl-isoxazol-4-yl)−/−(6-trifluoromethyl-pyridin-3-yl)-thiazolo[5,4-d]pyrimidine-2,7-diamine;
5-Methyl-N²-(5-methyl-3-phenyl-isoxazol-4-yl)-N⁷-(4-trifluoromethyl-phenyl)-thiazolo[5,4-d]pyrimidine-2,7-diamine;
N²-(2,6-Dimethyl-phenyl)-N⁷-(4-trifluoromethoxy-phenyl)-thiazolo[5,4-d]pyrimidine-2,7-diamine;
5-[2-(2,6-Dimethyl-phenylamino)-thiazolo[5,4-d]pyrimidin-7-ylamino]-pyridine-2-carbonitrile;
N⁷-(3-Chloro-4-trifluoromethyl-phenyl)-N²-(2,6-dichloro-phenyl)-5-methyl-thiazolo[5,4-d]pyrimidine-2,7-diamine;
4-[2-(2,6-Dimethyl-phenylamino)-thiazolo[5,4-d]pyrimidin-7-ylamino]-benzonitrile;
2-{4-[2-(2,6-Dimethyl-phenylamino)-thiazolo[5,4-d]pyrimidin-7-ylamino]-phenyl}-2-methyl-propionitrile;
N²-(2,6-Dimethyl-phenyl)-N⁷-(4-methoxy-phenyl)-thiazolo[5,4-d]pyrimidine-2,7-diamine;
N⁷-(3,4-Dichloro-phenyl)-N²-(2,6-dimethyl-phenyl)-thiazolo[5,4-d]pyrimidine-2,7-diamine;
N²-(2,6-Dimethyl-phenyl)-N⁷-p-tolyl-thiazolo[5,4-d]pyrimidine-2,7-diamine;
N²-(2,6-Dimethyl-phenyl)-N⁷-(2-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzoic acid methyl ester;
4-{[2-(2,6-Dimethyl-phenylamino)-thiazolo[5,4-d]pyrimidin-7-ylamino]-methyl}-2-methoxy-phenol;
N⁷-(3,4-Dichloro-benzyl)-N²-(2,6-dimethyl-phenyl)-thiazolo[5,4-d]pyrimidine-2,7-diamine;
N²-(2,6-Dimethyl-phenyl)-N⁷-(4-trifluoromethylsulfanyl-phenyl)-thiazolo[5,4-d]pyrimidine-2,7-diamine;
N²-(2,6-Dimethyl-phenyl)-N⁷-indan-2-yl-thiazolo[5,4-d]pyrimidine-2,7-diamine;
N²-(2,6-Dimethyl-phenyl)-N⁷-(3-trifluoromethyl-phenyl)-thiazolo[5,4-d]pyrimidine-2,7-diamine;
N⁷-Benzyl-N2-(2,6-dimethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzenesulfonamide;
N²-(2,6-Dimethyl-phenyl)-N⁷-(4-ethyl-phenyl)-thiazolo[5,4-d]pyrimidine-2,7-diamine;
N²-(2,6-Dimethyl-phenyl)-N⁷-(4-isopropyl-phenyl)-thiazolo[5,4-d]pyrimidine-2,7-diamine;
N²-(2,6-Dimethyl-phenyl)-N⁷-(5-methyl-furan-2-ylmethyl)-thiazolo[5,4-d]pyrimidine-2,7-diamine;
4-Methyl-3-[7-(4-trifluoromethyl-phenylamino)-thiazolo[5,4-d]pyrimidin-2-ylamino]-thiophene-2-carboxylic acid methyl ester;
4-Methyl-3-[7-(6-trifluoromethyl-pyridin-3-ylamino)-thiazolo[5,4-d]pyrimidin-2-ylamino]-thiophene-2-carboxylic acid methyl ester;
N⁷-(3-Chloro-4-trifluoromethyl-phenyl)-N²-(3,5-dimethyl-isoxazol-4-yl)thiazolo[5,4d]pyrimidine-2,7-diamine;
N⁷-(4-tert-Butyl-phenyl)-N²-(3,5-dimethyl-isoxazol-4-yl)-thiazolo[5,4-d]pyrimidine-2,7-diamine;
N²-(2,6-Dichloro-phenyl)-5-methylsulfanyl-N⁷-(4-trifluoromethyl-phenyl)-thiazolo[5,4-d]pyrimidine-2,7-diamine;
N²-(2,6-Dichloro-phenyl)-5-methanesulfonyl-N⁷-(4-trifluoromethyl-phenyl)-thiazolo[5,4-d]pyrimidine-2,7-diamine;
N²-(2,6-Dichloro-phenyl)-5-piperidin-1-yl-N⁷-(4-trifluoromethyl-phenyl)-thiazolo[5,4-d]pyrimidine-2,7-diamine;
N²-(2,6-Dichloro-phenyl)-5-methoxy-N⁷-(4-trifluoromethyl-phenyl)-thiazolo[5,4d]pyrimidine-2,7-diamine;
N²-(2,6-Dichloro-phenyl)-N⁵, N⁵-dimethyl-N⁷-(4-trifluoromethyl-phenyl)-thiazolo[5,4-d]pyrimidine-2,5,7-triamine;
5-Azepan-1-yl-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-pyrrolidin-1-yl-N⁷-(4-trifluoromethyl-phenyl)-thiazolo[5,4-d]pyrimidine-2,7-diamine;
5-Azetidin-1-yl-N²-(2,6-dichloro-phenyl)-N⁷-(4-trifluoromethyl-phenyl)-thiazolo[5,4-d]pyrimidine-2,7-diamine;
N²-(2,6-Bis-methanesulfonyl-phenyl)-5-methyl-N⁷-(4-trifluoromethyl-phenyl)-thiazolo[5,4-d]pyrimidine-2,7-diamine;
N²-(2,6-Dichloro-phenyl)-N⁵-(2-methoxy-ethyl)-N⁷-(4-trifluoromethyl-phenyl)-thiazolo[5,4-d]pyrimidine-2,5,7-triamine;
N⁵-Cyclopropylmethyl-N²-(2,6-dichloro-phenyl)-N⁷-(4-trifluoromethyl-phenyl)-thiazolo[5,4-d]pyrimidine-2,5,7-triamine;
N²-(2,6-Dichloro-phenyl)-N⁵-(2-methoxy-ethyl)-N⁵-methyl-N⁷-(4-trifluoromethyl-phenyl)-thiazolo[5,4-d]pyrimidine-2,5,7-triamine;
N²-(2,6-Dichloro-phenyl)-5-morpholin-4-yl-N⁷-(4-trifluoromethyl-phenyl)-thiazolo[5,4-d]pyrimidine-2,7-diamine;
N²-(2,6-Dichloro-phenyl)-N⁷-(5-trifluoromethyl-pyridin-2-yl)-thiazolo[5,4-d]pyrimidine-2,7-diamine;
N²-(2,6-Dichloro-phenyl)-5-methyl-N⁷-(5-trifluoromethyl-pyridin-2-yl)-thiazolo[5,4-d]pyrimidine-2,7-diamine;
N²-(2,6-Dichloro-phenyl)-5-phenoxy-N⁷-(4-trifluoromethyl-phenyl)-thiazolo[5,4-d]pyrimidine-2,7-diamine;
N²-(2,6-Dichloro-phenyl)-N⁵-phenyl-N⁷-(4-trifluoromethyl-phenyl)-thiazolo[5,4-d]pyrimidine-2,5,7-triamine;
N²-(2,6-Dichloro-phenyl)-5-(4-isopropyl-piperazin-1-yl)-N⁷-(4-trifluoromethyl-phenyl)-thiazolo[5,4-d]pyrimidine-2,7-diamine;
N²-(2,6-Dichloro-phenyl)-5-phenyl-N⁷-(4-trifluoromethyl-phenyl)-thiazolo[5,4-d]pyrimidine-2,7-diamine;
N²-(2,6-Dichloro-phenyl)-5-isopropyl-N⁷-(4-trifluoromethyl-phenyl)-thiazolo[5,4-d]pyrimidine-2,7-diamine; and
N²-(3,5-Dichloro-pyridin-4-yl)-N⁷-(4-trifluoromethyl-phenyl)-thiazolo[5,4-d]pyrimidine-2,7-diamine;
and pharmaceutically acceptable salts thereof.

43. A method according to claim 41, wherein said active agent is selected from the group consisting of:
N²-(2,6-Dichloro-phenyl)-5-methyl-N⁷-[4-(pyrrolidine-1-sulfonyl)-phenyl]-thiazolo[5,4-d]pyrimidine-2,7-diamine;
2-{4-[2-(2,6-Dichloro-phenylamino)-5-methyl-thiazolo[5,4-d]pyrimidin-7-ylamino]-phenyl}-propan-2-ol;
4-[2-(2,6-Dichloro-phenylamino)-thiazolo[5,4-d]pyrimidin-7-ylamino]-N,N-dimethyl-benzenesulfonamide;
N²-(2,6-Dichloro-phenyl)-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-(4-trifluoromethanesulfonyl-phenyl)-thiazolo[5,4-d]pyrimidine-2,7-diamine;
N²-(2,6-Dichloro-phenyl)-N⁷-(4-methanesulfonyl-phenyl)-5-methyl-thiazolo[5,4-d]pyrimidine-2,7-diamine;
N²-(2,6-Dichloro-phenyl)-N⁵-isobutyl-N⁷-(4-trifluoromethyl-phenyl)-thiazolo[5,4-d]pyrimidine-2,5,7-triamine;
N²-(2,6-Dichloro-phenyl)-N⁷-[4-(morpholine-4-sulfonyl)-phenyl]-thiazolo[5,4-d]pyrimidine-2,7-diamine;
4-[2-(2,6-Dichloro-phenylamino)-5-methyl-thiazolo[5,4-d]pyrimidin-7-ylamino]-N,N-dimethyl-benzenesulfonamide;
N²-(2,6-Dichloro-phenyl)-N⁷-(3-fluoro-4-methanesulfonyl-phenyl)-thiazolo[5,4-d]pyrimidine-2,7-diamine;
N⁷-[4-(Pyrrolidine-1-sulfonyl)-phenyl]-N²-o-tolyl-thiazolo[5,4-d]pyrimidine-2,7-diamine;
N²-(2,6-Dichloro-phenyl)-N⁷-(4-isopropyl-phenyl)-5-methyl-thiazolo[5,4-d]pyrimidine-2,7-diamine;
4-[2-(2,6-Dimethyl-phenylamino)-5-methyl-thiazolo[5,4-d]pyrimidin-7-ylamino]-N,N-dimethyl-benzenesulfonamide;
1-{4-[2-(2,6-Dichloro-phenylamino)-5-methyl-thiazolo[5,4-d]pyrimidin-7-ylamino]-phenyl}-ethanone;
N²-(2,6-Dichloro-phenyl)-N⁷-(4-methanesulfonyl-phenyl)-thiazolo[5,4-d]pyrimidine-2,7-diamine;
N²-(2,6-Dichloro-phenyl)-N⁷-[4-(4-methyl-piperazine-1-sulfonyl)-phenyl]-thiazolo[5,4-d]pyrimidine-2,7-diamine;
(racemic)-N²-(2,6-Dichloro-phenyl)-5-(2-isopropyl-pyrrolidin-1-yl)-N⁷-(4-trifluoromethyl-phenyl)-thiazolo[5,4-d]pyrimidine-2,7-diamine;
N²-(2,6-Dimethyl-phenyl)-5-methyl-N⁷-(4-trifluoromethanesulfonyl-phenyl)-thiazolo[5,4-d]pyrimidine-2,7-diamine;
N²-(2,6-Dimethyl-phenyl)-N⁷-[4-(morpholine-4-sulfonyl)-phenyl]-thiazolo[5,4-d]pyrimidine-2,7-diamine;
N²-(2,6-Dimethyl-phenyl)-5-methyl-N⁷-[4-(pyrrolidine-1-sulfonyl)-phenyl]-thiazolo[5,4-d]pyrimidine-2,7-diamine;
N²-(2,6-Dichloro-phenyl)-5-methyl-N⁷-[4-(propane-2-sulfonyl)-phenyl]-thiazolo[5,4-d]pyrimidine-2,7-diamine;
N²-(2,6-Dichloro-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-5-methyl-thiazolo[5,4-d]pyrimidine-2,7-diamine;
4-[2-(2,6-Dichloro-phenylamino)-5-methyl-thiazolo[5,4-d]pyrimidin-7-ylamino]-benzonitrile;
N²-(2,6-Dimethyl-phenyl)-N⁷-(3-fluoro-4-methanesulfonyl-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzenesulfonamide;
N²-(2,6-Dimethyl-phenyl)-N⁷-(4-trifluoromethanesulfonyl-phenyl)-thiazolo[5,4-d]pyrimidine-2,7-diamine;
N²-(2,6-Dichloro-phenyl)-N⁵-(3-morpholin-4-yl-propyl)-N⁷-(4-trifluoromethyl-phenyl)-thiazolo[5,4-d]pyrimidine-2,5,7-triamine;
N²-(2,6-Dichloro-phenyl)-N⁵-isopropyl-N⁷-(4-trifluoromethyl-phenyl)-thiazolo[5,4-d]pyrimidine-2,5,7-triamine;
N²-(2,6-Dimethyl-phenyl)-5-methyl-N⁷-[4-(propane-2-sulfonyl)-phenyl]-thiazolo[5,4-d]pyrimidine-2,7-diamine;
N²-(2,6-Dichloro-phenyl)-N⁷-(4-isopropylsulfanyl-phenyl)-5-methyl-thiazolo[5,4-d]pyrimidine-2,7-diamine;
N²-(2,6-Dimethyl-phenyl)-N⁷-[4-(pyrrolidine-1-sulfonyl)-phenyl]-thiazolo[5,4-d]pyrimidine-2,7-diamine;
(racemic)-N²-(2,6-Dichloro-phenyl)-5-(2-methyl-pyrrolidin-1-yl)-N⁷-(4-trifluoromethyl-phenyl)-thiazolo[5,4-d]pyrimidine-2,7-diamine;
N²-(2,6-Dimethyl-phenyl)-N⁷-(4-isopropylsulfanyl-phenyl)-5-methyl-thiazolo[5,4-d]pyrimidine-2,7-diamine;
N²-(2,6-Dichloro-phenyl)-5-methyl-N⁷-(1,4,4-trimethyl-1,2,3,4-tetrahydro-quinolin-7-yl)-thiazolo[5,4-d]pyrimidine-2,7-diamine;
N²-(2,6-Dimethyl-phenyl)-N⁷-(3-fluoro-4-trifluoromethyl-phenyl)-thiazolo[5,4-d]pyrimidine-2,7-diamine;
N²-(2-Chloro-phenyl)-N⁷-[4-(pyrrolidine-1-sulfonyl)-phenyl]-thiazolo[5,4-d]pyrimidine-2,7-diamine;
N²-(2,6-Dimethyl-phenyl)-N⁷-[4-(4-methyl-piperazine-1-sulfonyl)-phenyl]-thiazolo[5,4-d]pyrimidine-2,7-diamine;
N²-(2,6-Dichloro-phenyl)-5-methyl-N⁷-(4-trifluoromethoxy-phenyl)-thiazolo[5,4-d]pyrimidine-2,7-diamine;
N²-(2,6-Dimethyl-phenyl)-N⁷-(4-isopropylsulfanyl-phenyl)-thiazolo[5,4-d]pyrimidine-2,7-diamine;
4-[2-(2-Chloro-phenylamino)-thiazolo[5,4-d]pyrimidin-7-ylamino]-N,N-dimethyl-benzenesulfonamide;
N²-(2,6-Dimethyl-phenyl)-5-methyl-N⁷-(4-methylsulfanyl-phenyl)-thiazolo[5,4-d]pyrimidine-2,7-diamine;
N²-(2,6-Dimethyl-phenyl)-N⁷-(4-methanesulfonyl-phenyl)-thiazolo[5,4-d]pyrimidine-2,7-diamine;
N⁷-(4-Methanesulfonyl-phenyl)-N²-o-tolyl-thiazolo[5,4-d]pyrimidine-2,7-diamine;
(racemic)-N²-(2,6-Dichloro-phenyl)-N⁷-(4-methanesulfonyl-phenyl)-5-(2-methyl-pyrrolidin-1-yl)-thiazolo[5,4-d]pyrimidine-2,7-diamine;
(racemic)-N⁷-(3-Chloro-4-trifluoromethyl-phenyl)-N²-(2,6-dichloro-phenyl)-5-(2-isopropyl-pyrrolidin-1-yl)-thiazolo[5,4-d]pyrimidine-2,7-diamine;
N⁷-(6-Chloro-pyridin-3-yl)-N²-(2,6-dimethyl-phenyl)-thiazolo[5,4-d]pyrimidine-2,7-diamine;
N²-(2,6-Dimethyl-phenyl)-N⁷-(4-methylsulfanyl-phenyl)-thiazolo[5,4-d]pyrimidine-2,7-diamine;
N²-(2,6-Dimethyl-phenyl)-N⁷-(3-fluoro-4-trifluoromethyl-phenyl)-5-methyl-thiazolo[5,4-d]pyrimidine-2,7-diamine;
N²-(2,6-Dimethyl-phenyl)-N⁷-[4-(propane-2-sulfonyl)-phenyl]-thiazolo[5,4-d]pyrimidine-2,7-diamine;
N⁷-(4-Bromo-phenyl)-N²-(2,6-dimethyl-phenyl)-thiazolo[5,4-d]pyrimidine-2,7-diamine;
N⁷-(3-Chloro-4-methylsulfanyl-phenyl)-N²-(2,6-dichloro-phenyl)-thiazolo[5,4-d]pyrimidine-2,7-diamine;
N²-(2,6-Dimethyl-phenyl)-N⁷-(4-isopropyl-phenyl)-5-methyl-thiazolo[5,4-d]pyrimidine-2,7-diamine;
N-(2-Chloro-phenyl)-N⁷-(4-methanesulfonyl-phenyl)-thiazolo[5,4-d]pyrimidine-2,7-diamine;
4-[2-(2,6-Dichloro-phenylamino)-5-methylsulfanyl-thiazolo[5,4-d]pyrimidin-7-ylamino]-N,N-dimethyl-benzenesulfonamide;
1-{4-[2-(2,6-Dimethyl-phenylamino)-thiazolo[5,4-d]pyrimidin-7-ylamino]-phenyl}-ethanone;
N²-(2,6-Dichloro-phenyl)-N⁷-(4-methanesulfonyl-phenyl)-5-piperidin-1-yl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-pyrrolidin-1-yl)-thiazolo[5,4-d]pyrimidine-2,7-diamine;
N⁷-(3-Chloro-4-trifluoromethylsulfanyl-phenyl)-N²-(2,6-dimethyl-phenyl)-5-methyl-thiazolo[5,4-d]pyrimidine-2,7-diamine;
N⁷-(4-Chloro-phenyl)-N²-(2,6-dimethyl-phenyl)-thiazolo[5,4-d]pyrimidine-2,7-diamine;
N²-(2,6-Dimethyl-phenyl)-N⁷-(3-fluoro-4-methyl-phenyl)-5-methyl-thiazolo[5,4-d]pyrimidine-2,7-diamine;
N²-(2,6-Dichloro-phenyl)-N⁷-[4-(piperazine-1-sulfonyl)-phenyl]-thiazolo[5,4-d]pyrimidine-2,7-diamine;
N²-(3-Chloro-4-trifluoromethylsulfanyl-phenyl)-N⁷-(2,6-dichloro-phenyl)-5-methyl-thiazolo[5,4-d]pyrimidine-2,7-diamine;
(racemic)-N⁷-(3-Chloro-4-trifluoromethyl-phenyl)-N²-(2,6-dichloro-phenyl)-5-(2-methyl-piperidin-1-yl)-thiazolo[5,4-d]pyrimidine-2,7-diamine;
N²-(2,6-Dimethyl-phenyl)-N⁷-(4-iodo-phenyl)-thiazolo[5,4-d]pyrimidine-2,7-diamine;
N²-(2,6-Dimethyl-phenyl)-5-methyl-N⁷-p-tolyl-thiazolo[5,4-d]pyrimidine-2,7-diamine;
N²-(2,6-Dichloro-phenyl)-5-methyl-N⁷-(1-methyl-1,2,3,4-tetrahydro-quinolin-7-yl)-thiazolo[5,4-d]pyrimidine-2,7-diamine;
(racemic)-1-{4-[2-(2,6-Dimethyl-phenylamino)-thiazolo[5,4-d]pyrimidin-7-ylamino]-phenyl}-ethanol;
N²-(2,6-Dimethyl-phenyl)-5-methyl-N⁷-phenyl-thiazolo[5,4-d]pyrimidine-2,7-diamine;
2-Chloro-4-[2-(2,6-dimethyl-phenylamino)-thiazolo[5,4-d]pyrimidin-7-ylamino]-benzonitrile;
(racemic)-N²-(2,6-Dimethyl-phenyl)-N⁷-(4-methanesulfinyl-phenyl)-5-methyl-thiazolo[5,4-d]pyrimidine-2,7-diamine;
N²-(2,6-Dimethyl-phenyl)-N⁷-(4-fluoro-phenyl)-thiazolo[5,4-d]pyrimidine-2,7-diamine;
4-[2-(2,6-Dimethyl-phenylamino)-thiazolo[5,4-d]pyrimidin-7-ylamino]-N,N-dimethyl-benzamide;
(racemic)-{4-[2-(2,6-Dichloro-phenylamino)-7-(4-trifluoromethyl-phenylamino)-thiazolo[5,4-d]pyrimidin-5-yl]-morpholin-2-yl}-methanol;
N²-(2,6-Dimethyl-phenyl)-N⁷-phenyl-thiazolo[5,4-d]pyrimidine-2,7-diamine;
N²-(2,6-Dimethyl-phenyl)-N⁷-(3-fluoro-4-methyl-phenyl)-thiazolo[5,4-d]pyrimidine-2,7-diamine;
4-[2-(2,6-Dimethyl-phenylamino)-thiazolo[5,4-d]pyrimidin-7-ylamino]-2-trifluoromethyl-benzonitrile;
N⁷-(2,3-Dihydro-benzo[1,4]dioxin-6-yl)-N²-(2,6-dimethyl-phenyl)-thiazolo[5,4-d]pyrimidine-2,7-diamine;
N²-(2,6-Dimethyl-phenyl)-N⁷-[4-(piperazine-1-sulfonyl)-phenyl]-thiazolo[5,4-d]pyrimidine-2,7-diamine;
N-{4-[2-(2,6-Dichloro-phenylamino)-5-methyl-thiazolo[5,4-d]pyrimidin-7-ylamino]-phenyl}-N-methyl-methanesulfonamide;
N²-(2,6-Dichloro-phenyl)-N⁵-[3-(4-methyl-piperazin-1-yl)-propyl]-N⁷-(4-trifluoromethyl-phenyl)-thiazolo[5,4-d]pyrimidine-2,5,7-diamine;
(racemic)-N²-(2,6-Dimethyl-phenyl)-N⁷-[4-(tetrahydro-furan-3-yloxy)-phenyl]-thiazolo[5,4-d]pyrimidine-2,7-diamine;
(racemic)-{4-[7-(3-Chloro-4-trifluoromethyl-phenylamino)-2-(2,6-dichloro-phenylamino)-thiazolo[5,4-d]pyrimidin-5-yl]-morpholin-2-yl}-methanol;
Cyclopentyl-{4-[2-(2,6-dichloro-phenylamino)-5-methyl-thiazolo[5,4-d]pyrimidin-7-ylamino]-phenyl}-methanone;
4-[2-(2,6-Dichloro-phenylamino)-5-methyl-thiazolo[5,4-d]pyrimidin-7-ylamino]-N,N-dimethyl-benzamide;
2-[2-(2,6-Dimethyl-phenylamino)-thiazolo[5,4-d]pyrimidin-7-ylamino]-5-methyl-phenol;
N²-(2,6-Dimethyl-phenyl)-N⁷-(2-methyl-4-trifluoromethyl-phenyl)-thiazolo[5,4-d]pyrimidine-2,7-diamine;
5-[2-(2,6-Dimethyl-phenylamino)-thiazolo[5,4-d]pyrimidin-7-ylamino]-2-methyl-phenol;
N-{4-[2-(2,6-Dimethyl-phenylamino)-thiazolo[5,4-d]pyrimidin-7-ylamino]-phenyl}-N-methyl-methanesulfonamide;
N⁷-(3-Chloro-4-trifluoromethyl-phenyl)-N²-(2,6-dichloro-phenyl)-5-piperazin-1-yl-thiazolo[5,4-d]pyrimidine-2,7-diamine;
N²-(2,6-Dichloro-phenyl)-5-piperazin-1-yl-N⁷-(4-trifluoromethyl-phenyl)-thiazolo[5,4-d]pyrimidine-2,7-diamine;
N-{4-[2-(2,6-Dimethyl-phenylamino)-5-methyl-thiazolo[5,4-d]pyrimidin-7-ylamino]-phenyl}-N-methyl-methanesulfonamide;
N²-(2,6-Dimethyl-phenyl)-N²-(3,4-dimethyl-phenyl)-thiazolo[5,4-d]pyrimidine-2,7-diamine;
N²-(2,6-Dichloro-phenyl)-5-methyl-N⁷-pyridin-3-yl-thiazolo[5,4-d]pyrimidine-2,7-diamine;
N⁷-(2-Chloro-4-trifluoromethyl-phenyl)-N²-(2,6-dimethyl-phenyl)-thiazolo[5,4-d]pyrimidine-2,7-diamine;
N²-(2,6-Dimethyl-phenyl)-N⁷-(4-methoxy-3-trifluoromethyl-phenyl)-thiazolo[5,4-d]pyrimidine-2,7-diamine;
4-[2-(2,6-Dimethyl-phenylamino)-thiazolo[5,4-d]pyrimidin-7-ylamino]-benzamide;
N²-(2,6-Dichloro-phenyl)-5-methyl-N⁷-phenyl-thiazolo[5,4-d]pyrimidine-2,7-diamine;
N²-(2,6-Dimethyl-phenyl)-5-methyl-N⁷-pyridin-3-yl-thiazolo[5,4-d]pyrimidine-2,7-diamine;
4-[2-(2,6-Dimethyl-phenylamino)-thiazolo[5,4-d]pyrimidin-7-ylamino]-benzoic acid;
N-{4-[2-(2,6-Dichloro-phenylamino)-5-methyl-thiazolo[5,4-d]pyrimidin-7-ylamino]-phenyl}-dimethanesulfonamide;
N-{4-[2-(2,6-Dimethyl-phenylamino)-5-methyl-thiazolo[5,4-d]pyrimidin-7-ylamino]-phenyl}-methanesulfonamide;
N-{4-[2-(2,6-Dimethyl-phenylamino)-thiazolo[5,4-d]pyrimidin-7-ylamino]-phenyl}-methanesulfonamide;
N²-(2,6-Dimethyl-phenyl)-N⁷-(5-trifluoromethyl-pyridin-2-yl)-thiazolo[5,4-d]pyrimidine-2,7-diamine;
(racemic)-N²-(2,6-Dichloro-phenyl)-5-(2-isopropyl-pyrrolidin-1-yl)-N⁷-(4-methanesulfonyl-phenyl)-thiazolo[5,4-d]pyrimidine-2,7-diamine;
N²-(2,6-Dichloro-phenyl)-N⁷-(4-methanesulfonyl-phenyl)-5-morpholin-4-yl-thiazolo[5,4-d]pyrimidine-2,7-diamine;
(racemic)-N²-(2,6-Dichloro-phenyl)-N⁷-(4-methanesulfonyl-phenyl)-5-(2-methyl-piperidin-1-yl)-thiazolo[5,4-d]pyrimidine-2,7-diamine;
N²-(2,6-Dichloro-phenyl)-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁷-(2-methylamino-ethyl)-N⁷-(4-trifluoromethyl-phenyl)-thiazolo[5,4-d]pyrimidine-2,5,7-triamine;
N²-(2,6-Dichloro-phenyl)-N⁷-(2-dimethylamino-ethyl)-N⁵-methyl-N⁷-(4-trifluoromethyl-phenyl)-thiazolo[5,4-d]pyrimidine-2,5,7-triamine;
(3R)-N-(2,6-Dichloro-phenyl)-5-(3-methylamino-pyrrolidin-1-yl)-N⁷-(4-trifluoromethyl-phenyl)-thiazolo[5,4-d]pyrimidine-2,7-diamine;
N⁵-Cyclopropylmethyl-N²-(2,6-dichloro-phenyl)-N⁷-(4-methanesulfonyl-phenyl)-thiazolo[5,4-d]pyrimidine-2,5,7-triamine;
N²-(2,6-Dichloro-phenyl)-5-methyl-N⁷-(6-methylsulfanyl-pyridin-3-yl)-thiazolo[5,4-d]pyrimidine-2,7-diamine;
(racemic)-2-[2-(2,6-Dichloro-phenylamino)-7-(4-trifluoromethyl-phenylamino)-thiazolo[5,4-d]pyrimidin-5-ylamino]-propan-1-ol;
N²-(2,6-Dichloro-phenyl)-5-(4-methyl-piperazin-1-yl)-N⁷-(4-trifluoromethyl-phenyl)-thiazolo[5,4-d]pyrimidine-2,7-diamine;
N²-(2,6-Dichloro-phenyl)-N⁵,N⁵-diethyl-N⁷-(4-trifluoromethyl-phenyl)-thiazolo[5,4-d]pyrimidine-2,5,7-triamine;
5-Butoxy-N²-(2,6-dichloro-phenyl)-N⁷-(4-trifluoromethyl-phenyl)-thiazolo[5,4-d]pyrimidine-2,7-diamine;
N²-(2,6-Dichloro-phenyl)-5-(4-methyl-piperidin-1-yl)-N²-(4-trifluoromethyl-phenyl)-thiazolo[5,4-d]pyrimidine-2,7-diamine;
(racemic)-N²-(2,6-Dichloro-phenyl)-5-(2-methyl-piperidin-1-yl)-N⁷-(4-trifluoromethyl-phenyl)-thiazolo[5,4-d]pyrimidine-2,7-diamine;
(3S)-N²-(2,6-Dichloro-phenyl)-5-(3-methyl-morpholin-4-yl)-N⁷-(4-trifluoromethyl-phenyl)-thiazolo[5,4-d]pyrimidine-2,7-diamine;
(2S)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
(2R)-N²-(2,6-Dichloro-phenyl)-5-(2-methoxymethyl-pyrrolidin-1-yl)-N⁷-(4-trifluoromethyl-phenyl)-thiazolo[5,4-d]pyrimidine-2,7-diamine;
5-Methyl-N²-(2-methylsulfanyl-phenyl)-N⁷-(4-trifluoromethyl-phenyl)-thiazolo[5,4-d]pyrimidine-2,7-diamine;
N²-(2-Methylsulfanyl-phenyl)-N⁷-(4-trifluoromethyl-phenyl)-thiazolo[5,4-d]pyrimidine-2,7-diamine;
N²-(2-Methanesulfonyl-phenyl)-5-methyl-N⁷-(4-trifluoromethylphenyl)-thiazolo[5,4-d]pyrimidine-2,7-diamine;
N²-(2-Methanesulfonyl-phenyl)-N⁷-(4-methanesulfonyl-phenyl)-thiazolo[5,4-d]pyrimidine-2,7-diamine;
N²-(2-Methanesulfonyl-phenyl)-N⁷-(6-trifluoromethyl-pyridin-3-yl)-thiazolo[5,4-d]pyrimidine-2,7-diamine;
N²-(2-Methanesulfonyl-phenyl)-N⁷-(4-trifluoromethanesulfonyl-phenyl)-thiazolo[5,4-d]pyrimidine-2,7-diamine;
N²-(2-Methanesulfonyl-phenyl)-N⁷-(4-trifluoromethyl-phenyl)-thiazolo[5,4-d]pyrimidine-2,7-diamine;
N²-(2-Chloro-phenyl)-5-methyl-N⁷-(4-trifluoromethyl-phenyl)-thiazolo[5,4-d]pyrimidine-2,7-diamine;
N²-(2-Chloro-phenyl)-N⁷-(4-trifluoromethanesulfonyl-phenyl)-thiazolo[5,4-d]pyrimidine-2,7-diamine;
N²-(2,6-Dichloro-phenyl)-N⁷-phenyl-thiazolo[5,4-d]pyrimidine-2,7-diamine;
N²-Benzo[1,2,5]thiadiazol-4-yl-5-methyl-N⁷-(4-trifluoromethyl-phenyl)-thiazolo[5,4-d]pyrimidine-2,7-diamine;
5-Methyl-N²-(2-nitro-phenyl)-N⁷-(4-trifluoromethyl-phenyl)-thiazolo[5,4-d]pyrimidine-2,7-diamine;
3-[7-(3-Chloro-4-trifluoromethyl-phenylamino)-thiazolo[5,4-d]pyrimidin-2-ylamino]-4-methyl-thiophene-2-carboxylic acid methyl ester;
N²-(3,5-Dimethyl-isoxazol-4-yl)-5-methyl-N⁷-(4-trifluoromethyl-phenyl)thiazolo[5,4-d]pyrimidine-2,7-diamine;
N⁷-(4-tert-Butyl-phenyl)-N²-(3,5-dimethyl-isoxazol-4-yl)-5-methyl-thiazolo[5,4-d]pyrimidine-2,7-diamine;
N²-(3-Methyl-pyridin-2-yl)-N⁷-[4-(pyrrolidine-1-sulfonyl)-phenyl]-thiazolo[5,4-d]pyrimidine-2,7-diamine;
5-Methyl-N²-(3-methyl-pyridin-2-yl)-N⁷-(4-trifluoromethyl-phenyl)-thiazolo[5,4-d]pyrimidine-2,7-diamine;
N,N-Dimethyl-4-[5-methyl-2-(3-methyl-pyridin-2-ylamino)-thiazolo[5,4-d]pyrimidin-7-ylamino]-benzenesulfonamide;
N²-(3-Methyl-pyridin-2-yl)-N⁷-(4-trifluoromethyl-phenyl)-thiazolo[5,4-d]pyrimidine-2,7-diamine;
N²-(3,5-Dichloro-pyridin-4-yl)-N⁷-[4-(pyrrolidine-1-sulfonyl)-phenyl]-thiazolo[5,4-d]pyrimidine-2,7-diamine;
N²-(2,6-Dichloro-phenyl)-N⁷-(3-fluoro-4-trifluoromethyl-phenyl)-thiazolo[5,4-d]pyrimidine-2,7-diamine;
N²-(2-Chlorophenyl)-N⁷-[4-(morpholin-4-ylsulfonyl)phenyl][1,3]thiazolo[5,4-d]pyrimidine-2,7-diamine;
N²-(2-Methylphenyl)-N⁷-[4-(morpholin-4-ylsulfonyl)phenyl][1,3]thiazolo[5,4-d]pyrimidine-2,7-diamine,
N²-(2-Methylphenyl)-N⁷-[6-(trifluoromethyl)pyridin-3-yl][1,3]thiazolo[5,4-d]pyrimidine-2,7-diamine;
N²-[2-(Trifluoromethyl)phenyl]-N⁷-[6-(trifluoromethyl)pyridin-3-yl][1,3]thiazolo[5,4-d]pyrimidine-2,7-diamine;
N²-(2-Chlorophenyl)-N⁷-[6-(trifluoromethyl)pyridin-3-yl][1,3]thiazolo[5,4-d]pyrimidine-2,7-diamine;
N²-(3,5-Dimethylisoxazol-4-yl)-N⁷-[4-(morpholin-4-ylsulfonyl)phenyl][1,3]thiazolo[5,4-d]pyrimidine-2,7-diamine;
Methyl 2-[4-({2-[(3,5-dimethylisoxazol-4-yl)amino][1,3]thiazolo[5,4-d]pyrimidin-7-yl}amino)phenyl]-2-methylpropanoate;
2-[4-({2-[(3,5-Dimethylisoxazol-4-yl)amino][1,3]thiazolo[5,4-d]pyrimidin-7-yl}amino)phenyl]-2-methylpropanenitrile;
N²-(3,5-Dimethylisoxazol-4-yl)-N⁷-[4-(methylsulfonyl)phenyl][1,3]thiazolo[5,4-d]pyrimidine-2,7-diamine;
N²-[2-(Trifluoromethyl)phenyl]-N⁷-[4-(trifluoromethyl)phenyl][1,3]thiazolo[5,4-d]pyrimidine-2,7-diamine;
N⁷-[4-(Methylsulfonyl)phenyl]-N²-[2-(trifluoromethyl)phenyl][1,3]thiazolo[5,4-d]pyrimidine-2,7-diamine;
4-({2-[(2,6-Dichlorophenyl)amino][1,3]thiazolo[5,4-d]pyrimidin-7-yl}amino)benzene-1,2-diol;
2-[4-({2-[(2,6-Dichlorophenyl)amino][1,3]thiazolo[5,4-d]pyrimidin-7-yl}amino)phenyl]-2-methylpropanenitrile;
Methyl 2-[4-({2-[(2,6-dichlorophenyl)amino][1,3]thiazolo[5,4-d]pyrimidin-7-yl}amino)phenyl]-2-methylpropanoate;
2-[4-({2-[(2,6-Dichlorophenyl)amino][1,3]thiazolo[5,4-d]pyrimidin-7-yl}amino)phenyl]-2-methylpropanoic acid;
1-Methylethyl 2-[4-({2-[(2,6-dichlorophenyl)amino][1,3]thiazolo[5,4-d]pyrimidin-7-yl}amino)phenyl]-2-methylpropanoate;
N²-Cyclohexyl-N⁷-[4-(trifluoromethyl)phenyl][1,3]thiazolo[5,4-d]pyrimidine-2,7-diamine;
N²-Cyclohexyl-N⁷-[6-(trifluoromethyl)pyridin-3-yl][1,3]thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzonitrile;
3,5-Dichloro-4-[7-(4-trifluoromethyl-phenylamino)-thiazolo[5,4-d]pyrimidin-2-ylamino]-benzamide;
3,5-Dichloro-4-[7-(4-methanesulfonyl-phenylamino)-thiazolo[5,4-d]pyrimidin-2-ylamino]-benzonitrile;
3,5-Dichloro-4-[7-(4-methanesulfonyl-phenylamino)-thiazolo[5,4-d]pyrimidin-2-ylamino]-benzamide;
N²-(2,6-Dichloro-4-morpholin-4-ylmethyl-phenyl)-N⁷-(4-trifluoromethyl-phenyl)-thiazolo[5,4-d]pyrimidine-2,7-diamine;
N²-(4-Azetidin-1-ylmethyl-2,6-dichloro-phenyl)-N⁷-(4-trifluoromethyl-phenyl)-thiazolo[5,4-d]pyrimidine-2,7-diamine;
N²-(4-Aminomethyl-2,6-dichloro-phenyl)-N⁷-(4-trifluoromethyl-phenyl)-thiazolo[5,4-d]pyrimidine-2,7-diamine;
3,5-Dichloro-4-[7-(4-trifluoromethyl-phenylamino)-thiazolo[5,4-d]pyrimidin-2-ylamino]-benzoic acid methyl ester;
{3,5-Dichloro-4-[7-(4-trifluoromethyl-phenylamino)-thiazolo[5,4-d]pyrimidin-2-ylamino]-phenyl}-methanol;
3,5-Dichloro-4-[7-(4-trifluoromethyl-phenylamino)-thiazolo[5,4-d]pyrimidin-2-ylamino]-benzoic acid;
N⁷-(4-tert-Butyl-phenyl)-N²-(2,6-dimethyl-phenyl)-5-methyl-thiazolo[5,4-d]pyrimidine-2,7-diamine;
N²-(2,6-Dimethyl-phenyl)-5-methyl-N⁷-(4-trifluoromethyl-phenyl)-thiazolo[5,4-d]pyrimidine-2,7-diamine;
N²-(2,6-Dimethyl-phenyl)-5-methyl-N⁷-(6-trifluoromethyl-pyridin-3-yl)-thiazolo[5,4-d]pyrimidine-2,7-diamine;
N⁷-(3-Chloro-4-trifluoromethyl-phenyl)-N²-(2,6-dimethyl-phenyl)-5-methyl-thiazolo[5,4-d]pyrimidine-2,7-diamine;
N⁷-(4-tert-Butyl-cyclohexyl)-N²-(2,6-dimethyl-phenyl)-thiazolo[5,4-d]pyrimidine-2,7-diamine;
N²-(4-tert-Butyl-cyclohexyl)-N⁷-(2,6-dimethyl-phenyl)-5-methyl-thiazolo[5,4-d]pyrimidine-2,7-diamine;
(R)-1-[2-(2,6-Dichloro-phenylamino)-7-(4-trifluoromethyl-phenylamino)-thiazolo[5,4-d]pyrimidin-5-ylamino]-propan-2-ol;
1-[2-(2,6-Dichloro-phenylamino)-7-(4-trifluoromethyl-phenylamino)-thiazolo[5,4-d]pyrimidin-5-ylamino]-2-methyl-propan-2-ol;
(racemic)-{1-[2-(2,6-Dichloro-phenylamino)-7-(4-trifluoromethyl-phenylamino)-thiazolo[5,4-d]pyrimidin-5-yl]-pyrrolidin-2-yl}-methanol;
N²-(2,6-Dichloro-phenyl)-N⁵-methyl-N⁵-(2-piperidin-1-yl-ethyl)-N⁷-(4-trifluoromethyl-phenyl)-thiazolo[5,4-d]pyrimidine-2,5,7-triamine;
N²-(2,6-Dichloro-phenyl)-N⁷-(6-methanesulfonyl-pyridin-3-yl)-thiazolo[5,4-d]pyrimidine-2,7-diamine;
2-{4-[2-(2,6-Dichloro-phenylamino)-thiazolo[5,4-d]pyrimidin-7-ylamino]-phenyl}-isobutyramide;
(racemic)-1-[2-(2,6-Dichloro-phenylamino)-7-(4-trifluoromethyl-phenylamino)-thiazolo[5,4-d]pyrimidin-5-ylamino]-propan-2-ol;
(racemic)-3-[2-(2,6-Dichloro-phenylamino)-7-(4-trifluoromethyl-phenylamino)-thiazolo[5,4-d]pyrimidin-5-ylamino]-propane-1,2-diol; and
N²-(2,6-Dichloro-phenyl)-N⁵-(2-pyrrolidin-1-yl-ethyl)-N⁷-(4-trifluoromethyl-phenyl)-thiazolo[5,4-d]pyrimidine-2,5,7-triamine;
and pharmaceutically acceptable salts thereof.

44. A method according to claim 41, wherein the disease, disorder, or condition is pain; itch or an inflammatory disorder; an inner-ear disorder; fever or another condition or disorder of thermoregulation; tracheobronchial or diaphragmatic dysfunction; a gastrointestinal or urinary tract disorder; or a disorder associated with reduced blood flow to the central nervous system or CNS hypoxia.

45. A method according to claim 44, wherein the disease, disorder, or condition is pain.

46. A method according to claim 44, wherein the disease, disorder, or condition is arthritis.

47. A method according to claim 44, wherein the disease, disorder, or condition is itch.

48. A method according to claim 44, wherein the disease, disorder, or condition is cough.

49. A method according to claim 44, wherein the disease, disorder, or condition is asthma.

50. A method according to claim 44, wherein the disease, disorder, or condition is inflammatory bowel disease.

51. A method according to claim 44, wherein the disease, disorder, or condition is an inner ear disorder.
